(19) United States
(12) Patent Application Publication
(10) Pub. No.: US 2026/0152504 A1
(43) Pub. Date: Jun. 4, 2026

(54) PYRIMIDINE SGC STIMULATORS

(71) Applicant: Tisento Therapeutics Inc., Cambridge, MA (US)

(72) Inventors: Marta Cavero Tomas, Wayland, MA (US); Peter Germano, Newton, MA (US)

(21) Appl. No.: 19/122,608

(22) PCT Filed: Oct. 17, 2023
(86) PCT No.: PCT/US2023/035342

§ 371 Date: Apr. 18, 2025

Related U.S. Application Data
(60) Provisional application No. 63/417,180, filed on Oct. 18, 2022.

(51) Int. Cl. C07D 487/04; A61K 31/506

(52) U.S. Cl. CPC C07D 487/04; A61K 31/506

(57) ABSTRACT

The present disclosure relates to stimulators of soluble guanylate cyclase (sGC), pharmaceutically acceptable salts thereof and pharmaceutical formulations comprising them and their uses thereof, alone or in combination with one or more additional agents, for treating various diseases, wherein an increase in the concentration of nitric oxide (NO) and/or an increase in the concentration of cyclic Guanosine Monophosphate (cGMP), or both, or an uregulation of the NO pathway is desirable. In some embodiments, the compounds are those of Formula I or a pharmaceutically acceptable salt thereof. (I)

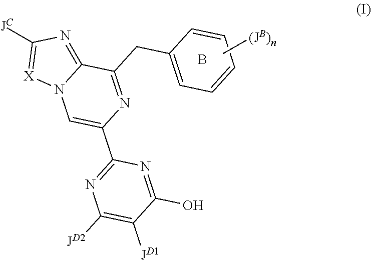

### RELATED APPLICATIONS

This application claims priority to U.S. Provisional Application No. 63/417,180, filed on Oct. 18, 2022. The entire contents of the foregoing application are expressly incorporated herein by reference.

### FIELD OF THE INVENTION

The present disclosure relates to stimulators of soluble guanylate cyclase (sGC) and pharmaceutically acceptable salts thereof. It also relates to pharmaceutical formulations and dosage forms comprising them and their uses thereof, alone or in combination with one or more additional agents, for treating various diseases. The disease are ones that would benefit from sGC stimulation or from an increase in the concentration of nitric oxide (NO) and/or cyclic guanosine monophosphate (cGMP).

### BACKGROUND OF THE INVENTION

sGC is the primary receptor for NO in vivo. Upon binding to sGC, NO activates its catalytic domain and results in the conversion of guanosine-5′-triphosphate (GTP) into the secondary messenger cGMP. The increased level of cGMP, in turn, modulates the activity of downstream effectors including protein kinases, phosphodiesterases (PDEs) and ion channels. In the body, NO is synthesized from arginine and oxygen by various nitric oxide synthase (NOS) enzymes and by sequential reduction of inorganic nitrate. Experimental and clinical evidence indicates that reduced NO concentrations, reduced NO bioavailability and/or reduced responsiveness to endogenously produced NO contributes to the development of numerous diseases. sGC stimulators are heme-dependent agonists of the sGC enzyme that work synergistically with varying amounts of NO to increase its enzymatic conversion of GTP to cGMP. sGC stimulators are clearly differentiated from and structurally unrelated to another class of NO-independent, heme-independent agonists of sGC known as sGC activators.

Therapies that improve or restore the function of sGC offer considerable advantages over current alternative therapies that either target or otherwise benefit from the upregulation of the NO-sGC-cGMP pathway. There is an urgent need to develop new and safe therapies for patients with dysfunctional NO-sGC-cGMP pathway.

### SUMMARY OF THE INVENTION

In a first aspect, the compound of the invention is represented by Formula I:

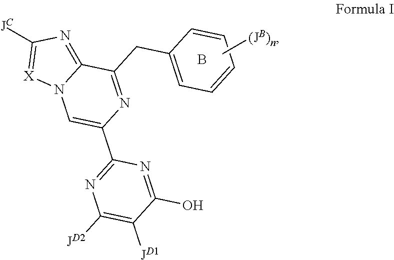

or a pharmaceutically acceptable salt thereof, wherein:
X is N or C(J^{C1});
J^{C} is selected from the group consisting of hydrogen, halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted with 1 to 3 substituents independently selected from the group consisting of halogen, —OH, —OR, —SR and —C(O)R;
J^{C1} is selected from the group consisting of hydrogen, halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted by 1 to 3 substituents independently selected from the group consisting of halogen, —OH, —OR, —SR and —C(O)R;
n is an integer selected from 0, 1, 2 or 3;
each J^{B} is independently selected from the group consisting of halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted by 1 to 3 substituents independently selected from the group consisting of halogen, —OH, —OR, —SR and —C(O)R;
J^{D1} is selected from the group consisting of hydrogen, halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted by 1 to 3 substituents independently selected from the group consisting of halogen, —OH, —OR, —SR and —C(O)R;
J^{D2} is selected from the group consisting of hydrogen, halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted by 1 to 3 substituents independently selected from the group consisting of halogen, —OH, —OR, —SR and —C(O)R;
R, for each occurrence, is independently C₁₋₄ alkyl optionally substituted with 1 to 3 independently selected halogen atoms;
wherein, when J^{D2} is hydrogen and n is 1, 2 or 3, then at least one of J^{C}, J^{C1}, J^{D1}, and J^{B} is —OH, —OR, —SH, —SR, —CN, —C(O)R, C₃₋₅ cycloalkyl optionally and independently substituted with 1 to 3 halogen atoms, or a C₁₋₆ alkyl substituted with 1 to 3 substituents independently selected from the group consisting of —OH, —OR, —SR and —C(O)R;
when J^{D2} is hydrogen and n is 0, then at least one of J^{C}, J^{C1} and J^{D1} is —OH, —OR, —SH, —SR, —CN, —C(O)R, C₃₋₅ cycloalkyl optionally and independently substituted with 1 to 3 halogen atoms, or a C₁₋₆ alkyl substituted with 1 to 3 substituents independently selected from the group consisting of —OH, —OR, —SR and —C(O)R; and
provided that the compound is not one of the following:

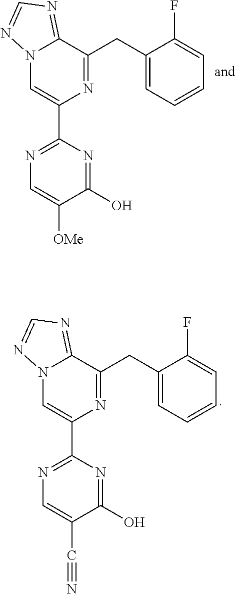 [SMILES: COc1cnc(-c2cn3ncnc3c(Cc3ccccc3F)n2)nc1O ; N#Cc1cnc(-c2cn3ncnc3c(Cc3ccccc3F)n2)nc1O]

In a second aspect, the invention relates to pharmaceutical compositions comprising a compound of Formula I, or a pharmaceutically acceptable salt thereof and at least one pharmaceutically acceptable excipient or carrier. In one embodiment of the second aspect, the invention relates to pharmaceutical dosage forms comprising said pharmaceutical compositions.

In a third aspect, the invention relates to a method of treating a disease in a subject in need thereof, comprising administering, alone or in combination therapy, a therapeutically effective amount of a compound of Formula I, or a pharmaceutically acceptable salt thereof, or pharmaceutical composition thereof, to the subject; wherein the disease is one that would benefit from sGC stimulation or from an increase in the concentration of NO and/or cGMP.

### DETAILED DESCRIPTION OF THE INVENTION

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls.

### Definitions and General Terminology Related to the Compounds

For purposes of this disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry”, Thomas Sorrell, University Science Books, Sausalito: 1999, and “March's Advanced Organic Chemistry”, 5^{th} Ed., Smith, M. B. and March, J., eds. John Wiley & Sons, New York: 2001, which are herein incorporated by reference in their entirety.

In general, the term “substituted” refers to the replacement of one or more hydrogen radicals of a given structure with another specified radical substituent, different from hydrogen (some non-limiting examples would be a hydroxy, a phenyl, or an alkyl radical). If a structure is “optionally substituted” it may be substituted or unsubstituted. When a structure is substituted, the substituent is permitted at any substitutable atom. A “substitutable atom” is any atom (e.g., carbon, nitrogen, oxygen, or sulfur) bonded to at least one hydrogen atom. When a certain ring is optionally substituted, it will be understood that it may be substituted at one or some or all of its substitutable ring atoms, depending on the number of substituents allowed. A “substitutable ring atom” is any ring atom (e.g., carbon or nitrogen) bonded to at least one hydrogen atom, does not include ring atoms wherein the structure depicts that they are already attached to one or more moieties or substituents other than hydrogen and no more hydrogens are available for substitution.

When one or more position(s) of a structure can be substituted with one or more than one substitutent selected from a specified group or list, the substituent or substitutents at each position may be “independently selected” to be equal or the same at each position and for each instance, unless otherwise specified. For example, if a phenyl is substituted with two instances of R¹⁰⁰, and each R¹⁰⁰ is independently selected from halogen and methyl, that means that each instance of R¹⁰⁰ is separately selected from halogen or methyl; for instance, one R¹⁰⁰ may be fluoro and one may be methyl, or both may be chloro, or one may be fluoro and the other chloro, or both may be methyl, etc. Similarly, if a substitutable atom is bonded to more than one hydrogen (e.g., CH₃ or NH₂), the substitutents may be “independently selected” to be equal or the same for each instance of hydrogen, unless otherwise specified. For example, if a methyl (e.g., CH₃) is substituted with two instances of R¹⁰⁰, and each R¹⁰⁰ is independently selected from halogen and methyl, that means that each instance of R¹⁰⁰ is separately selected from halogen or methyl; for instance, one R¹⁰⁰ (i.e., one of the hydrogens attached to C in the CH₃ group) may be replaced by fluoro and one may be replaced by methyl (e.g., CHF(CH₃)), or both may be replaced by chloro (e.g., CHCl₂), etc.

Selection of substituents and combinations envisioned by this disclosure are only those that result in the formation of stable or chemically feasible compounds. Such choices and combinations will be apparent to those of ordinary skill in the art and may be determined without undue experimentation. The term “stable”, as used herein, refers to compounds that are not substantially altered when subjected to conditions to allow for their production, detection, and, in some embodiments, their recovery, purification, and use for one or more of the purposes disclosed herein. A chemically feasible compound is a compound that can be prepared by a person skilled in the art based on the disclosures herein, supplemented, if necessary, by relevant knowledge of the art.

The phrase “up to”, as used herein, refers to zero or any integer number that is equal or less than the number following the phrase. For example, “up to 3” means any one of 0, 1, 2, or 3.

As described herein, a specified number range of atoms or of substituents includes any integer therein. For example, a group having from 1-4 atoms could have 1, 2, 3 or 4 atoms. When any variable occurs more than one time at any position, its definition on each occurrence is independent from every other occurrence. When a moiety is substituted with 0 instances of a certain variable, this means the moiety is unsubstituted (i.e. it may only have hydrogen radicals attached to any subtitutable atom).

Unless otherwise stated, all tautomeric forms of the compounds of the present disclosure are also within the scope of the invention, independently of how a specific compound is drawn.

In one embodiment, the present disclosure may include replacement of hydrogen with deuterium (i.e., ²H), which may afford certain therapeutic advantages resulting from greater metabolic stability (e.g., increased in vivo half-life or reduced dosage requirements) and hence may be preferred in some circumstances. Deuterium labeled compounds of the present invention can generally be prepared by following procedures analogous to those disclosed in the Schemes and/or in the Examples herein below, by substituting a deuterated reagent for a non-deuterated reagent.

The term “alkyl” as in, for example, “alkyl chain” or “alkyl group”, as used herein, refers to a saturated unbranched (e.g., linear) or branched monovalent hydrocarbon radical. A Cₓ alkyl is an alkyl chain containing x carbon atoms, wherein x is an integer different from 0. A “C_{x-y} alkyl”, wherein x and y are two different integers, both different from 0, is an alkyl chain containing between x and y number of carbon atoms, both inclusive. For example, a C₁₋₆ alkyl is an alkyl as defined above containing any number of between 1 and 6 carbon atoms. Examples of alkyl groups include, but are not limited to, methyl (i.e., C₁ alkyl), ethyl (i.e., C₂ alkyl), n-propyl (a C₃ alkyl), isopropyl (a different C₃ alkyl), n-butyl, isobutyl, s-butyl, t-butyl, pentyl, hexyl, heptyl, octyl and the like.

The term “cycloalkyl”, as in “cycloalkyl ring” or “cycloalkyl group”, as used herein, refers to a ring system formed only by carbon and hydrogen atoms which is completely saturated. Suitable cycloalkyl groups include, but are not limited to, cyclopropyl, cyclobutyl, cyclopentyl, cyclohexyl, cycloheptyl, cycloheptenyl, norbornyl, cyclooctyl, cyclononyl, cyclodecyl, cycloundecyl, cyclododecyl, and the like. A cycloalkyl ring will be represented by the term “C_{x-y} cycloalkyl”; wherein x and y are the minimum and the maximum number of carbon atoms forming the cycloalkyl ring.

As used herein, the terms “halogen” or “halo” mean F, Cl, Br, or I.

The compounds of the invention are defined herein by their chemical structures and/or chemical names. Where a compound is referred to by both a chemical structure and a chemical name, and the chemical structure and chemical name conflict, the chemical structure is determinative of the compound's identity.

Substituents, such as for example, J^{C}, J^{C1}, J^{B}, and J^{D} are generally defined when introduced and retain that definition throughout the specification and in all independent claims, unless otherwise specified.

The compounds of the invention are defined herein by their chemical structures and/or chemical names. Where a compound is referred to by both a chemical structure and a chemical name, and the chemical structure and chemical name conflict, the chemical structure is determinative of the compound's identity.

### Compound/Composition Embodiments

The present invention is based on the discovery that the compounds disclosed herein are sGC stimulators. Compounds with similar structural features, particularly, with a 4-OH substituent on the pyrimidine ring, were previously known only as synthetic intermediates that could be used in the preparation of sGC stimulators having 4-amino substituents on that said pyrimidine ring. It was unexpectedly found that the compounds of the present disclosure have potent sGC stimulatory activities and therefore have the potential to be useful for the treatment of diseases which can benefit from stimulation of the NO-sGC-cGMP pathway.

In a first embodiment of the first aspect, the compound of the invention is represented by Formula I:

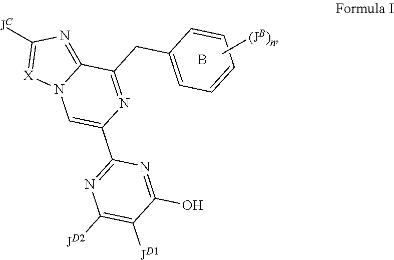

or a pharmaceutically acceptable salt thereof, wherein:
X is N or C(J^{C1});
J^{C} is selected from the group consisting of hydrogen, halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted with 1 to 3 substituents independently selected from the group consisting of halogen, —OH, —OR, —SR and —C(O)R;
J^{C1} is selected from the group consisting of hydrogen, halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted by 1 to 3 substituents independently selected from the group consisting of halogen, —OH, —OR, —SR and —C(O)R;
n is an integer selected from 0, 1, 2 or 3;
each J^{B} is independently selected from the group consisting of halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted by 1 to 3 substituents independently selected from the group consisting of halogen, —OH, —OR, —SR and —C(O)R;
J^{D1} is selected from the group consisting of hydrogen, halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted by 1 to 3 substituents independently selected from the group consisting of halogen, —OH, —OR, —SR and —C(O)R;
J^{D2} is selected from the group consisting of hydrogen, halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted by 1 to 3 substituents independently selected from the group consisting of halogen, —OH, —OR, —SR and —C(O)R;
R, for each occurrence, is independently C₁₋₄ alkyl optionally substituted with 1 to 3 independently selected halogen atoms;
wherein, when J^{D2} is hydrogen and n is 1, 2 or 3, then at least one of J^{C}, J^{C1}, J^{D1} and J^{B} is —OH, —OR, —SH, —SR, —CN, —C(O)R, C₃₋₅ cycloalkyl optionally and independently substituted with up to 3 halogen atoms, or a C₁₋₆ alkyl substituted with 1 to 3 substituents independently selected from the group consisting of —OH, —OR, —SR and —C(O)R;
when J^{D2} is hydrogen and n is 0, then at least one of J^{C}, J^{C1} and J^{D1} is —OH, —OR, —SH, —SR, —CN, —C(O)R, C₃₋₅ cycloalkyl optionally and independently substituted with up to 3 halogen atoms, or a C₁₋₆ alkyl substituted with 1 to 3 substituents independently selected from the group consisting of —OH, —OR, —SR and —C(O)R; and
provided that the compound is not one of the following:

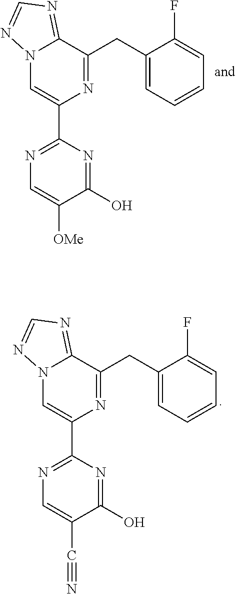 [SMILES: COc1cnc(-c2cn3ncnc3c(Cc3ccccc3F)n2)nc1O ; N#Cc1cnc(-c2cn3ncnc3c(Cc3ccccc3F)n2)nc1O]

In a second embodiment, the compound of Formula I is a compound of Formula IA:

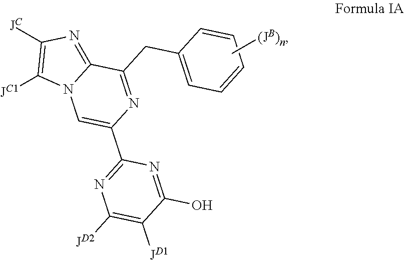

or a pharmaceutically acceptable salt thereof, wherein the variables are as described above for Formula I;
wherein, when J^{D2} is hydrogen and n is 1, 2 or 3, then at least one of J^{C}, J^{C1}, J^{D1}, and J^{B} is —OH, —OR, —SH, —SR, —CN, —C(O)R, C₃₋₅ cyloalkyl optionally and independently substituted with 1 to 3 halogen atoms, or a C₁₋₆ alkyl substituted with 1 to 3 substituents independently selected from the group consisting of —OH, —OR, —SR and —C(O)R; and
when J^{D2} is hydrogen and n is 0, then at least one of J^{C}, J^{C1} and J^{D1} is —OH, —OR, —SH, —SR, —CN, —C(O)R, C₃₋₅ cycloalkyl optionally and independently substituted with 1 to 3 halogen atoms, or a C₁₋₆ alkyl substituted with 1 to 3 substituents independently selected from the group consisting of —OH, —OR, —SR and —C(O)R.

In a third embodiment, for the compounds of Formula I or Formula IA, or a pharmaceutically acceptable salt thereof, J^{C1} is selected from the group consisting of hydrogen, halogen, C₁₋₃ alkyl, —CN, —SH, —SR, —OR, and —C(O)R, wherein the C₁₋₃ alkyl is optionally substituted by 1 to 3 substituents independently selected from the group consisting of halogen, —OH and —OR; and the remaining variables are as defined in the first or second embodiment.

In a fourth embodiment, for the compounds of Formula I or Formula IA, or a pharmaceutically acceptable salt thereof, J^{C1} is selected from the group consisting of hydrogen, —F, —Cl, —CN, —CH₃, —CH₂F, —SH, —CH₂OH, —CH₂OCH₃, —SCH₃, —CH(OH)CH₃, —C(O)CH₃, and —OCH₃; and the remaining variables are defined in the first, second or third embodiment.

In a fifth embodiment, for the compounds of Formula I or Formula IA, or a pharmaceutically acceptable salt thereof, J^{C1} is selected from the group consisting of hydrogen, halogen, and C₁₋₃ alkyl, wherein the C₁₋₃ alkyl is optionally substituted by 1 to 3 independently selected halogen substituents; and the remaining variables are defined in the first, second, third or fourth embodiment.

In a sixth embodiment, for the compounds of Formula I or Formula IA, or a pharmaceutically acceptable salt thereof, J^{C1} is selected from the group consisting of hydrogen, —F, —Cl, —CH₃, and —CH₂F; and the remaining variables are defined in the first, second, third, fourth, or fifth embodiment.

In a seventh embodiment, for the compounds of Formula I or Formula IA, or a pharmaceutically acceptable salt thereof, J^{C1} is hydrogen or —F; and the remaining variables are defined in the first, second, third, fourth, fifth, or sixth embodiment.

In an eighth embodiment, the compound of Formula I is a compound of Formula IB:

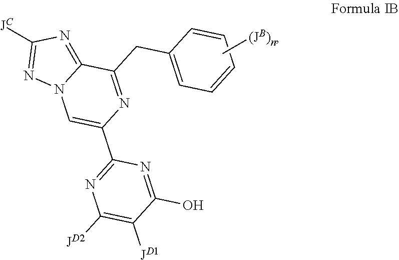

or a pharmaceutically acceptable salt thereof, wherein the variables are as described above for Formula I,
wherein, when J^{D2} is hydrogen and n is 1, 2 or 3, then at least one of J^{C}, J^{D1} and J^{B} is —OH, —OR, —SH, —SR, —CN, —C(O)R, a C₃₋₅ cycloalkyl ring optionally and independently substituted with 1 to 3 halogen atoms, or a C₁₋₆ alkyl substituted with 1 to 3 substituents independently selected from the group consisting of —OH, —OR, —SR and —C(O)R;
when J^{D2} is hydrogen and n is 0, then at least one of J^{C} and J^{D1} is —OH, —OR, —SH, —SR, —CN, —C(O)R, a C₃₋₅ cycloalkyl ring optionally and independently substituted with 1 to 3 halogen atoms, or a C₁₋₆ alkyl substituted with 1 to 3 substituents independently selected from the group consisting of —OH, —OR, —SR and —C(O)R; and
provided that the compound is not one of the following:

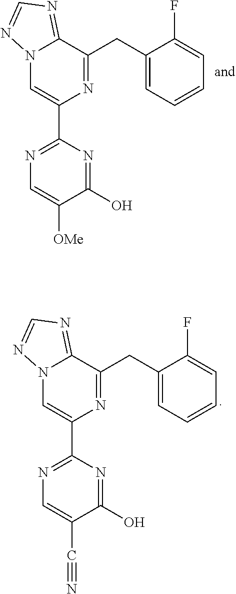 [SMILES: COc1cnc(-c2cn3ncnc3c(Cc3ccccc3F)n2)nc1O ; N#Cc1cnc(-c2cn3ncnc3c(Cc3ccccc3F)n2)nc1O]

In a ninth embodiment, for the compound of Formula I, IA or IB, or a pharmaceutically acceptable salt thereof, n is 2 or 3, and the remaining variables are as described in the first, second, third, fourth, fifth, sixth, seventh, or eighth embodiment. In some embodiments, n is 2. In other embodiments, n is 3.

In a tenth embodiment, for the compound of Formula I, IA or IB, or a pharmaceutically acceptable salt thereof, n is 0 or 1, and the remaining variables are as described in the first, second, third, fourth, fifth, sixth, seventh, or eighth embodiment. In some embodiments, n is 1. In other embodiments, n is 0.

In an eleventh embodiment, for the compound of Formula I, IA or IB, or a pharmaceutically acceptable salt thereof, each J^{B} is independently selected from a group consisting of halogen, —CN, —OH, —OR, —SR, —C(O)R and C₁₋₃ alkyl, wherein the C₁₋₃ alkyl is optionally substituted by 1 to 3 substituents independently selected form halogen, —OH and —OR; and the remaining variables are as described in the first, second, third, fourth, fifth, sixth, seventh, eighth, ninth, or tenth embodiment.

In a twelfth embodiment, for the compound of Formula I, IA or IB, or a pharmaceutically acceptable salt thereof, J^{B} is independently selected from a group consisting of —F, —Cl, —Br, —CN, —CH₃, —CF₃, —CH₂F, —CHFCH₃, —CH₂CH₂F, —C(O)CH₃, —CH(OH)CH₃, —CH₂CH₃, —SCH₃, —OCH₃, —OH, —CH₂OCH₃, and —CH₂OH; and the remaining variables are as described in the first, second, third, fourth, fifth, sixth, seventh, eighth, ninth, tenth, or eleventh embodiment.

In a thirteenth embodiment, for the compound of Formula I, IA or IB, or a pharmaceutically acceptable salt thereof, n is 2 or 3, and each J^{B} is independently selected from the group consisting of halogen, —OR, —CN, —OH, and C₁₋₃ alkyl, wherein the C₁₋₃ alkyl is optionally substituted by 1 to 3 independently selected halogen substituents; and the remaining variables are as described in the first, second, third, fourth, fifth, sixth, seventh, eighth, ninth, or eleventh embodiment.

In a fourteenth embodiment, for the compound of Formula I, IA or IB, or a pharmaceutically acceptable salt thereof, n is 2 or 3, and each J^{B} is independently selected from the group consisting of —F, —Cl, —CH₃, —CF₃, —CN, —OH and —OCH₃; and the remaining variables are as described in the first, second, third, fourth, fifth, sixth, seventh, eighth, ninth, eleventh, twelfth, or thirteenth embodiment.

In fifteenth embodiment, for the compound of Formula I, IA or IB, or a pharmaceutically acceptable salt thereof, n is 2; each J^{B} is independently selected form the group consisting of —F, —CH₃ and —CF₃; and the remaining variables are as described in the first, second, third, fourth, fifth, sixth, seventh, eighth, ninth, eleventh, twelfth, thirteenth, or fourteenth embodiment. In some embodiments, one J^{B} is —CH₃ or —CF₃ and the other is —F. In other embodiments both J^{B}s are —F.

In a sixteenth embodiment, for the compound of Formula I, IA or IB, or a pharmaceutically acceptable salt thereof, n is 3; each J^{B} is independently selected form the group consisting of —F, —OH, —CH₃, —CH₂F and —CF₃; and the remaining variables are as described in first, second, third, fourth, fifth, sixth, seventh, eighth, ninth, eleventh, twelfth or thirteenth embodiment. In some embodiments, one J^{B} is —F, a second one is —CH₃ or —CH₂F and the other is —F, —OH, —CH₃ or —CH₂F.

In a seventeenth embodiment, for the compound of Formula I, IA or IB, or a pharmaceutically acceptable salt thereof, n is 1; and J^{B} is selected from the group consisting of halogen, —OR, and C₁₋₃ alkyl optionally substituted with 1 to 3 independently selected halogen substituents; and the remaining variables are as described in the first, second, third, fourth, fifth, sixth, seventh, eighth, tenth, or eleventh embodiment.

In an eighteenth embodiment, for the compound of Formula I, IA or IB, or a pharmaceutically acceptable salt thereof, n is 1; and J^{B} is selected from the group consisting of —F, —OR, and C₁₋₃ alkyl optionally substituted with 1 to 3 independently selected halogen substituents; and the remaining variables are as described in the first, second, third, fourth, fifth, sixth, seventh, eighth, tenth or eleventh embodiment.

In a nineteenth embodiment, for the compound of Formula I, IA or IB, or a pharmaceutically acceptable salt thereof, n is 1; and J^{B} is selected from the group consisting of —F, —CH₃, —CF₃ and —OCH₃; and the remaining variables are as described in the first, second, third, fourth, fifth, sixth, seventh, eighth, tenth or eleventh embodiment.

In a twentieth embodiment, for the compound of Formula I, IA or IB, or a pharmaceutically acceptable salt thereof, n is 0; and the remaining variables are as described in the first, second, third, fourth, fifth, sixth, seventh or eighth embodiment.

In a twenty-first embodiment, for the compound of Formula I, IA or IB, or a pharmaceutically acceptable salt thereof, J^{D2} is selected from the group consisting of hydrogen, halogen, —OH, —OR, —SR, —C(O)R, and C₁₋₃ alkyl optionally substituted by 1 to 3 substituents independently selected from the group consisting of halogen, —OH, —OR and —C(O)R; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a twenty-second embodiment, for the compound of Formula I, IA or IB, or a pharmaceutically acceptable salt thereof, J^{D2} is selected from the group consisting of hydrogen, —F, —Cl, —Br, —CH₃, —SCH₃, —OH, —CH₂F, —CH₂OCH₂CH₃, —OCH₃, —CH₂OCH₃, and —CH₂CH₂OH; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, or twenty-first embodiment.

In a twenty-third embodiment, for the compound of Formula I, IA or IB, or a pharmaceutically acceptable salt thereof, J^{D2} is selected from the group consisting of hydrogen, halogen, —OR and C₁₋₃ alkyl optionally substituted with 1 to 3 substituents independently selected from halogen and —C(O)R; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth or twenty-first embodiment. In some embodiments J^{D2} is selected from the group consisting of hydrogen, —F and —Cl. In other embodiments, it is hydrogen or —F. In still other embodiments, it is hydrogen.

In a twenty-fourth embodiment, for the compound of Formula I, IA or IB, or a pharmaceutically acceptable salt thereof, J^{C} is selected from the group consisting of hydrogen, halogen, —OR, —SR, —C(O)R, —CN and C₁₋₃ alkyl optionally substate with 1 to 3 substituents independently selected from halogen, —OR and —OH; and wherein R is C₁₋₃ alkyl optionally substituted with 1 to 3 fluoro; and the remaining variables in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-fifth embodiment, for the compound of Formula I, IA or IB, or a pharmaceutically acceptable salt thereof, J^{C} is selected from the group consisting of hydrogen, —F, —Cl, —CH₃, —CH₂F, —OCH₃, —SCH₃, —C(O)CH₃, —CH₂OCH₃, —CH₂OH and —CN; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment. In some embodiments, J^{C} selected from the group consisting of is hydrogen, —F, —Cl, —CH₃ and —CH₂F.

In a twenty-sixth embodiment, for the compound of Formula I or Formula IA, the compound is one of Formula IIA, or a pharmaceutically acceptable salt thereof:

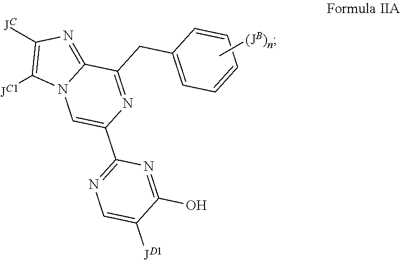

wherein the variables are as described in the first, second, third, fourth, fifth, sixth, seventh, ninth, tenth, eleventh, twelfth, thirteenth, fourteenth, fifteenth, sixteenth, seventeenth, eighteenth, nineteenth, twentieth, twenty-fourth, or twenty-fifth embodiment; and
wherein, when n is 1, 2 or 3, then at least one of J^{C}, J^{C1}, J^{D} and J^{B} is —OH, —OR, —SH, —SR, —CN, —C(O)R, C₃₋₅ cycloalkyl optionally and independently substituted with 1 to 3 independently selected halogen atoms, or a C₁₋₆ alkyl substituted with 1 to 3 substituents independently selected from the group consisting of —OH, —OR, —SR and —C(O)R; and
when n is 0, then at least one of J^{C}, J^{C1} and J^{D1} is —OH, —OR, —SH, —SR, —CN, —C(O)R, C₃₋₅ cycloalkyl optionally and independently substituted with 1 to 3 independently selected halogen atoms, or a C₁₋₆ alkyl substituted with 1 to 3 substituents independently selected from the group consisting of —OH, —OR, —SR and —C(O)R.

In a twenty-seventh embodiment, for the compound of Formula I or Formula IB, the compound is one of Formula IIB, or a pharmaceutically acceptable salt thereof:

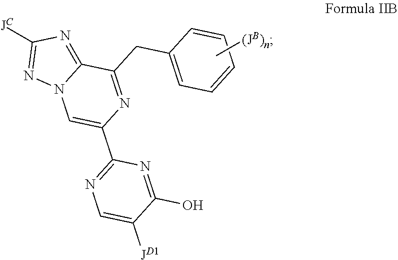

wherein all other variables are as described in the first, eighth, ninth, tenth, eleventh, twelfth, thirteenth, fourteenth, fifteenth, sixteenth, seventeenth, eighteenth, nineteenth, twentieth, twenty-fourth or twenty-fifth embodiment; and
wherein, when n is 1, 2 or 3, then at least one of J^{C}, J^{D1} and J^{B} is —OH, —OR, —SH, —SR, —CN, —C(O)R, C₃₋₅ cycloalkyl optionally and independently substituted with 1 to 3 independently selected halogen atoms, or a C₁₋₆ alkyl substituted with 1 to 3 substituents independently selected from the group consisting of —OH, —OR, —SR and —C(O)R;
when n is 0, then at least one of J^{C} and J^{D1} is —OH, —OR, —SH, —SR, —CN, —C(O)R, C₃₋₅ cycloalkyl optionally and independently substituted with 1 to 3 independently selected halogen atoms, or a C₁₋₆ alkyl substituted with 1 to 3 substituents independently selected from the group consisting of —OH, —OR, —SR and —C(O)R; and
provided that the compound is not one of the following:

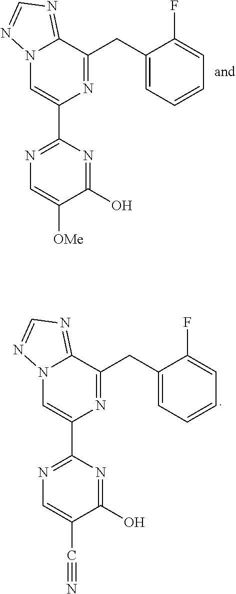 [SMILES: COc1cnc(-c2cn3ncnc3c(Cc3ccccc3F)n2)nc1O ; N#Cc1cnc(-c2cn3ncnc3c(Cc3ccccc3F)n2)nc1O]

In a twenty-eighth embodiment, for the compound of Formula I, IA, IB, IIA or IIB or a pharmaceutically acceptable salt thereof, J^{D1} is selected from the group consisting of hydrogen, halogen, —CN, —OH, —OR, —SR, —C(O)R, and C₁₋₃ alkyl optionally substituted by 1 to 3 substituents independently selected from the group consisting of halogen, —OH and —OR; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, or twenty-seventh embodiment.

In a twenty-ninth embodiment, for the compound of Formula I, IA, IB, IIA or IIB or a pharmaceutically acceptable salt thereof, J^{D1} is selected from the group consisting of hydrogen, —F, —CH₃, —CN, —SCH₃, —CHF₂, —OCH₃, —C(O)CH₃, —CH(OH)CH₃, or —CH₂CH₂OH; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, or twenty-eighth embodiment.

In a thirtieth embodiment, for the compound of Formula I, IA, IB, IIA or IIB or a pharmaceutically acceptable salt thereof, J^{D1} is selected from the group consisting of hydrogen, halogen, —OR and C₁₋₃ alkyl optionally substituted with 1 to 3 substituents independently selected from halogen and —C(O)R; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, or twenty-eighth embodiment. In some embodiments, J^{D1} is selected from the group consisting of hydrogen, —F and —Cl. In some embodiments, J^{D1} is halogen. In other embodiments, it is —F.

In a thirty-first embodiment, for the compound of Formula I, IA, IB, IIA or IIB or a pharmaceutically acceptable salt thereof, R, for each occurrence, is independently C₁₋₄ alkyl; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, or thirtieth embodiment. In some embodiments, R, for each occurrence is independently C₁₋₂ alkyl. In some embodiments, R is methyl.

In a thirty-second embodiment, the present invention is directed to sGC stimulator compounds of Table I, and their pharmaceutically acceptable salts thereof.

TABLE I
Exemplary sGC stimulators the invention.

| | |
|---|---|
| 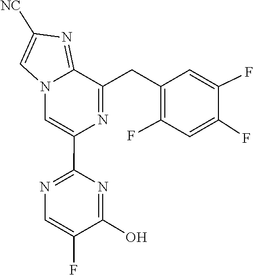 [SMILES: N#Cc1cn2cc(-c3ncc(F)c(O)n3)nc(Cc3cc(F)c(F)cc3F)c2n1] | I-3 |
| 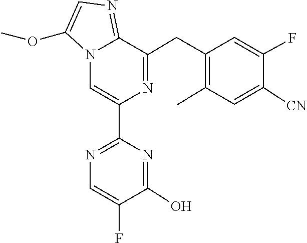 [SMILES: COc1cnc2c(Cc3cc(F)c(C#N)cc3C)nc(-c3ncc(F)c(O)n3)cn12] | I-32 |
| 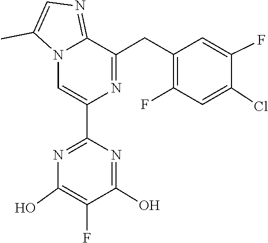 [SMILES: Cc1cnc2c(Cc3cc(F)c(Cl)cc3F)nc(-c3nc(O)c(F)c(O)n3)cn12] | I-6 |
| 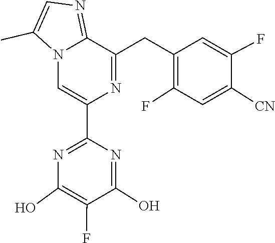 [SMILES: Cc1cnc2c(Cc3cc(F)c(C#N)cc3F)nc(-c3nc(O)c(F)c(O)n3)cn12] | I-42 |
| 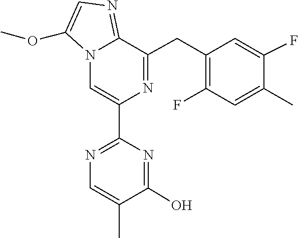 [SMILES: COc1cnc2c(Cc3cc(F)c(C)cc3F)nc(-c3ncc(C)c(O)n3)cn12] | I-7 |
| 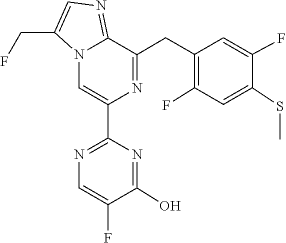 [SMILES: CSc1cc(F)c(Cc2nc(-c3ncc(F)c(O)n3)cn3c(CF)cnc23)cc1F] | I-11 |
| 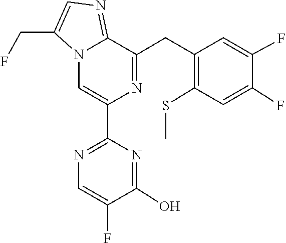 [SMILES: CSc1cc(F)c(F)cc1Cc1nc(-c2ncc(F)c(O)n2)cn2c(CF)cnc12] | I-40 |
| 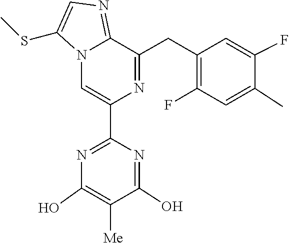 [SMILES: CSc1cnc2c(Cc3cc(F)c(C)cc3F)nc(-c3nc(O)c(C)c(O)n3)cn12] | I-38 |
| 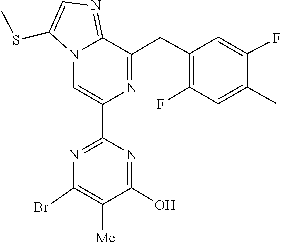 [SMILES: CSc1cnc2c(Cc3cc(F)c(C)cc3F)nc(-c3nc(O)c(C)c(Br)n3)cn12] | I-12 |
| 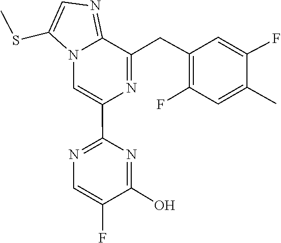 [SMILES: CSc1cnc2c(Cc3cc(F)c(C)cc3F)nc(-c3ncc(F)c(O)n3)cn12] | I-13 |
| 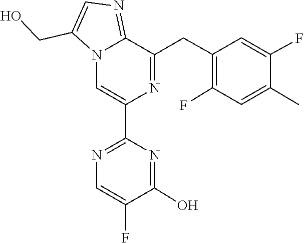 [SMILES: Cc1cc(F)c(Cc2nc(-c3ncc(F)c(O)n3)cn3c(CO)cnc23)cc1F] | I-35 |
| 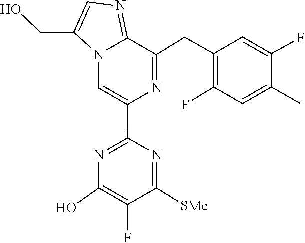 [SMILES: CSc1nc(-c2cn3c(CO)cnc3c(Cc3cc(F)c(C)cc3F)n2)nc(O)c1F] | I-15 |
| 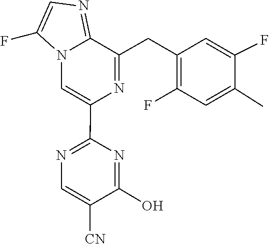 [SMILES: Cc1cc(F)c(Cc2nc(-c3ncc(C#N)c(O)n3)cn3c(F)cnc23)cc1F] | I-17 |
| 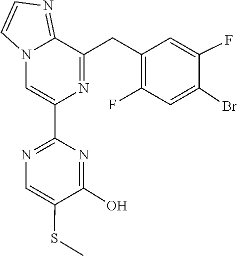 [SMILES: CSc1cnc(-c2cn3ccnc3c(Cc3cc(F)c(Br)cc3F)n2)nc1O] | I-36 |
| 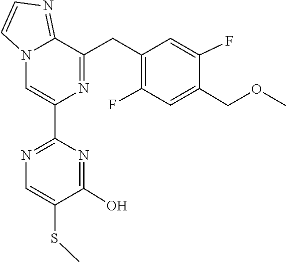 [SMILES: COCc1cc(F)c(Cc2nc(-c3ncc(SC)c(O)n3)cn3ccnc23)cc1F] | I-18 |
| 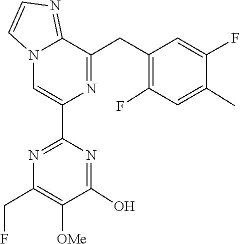 [SMILES: COc1c(O)nc(-c2cn3ccnc3c(Cc3cc(F)c(C)cc3F)n2)nc1CF] | I-21 |
| 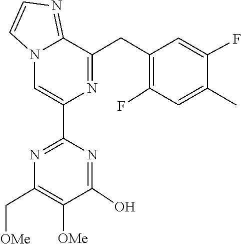 [SMILES: COCc1nc(-c2cn3ccnc3c(Cc3cc(F)c(C)cc3F)n2)nc(O)c1OC] | I-27 |
| 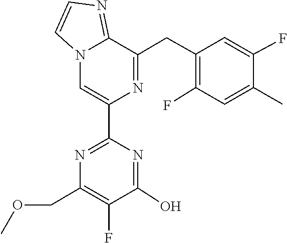 [SMILES: COCc1nc(-c2cn3ccnc3c(Cc3cc(F)c(C)cc3F)n2)nc(O)c1F] | I-22 |
| 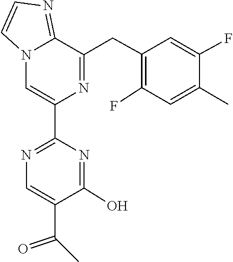 [SMILES: CC(=O)c1cnc(-c2cn3ccnc3c(Cc3cc(F)c(C)cc3F)n2)nc1O] | I-24 |
| 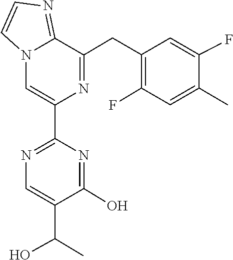 [SMILES: Cc1cc(F)c(Cc2nc(-c3ncc(C(C)O)c(O)n3)cn3ccnc23)cc1F] | I-28 |
| 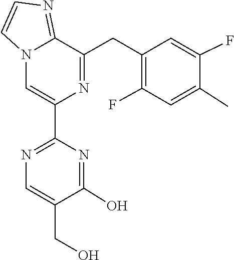 [SMILES: Cc1cc(F)c(Cc2nc(-c3ncc(CO)c(O)n3)cn3ccnc23)cc1F] | I-25 |
| 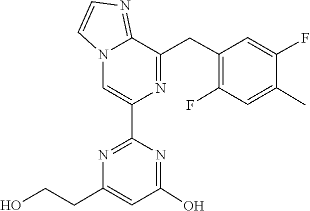 [SMILES: Cc1cc(F)c(Cc2nc(-c3nc(O)cc(CCO)n3)cn3ccnc23)cc1F] | I-26 |
| 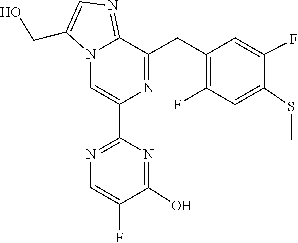 [SMILES: CSc1cc(F)c(Cc2nc(-c3ncc(F)c(O)n3)cn3c(CO)cnc23)cc1F] | I-39 |
| 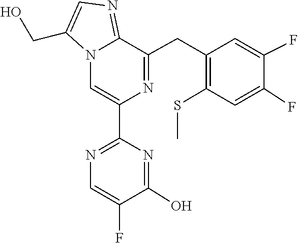 [SMILES: CSc1cc(F)c(F)cc1Cc1nc(-c2ncc(F)c(O)n2)cn2c(CO)cnc12] | I-41 |
| 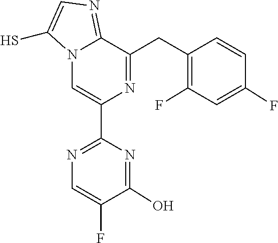 [SMILES: Oc1nc(-c2cn3c(S)cnc3c(Cc3ccc(F)cc3F)n2)ncc1F] | I-2 |
| 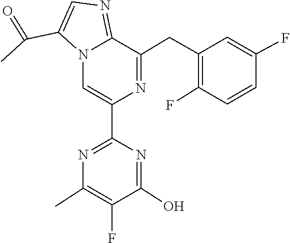 [SMILES: CC(=O)c1cnc2c(Cc3cc(F)ccc3F)nc(-c3nc(C)c(F)c(O)n3)cn12] | I-8 |
| 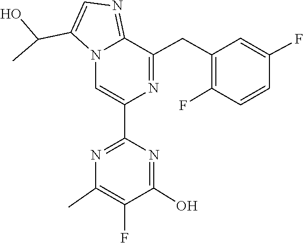 [SMILES: Cc1nc(-c2cn3c(C(C)O)cnc3c(Cc3cc(F)ccc3F)n2)nc(O)c1F] | I-31 |
| 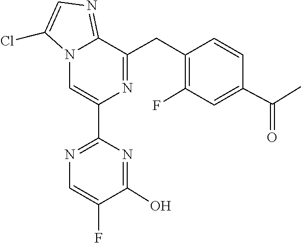 [SMILES: CC(=O)c1ccc(Cc2nc(-c3ncc(F)c(O)n3)cn3c(Cl)cnc23)c(F)c1] | I-9 |
| 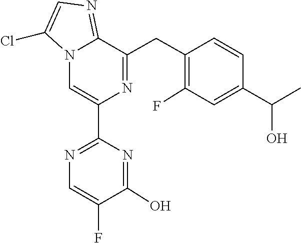 [SMILES: CC(O)c1ccc(Cc2nc(-c3ncc(F)c(O)n3)cn3c(Cl)cnc23)c(F)c1] | I-33 |
| 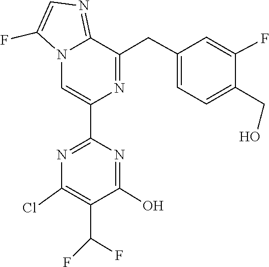 [SMILES: OCc1ccc(Cc2nc(-c3nc(O)c(C(F)F)c(Cl)n3)cn3c(F)cnc23)cc1F] | I-20 |
| 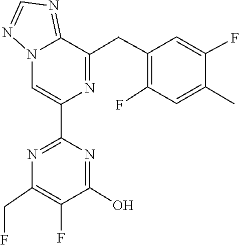 [SMILES: Cc1cc(F)c(Cc2nc(-c3nc(O)c(F)c(CF)n3)cn3ncnc23)cc1F] | I-5 |
| 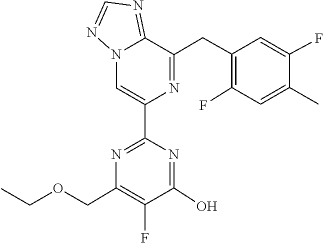 [SMILES: CCOCc1nc(-c2cn3ncnc3c(Cc3cc(F)c(C)cc3F)n2)nc(O)c1F] | I-44 |
| 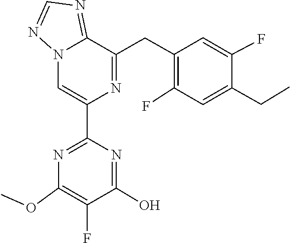 [SMILES: CCc1cc(F)c(Cc2nc(-c3nc(O)c(F)c(OC)n3)cn3ncnc23)cc1F] | I-10 |
| 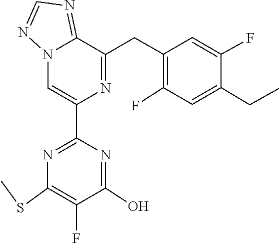 [SMILES: CCc1cc(F)c(Cc2nc(-c3nc(O)c(F)c(SC)n3)cn3ncnc23)cc1F] | I-34 |
| 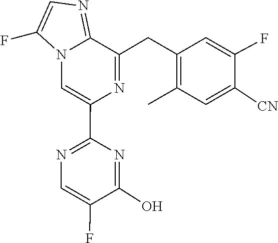 [SMILES: Cc1cc(C#N)c(F)cc1Cc1nc(-c2ncc(F)c(O)n2)cn2c(F)cnc12] | I-4 |
| 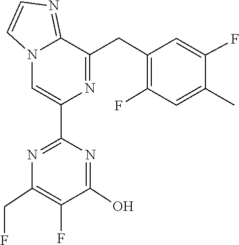 [SMILES: Cc1cc(F)c(Cc2nc(-c3nc(O)c(F)c(CF)n3)cn3ccnc23)cc1F] | I-45 |
| 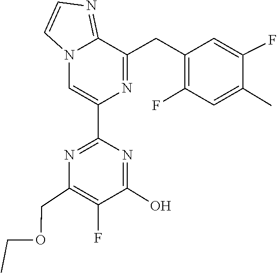 [SMILES: CCOCc1nc(-c2cn3ccnc3c(Cc3cc(F)c(C)cc3F)n2)nc(O)c1F] | I-43 |
| 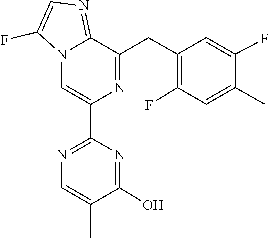 [SMILES: Cc1cc(F)c(Cc2nc(-c3ncc(C)c(O)n3)cn3c(F)cnc23)cc1F] | I-46 |
| 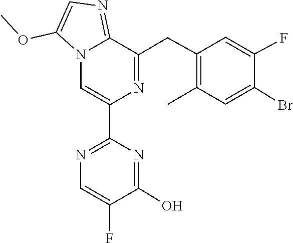 [SMILES: COc1cnc2c(Cc3cc(F)c(Br)cc3C)nc(-c3ncc(F)c(O)n3)cn12] | I-32-1 |
| 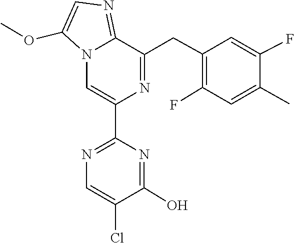 [SMILES: COc1cnc2c(Cc3cc(F)c(C)cc3F)nc(-c3ncc(Cl)c(O)n3)cn12] | I-29 |
| 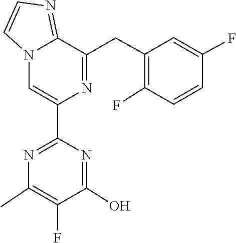 [SMILES: Cc1nc(-c2cn3ccnc3c(Cc3cc(F)ccc3F)n2)nc(O)c1F] | I-8-7 |
| 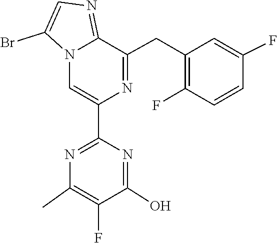 [SMILES: Cc1nc(-c2cn3c(Br)cnc3c(Cc3cc(F)ccc3F)n2)nc(O)c1F] | I-8-8 |
| 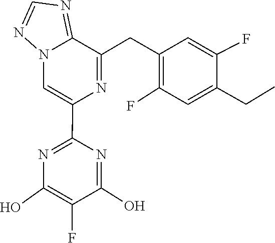 [SMILES: CCc1cc(F)c(Cc2nc(-c3nc(O)c(F)c(O)n3)cn3ncnc23)cc1F] | I-10-7 |
| 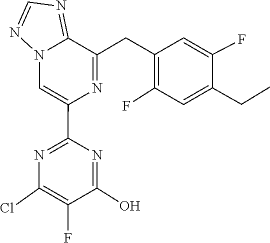 [SMILES: CCc1cc(F)c(Cc2nc(-c3nc(O)c(F)c(Cl)n3)cn3ncnc23)cc1F] | I-10-9 |
| 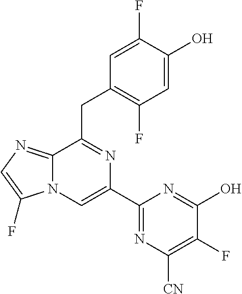 [SMILES: N#Cc1nc(-c2cn3c(F)cnc3c(Cc3cc(F)c(O)cc3F)n2)nc(O)c1F] | I-16 |
| 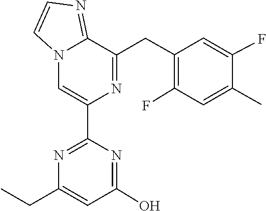 [SMILES: CCc1cc(O)nc(-c2cn3ccnc3c(Cc3cc(F)c(C)cc3F)n2)n1] | I-30 |
| 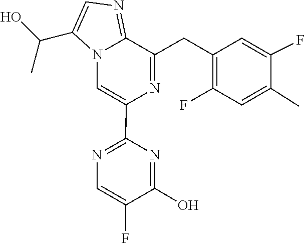 [SMILES: Cc1cc(F)c(Cc2nc(-c3ncc(F)c(O)n3)cn3c(C(C)O)cnc23)cc1F] | I-47 |

A thirty-third embodiment of the invention is a compound of Table II or a pharmaceutically acceptable salt thereof.

TABLE II
Exemplary sGC stimulators of the invention.

| | |
|---|---|
| 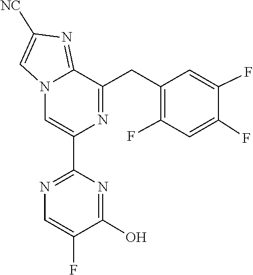 [SMILES: N#Cc1cn2cc(-c3ncc(F)c(O)n3)nc(Cc3cc(F)c(F)cc3F)c2n1] | I-3 |
| 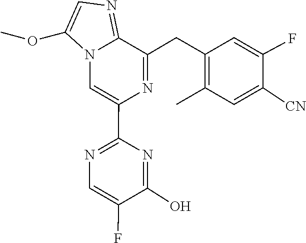 [SMILES: COc1cnc2c(Cc3cc(F)c(C#N)cc3C)nc(-c3ncc(F)c(O)n3)cn12] | I-32 |
| 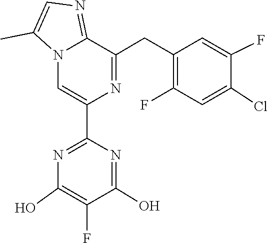 [SMILES: Cc1cnc2c(Cc3cc(F)c(Cl)cc3F)nc(-c3nc(O)c(F)c(O)n3)cn12] | I-6 |
| 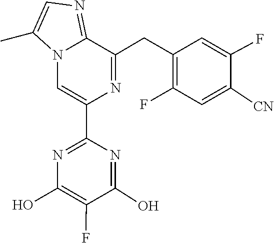 [SMILES: Cc1cnc2c(Cc3cc(F)c(C#N)cc3F)nc(-c3nc(O)c(F)c(O)n3)cn12] | I-42 |
| 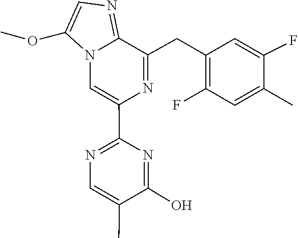 [SMILES: COc1cnc2c(Cc3cc(F)c(C)cc3F)nc(-c3ncc(C)c(O)n3)cn12] | I-7 |
| 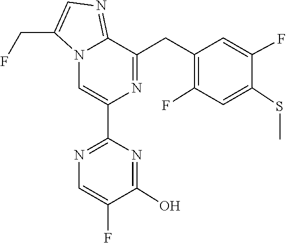 [SMILES: CSc1cc(F)c(Cc2nc(-c3ncc(F)c(O)n3)cn3c(CF)cnc23)cc1F] | I-11 |
| 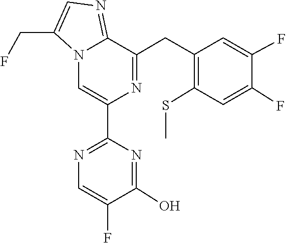 [SMILES: CSc1cc(F)c(F)cc1Cc1nc(-c2ncc(F)c(O)n2)cn2c(CF)cnc12] | I-40 |
| 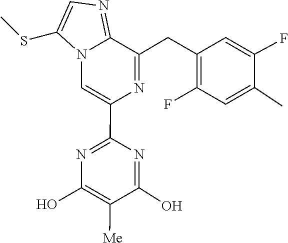 [SMILES: CSc1cnc2c(Cc3cc(F)c(C)cc3F)nc(-c3nc(O)c(C)c(O)n3)cn12] | I-38 |
| 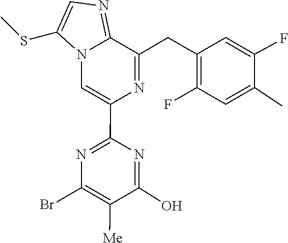 [SMILES: CSc1cnc2c(Cc3cc(F)c(C)cc3F)nc(-c3nc(O)c(C)c(Br)n3)cn12] | I-12 |
| 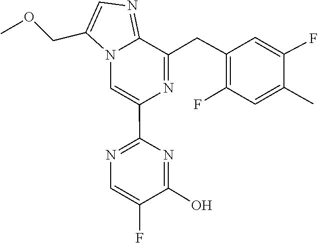 [SMILES: COCc1cnc2c(Cc3cc(F)c(C)cc3F)nc(-c3ncc(F)c(O)n3)cn12] | I-13 |
| 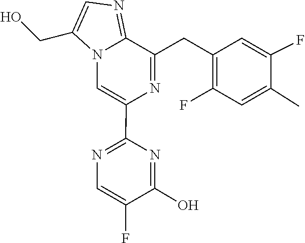 [SMILES: Cc1cc(F)c(Cc2nc(-c3ncc(F)c(O)n3)cn3c(CO)cnc23)cc1F] | I-35 |
| 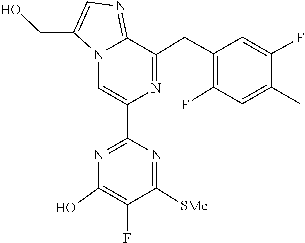 [SMILES: CSc1nc(-c2cn3c(CO)cnc3c(Cc3cc(F)c(C)cc3F)n2)nc(O)c1F] | I-15 |
| 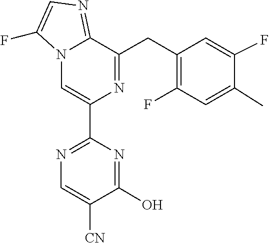 [SMILES: Cc1cc(F)c(Cc2nc(-c3ncc(C#N)c(O)n3)cn3c(F)cnc23)cc1F] | I-17 |
| 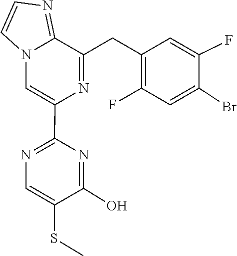 [SMILES: CSc1cnc(-c2cn3ccnc3c(Cc3cc(F)c(Br)cc3F)n2)nc1O] | I-36 |
| 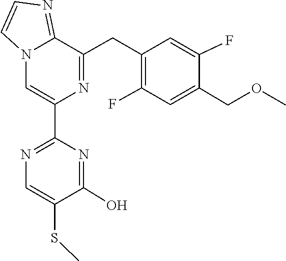 [SMILES: COCc1cc(F)c(Cc2nc(-c3ncc(SC)c(O)n3)cn3ccnc23)cc1F] | I-18 |
| 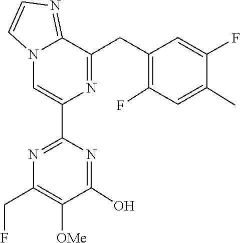 [SMILES: COc1c(O)nc(-c2cn3ccnc3c(Cc3cc(F)c(C)cc3F)n2)nc1CF] | I-21 |
| 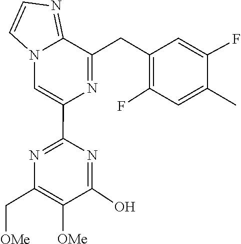 [SMILES: COCc1nc(-c2cn3ccnc3c(Cc3cc(F)c(C)cc3F)n2)nc(O)c1OC] | I-27 |
| 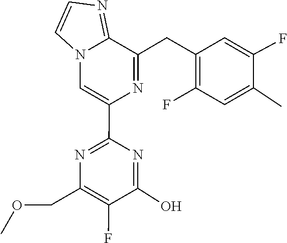 [SMILES: COCc1nc(-c2cn3ccnc3c(Cc3cc(F)c(C)cc3F)n2)nc(O)c1F] | I-22 |
| 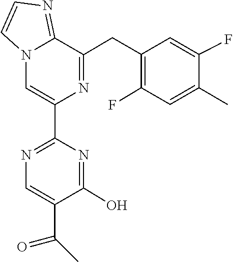 [SMILES: CC(=O)c1cnc(-c2cn3ccnc3c(Cc3cc(F)c(C)cc3F)n2)nc1O] | I-24 |
| 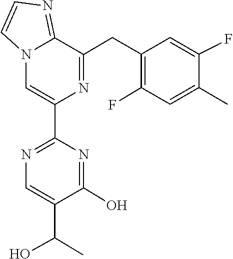 [SMILES: Cc1cc(F)c(Cc2nc(-c3ncc(C(C)O)c(O)n3)cn3ccnc23)cc1F] | I-28 |
| 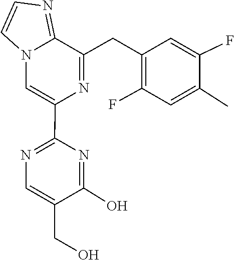 [SMILES: Cc1cc(F)c(Cc2nc(-c3ncc(CO)c(O)n3)cn3ccnc23)cc1F] | I-25 |
| 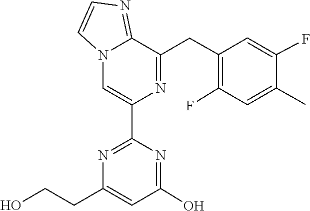 [SMILES: Cc1cc(F)c(Cc2nc(-c3nc(O)cc(CCO)n3)cn3ccnc23)cc1F] | I-26 |
| 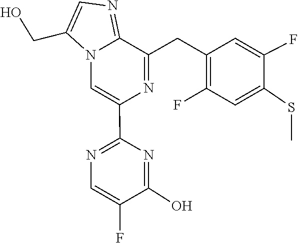 [SMILES: CSc1cc(F)c(Cc2nc(-c3ncc(F)c(O)n3)cn3c(CO)cnc23)cc1F] | I-39 |
| 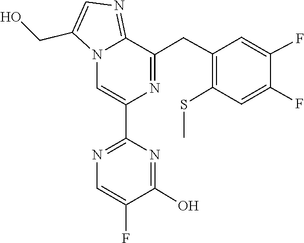 [SMILES: CSc1cc(F)c(F)cc1Cc1nc(-c2ncc(F)c(O)n2)cn2c(CO)cnc12] | I-41 |
| 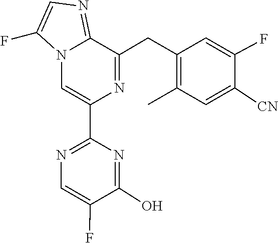 [SMILES: Cc1cc(C#N)c(F)cc1Cc1nc(-c2ncc(F)c(O)n2)cn2c(F)cnc12] | I-4 |
| 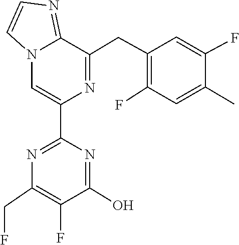 [SMILES: Cc1cc(F)c(Cc2nc(-c3nc(O)c(F)c(CF)n3)cn3ccnc23)cc1F] | I-45 |
| 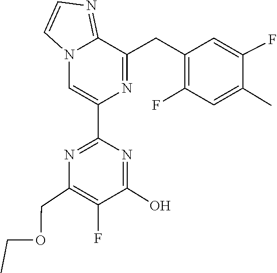 [SMILES: CCOCc1nc(-c2cn3ccnc3c(Cc3cc(F)c(C)cc3F)n2)nc(O)c1F] | I-43 |
| 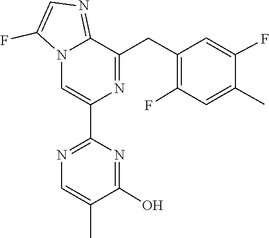 [SMILES: Cc1cc(F)c(Cc2nc(-c3ncc(C)c(O)n3)cn3c(F)cnc23)cc1F] | I-46 |
| 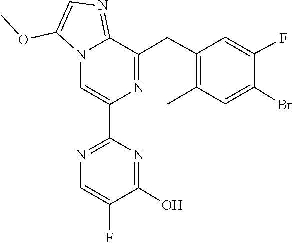 [SMILES: COc1cnc2c(Cc3cc(F)c(Br)cc3C)nc(-c3ncc(F)c(O)n3)cn12] | I-32-1 |
| 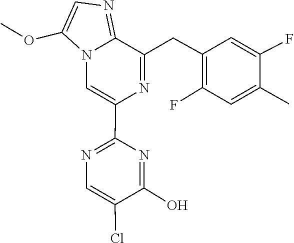 [SMILES: COc1cnc2c(Cc3cc(F)c(C)cc3F)nc(-c3ncc(Cl)c(O)n3)cn12] | I-29 |
| 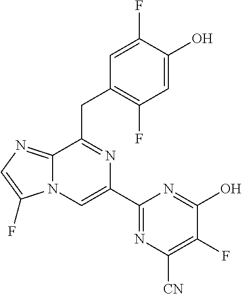 [SMILES: N#Cc1nc(-c2cn3c(F)cnc3c(Cc3cc(F)c(O)cc3F)n2)nc(O)c1F] | I-16 |

In a thirty-fourth embodiment, the compound of the invention is selected from those in Table III, or a pharmaceutically acceptable salt thereof:

TABLE III
Exemplary sGC stimulators of the invention.

| | |
|---|---|
| 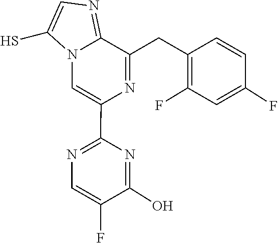 [SMILES: Oc1nc(-c2cn3c(S)cnc3c(Cc3ccc(F)cc3F)n2)ncc1F] | I-2 |
| 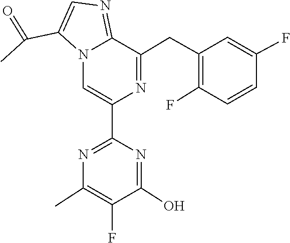 [SMILES: CC(=O)c1cnc2c(Cc3cc(F)ccc3F)nc(-c3nc(C)c(F)c(O)n3)cn12] | I-8 |
| 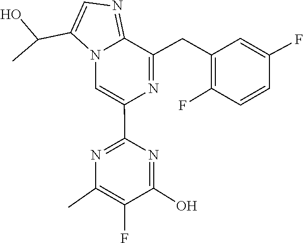 [SMILES: Cc1nc(-c2cn3c(C(C)O)cnc3c(Cc3cc(F)ccc3F)n2)nc(O)c1F] | I-31 |
| 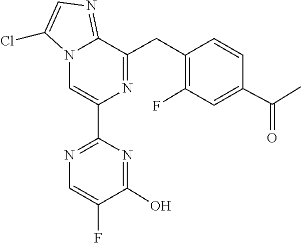 [SMILES: CC(=O)c1ccc(Cc2nc(-c3ncc(F)c(O)n3)cn3c(Cl)cnc23)c(F)c1] | I-9 |
| 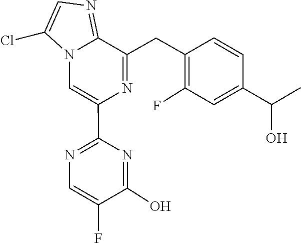 [SMILES: CC(O)c1ccc(Cc2nc(-c3ncc(F)c(O)n3)cn3c(Cl)cnc23)c(F)c1] | I-33 |
| 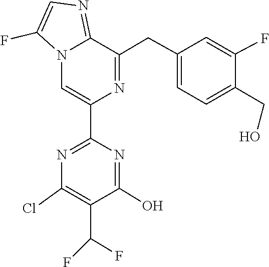 [SMILES: OCc1ccc(Cc2nc(-c3nc(O)c(C(F)F)c(Cl)n3)cn3c(F)cnc23)cc1F] | I-20 |
| 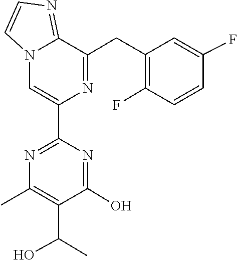 [SMILES: Cc1nc(-c2cn3ccnc3c(Cc3cc(F)ccc3F)n2)nc(O)c1C(C)O] | I-8-7 |
| 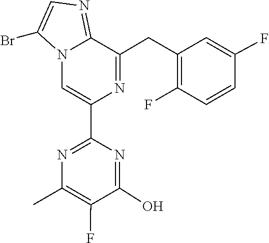 [SMILES: Cc1nc(-c2cn3c(Br)cnc3c(Cc3cc(F)ccc3F)n2)nc(O)c1F] | I-8-8 |
| 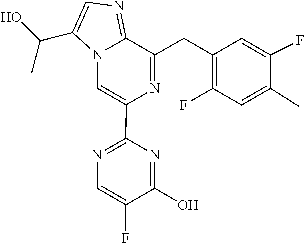 [SMILES: Cc1cc(F)c(Cc2nc(-c3ncc(F)c(O)n3)cn3c(C(C)O)cnc23)cc1F] | I-47 |
| 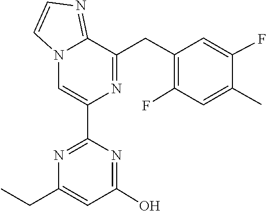 [SMILES: CCc1cc(O)nc(-c2cn3ccnc3c(Cc3cc(F)c(C)cc3F)n2)n1] | I-30 |

In a thirty-fifth embodiment, the compound of the invention is selected from those in Table IV, or a pharmaceutically acceptable salt thereof:

TABLE IV
Exemplary sGC stimulators of the invention.

| | |
|---|---|
| 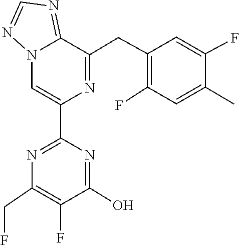 [SMILES: Cc1cc(F)c(Cc2nc(-c3nc(O)c(F)c(CF)n3)cn3ncnc23)cc1F] | I-5 |
| 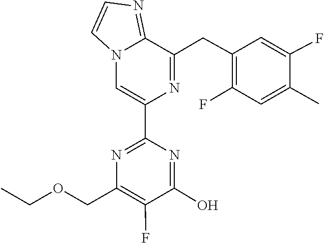 [SMILES: CCOCc1nc(-c2cn3ccnc3c(Cc3cc(F)c(C)cc3F)n2)nc(O)c1F] | I-44 |
| 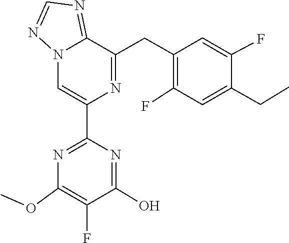 [SMILES: CCc1cc(F)c(Cc2nc(-c3nc(O)c(F)c(OC)n3)cn3ncnc23)cc1F] | I-10 |
| 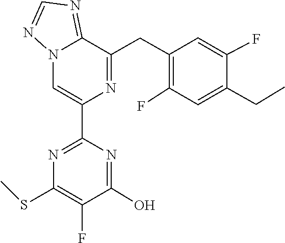 [SMILES: CCc1cc(F)c(Cc2nc(-c3nc(O)c(F)c(SC)n3)cn3ncnc23)cc1F] | I-34 |
| 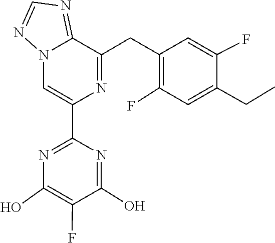 [SMILES: CCc1cc(F)c(Cc2nc(-c3nc(O)c(F)c(O)n3)cn3ncnc23)cc1F] | I-10-7 |
| 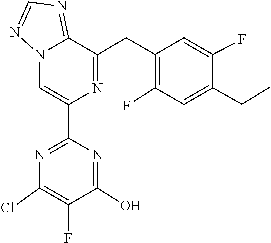 [SMILES: CCc1cc(F)c(Cc2nc(-c3nc(O)c(F)c(Cl)n3)cn3ncnc23)cc1F] | I-10-9 |

### Pharmaceutically Acceptable Salts of the Invention

A “pharmaceutically acceptable salt” of the compounds described herein include those derived from said compounds when mixed with inorganic or organic acids or bases. In some embodiments, the salts can be prepared in situ during the final isolation and purification of the compounds. In other embodiments the salts can be prepared from the free form of the compound in a separate synthetic step. The preparation of the pharmaceutically acceptable salts described above and other typical pharmaceutically acceptable salts is more fully described by Berg et al., “Pharmaceutical Salts,” J. Pharm. Sci., 1977:66:1-19, incorporated here by reference in its entirety. The pharmaceutically acceptable salts of a compound of Table I are those that may be used in medicine. Salts that are not pharmaceutically acceptable may, however, be useful in the preparation of a compound of Table I or of their pharmaceutically acceptable salts.

When a compound described herein (e.g., a compound of Table I-IV or a compound represented by Formula I, or Formula IA, IIA, IB or IIB) is acidic, suitable “pharmaceutically acceptable salts” refers to salts prepared from pharmaceutically acceptable non-toxic bases including inorganic and organic bases. Salts derived from inorganic bases include aluminum, ammonium, calcium, copper, ferric, ferrous, lithium, magnesium, manganic, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines, arginine, betaine, caffeine, choline, N, N¹-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

In some embodiments, compounds of the present invention have an acidic OH group that can react with a base (e.g., a pharmaceutically acceptable non-toxic base) to form a salt (e.g., a pharmaceutically acceptable salt). In some embodiments, the salt is an ammonium, calcium, magnesium, potassium or sodium salt. In some embodiments, the salt is a sodium salt.

When a compound described herein (e.g., a compound of Table I-IV or a compound represented by Formula I, IA, IIA, IB, IIB) is basic, salts may be prepared from pharmaceutically acceptable non-toxic acids, including inorganic and organic acids. Such acids include acetate, acetic, acid citrate, acid phosphate, ascorbate, benzenesulfonic, benzenesulfonate, benzoic, benzoate, bromide, bisulfate, bitartrate, camphorsulfonic, chloride, citrate, citric, ethanesulfonate, ethanesulfonic, formate, fumarate, fumaric, gentisinate, gluconate, gluconic, glucuronate, glutamate, glutamic, hydrobromic, hydrochloric, iodide, isethionic, isonicotinate, lactate, lactic, maleate, maleic, malic, mandelic, methanesulfonic, methanesulfonate, mucic, nitrate, nitric, oleate, oxalate, pamoic, pamoate (i.e., 1,1′-methylene-bis-(2-hydroxy-3-naphthoate)), pantothenic, pantothenate, phosphate, phosphoric, saccharate, salicylate, succinic, succinate, sulfuric, sulfate, tannate, tartrate, tartaric, p-toluenesulfonate, p-toluenesulfonic acid and the like. Particular embodiments include citric, hydrobromic, hydrochloric, maleic, phosphoric, sulfuric and tartaric acids.

In addition to the compounds described herein, their pharmaceutically acceptable salts may also be employed in compositions to treat or prevent the herein identified diseases.

### Pharmaceutical Compositions and Methods of Administration

In a second aspect, the invention relates to pharmaceutical compositions comprising a compound described herein (e.g., a compound of Tables I-IV or a compound represented by Formula I, IA, IIA, IB or IIB, or a pharmaceutically acceptable salt thereof) and at least one pharmaceutically acceptable excipient or carrier.

In some embodiments of the second aspect, the pharmaceutical composition of the present invention comprises a compound or a pharmaceutically acceptable salt thereof, as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, or thirty-fifth embodiment, and at least one pharmaceutically acceptable excipient or carrier.

The compounds herein disclosed, and their pharmaceutically acceptable salts thereof may be formulated as pharmaceutical compositions or “formulations”.

A typical formulation is prepared by mixing a compound described herein (e.g., a compound of Tables I-IV or a compound represented by Formula I, IA, IB, IIA, IIB or a pharmaceutically acceptable salt thereof), and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which a compound described herein (e.g., a compound of Tables I-IV or a compound represented by Formula I, IA, IB, IIA, IIB or a pharmaceutically acceptable salt thereof) is being formulated. Solvents are generally selected based on solvents recognized by persons skilled in the art as safe (GRAS-Generally Regarded as Safe) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g. enteric or slow release) preservatives, antioxidants, opaquing agents, glidants, processing aids, colorants, sweeteners, perfuming agents, flavoring agents and other known additives to provide an elegant presentation of the drug (i.e., a compound of Tables I-IV, a compound represented by Formula I, IA, IB, IIA or IIB or pharmaceutical composition thereof) or aid in the manufacturing of the pharmaceutical product (i.e., medicament).

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmethacylate) microcapsules, respectively; in colloidal drug delivery systems (for example, liposomes, albumin microspheres, microemulsions, nano-particles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

The formulations may be prepared using conventional dissolution and mixing procedures.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically effective amount of the compound to be administered will be governed by such considerations, and is the minimum amount necessary to ameliorate, cure or treat the disease, or one or more of its symptoms.

The terms “administer”, “administering” or “administration” in reference to a compound, composition or dosage form of the invention means introducing the compound, composition or dosage form into the system of the subject or patient in need of treatment. When a compound of the invention is provided in combination with one or more other active agents, “administration” and its variants are each understood to include concurrent and/or sequential introduction of the compound, composition or dosage form and the other active agents.

The compositions described herein may be administered systemically or locally, e.g. orally (including, but not limited to solid dosage forms including hard or soft capsules (e.g. gelatin capsules), tablets, pills, powders, sublingual tablets, troches, lozenges, and granules; and liquid dosage forms including, but are not limited to, pharmaceutically acceptable emulsions, microemulsions, aqueous or oil solutions, suspensions, syrups and elixirs, by inhalation (e.g. with an aerosol, gas, inhaler, nebulizer or the like), to the ear (e.g. using ear drops), topically (e.g. using creams, gels, inhalants, liniments, lotions, ointments, patches, pastes, powders, solutions, sprays, transdermal patches, etc.), ophthalmically (e.g. with eye drops, ophthalmic gels, ophthalmic ointments), rectally (e.g. using enemas or suppositories), nasally, buccally, vaginally (e.g. using douches, intrauterine devices, vaginal suppositories, vaginal rings or tablets, etc.), via ear drops, via an implanted reservoir or the like, or parenterally depending on the severity and type of the disease being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Formulations of a compound intended for oral use may be prepared according to any method known in the art for the manufacture of pharmaceutical compositions.

In solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time delay material such as glyceryl monostearate or glyceryl distearate alone or with a wax may be employed. A water soluble taste masking material such as hydroxypropyl-methylcellulose or hydroxypropyl-cellulose may be employed.

In addition to the active compounds, liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Oral compositions (either solid or liquid) can also include excipients and adjuvants such as dispersing or wetting agents, such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate); emulsifying and suspending agents, such as sodium carboxymethylcellulose, croscarmellose, povidone, methylcellulose, hydroxypropyl methylcelluose, sodium alginate, polyvinylpyrrolidone, gum tragacanth and gum acacia; sweetening, flavoring, and perfuming agents; and/or one or more preservatives such as ethyl or n-propyl p-hydroxy-benzoate, one or more coloring agents, one or more flavoring agents and one or more sweetening agents, such as sucrose or saccharin.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration have a particle size for example in the range of 0.1 to 500 micros (including particles in a range between 0.1 and 500 microns in increments microns such as 0.5, 1, 30, 35 microns, etc.) which is administered by rapid inhalation through the nasal passage or by inhalation through the mouth so as to reach the alveolar sacs.

The pharmaceutical compositions described herein may also be administered topically, especially when the target of treatment includes areas or organs readily accessible by topical application, including diseases of the eye, the ear, the skin, or the lower intestinal tract. Suitable topical formulations are readily prepared for each of these areas or organs. The active component is admixed under sterile conditions with a pharmaceutically acceptable carrier and any needed preservatives or buffers as may be required.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

Alternatively, the active ingredients may be formulated in a cream with an oil-in-water cream base. If desired, the aqueous phase of the cream base may include a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using a compound of the invention may be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of a compound of of the invention include Tween™-60, Span™-80, cetostearyl alcohol, benzyl alcohol, myristyl alcohol, glyceryl mono-stearate and sodium lauryl sulfate.

Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s) in an amount of, for example, 0.075 to 20% w/w. When formulated in an ointment, the active ingredients may be employed with either an oil-based, paraffinic or a water-miscible ointment base.

Compositions for rectal or vaginal administration are preferably suppositories which can be prepared by mixing the compounds described herein with suitable non-irritating excipients or carriers such as cocoa butter, beeswax, polyethylene glycol or a suppository wax which are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

Sterile injectable forms of the compositions described herein (e.g. for parenteral administration) may be aqueous or oleaginous suspension. These suspensions may be formulated according to techniques known in the art using suitable dispersing or wetting agents and suspending agents (including those described in the preceding paragraph). The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as a vegetable oil, for example arachis oil, olive oil, sesame oil or coconut oil, especially in their polyoxyethylated versions, or in mineral oil such as liquid paraffin. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of injectable formulations. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

In other embodiments of the second aspect, a compound of the invention or a pharmaceutically acceptable salt thereof may be formulated in a veterinary composition comprising a veterinary carrier. Veterinary carriers are materials useful for the purpose of administering the composition and may be solid, liquid or gaseous materials which are otherwise inert. In the veterinary art and are compatible with the active ingredient. These veterinary compositions may be administered parenterally, orally or by any other desired route.

### Therapeutic Methods

In a third aspect, the invention also provides a method of treating a disease in a subject in need thereof, comprising administering, alone or in combination therapy, a therapeutically effective amount of a compound of Formula I, IA, IIA, IB or IIB or a compound of Tables I-IV or a pharmaceutically acceptable salt thereof to the subject; wherein the disease is one that benefits from sGC stimulation or from an increase in the concentration of NO or cGMP or both, or from the upregulation of the NO-sGC-cGMP pathway. In another embodiment of the third aspect, the invention also provides a method of treating a disease in a subject in need thereof, comprising administering, alone or in combination therapy, a pharmaceutical composition or a dosage form comprising a compound of Formula I, IA, IB, IIA, IIB or Tables I-IV, or a pharmaceutically acceptable salt thereof to the subject, wherein the disease is one that benefits from sGC stimulation or from an increase in the concentration of NO or cGMP or both, or from the upregulation of the NO-sGC-cGMP pathway.

In a fourth aspect, the invention provides the use of a compound of Formula I, IA, IB, IIA, IIB, Tables I-IV or any of the compounds of the first to thirty-fifth embodiments, or a pharmaceutically acceptable salt thereof, or a pharmaceutical composition or a dosage form comprising a compound of Formula I, IA, IB, IIA, IIB or Tables I-IV or any of the compounds of the first to thirty-fifth embodiments, or a pharmaceutically acceptable salt thereof in the treatment of one of the diseases disclosed herein in a subject in need of the treatment.

In a fifth aspect, the invention provides the use of a compound of Formula I, IA, IB, IIA, IIB, Tables I-IV or any of the first to thirty-fifth embodiments, or a pharmaceutically acceptable salt thereof, or a pharmaceutically acceptable salt thereof, or a pharmaceutical composition or a dosage form comprising a compound of Formula I, IA, IB, IIA, IIB or Tables I-IV or any of the compounds of the first to thirty-fifth embodiments, or a pharmaceutically acceptable salt thereof, for the manufacture of a medicament for treating one of the diseases disclosed herein in a subject in need of the treatment.

The invention further provides a method of making or manufacturing a medicament useful for treating one of the diseases disclosed herein comprising using a compound of Formula I, IA, IIA, IB, IIB, Tables I-IV, or any of the compounds of the first to thirty-fifth embodiments or a pharmaceutically acceptable salt thereof or a pharmaceutical composition or a dosage form comprising a compound of Formula I, IA, IB, IIA, IIB or Tables I-IV or any of the compounds of the first to thirty-fifth embodiments, or a pharmaceutically acceptable salt thereof.

An embodiment of the third to fifth aspects of the invention is a method of treating a disease in a subject in need of treatment, comprising administering a therapeutically effective amount of a compound described herein (e.g., a compound or a pharmaceutically acceptable salt thereof, as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, or thirty-fifth embodiment), or a pharmaceutical composition as comprising a compound described herein (e.g., a compound or a pharmaceutically acceptable salt thereof as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, or thirty-fifth embodiment), to the subject in need of treatment.

In some embodiments of the third to fifth aspects, the compounds disclosed herein are sGC stimulators that may be useful in the prevention and/or treatment of diseases characterized by undesirable reduced bioavailability of and/or sensitivity to NO, such as those associated with conditions of oxidative stress or nitrosative stress.

Increased concentration of cGMP through the NO-sGC-cGMP pathway, leads to vasodilation, inhibition of platelet aggregation and adhesion, anti-hypertensive effects, anti-remodeling effects, anti-apoptotic effects, anti-inflammatory, anti-fibrotic effects, metabolic effects, neuronal signal transmission effects and mitochondrial effects. Thus, sGC stimulators may be used to treat and/or prevent a range of diseases.

Specific diseases or disorders which may be treated and/or prevented by administering an sGC stimulator of the invention according to the third to fifth aspects (e.g., a compound of Formula I, IA, IB, IIA, IIB, Tables I-IV or any compound of the first to thirty-fifth embodiment and pharmaceutically acceptable salts thereof), include but are not limited to:

Abetalipoproteinemia, achalasia (e.g., esophageal achalasia), acute respiratory distress syndrome (ARDS), adhesive capsulitis, age-related learning and memory disturbances, age-related memory loss, alcoholism, alopecia or hair loss, altitute sickness, Alzheimer's disease (including pre-Alzheimer's disease, mild to moderate Alzheimer's disease and moderate to severe Alzheimer's disease), amyotrophic lateral sclerosis (ALS or Lou Gehrig's disease), anal fissures, aneurysm, angina (e.g., stable or unstable angina pectoris, variant angina, Prinzmetal's angina, microvascular angina), anxiety or anxiety disorders, arginosuccinic aciduria, arterial and venous thromboses, arthritis, Asperger's syndrome, asthma and asthmatic diseases, ataxia, telangliectasia, atherosclerosis (e.g., atherosclerosis associated with endothelial injury, platelet and monocyte adhesion and aggregation, smooth muscle proliferation or migration), atrophic vaginitis, attention deficit disorder (ADD) and attention deficit hyperactivity disorder (ADHD), autism and disorders in the autism spectrum, benign prostatic hyperplasia (BPH) or hypertrophy or enlargement, bipolar disorder, bladder outlet obstruction, bladder pain syndrome (BPS), blepharitis, bone and carbohydrate metabolism disturbances, bone healing (e.g. bone healing following osteoclastic bone remodeling, osteoclastic bone resorption, new bone formation), brain aneurism, brain hypoxia, cancer metastasis, cerebral amyloid angiopathy (CAA) or congophilic angiopathy, cerebral autosomal-dominant arteriopathy with subcortical infarcts and leukoencephalopathy (CADASIL or CADASIL syndrome), cerebral perfusion, cerebral small vessel disease, cerebral vasospasm, chemo-brain, childhood disintegrative disorder, chronic bronchitis, chronic fatigue, chronic traumatic encephalopathy (CTE), ciliopathies, cirrhosis (e.g., liver cirrhosis, liver cirrhosis associated with chronic liver disease, primary biliary cirrhosis), CNS-disease related sexual dysfunction, CNS-disease related sleep disturbances, cognitive defect associated with Huntington's Disease, cognitive dysfunction, cognitive impairment (e.g., vascular cognitive impairment, mild cognitive impairment, cognitive impairment associated with diabetes, cognitive impairment associated with Multiple Sclerosis, cognitive impairment associated with obstructive sleep apnea, cognitive impairment associated with schizophrenia (CIAS), cognitive impairment associated with sickle cell disease, concussion, congenital myasthenic syndrome, connective tissue disease, consequences of cerebral infarction (apoplexia cerebri), conservation of blood substituents in trauma patients, CREST syndrome, Crohn's disease, cystic fibrosis (CF), delusional disorder, dementia (e.g., vascular dementia, post-stroke dementia, Lewy body dementia, dementia with frontal lobe degeneration, dementia with frontotemporal lobar degeneration, dementia with corticobasal degeneration, Creutzfeldt-Jakob dementia, HIV-dementia, multi-infarct dementia, post-operative dementia, strategic single-infarct dementia, HIV-associated dementia (including asymptomatic neurocognitive impairment (ANI), minor neurocognitive disorder (MND), HIV-associated dementia (HAD, also called AIDS dementia complex [ADC] or HIV encephalopathy), pre-senile dementia (mild cognitive impairment, MCI), mixed dementia, Binswanger's dementia (subcortical arteriosclerotic encephalopathy), Parkinson's Dementia), demyelination, depression, depressive disorder, dermatomyositis, diabetic angiopathy, diabetic macular edema, diabetic microangiopathies, diabetic ulcers or wounds (e.g., diabetic food ulcer), diseases associated with or related to metabolic syndrome (e.g. obesity, diabetes, insulin resistance, elevated fasting glucose, elevated fasting insulin, elevated lipids), diseases involving downregulated neurotransmitters, diseases involving impaired cerebral blood flow, diseases involving impaired neurodegeneration, diseases involving impaired synaptic function, diseases involving neuroinflammation, diseases involving neurotoxicity, diseases of the organs of the male and female urogenital system (benign and malignant), disturbances of concentration in children with learning and memory problems, Down syndrome, drug addiction, drug-induced psychosis, dry eye syndrome, Duchenne muscular dystrophy, Dupuytren's contracture, dyskinesia (e.g., acute dyskinesia, chronic or tardive dyskinesia, non-motor dyskinesia, levo-dopa induced dyskinesia (LID)), dysmenhorrea (e.g., primary dysmenhorrea, secondary dysmenhorrea), dyspaneuria, dysphagia, dystonia (e.g., generalized dystonia, focal dystonia, segmental dystonia, sexual dystonia, intermediate dystonia, acute dystonic reaction, genetic or primary dystonia), edema, elecrolyte disturbances (e.g., herkalemia, hyponatremia), emphysema, endometriosis, endothelial dysfunction or injury and diseases associated with endothelial dysfunction, erectile dysfunction, esophageal achalasia, Fabry Disease, female sexual dysfunction (e.g., female sexual arousal dysfunction), fibromyalgia, fibrosis (e.g., endomyocardial fibrosis, atrial fibrosis, cardiac interstitial fibrosis, cardiac fibrosis, pulmonary fibrosis, eye fibrosis, skin fibrosis, intestinal fibrosis, renal or kidney fibrosis, interstitial renal fibrosis, pulmonary fibrosis, idiopathic pulmonary fibrosis, progressive massive fibrosis of the lungs, liver fibrosis, mediastinal fibrosis, retroperitoneal fibrosis, arthrofibrosis, bone marrow fibrosis, myelofibrosis, osteomyelofibrosis, radiation-induced fibrosis, pancreatic fibrosis), Fragile X, functional dyspepsia, gastroparesis, Gaucher Disease, general disturbances of concentration, general psychosis, glaucoma, glioblastoma, glomerulopathies (e.g., glomerulonephritis, acute glomerulonephritis, glomerulosclerosis, focal segmental glomerulosclerosis), granulomas, head injury, hearing impairment (e.g., partial hearing loss, total hearing loss, partial deafness, total deafness, noise-induced hearing loss), heart disease (e.g., left ventricular myocardial remodeling, left ventricular systolic dysfunction, ischemic cardiomyopathy, dilatated cardiomyopathy, alcoholic cardiomyopathy, storage cardiomyopathies, congenital heart deffects, decreased coronary blood flow, diastolic or systolic dysfunction, coronary insufficiency, acute coronary syndrome, coronary artery disease, arrhythmias, reduction of ventricular preload, cardiac hypertrophy, right heart hypertrophy, disturbances of atrial and ventricular rhythm and heart conduction disturbances, atrioventricular blocks of degree I-III (AVB I-III), supraventricular tachyarrhythmia, premature ventricular contraction, atrial fibrillation, atrial flutter, ventricular fibrillation, ventricular flutter, ventricular tachyarrhythmia, torsade-de-pointes tachycardia, atrial and ventricular extrasystoles, AV-junction extrasystoles, sick-sinus syndrome, AV-node reentry tachycardia, Wolff-Parkinson-White syndrome, myocardial insufficiency, chronic, acute or viral myocarditis, cardiogenic shock, cardiac remodeling), heart failure (HF; e.g.: Heart failure with preserved ejection fraction (HFPEF), Heart failure with reduced ejection fraction (HFREF), acute heart failure, chronic heart failure, acute phases of an existing chronic heart failure (worsening HF), transient heart failure, post-acute heart failure, systolic heart failure, diastolic heart failure, congestive heart failure, acute decompensated heart failure, right ventricular failure, total heart failure, high output heart failure, heart failure with valvular defects, diabetic heart failure, heart failure/cardiorenal syndrome, right heart failure), high concentration of plasminogen activator inhibitor 1 (PA-1), high levels of fibrinogen and low density DLD, histiocytosis X, Huntington's disease or chorea (HD), hyperammonemia and related, hypertension (e.g., arterial hypertension, resistant hypertension, diabetic hypertension, idiopathic hypertension, essential hypertension, secondary hypertension, gestational hypertension, portal hypertension, systemic hypertension, pre-eclampsia, increased acute and chronic coronary blood pressure), hypertonia, hypertrophic scars, hypoactive sexual arousal disorder, hypoperfusion, impotence, Inflammaroty bowel disease (e.g., Crohn's disease, Ulcerative Colitis), inflammation caused by cerebral malaria, inflammation caused by infectious disease, inflammatory response in perioprative care, platelet aggregation, intellectual disability, intermittent claudication, interstitial cystitis (IC), intradialytic hypotension, ischemia (e.g., cerebral ischemia, myocardial ischemia, thromboembolic ischemia, critical limb ischemia), keloids, kidney disease (e.g., chronic kidney disease, acute and chronic renal failure, acute and chronic renal insufficiency, sequelae of renal insufficiency, renal-insufficiency related to pulmonary enema, renal-insufficiency related to HF, renal-insufficiency related to uremia or anemia, primary kidney disease, congenital kidney disease, polycystic kidney disease progression, kidney transplant rejection, immune complex-induced kidney disease, abnormally reduced creatinine and/or water excretion, abnormally increased blood concentrations of urea, nitrogen, potassium and/or creatinine, altered activity of renal enzymes (e.g. glutamyl synthetase), altered urine osmolarity or urine volume, increased microalbuminuria, macroalbuminuria, lesions of glomeruli and arterioles, tubular dilatation, hyperphosphatemia, vascular kidney disease, renal cysts, renal edema due to HF), Korsakoff psychosis, leukocyte activation, levo-dopa induced addictive behavior, lichen sclerosus, lipid related disorders (e.g., excessive adiposity, excessive subcutaneous fat, hyperlipidemias, dyslipidemia, hypercholesterolemias, decreased high-density lipoprotein cholesterol (HDL-cholesterol), moderately elevated low-density lipoprotein cholesterol (LDL-cholesterol) levels, hypertriglyceridemias, hyperglyceridemia, hypolipoproteinanemias, sitosterolemia, fatty liver disease, liver steatosis or abnormal lipid accumulation in the liver, steatosis of the heart, kidney or muscle, sitosterolemia, xanthomatosis, Tangier disease), liver diseases (e.g., vascular liver disease, hepatic stellate cell activation, hepatic fibrous collagen and total collagen accumulation, liver disease of necro-inflammatory and/or of immunological, cholestatic liver disease associated with granulomatous liver diseases, cholestatic liver disease associated with liver malignancies, cholestatic liver disease associated with intrahepatic cholestasis of pregnancy, cholestatic liver disease associated with hepatitis, cholestatic liver disease associated with sepsis, cholestatic liver disease associated with drugs or toxins, cholestatic liver disease associated with graft-versus-host disease, cholestatic liver disease associated with post-liver transplantation, cholestatic liver disease associated with choledocholithiasis, cholestatic liver disease associated with bile duct tumors, cholestatic liver disease associated with pancreatic carcinoma, cholestatic liver disease associated with Mirizzi's syndrome, cholestatic liver disease associated with AIDS, cholangiopathy, cholestatic liver disease associated with parasites, cholestatic liver disease associated with schistosomiasis, hepatitis, non-alcoholic steatohepatitis (NASH), non-alcoholic fatty liver disease (NAFLD), hepatic vaso-occlusive disease (VOD), hepatic sinusoidal obstruction syndrome (SOS), hepatic encephalopathy), localized thrombosis, lower urinary tract syndromes (LUTS), lumbar spinal canal stenosis, lupus nephritis, lupus or Systemic Lupus Erythematosus, microalbuminuria, microcirculation abnormalities, migraines, minor neurocognitive disorder (MND), morphea, moyamoya, multiple lacunar infarction, multiple organ dysfunction syndrome (MODS), multiple organ failure (MOF), multiple sclerosis (MS, including clinically isolated syndrome (CIS), relapsing-remitting MS (RRMS), primary progressive MS (PPMS), secondary progressive MS (SPMS)), multiple system atrophy (MSA), myocardial infarction or heart attack (e.g., ST-segment elevation myocardial infarction, Non-ST-segment elevation myocardial infarction, old myocardial infarction), myopic choroidal neovascularization, naevi, narcotic dependence, nephropathies (e.g., diabetic nephropathy, non-diabetic nephropathy, nephritis, nephropathy induced by toxins, contrast medium induced nephropathy, diabetic or non-diabetic nephrosclerosis, nephrotic syndrome, pyelonephritis, nephrogenic fibrosis), neurodegenerative diseases, neurogenic bladder and incontinence, neuroinflammation, neurologic disorders associated with decreased nitric oxide production, neuromuscular diseases (e.g., Duchenne muscular dystrophy (DMD), Becker muscular dystrophy (BMD), limb girdle muscular dystrophies, distal myopathies, type I and type II myotonic dystrophies, facio-scapulo-peroneal muscular dystrophy, autosomal and X-linked Emery-Dreifuss muscular dystrophy, oculopharyngeal muscular dystrophy, amyotrophic lateral sclerosis, spinal muscle atrophy (SMA)), neuromyelitis optica, neuropathies (e.g., peripheral neuropathy, autonomic neuropathy, central nervous system neuropathy, chemotherapy induced neuropathy, diabetic neuropathy, painful neuropathies, neuropathic pain, non-painful neuropathies, painful diabetic neuropathy, non-painful diabetic neuropathy, neuropathies associated to a CNS disease (e.g., Multiple sclerosis, MS), radiation-induced neuropathy), neuropathic pain associated with shingles, neuropathic pain associated with spine surgery), obsessive compulsive disorder (OCD), obstructive thromboanginitis, obstructive uropathy, oesinophilic fasciitis, osteoporosis, overactive bladder, pain (e.g., acute pain, central pain syndrome, inflammatory pain, post-operative pain, tonic pain, visceral pain, claudication pain, orphan pain indications (e.g., Acetazolamide-responsive myotonia, Autoerythrocyte sensitization syndrome, Autosomal dominant Charcot-Marie-Tooth disease type 2V, Autosomal dominant intermediate Charcot-Marie-Tooth disease with neuropathic pain, Autosomal recessive limb-girdle muscular dystrophy type 2A, Channelopathy-associated congenital insensitivity to pain, Chronic pain requiring intraspinal analgesia, Complex regional pain syndrome, Complex regional pain syndrome type 1, Complex regional pain syndrome type 2, Congenital insensitivity to pain with hyperhidrosis, Congenital insensitivity to pain with severe intellectual disability, Congenital insensitivity to pain-hypohidrosis syndrome, Diffuse palmoplantar keratoderma with painful fissures, Familial episodic pain syndrome, Familial episodic pain syndrome with predominantly lower limb involvement, Familial episodic pain syndrome with predominantly upper body involvement, Hereditary painful callosities, Hereditary sensory and autonomic neuropathy type 4, Hereditary sensory and autonomic neuropathy type 5, Hereditary sensory and autonomic neuropathy type 7, Interstitial cystitis, Painful orbital and systemic neurofibromas-marfanoid habitus syndrome, Paroxysmal extreme pain disorder, Persistent idiopathic facial pain, Qualitative or quantitative defects of calpain, Tolosa-Hunt syndrome.)), pancreatitis, panic disorder, Parkinson's disease, Parkinsonism Plus, Parkinson's Dysphagia, pathological eating disorders, pelvic pain, peripheral vascular disease (e.g., peripheral arterial disease, peripheral arterial occlusive disease, peripheral embolism, peripheral perfusion disturbances), peritonitis, pervasive development disorder, Peyronie's disease, Picks syndrome, polychondritis, polymyositis, post herpetic neuralgia, post-traumatic head injury, post-traumatic stress disorder (PTSD), premature ejaculation, progressive nuclear palsy, prostate hypertrophy, pulmonary disease (e.g., plexogenic pulmonary arteriopathy, bronchoconstriction or pulmonary bronchoconstriction, vascular disease of the lung, chronic obstructive pulmonary disease (COPD), pulmonary capillary hemangiomatosis, lymphangiomatosis and compressed pulmonary vessels (e.g., due to adenopathy, tumor or fibrosing mediastinitis), pulmonary vascular remodeling, pulmonary hypertonia), pulmonary hypertension (PH, e.g., pulmonary arterial hypertension (PAH), primary PH, secondary PH, sporatid PH, pre-capically PH, idiopathic PH, PH associated with left ventricular disease, PH associated with HIV, PH associated with SCD, PH associated with thromoboembolism (chronic thromboembolic PH or CTEPH), PH associated with sarcoidosis, PH associated with chronic obstructive pulmonary disease, PH associated with acute respiratory distress syndrome (ARDS), PH associated with acute lung injury, PH associated with alpha-1-antitrypsin deficiency (AATD), PH associated with pulmonary emphysema (e.g., smoking induced emphysema), PH associated with lung disease, PH associated with hypoxemia, PH associated with scleroderma, PH associated with cystic fibrosis (CF), PH associated with left ventricular dysfunction, PH associated with hypoxemia, PH (WHO groups I, II, III, IV and V), PH associated with mitral valve disease, PH associated with pericarditis, PH associated with constrictive pericarditis, PH associated with aortic stenosis, PH associated with dilated cardiomyopathy, PH associated with hyperthrophic cardiomyopathy, PH associated with restrictive cardiomyopathy, PH associated with mediastinal fibrosis, PH associated with pulmonary fibrosis, PH associated with anomalous pulmonary venous drainage, PH associated with pulmonary veno-occlusive disease, PH associated with pulmonary vasculitis, PH associated with collagen vascular disease, PH associated with congenital heart disease, PH associated with pulmonary venous hypertension, PH associated with interstitial lung disease, PH associated with sleep-disordered breathing, PH associated with chronic airflow obstruction, PH associated with obstructive sleep apnea, PH associated with central sleep apnea, PH associated with mixed sleep apnea, PH associated with alveolar hypoventilation disorders, PH associated with chronic exposure to high altitude, PH associated with neonatal lung disease, PH associated with alveolar-capillary dysplasia, PH associated with sickle cell disease, PH associated with other coagulation disorders, PH associated with chronic thromboembolism), radiculopathy, Raynaud's disease, Raynaud's syndrome (primary or secondary), refractory epilepsy, Renpennings's syndrome, reperfusion injury (e.g., ischemia-reperfusion damage, ischemia-reperfusion associated with organ transplant), restenosis (e.g., restenosis developed after thrombolysis therapies, after percutaneous transluminal angioplasties (PTAs), after transluminal coronary angioplasties (PTCAs), after heart transplant or after bypass operations), retinopathies (e.g., diabetic retinopathy, non-diabetic retinopathy, non-proliferative diabetic retinopathy, proliferative vitroretinopathy, peripheral retinal degeneration, retinal vein occlusion), Rhett's disorder, rheumatoid or rheumatic disease (e.g., arthritis, rheumatoid arthritis), sarcoidosis, sarcoids, schistosomiasis, schizoaffective disorder, schizophrenia, schizophrenia with dementia, scleroderma (e.g., localized scleroderma or circumscribed scleroderma, systemic scleroderma), sclerosis (e.g. renal sclerosis, progressive sclerosis, liver sclerosis, primary sclerosing cholanginitis, sclerosis of the gastro-intestinal tract, hippocampal sclerosis, focal sclerosis, primary lateral sclerosis, osteosclerosis, otosclerosis, atherosclerosis, tuberous sclerosis, systemic sclerosis), sepsis or septic shock or anaphylactic shock, Sickle Cell Anemia, Sickle Cell Disease, Sjogren's syndrome, sleep-wake disorders, Sneddon's syndrome, spasms (e.g., coronary spasms, vascular spasms, spasms of the peripheral arteries), spinal cord injury, spinal muscular atrophy, spinal subluxations, spinocerebellar ataxias, Steel-Richardson-Olszewski disease (progressive supranuclear palsy), stroke, subarachnoid hemorrhage, subcortical arteriosclerotic encephalopathy, syncopes, tauopathies, tension, thalamic degeneration, thromboembolic or thrombogenic disorders, transient ischemic attacks (TIAs), traumatic brain injury, tubulointerstitial diseases, ulcers, uterine fibroids, vaginal atrophy, valve deffects (e.g., mitral valve stenosis, mitral valve regurgitation, insufficiency or incompetence, aortic valve stenosis, aortic valve insufficiency, tricuspic valve insufficiency, pulmonary valve stenosis, pulmonar valve insufficiency, combined valcular deffects), vascular disease of the brain, vascular disorder resulting from cardiac and renal complications, vascular leakage or permeability, vasculitis (e.g., thrombotic vasculitis, occlusive thrombotic vasculitis, Kawasaki disease, arteritis, aortitis), vaso-occlusive crisis, venus graft failure, wet age-related macular degeneration and Williams syndrome.

In a specific embodiment, the disease that can be treated with an sGC stimulator of the invention (e.g. a compound of any one of Formulae I, IA, IB, IIA, IIB or Tables I-IV or any of the first to thirty-fifth embodiments) is a CNS (central nervous system) disease. In another embodiment, the disease is a mitochondrial disease.

In one embodiment of the third to fifth aspects, the compounds disclosed herein are sGC stimulators that may be useful in the prevention and/or treatment of diseases and disorders characterized by increased neuroinflammation. One embodiment of the invention is a method of decreasing neuroinflammation in a subject in need thereof by administering to the subject any one of the compounds of Formula I, IA, IIA, IB, IIB, Tables I-IV or any of the compounds of the first to thirty-fifth embodiments or a pharmaceutically acceptable salt thereof, or a pharmaceutical composition or a dosage form comprising them.

In another embodiment of the third to fifth aspects of the invention, the compounds here disclosed are sGC stimulators that may be useful in the prevention and/or treatment of diseases and disorders characterized by increased neurotoxicity. One embodiment of the invention is a method of reducing or compensating the negative effects neurotoxicity in a subject in need thereof by administering to the subject any one of the compounds of Formula I, IA, IIA, IB, IIB, Tables I-IV or any of the compounds of the first to thirty-fifth embodiments a pharmaceutically acceptable salt thereof, or a pharmaceutical composition or a dosage form comprising them.

In another embodiment of the third to fifth aspects of the invention, the compounds here disclosed are sGC stimulators that may be useful in the prevention and/or treatment of diseases and disorders characterized by impaired neuroregeneration. One embodiment of the invention is a method of restoring neuroregeneration in a subject in need thereof by administering to the subject any one of the compounds of Formula I, IA, IIA, IB, IIB, Tables I-IV or any of the compounds of the first to thirty-fifth embodiments or a pharmaceutically acceptable salt thereof, or a pharmaceutical composition or a dosage form comprising them.

In another embodiment of the third to fifth aspects of the invention, the compounds here disclosed are sGC stimulators that may be useful in the prevention and/or treatment of diseases and disorders characterized by impaired synaptic function. One embodiment of the invention is a method of restoring synaptic function in a subject in need thereof by administering to the subject any one of the compounds of Formula I, IA, IIA, IB, IIB, Tables I-IV or any of the compounds of the first to thirty-fifth embodiments or a pharmaceutically acceptable salt thereof, or a pharmaceutical composition or a dosage form comprising them.

In another embodiment of the third to fifth aspects of the invention, the compounds here disclosed are sGC stimulators that may be useful in the prevention and/or treatment of diseases and disorders characterized by downregulated neurotransmitters. One embodiment of the invention is a method of normalizing neurotransmitter in a subject in need thereof by administering to the subject any one of the compounds of Formula I, IA, IIA, IB, IIB, Tables I-IV or any of the compounds of the first to thirty-fifth embodiments or a pharmaceutically acceptable salt thereof, or a pharmaceutical composition or a dosage form comprising them.

In another embodiment of the third to fifth aspects of the invention, the compounds here disclosed are sGC stimulators that may be useful in the prevention and/or treatment of diseases and disorders characterized by impaired cerebral blood flow. One embodiment of the invention is a method of restoring cerebral blood flow in a subject in need thereof by administering to the subject any one of the compounds of Formula I, IA, IIA, IB, IIB, Tables I-IV or any of the compounds of the first to thirty-fifth embodiments or a pharmaceutically acceptable salt thereof, or a pharmaceutical composition or a dosage form comprising them.

In another embodiment of the third to fifth aspects of the invention, the compounds here disclosed are sGC stimulators that may be useful in the prevention and/or treatment of diseases and disorders characterized by increased neurodegeneration. One embodiment of the invention is a method of decreasing neurodegeneration in a subject in need thereof by administering to the subject any one of the compounds of Formula I, IA, IIA, IB, IIB, Tables I-IV or any of the compounds of the first to thirty-fifth embodiments or a pharmaceutically acceptable salt thereof, or a pharmaceutical composition or a dosage form comprising them.

In another embodiment of the third to fifth aspects of the invention, the compounds here disclosed are sGC stimulators that may be useful in the prevention and/or treatment of diseases and disorders characterized by cognition impairment. One embodiment of the invention is a method of improving cognition in a subject in need thereof by administering to the subject any one of the compounds of Formula I, IA, IIA, IB, IIB, Tables I-IV or any of the compounds of the first to thirty-fifth embodiments or a pharmaceutically acceptable salt thereof, or a pharmaceutical composition or a dosage form comprising them. In some embodiments, the treatment improves memory. In other embodiments, the treatment improves attention. In other embodiments, the treatment improves executive function.

In another embodiment of the third to fifth aspects of the invention, the compounds here disclosed are sGC stimulators that are neuroprotective. In particular, the compounds of Formula I, IA, IIA, IB, IIB, Tables I-IV or any of the compounds of the first to thirty-fifth embodiments or a pharmaceutically acceptable salt thereof, or a pharmaceutical composition or a dosage form comprising them may be useful protect the neurons in a subject in need thereof.

In another embodiment of the third to fifth aspects of the invention, the CNS disease, health condition or disorder is selected from the group consisting of: Alzheimer's disease (AD), vascular dementia (VD), vascular cognitive impairment, mixed dementia, Binswanger's dementia (subcortical arteriosclerotic encephalopathy), cerebral autosomal-dominant arteriopathy with subcortical infarets and leukoencephalopathy (CADASIL or CADASIL syndrome), frontotemporal lobar degeneration or dementia (FTD), asymptomatic neurocognitive impairment (ANI), subjective cognitive impairment or decline (SCD), cognitive ageing, minor neurocognitive disorder (MND), HIV-associated dementia (HAD) (also called AIDS dementia complex [ADC] or HIV encephalopathy), Lewy body dementia, pre-senile dementia or mild cognitive impairment (MCI)

In another embodiment of the third to fifth aspects of the invention, the disease, health condition or disorder is a CNS disorder or condition selected from the group consisting of sleep wake disorders, and neurological abnormalities associated with Sneddon's syndrome.

In another embodiment of the third to fifth aspects of the invention, the disease, health condition or disorder is a CNS disorder or condition selected from the group consisting of Alzheimer's disease or pre-Alzheimer's disease, mild to moderate Alzheimer's disease or moderate to severe Alzheimer's disease.

In another embodiment of the third to fifth aspects of the invention, the CNS disease is selected from the group consisting of glaucoma, Huntington's disease (or Huntington's chorea, HD), multiple sclerosis (MS), multiple system atrophy (MSA), Parkinson's disease (PD), Parkinsonism Plus, spinocerebellar ataxias (SCA), Steel-Richardson-Olszewski disease (progressive supranuclear palsy), amyotrophic lateral sclerosis (ALS or Lou Gehrig's disease) or Down's syndrome.

In another embodiment of the third to fifth aspects of the invention, the CNS disease is selected from the group consisting of attention deficit disorder (ADD) and attention deficit hyperactivity disorder (ADHD).

In another embodiment of the third to fifth aspects of the invention, the CNS disorder is selected from the group consisting of traumatic (closed or open) penetrating head injuries, traumatic brain injury (TBI), nontraumatic stroke (in particular, ischemic stroke), aneurism, hypoxia, or other injuries to the brain.

In other embodiments of the third to fifth aspects of the invention, the CNS disorder is a psychiatric, mental, mood or affective disorder selected the group consisting of bipolar disorder, schizophrenia, general psychosis, drug-induced psychosis, a delusional disorder, a schizoaffective disorder, obsessive compulsive disorder (OCD), a depressive disorder, an anxiety disorder, a panic disorder, or post-traumatic stress disorder (PTSD).

In other embodiments of the third to fifth aspects of the invention, the CNS disease or disorder is selected from the group consisting of a dystonia, including for example, generalized, focal, segmental, sexual, intermediate, genetic/primary dystonia or acute dystonic reaction; or a dyskinesia, including for example, acute, chronic/tardive, and non-motor and levo-dopa induced dyskinesia (LID).

In other embodiments of the third to fifth aspects, the CNS disease or disorder is selected from disorders characterized by a relative reduction in synaptic plasticity and synaptic processes including, for example, Fragile X, Rhett's disorder, Williams syndrome, Renpenning's syndrome, autism spectrum disorders (ASD), autism, Asperger's syndrome, pervasive development disorder or childhood disintegrative disorder.

In other embodiments of the third to fifth aspects, the CNS disorder is selected from the group consisting of chemo brain, levo-dopa induced addictive behavior, alcoholism, narcotic dependence (including but not limited to amphetamine, opiates or other substances) and substance abuse.

In one embodiment of the third to fifth aspects, the CNS disease is cognitive impairment or dysfunction resulting from brain injuries, psychiatric disorders, neurodevelopmental disorders or neurodegenerative disorders.

In some embodiments of the third to fifth aspects, cognitive impairment, either as MCI or dementia, is associated with Alzheimer's disease (AD), vascular dementia, mixed dementia, AD with vascular pathology (ADv), cerebral infarction, cerebral ischemia, stroke, head injury, traumatic head injury, learning disabilities, autism, attention deficit disorder, depression, spinocerebellar ataxia, Lewy body dementia, dementia with frontal lobe degeneration, Pick's syndrome, Parkinson's disease, progressive nuclear palsy, dementia with corticobasal degeneration, amyotrophic lateral sclerosis (ALS), Huntington's disease, demyelination diseases, multiple sclerosis (MS), thalamic degeneration, Creutzfeldt-Jakob dementia, HIV-dementia, schizophrenia, Korsakoff psychosis, post-operative cognitive decline in the elderly, bipolar disorder or mitochondrial disease. In other embodiments, cognitive impairment is associated with sickle cell disease.

In some embodiments of the third to fifth aspects, MCI, dementia, sub-clinical cognitive impairment, or SCD is associated with cognitive aging, post-operative cognitive decline, medication side-effects, metabolic imbalances, hormonal problems, vitamin or nutrient deficiencies, delirium, psychiatric illness, damage to brain neurons due to an injury (for example in stroke or other cerebral vessel diseases or due to a traumatic brain injury), early stages of a neurodegenerative process, exposure to toxins, or viral or bacterial infections.

In other embodiments of the third to fifth aspects, the compounds here disclosed are sGC stimulators that may be useful in the prevention and/or treatment of orphan pain indications. One embodiment of the invention is a method of treating an orphan pain indication in a subject in need thereof by administering to the subject any one of the compounds of Formula I, IA, IB, IIA, IIB, Tables I-IV or any compound of the first to thirty-fifth embodiments of the first aspect or a pharmaceutically acceptable salt thereof, or a pharmaceutical composition or a dosage form comprising them.

In particular, the orphan pain indication is selected from the group consisting of Acetazolamide-responsive myotonia, Autoerythrocyte sensitization syndrome, Autosomal dominant Charcot-Marie-Tooth disease type 2V, Autosomal dominant intermediate Charcot-Marie-Tooth disease with neuropathic pain, Autosomal recessive limb-girdle muscular dystrophy type 2A, Channelopathy-associated congenital insensitivity to pain, Chronic pain requiring intraspinal analgesia, Complex regional pain syndrome, Complex regional pain syndrome type 1, Complex regional pain syndrome type 2, Congenital insensitivity to pain with hyperhidrosis, Congenital insensitivity to pain with severe intellectual disability, Congenital insensitivity to pain-hypohidrosis syndrome, Diffuse palmoplantar keratoderma with painful fissures, Familial episodic pain syndrome, Familial episodic pain syndrome with predominantly lower limb involvement, Familial episodic pain syndrome with predominantly upper body involvement, Hereditary painful callosities, Hereditary sensory and autonomic neuropathy type 4, Hereditary sensory and autonomic neuropathy type 5, Hereditary sensory and autonomic neuropathy type 7, Interstitial cystitis, Painful orbital and systemic neurofibromas-marfanoid habitus syndrome, Paroxysmal extreme pain disorder, Persistent idiopathic facial pain, Qualitative or quantitative defects of calpain, and Tolosa-Hunt syndrome.

In other embodiments, the CNS disorder is neuropathic pain. In some embodiment the pain is neuropathic pain associated with a CNS disease.

In further embodiments, the disease or condition is selected from acute pain, central pain syndrome, chemotherapy induced neuropathy, diabetic neuropathy, fibromyalgia, inflammatory pain, painful diabetic peripheral neuropathy, post-operative pain, tonic pain, and visceral pain.

In other embodiments of the third to fifth aspects of the invention, the compounds here disclosed are sGC stimulators that may be useful in the prevention and/or treatment of altitude (mountain) sickness, cerebral small vessel disease, cerebral vasculitis, cerebral vasospasm, hepatic encephalopathy, moyamoya, Parkinson's Dysphagia, ataxia telangliectasia, autism spectrum disorder, chronic fatigue, chronic traumatic encephalopathy (CTE), cognitive impairment associated with diabetes, cognitive impairment associated with multiple sclerosis, cognitive impairment associated with obstructive sleep apnea, cognitive impairment associated with schizophrenia (CIAS), cognitive impairment associated with sickle cell disease, concussion, retinopathy, diabetic retinopathy (including proliferative and non-proliferative), dysphagia.

In other embodiments of the third to fifth aspects, the compounds here disclosed are sGC stimulators that may be useful in the prevention and/or treatment of eye fibrosis, Fabry Disease, Gaucher Disease, glioblastoma, brain inflammation caused by cerebral malaria (SoC), brain inflammation caused by infectious disease, intellectual disability, myopic choroidal neovascularization, neuromyelitis optica, neuropathic pain with multiple sclerosis, neuropathic pain with shingles (herpes zoster), neuropathic pain with spine surgery, Parkinson's Dementia, peripheral and autonomic neuropathies, peripheral retinal degeneration, post-traumatic stress syndrome, post herpetic neuralgia, post-operative dementia, proliferative vitroretinopathy, radiation induced brain fibrosis, radiculopathy, refractory epilepsy, retinal vein occlusion, spinal cord injury, spinal muscular atrophy, spinal subluxations. tauopathies, and wet age-related macular degeneration.

The CNS diseases that may benefit from treatment with an sGC stimulator of the invention are those CNS diseases wherein an increase in the concentration of NO or an increase in the concentration of cGMP or both, or an upregulation of the NO-sGC-cGMP pathway might be desirable.

The compounds described herein, as well as pharmaceutically acceptable salts thereof, as stimulators of sGC that are able to cross the blood-brain barrier (BBB), are useful in the prevention and/or treatment of CNS diseases, conditions and disorders which can benefit from sGC stimulation in the brain.

In some embodiments of the third to fifth aspects, the compounds of the invention are able to stimulate sGC in the brain without producing a large blood pressure (BP) reduction in the patient. In some embodiments, the compound reduces the BP in the patient an average of less than 5 mm Hg for a dose that results in desired CNS effects. In other embodiments, an average of less than 10 mm Hg. In other embodiments, the reduction of BP in the patient is not clinically significant. In still other embodiments, the methods and uses of the invention do not result in a significant incidence of adverse events (AEs) associated with symptomatic hypotension when the patient is treated for a CNS disease.

In some embodiments of the third to fifth aspects the compounds of the invention are useful in the treatment of a mitochondrial disease of genetic origin.

Specific mitochondrial disease which may be treated and/or prevented by administering an sGC stimulator of the invention (e.g., a sGC stimulator of Formula I, IA, IB, IIA, IIB, Tables I-IV or any of embodiments first to thirty-fifth of the first aspect, or a pharmaceutically acceptable salt thereof), include but are not limited to: Alpers Disease, Autosomal Dominant Optic Atrophy (ADOA), Barth Syndrome/LIC (Lethal Infantile Cardiomyopathy), Beta-oxidation defects, Systemic Primary Carnitine Deficiency, Long Chain Fatty Acid Transport Deficiency, Carnitine Palmitoyl Transferase Deficiency, Carnitine/Acylcarnitine Translocase Deficiency, Carnitine Palmitoyl Transferase I (CPT I) Deficiency, Carnitine Palmitoyl Transferase II (CPT II) Deficiency, Very Long-Chain Acyl-CoA Dehydrogenase Deficiency (VLCAD), Long-Chain Acyl-CoA Dehydrogenase Deficiency (LCAD), Long-Chain 3-Hydroxyacyl-CoA Dehydrogenase deficiency (LCHAD), Multiple Acyl-CoA Dehydrogenase Deficiency (MAD/Glutaric acidurioa Type II), Mitochondrial Trifunctional Protein Deficiency, Medium-Chain Acyl-CoA Dehydrogenase (MCAD) Deficiency, Short-Chain Acyl-CoA Dehydrogenase Deficiency (SCAD), Glutaric Aciduria Type II, (SCHAD) Deficiency, Short/Medium-Chain 3-Hydroxyacyl-CoA Dehydrogenase (S/MCHAD), Medium-Chain 3-Ketoacyl-CoA Thiolase Deficiency, 2,4-Dienoyl-CoA Reductase Deficiency, Mitochondrial Enoyl CoA Reductase Protein Associated Neurodegeneration (MEPAN), Carnitine Deficiency, Creatine Deficiency Syndromes, Co-Enzyme Q10 Deficiency, Complex I, II, III, IV, V Deficiency, Chronic Progressive External Ophthalmoplegia (CPEO), Friedreich's Ataxia, Kearns-Sayre syndrome, Leukodystrophy, Leigh Disease or Syndrome, LHON, LHON Plus, Luft Disease, MELAS (Mitochondrial myopathy, encephalomyopathy, lactic acidosis, stroke-like symptoms), Myoclonic Epilepsy with Ragged Red Fibers (MERRF), Mitochondrial Recessive Ataxia Syndrome (MIRAS), Mitochondrial Cytopathy, Mitochondrial DNA Depletion, Mitochondrial Encephalopathy, Mitochondrial Myopathy, Multiple Mitochondrial Dysfunction Syndrome, MNGIE (Myoneurogenic gastrointestinal encephalopathy), NARP (Neuropathy, ataxia, retinitis pigmentosa, and ptosis), Pearson Syndrome, Pyruvate Carboxylase Deficiency, Pyruvate Dehydrogenase Deficiency or Pyruvate Dehydrogenase Complex Deficiency (PDCD/PDH), and POLG Mutations.

In one embodiment, the mitochondrial disease is selected from Alpers, Carnitine-acyl-carnitine deficiency, Carnitine deficiency, Complex I, II, III, IV deficiency, CPEO, CPT II deficiency, Creatine deficiency syndrome, KSS, LCHAD, Leigh syndrome, Leukodystrophy, LHON, MELAS, MEPAN, MERRF, MIRAS, Mitochondrial DNA depletion, MNGIE, NARP, Pearson syndrome, and POLG mutations.

### Definitions and General Terminology Related to Methods of Use

The term “disease”, as used herein refers to any deviation from or interruption of the normal structure or function of any body part, organ, or system that is manifested by a characteristic set of symptoms and signs and whose etiology, pathology, and prognosis may be known or unknown. The term disease encompasses other related terms such as disorder and condition (or medical condition) as well as syndromes, which are defined as a combination of symptoms resulting from a single cause or so commonly occurring together as to constitute a distinct clinical picture. In some embodiments, the term disease refers to an sGC, cGMP and/or NO mediated medical or pathological disease.

“Treat”, “treating” or “treatment” with regard to a disorder, disease, condition, symptom or syndrome, refers to abrogating or improving the cause and/or the effects (i.e., the symptoms, physiological, physical, psychological, emotional or any other clinical manifestations, observations or measurements, or improving pathological assessments) of the disorder, disease, condition or syndrome.

As used herein, the terms “treat”, “treatment” and “treating” also refer to the delay or amelioration or prevention of the progression (i.e. the known or expected progression of the disease), severity and/or duration of the disease or delay or amelioration or prevention of the progression of one or more symptoms, clinical manifestations, observations or measurements, or preventing or slowing down the negative progression of pathological assessments (i.e. “managing” without “curing” the condition), resulting from the administration of one or more therapies.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In some embodiments, the subject is a non-human animal such as a farm animal (e.g., a horse, cow, pig or sheep), or a companion animal or pet (e.g., a dog, cat, mice, rats, hamsters, gerbils, guinea pig or rabbit). In some embodiments, the subject is a human.

The term “biological sample”, as used herein, refers to an in vitro or ex vivo sample, and includes, without limitation, cell cultures or extracts thereof; biopsied material obtained from a mammal or extracts thereof; blood, saliva, urine, faeces, semen, tears, lymphatic fluid, ocular fluid, vitreous humor, cerebrospinal fluid (CSF), or other body fluids or extracts thereof.

In other embodiments, the invention provides a method of stimulating sGC activity in a biological sample, comprising contacting said biological sample with a compound or composition of the invention. Use of a sGC stimulator in a biological sample is useful for a variety of purposes known to one of skill in the art. Examples of such purposes include, without limitation, biological assays and biological specimen storage.

### Combination Therapies

The compounds and pharmaceutical compositions described herein can be used alone or in combination therapy for the treatment of a disease mediated, regulated or influenced by sGC, cGMP and/or NO.

As used herein, the terms “in combination” (as in the sentence “in combination therapy”) or “co-administration” can be used interchangeably to refer to the use of more than one therapy. The use of the terms does not restrict the order in which therapies are administered to a subject.

The compounds and pharmaceutical compositions described herein can be used in combination therapy with one or more additional therapeutic agents. For combination treatment with more than one active agent, where the active agents are in separate dosage formulations, the active agents may be administered separately or in conjunction. In addition, the administration of one element may be prior to, concurrent to, or subsequent to the administration of the other agent.

When used in combination therapy with other agents, a “therapeutically effective amount” of the compounds and pharmaceutical compositions described herein and of the other agent or agents will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed.

In some embodiments, co-administration or combination therapy encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, such co administration also encompasses use of each compound in a sequential manner in either order.

When co-administration involves the separate administration of a first amount of a compound of Formula I, IA, IB, IIA, IIB, Tables I-IV, or any of the first to thirty-fifth embodiments of the first aspect and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound of Formula I, IA, IB, IIA, IIB, Tables I-IV, or any of the first to thirty-fifth embodiments of the first aspect and the second therapeutic agent can be administered in any order within about 24 hours of each other, within about 16 hours of each other, within about 8 hours of each other, within about 4 hours of each other, within about 1 hour of each other or within about 30 minutes of each other.

Examples of other therapeutic agents that may be combined with a compound of Formula I, IA, IB, IIA, IIB, Tables I-IV, or any of the first to thirty-fifth embodiments of the first aspect, or a pharmaceutically acceptable salt thereof, either administered separately or in the same pharmaceutical composition include, but are not limited to:
(1) Endothelium-derived releasing factor (EDRF) or NO gas.
(2) NO donors including, but not limited to: a nitrosothiol, a nitrite, a sydnonimine, a NONOate, a N-nitrosamine, a N-hydroxyl nitrosamine, a nitrosimine, nitrotyrosine, a diazetine dioxide, an oxatriazole 5-imine, an oxime, a hydroxylamine, a N-hydroxyguanidine, a hydroxyurea or a furoxan. Some examples of these types of compounds include: glyceryl trinitrate (also known as GTN, nitroglycerin, nitroglycerine, and trinitrogylcerin), the nitrate ester of glycerol; sodiumnitroprusside (SNP), wherein a molecule of nitric oxide is coordinated to iron metal forming a square bipyramidal complex; 3-morpholinosydnonimine (SIN-1), a zwitterionic compound formed by combination of a morpholine and a sydnonimine; S-nitroso-N-acetylpenicillamine (SNAP), an N-acetylated amino acid derivative with a nitrosothiol functional group; diethylenetriamine/NO (DETA/NO), a compound of nitric oxide covalently linked to diethylenetriamine; an m-nitroxymethyl phenyl ester of acetyl salicylic acid. More specific examples of some of these classes of NO donors include: the classic nitrovasodilators, such as organic nitrate and nitrite esters, including nitroglycerin, amyl nitrite, isosorbide dinitrate, isosorbide 5-mononitrate, and nicorandil; isosorbide; 3-morpholinosydnonimine; linsidomine chlorohydrate (“SIN-1”); S-nitroso-N-acetylpenicillamine (“SNAP”); S-nitrosoglutathione (GSNO), sodium nitroprusside, S-nitrosoglutathione mono-ethyl-ester (GSNO-ester), 6-(2-hydroxy-1-methyl-nitrosohydrazino)-N-methyl-1-hexanamine or diethylamine NONOate.
(3) Other substances that enhance cGMP concentrations, including, but not limited toprotoporphyrin IX, arachidonic acid and phenyl hydrazine derivatives.
(4) Nitric Oxide Synthase substrates, including, but not limited to L-arginine, n-hydroxyguanidine based analogs, such as N[G]-hydroxy-L-arginine (NOHA), 1-(3, 4-dimethoxy-2-chlorobenzylideneamino)-3-hydroxyguanidine, and PR5 (1-(3, 4-dimethoxy-2-chlorobenzylideneamino)-3-hydroxyguanidine); L-arginine derivatives (such as homo-Arg, homo-NOHA, N-tert-butyloxy- and N-(3-methyl-2-butenyl)oxy-L-arginine, canavanine, epsilon guanidine-carpoic acid, agmatine, hydroxyl-agmatine, and L-tyrosyl-L-arginine); N-alkyl-N′-hydroxyguanidines (such as N-cyclopropyl-N′-hydroxyguanidine and N-butyl-N′-hydroxyguanidine), N-aryl-N′-hydroxyguanidines (such as N-phenyl-N′-hydroxyguanidine and its para-substituted derivatives which bear —F, —Cl, -methyl, —OH substituents, respectively); guanidine derivatives such as 3-(trifluoromethyl) propylguanidine.
(5) Compounds which enhance eNOS transcription.
(6) NO independent heme-independent sGC activators, including, but not limited to BAY 58-2667 (described in patent publication DE19943635); HMR-1766 (ataciguat, described in patent publication WO2000002851); S 3448 (2-(4-chloro-phenylsulfonylamino)-4,5-dimethoxy-N-(4-(thiomorpholine-4-sulfonyl)-phenyl)-benzamide (described in patent publications DE19830430 and WO2000002851); and HMR-1069 (from Sanofi-Aventis).
(7) Heme-dependent, NO-independent sGC stimulators including, but not limited to YC-1 (see patent publications EP667345 and DE19744026); riociguat (BAY 63-2521, Adempas®, described in DE19834044); nelociguat (BAY 60-4552, described in WO 2003095451); vericiguat (BAY 1021189, described in U.S. Pat. No. 8,420,656); BAY 41-2272 (described in DE19834047 and DE19942809); BAY 41-8543 (described in DE19834044); etriciguat (described in WO 2003086407); CFM-1571 (described in patent publication WO2000027394); A-344905, its acrylamide analogue A-350619 and the aminopyrimidine analogue A-778935;
other sGC stimulators described in one of publications US20090209556, U.S. Pat. No. 8,455,638, US20110118282 (WO2009032249), US20100292192, US20110201621, U.S. Pat. Nos. 7,947,664, 8,053,455 (WO2009094242), US20100216764, U.S. Pat. No. 8,507,512, (WO2010099054) US20110218202 (WO2010065275), US20130012511 (WO2011119518), US20130072492 (WO2011149921), US20130210798 (WO2012058132), and Tetrahedron Letters (2003), 44(48): 8661-8663; and IW1973 (praliciguat), IW1701 (olinciguat) and CY6463 (previously IW-6463).
(8) Compounds that inhibit the degradation of cGMP and/or cAMP, including, but not limited to:
PDE1 inhibitors, PDE2 inhibitors, PDE-3 inhibitors such as, for example, amrinone, milrinone, enoximone, vesnarinone, pimobendan, and olprinone, PDE4 inhibitors, such as, for example, rolumilast, PDE5 inhibitors, such as, for example, sildenafil and related agents such as avanafil, lodenafil, mirodenafil, sildenafil citrate, tadalafil, vardenafil and udenafil; alprostadil; dipyridamole and PF-00489791; PDE6 inhibitors, PDE9 inhibitors, such as, for example, PF-04447943, PDE10 inhibitors such as, for example, PF-02545920 (PF-10), and PDE11 inhibitors.
(9) Anticoagulants, including but not limited to:
coumarines (Vitamin K antagonists) such as warfarin, cenocoumarol, phenprocoumon and phenindione;
heparin and derivatives such as low molecular weight heparin, fondaparinux and idraparinux;
direct thrombin inhibitors such as argatroban, lepirudin, bivalirudin, dabigatran and ximelagatran; and
tissue-plasminogen activators, used to dissolve clots and unblock arteries, such as alteplase.
(10) Antiplatelet drugs, including, but not limited to topidogrel, ticlopidine, dipyridamoleand aspirin.
(11) Supplemental oxygen therapy.
(12) Alpha-1-adrenoceptor antagonists, including, but not limited to prazosin, indoramin, urapidil, bunazosin, terazosin and doxazosin; atrial natriuretic peptide (ANP), ethanol, histamine-inducers, tetrahydrocannabinol (THC) and papaverine.
(13) Bronchodilators, including, but not limited to:
short acting β₂ agonists, such as albutamol or albuterol and terbutaline;
long acting β₂ agonists (LABAs) such as salmeterol and formoterol;
anticholinergics such as pratropium and tiotropium; and
theophylline, a bronchodilator and phosphodiesterase inhibitor.
(14) Corticosteroids, including, but not limited to beclomethasone, methylprednisolone, betamethasone, prednisone, prednisolone, triamcinolone, dexamethasone, fluticasone, flunisolide, hydrocortisone, and corticosteroid analogs such as budesonide.
(15) Dietary supplements, including but not limited to omega-3 oils; folic acid, niacin, zinc, copper, Korean red ginseng root, ginkgo, pine bark, Tribulus terrestris, arginine, Avena sativa, horny goat weed, maca root, muira puama, saw palmetto, and Swedish flower pollen; vitamin C, Vitamin E, Vitamin K2; testosterone supplements, testosterone transdermal patch; zoraxel, naltrexone, bremelanotide and melanotan II.
(16) PGD2 receptor antagonists.
(17) Immunosuppressants, including, but not limited to cyclosporine, tacrolimus, rapamycin and other FK-506 type immunosuppressants, mycophenolate, mycophenolate mofetil.
(18) Non-steroidal anti-asthmatics, including, but not limited to:
β2-agonists like terbutaline, metaproterenol, fenoterol, isoetharine, albuterol, salmeterol, bitolterol and pirbuterol;
β2-agonist-corticosteroid combinations such as salmeterol-fluticasone, formoterol-budesonide, theophylline, cromolyn, cromolyn sodium, nedocromil, atropine, ipratropium, ipratropium bromide; and
leukotriene biosynthesis inhibitors such as zileuton or veliflapon.
(19) Non-steroidal anti-inflammatory agents (NSAIDs), including, but not limited to:
propionic acid derivatives like alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen;
acetic acid derivatives such as indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin and zomepirac;
fenamic acid derivatives such as flufenamic acid, meclofenamic acid, mefenamic acid, niflumic acid and tolfenamic acid;
biphenylcarboxylic acid derivatives such as diflunisal and flufenisal;
oxicams such as isoxicam, piroxicam, sudoxicam and tenoxican;
salicylates such as acetyl salicylic acid and sulfasalazine; and
pyrazolones such as apazone, bezpiperylon, feprazone, mofebutazone, oxyphenbutazone and phenylbutazone.
(20) Cyclooxygenase-2 (COX-2) inhibitors, included, but not limited to celecoxib, rofecoxib, valdecoxib, etoricoxib, parecoxib and lumiracoxib; opioid analgesics such as codeine, fentanyl, hydromorphone, levorphanol, meperidine, methadone, morphine, oxycodone, oxymorphone, propoxyphene, buprenorphine, butorphanol, dezocine, nalbuphine and pentazocine.
(21) Adrenergic neuron blockers, including, but not limited to guanethidine and guanadrel.
(22) Imidazoline I-1 receptor agonists, including, but not limited to rimenidine dihydrogen phosphate and moxonidine hydrochloride hydrate.
(23) Potassium channel activators, including, but not limited to pinacidil.
(24) Dopamine D1 agonists, including, but not limited to fenoldopam mesilate; other dopamine agonists such as ibopamine, dopexamine and docarpamine.
(25) 5-HT2 antagonists, including, but not limited to ketanserin.
(26) Vasopressin antagonists, including, but not limited to tolvaptan.
(27) Calcium channel sensitizers, including, but not limited to levosimendan or activators such as nicorandil.
(28) Adenylate cyclase activators, including, but not limited to colforsin dapropate hydrochloride.
(29) Positive inotropic agents, including, but not limited to digoxin and metildigoxin; metabolic cardiotonic agents such as ubidecarenone; brain natriuretic peptides such as nesiritide.
(30) Drugs used for the treatment of erectile dysfunction, including, but not limited to alprostadil, aviptadil, and phentolamine mesilate.
(31) Drugs used for the treatment of Alzheimer's disease and dementias, including but not limited to:
acetyl cholinesterase inhibitors such as galantamine, rivastigmine, donepezil and tacrine; and
NMDA receptor antagonists such as memantine; and
oxidoreductase inhibitors such as idebenone.
(32) Psychiatric medications, including, but not limited to:
ziprasidone, risperidone, olanzapine, valproate;
dopamine D4 receptor antagonists such as clozapine;
dopamine D2 receptor antagonists such as nemonapride;
mixed dopamine D1/D2 receptor antagonists such as zuclopenthixol;
GABA A receptor modulators such as carbamazepine;
sodium channel inhibitors such as lamotrigine;
monoamine oxidase inhibitors such as moclobemide and indeloxazine; and primavanserin, and perospirone.
(33) Drugs used for the treatment of movement disorders or symptoms, including, but not limited to:
catechol-O-methyl transferase inhibitors such as entacapone;
monoamine oxidase B inhibitors such as selegiline;
dopamine receptor modulators such as levodopa;
dopamine D3 receptor agonists such as pramipexole;
decarboxylase inhibitors such as carbidopa;
other dopamine receptor agonists such as pergolide, ropinirole, cabergoline;
ritigonide, istradefylline, talipexole; zonisamide and safinamide; and
synaptic vesicular amine transporter inhibitors such as tetrabenazine.
(34) Drugs used for the treatment of mood or affective disorders or OCD such as the following types:
tricyclic antidepressants such as amitriptyline, desipramine, imipramine, amoxapine, nortriptyline, doxepin and clomipramine;
selective serotonin reuptake inhibitors (SSRIs) such as paroxetine, fluoxetine, sertraline, trazodone and citralopram;
atypical antidepressants such as agomelatine;
selective norepinephrine reuptake inhibitors (SNRIs) such as venlafaxine, reboxetine and atomoxetine; dopaminergic antidepressants such as bupropion and amineptine.
(35) Drugs for the enhancement of synaptic plasticity, including, but not limited to: nicotinic receptor antagonists such as mecamylamine; and
mixed 5-HT, dopamine and norepinephrine receptor agonists such as lurasidone.
(36) Drugs used for the treatment of ADHD such as amphetamine; 5-HT receptor modulators such as vortioxetine and alpha-2 adrenoceptor agonists such as clonidine.
(37) Nitric oxide synthase cofactors, including, but not limited to tetrahydrobiopterin, dihydrobiopterin and sapropterin.
(38) Blood glucose lowering medications (also referred as glycemic control medications or antidiabetic medications) including, but not limited to: biguanides such as metformin;
sulfonylureas such as glyburide, glybenclamide, glipizide, gliclazide, gliquidone, glimepiride, atorvastatin calcium combined with glimerpiride, meglinatide, tolbutamide, chlorpropamide, acetohexamide, and tolazimide;
alpha-glucosidase inhibitors such as acarbose, epalrestat, voglibose, and miglitol;
insulin secretagoges such as repaglinide, mitiglinide and nateglinide;
thiazolidinediones such as rosiglitazone, troglitazone, ciglitazone, pioglitazone, englitazone, lobeglitazone sulfate and balaglitazone;
DPP-4 inhibitors (or DPP-IV inhibitors) such as sitagliptin, vildagliptin, saxagliptin, alogliptin, linagliptin, alogliptin benzoate combined with metformin or metformin hydrochloride, anagliptin, teneligliptin, atorvastatin calcium and glimepiride, empagliflozin combined with linagliptin, gemigliptin, sitagliptin phosphate monohydrate combined with pioglitazone hydrochloride, sitagliptin combined with pioglitazone, sitagliptin combined with atorvastatin calcium, and (2S,4S)-1-[2-(1,1-dimethyl-3-oxo-3-pyrrolidin-1-yl-propylamino)acetyl]-4-fluoro-pyrrolidine-2-carbonitrile (DBPR-108);
GLP-1 receptor agonists or incretin mimetics such as exenatide, dulaglutide, liraglutide, semaglutide, lixisenatide, lixisenatide combined with insulin glargine, albiglutide and pegapamodutide (TT-401), LY3298176 (dual glucose-dependent insulinotropic polypeptide (GIP) and GLP-1 receptor agonist);
SGLT2 inhibitors (SGLT2is) such as empagliflozin, empaglifozin combined with linagliptin, empagliflozin combined with metformin, ipragliflozin, ipragliflozin L-proline, tofogliflozin, sergliflozin etabonate, remogliflozin etabonate, ertugliflozin, ertugliflozin combined with sitagliptin, ertugliflozin combined with metformin, sotagliflozin, canagliflozin, canagliflozin combined with metformin or metformin hydrochloride, dapagliflozin, dapagliflozin combined with metformin or metformin hydrochloride and luseoglifozin, dapagliflozin combined with saxagliptin;
SGLT1 inhibitors or combinations of SGLT1 and SGLT2 inhibitors such as sotagliflozin;
insulin therapy such one of the many types of insulin, like insulin glulisine, insulin degludec, insulin lispro, insulin aspart, insulin glargine, insulin detemir, insulin isophane, insulin mixtard (human insulin containing both fast-acting (soluble) and long-acting (isophane) insulin, insulin degludec combined with insulin aspart, insulin human (rDNA origin) inhalation powder, recombinant human insulin, hepatic-directed vesicle insulin, insulin tregopi (IN-105), insulin degludec combined with liraglutide, insulin peglispro (LY-2605541) and nodlin; and
tolimidone (a lyn kinase activator).
(39) Blood pressure lowering medications (also known as anti-hypertensive medications), including, but not limited to:
diuretics such as thiazide diuretics, chlorothiazide, chlorthalidone, hydrochlorothiazide, bendroflumethiazide, cyclopenthiazide, methyclothiazide, polythiazide, quinethazone, xipamide, metolazone, indapamide, cicletanine, furosemide, toresamide, amiloride, spironolactone, canrenoate potassium, eplerenone, triamterene, acetazolamid and carperitide;
beta blockers such as acebutolol, atenolol, metoprolol, and nebivolol;
angiotensin-converting enzyme (ACE) inhibitors such as sulfhydryl-containing agents (for example, captopril, zofenopril), dicarboxylate-containing agents (for example, enalapril, quinapril, ramipril, perindopril, lisinopropil, and benazepril), phosphonate-containing agents (for example fosinopril), naturally occurring ACE inhibitors (for example, casokinins, lactokinins, lactotripeptides Val-Pro-Pro and Ile-Pro-Pro), alacepril, delapril, cilazapril, imidapril, temocapril, moexipril, lisinopril, combinations of lisinopril with hydrochlorothiazide, trandolapril and spirapril;
angiotensin II receptor blockers (ARBs) such as candesartan, losartan, losafran potassiun-hydrochlorothiazide, valsartan, candesartan cilexetil, eprosaran, irbesartan, telmisartan, olmesartan medoxomil (or olmesartan), azilsartan medoxomil, azilsartan, amlodipine besylate combined with irbesartan, azilsartan combined with amlodipine besilate, cilnidipine combined with valsartan, fimasartan, irbesartan combined with atorvastatin, irbesartan combined with trichlormethiazide, losartan potassium combined with hydrochlorothiazide and/or amlodipine besylate, pratosartan, atorvastatin calcium combined with losartan potassium, nifedipine and candesartan cilexetil, sacubitril combined with valsartan or LCZ-696, angiotensin AT2 antagonist and TAK-591 and olmesartan medoxomil;
endothelin Receptor antagonists (ERAs) such as atrasentan, bosentan, sitaxentan, ambrisentan, actelion-1 (macitentan), Cyclo(D-trp-D-asp-L-pro-D-val-L-leu) (BQ-123), sparsentan and tezosentan disodium;
mineralocorticoid receptor antagonists (MRAs) such as spironolactone, amiloride hydrochloride combined with spironolactone, apararenone or MT-3995, eplerenone, and finerenone (BAY-94-8862);
calcium channel blockers such as amlodipine, aranidipine, azelnidipine, barnidipine, benidipine, cilnidipine, clevidipine, diltiazem, efonidipine, felodipine, lacidipine, lercanidipine, manidipine, nicardipine, nifedipine, nilvadipine, nimodipine, nisoldipine, nitrendipine, pranidipine, isradipine, verapamil, gallopamil, diltiazem, mibefradil, bepridil, fluspirilene and fendiline;
renin inhibitors such as aliskiren;
alpha blockers such as doxazosin and prazosin;
alpha-beta blockers such as carvedilol and labetalol;
central-acting agents such as clonidine, guanfacine and methyldopa;
vasodilators such as nitroglycerine, hydralazine and minoxidil; and
aldosterone antagonists such as finerenone, spironolactone and eplerenone.
(40) Anti-hyperlipidemic medications, including but not limited to:
statins such as atorvastatin fluvastatin, lovastatin, pitavastatin, pravastatin, rosuvastatin, and simvastatin;
combinations of statins with another agent such as amlodipine/atorvastatin, aspirin/pravastatin, ezetimibe/simvastatin, niacin/simvastatin, lovastatin/niacin, simvastatin/sitagliptin and atorvastatin/ezetimibe;
fibrates or fibric acid derivatives. Examples include, but are not limited to, fenofibrate, gemfibrozil, bezafibrate, ciprofibrate, clinofibrate and clofibrate;
niacin (or nicotinic acid);
bile acid sequestrants such as cholestyramine, colesevelam, colestilan and colestipol;
ezetimibe, lomitapide, phytosterols or orlistat; and
PCSK9 inhibitors such as alirocumab and evolocumab;
(41) Neprilysin inhibitors (also known as endopeptidase inhibitors or NEP inhibitors or enkephalinase inhibitors), including, but not limited to sacubitril, or the combination of sacubitril with valsartan; neprilysin inhibitors in development TD-1439 or TD-0714.
(42) Renoprotective drugs, including, but are not limited to:
Bardoxolone;
ACE inhibitors such as captopril;
ARBs such as losartan or irbesartan;
SGLT2 inhibitors such as canagliflozin,
GLP1 receptor agonists;
MRAs such as finerenone;
ERAs such as atrasentan; and apoptosis signal-regulating kinase 1 (ASK1) inhibitors such as selonsertib.
(43) Hydroxyurea (HU, hydroxycarbamide).
(44) Anti-sickling agents, including, but not limited to hydroxyurea, voxelotor or GBT-440.
(45) Anti-adhesion therapies, including, but not limited, to blocking antibodies to P-selectin, E-selectin, VLA-4, VCAM-1.
(46) Glutamine.
(47) Erythropoietin (EPO), also known as hematopoietin or hemopoietin, including all its forms such as exogenous erythropoietin, recombinant human erythropoietin (rhEPO) or other erythropoiesis-stimulating agents (ESA) two examples being epoetin alfa and epoetin beta.
(48) Antibiotics, including but not limited to:
penicilin and its derivatives, including, but not limited to penicillin, amoxicillin, ampicillin, azlocillin, cloxacillin, penicillin G, penicillin V, procaine penicillin or benzathine penicillin amongst others.
cephalosporins such as cephalexin, cefadroxil, cefaclor, cefuroxime and cefexime;
macrolides such as erythromycin, clarithromycin, azithromycin, and roxithromycin;
tetracycline and its derivatives such as demeclocycline, doxycycline, minocycline, oxytetracycline and tetracycline;
sulfonamides, including, but not limited to, mafenide, sulfacetamide, sulfadiazine, silver sulfadiazine, sulfadimethoxine, sulfamethizole, sulfamethoxazole, sulfasalazine, trimethoprim-sulfamethoxazole (Co-trimoxazole), and sulfisoxazole; and
quinolones, including, but not limited to ciprofloxacin, enoxacin, gatifloxacin, gemifloxacin, levofloxacin, lomefloxacin, moxifloxacin, ofloxacin, and nalidixic acid.
(49) FXR agonists, including but not limited to obeticholic acid, cenicriviroc, emricasan, GR-MD-02, selonsertib and elafibranor.
(50) Thyroid receptor-beta agonists, including, but not limited to MGL-3196.
(51) Acetyl-CoA carboxylase inhibitors, including but not limited to GS-0976.
(52) Treatments for mitochondrial disorders including, but not limited to, vitamins and supplements, including Coenzyme Q10; B complex vitamins, especially thiamine (B1) and riboflavin (B2); Alpha lipoic acid; L-carnitine (Carnitor); Creatine; Citrulline, and L-Arginine.
(53) Treatments for epilespsy or seizures including, but not limited to, phenytoin, valproic acid, phenobarbital, lamotrigine, carbamazepine, topiramate, oxcarbazepine, zonisamide, gabapentin, levetiracetam, pregabalin, clonazepam, lacosamide, rufinamide, and vigabatrin.

### Packaging and Kits

The pharmaceutical compositions (or formulations) for use may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The compounds and pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound described herein and a pharmaceutically acceptable carrier, vehicle or diluent; and b) a container or packaging. The kits may optionally comprise instructions describing a method of using the pharmaceutical compositions in one or more of the methods described herein (e.g. preventing or treating one or more of the diseases and disorders described herein). The kit may optionally comprise a second pharmaceutical composition comprising one or more additional agents described herein for co therapy use, a pharmaceutically acceptable carrier, vehicle or diluent. The pharmaceutical composition comprising the compound described herein and the second pharmaceutical composition contained in the kit may be optionally combined in the same pharmaceutical composition.

### EXAMPLES

All references provided in the Examples are herein incorporated by reference. As used herein, all abbreviations, symbols and conventions are consistent with those used in the contemporary scientific literature. See, e.g. Janet S. Dodd, ed., The ACS Style Guide: A Manual for Authors and Editors, 2^{nd} Ed., Washington, D.C.: American Chemical Society, 1997, herein incorporated in its entirety by reference.

Various embodiments of the invention can be described in the text below.

Definitions of the abbreviations used in the Examples section is provided in the table below.

| Abbreviation | Word or Phrase |
|---|---|
| DMF | N,N-dimethylformamide |
| DMSO | Dimethylsulphoxide |
| DMSO-d6 | Deuterated Dimethylsulphoxide |
| dppf | 1,1′-bis(diphenylphosphino)ferrocene |
| ES+ | Positive electron spray ionization |
| EtOAc | ethyl acetate |
| equiv. | Equivalent |
| FA | Formic Acid |
| h | hour(s) |
| HPLC | high performance liquid chromatography |
| i-PrOH | Isopropyl alcohol |
| LC-MS | Liquid Chromatography- Mass Spectrometry |
| MeOH | Methanol |
| mL | Milliliter |
| MHz | Megahertz |
| MS | Mass Spectrometry |
| NMR | Nuclear Magnetic Resonance |
| No. | Compound Number |
| Ph | Phenyl |
| ppm | parts per million |
| RP- HPLC | reversed phase high performance liquid chromatography |
| TFA | Trifluoroacetic acid |
| THF | Tetrahydrofuran |
| TMSCl | Trimethylsilyl chloride |
| DMA | Dimethylacetamide |
| CN | Cyano |
| PPh₃ | Triphenyl phosphate |
| PMBSH | p-methoxybenzylthiol |
| dba | dibenzylideneacetone |
| NaOMe | Sodium methoxide |
| BH₃ | Borane |
| PBr₃ | Phosphorous tribromide |
| Et₂O | Diethyl ether |
| NaH | Sodium hydride |
| LiAlH₄ | Lithium aluminium hydride |
| i-PrOH | Isopropyl alcohol |
| NH₄Cl | Ammonium chloride |
| NBS | N-bromosuccinimide |
| CHCl₃ | Chloroform |
| HCl | Hydrochloride |
| NaBH₄ | Sodium borohydride |
| NCS | N-chlorosuccinimide |
| POCl₃ | Phosphoryl chloride |
| CBr₄ | Carbon tetrabromide |
| MeSH | Methane thiol |
| NaSMe | Sodium methane thiol |
| DCM | Dichloromethane |
| DAST | Diethylaminosulfur trifluoride |
| POBr₃ | Phosphoryl bromide |
| MOMBr | Bromomethyl methyl ether |
| TEA | Triethyl amine |
| Aq. | Aqeous |
| TFA | Trifluoroacetic anhydride |
| AIBN | Azobisisobutyronitrile |
| MeCN | Acetonitrile |
| DIEA | N,N-Diisopropylethylamine |
| SFC | Supercritical fluid chromatography |

### Synthesis Section

### Example 1: Synthesis of Compounds of Tables I-IV

The present invention also provides methods for synthesizing the compounds of Tables I-IV, which represent another aspect of this invention. Compounds of this invention may be prepared according to the general and specific syntheses described herein, synthetic procedures reported in the chemical literature or methods known to a person of ordinary skill in the art. As could be appreciated by those of ordinary skill in the art, optimum reaction condition, which may be determined during the experimentation, may vary based on the reaction type and the specific reagents used in the reaction. As such, unless specifically described, reaction conditions such as pressure, temperature, relative ratio of the reagents, solvent, and reaction time may be readily selected and modified, without undue experimentation, by a person of ordinary skill in the art. Compounds and intermediates of this invention may be purified by purification methods known to a person of ordinary skill in the art. These methods include, but are not limited to, silica gel chromatography, recrystallization, reverse phase HPLC (RP-HPLC) and Supercritical Fluid Chromatography (SFC). Purification on RP-HPLC may be accomplished on a suitable reverse phase column (e.g., Waters XBridge OBD C18, 5 μm, 19×150 mm) using a suitable gradient selected from a range of 0% to 100% acetonitrile in water containing an additive such as 0.1% TFA or formic acid. Diastereomers may be separated by silica gel chromatography, RP-HPLC or chiral HPLC. Discrete enantiomers may be obtained from a mixture of enantiomers by resolution using a chiral HPLC. Reaction progress may be monitored by methods known to one of ordinary skill in the art such as thin layer chromatography, reverse phase HPLC, or tandem reverse phase HPLC-Mass Spectrometry (LC-MS).

Starting materials used in the syntheses described herein are available from commercial sources or may be prepared by a person of ordinary skill in the art using methods reported in the chemical literature or referenced herein.

The general methods described herein may be used to prepare compounds of Tables I-IV. The general and specific methods described herein are provided as illustrations of the enablement of the present invention. As such, they are not intended to impose any restrictions on the subject matter and the scope of the claimed compounds of this invention.

All references provided in the Examples are herein incorporated by reference. As used herein, all abbreviations, symbols and conventions are consistent with those used in the contemporary scientific literature. See, e.g. G. M. Banik, G. Baysinger, P. V. Kamat, N. J. Pienta, eds., The ACS Guide to Scholarly Communication, Washington, D.C.: American Chemical Society, 2020 (https;//pub.acs.org/doi/book/10.1021/acsguide), herein incorporated in its entirety by reference.

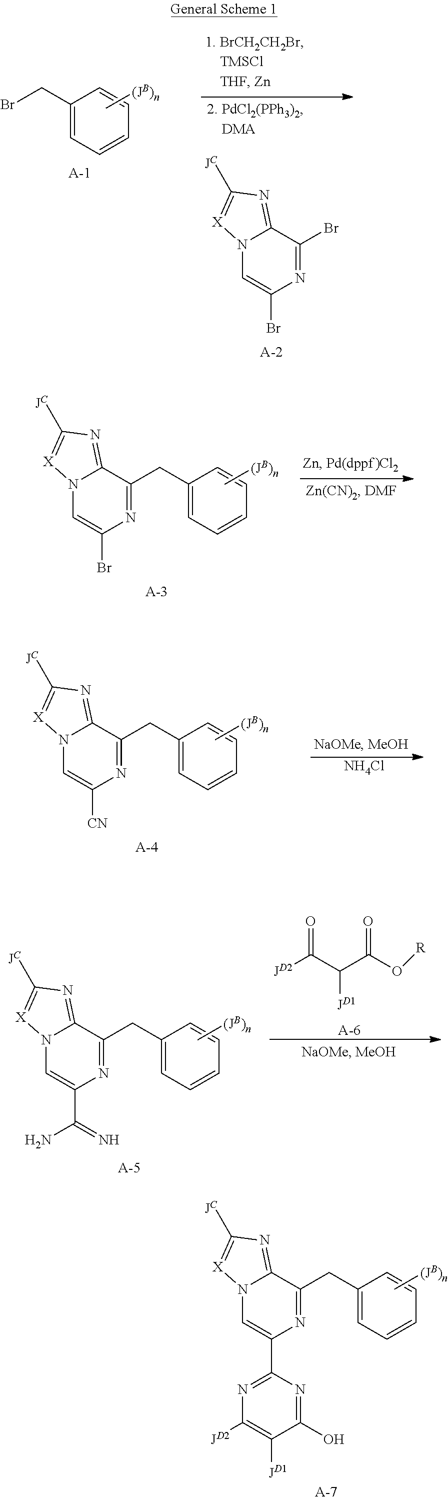

The compounds of Tables I-IV can be made using the general synthesis as described in Scheme 1. Each of X, J^{C}, J^{C1}, J^{B}, J^{D1}, and J^{D2} are as defined above, or a precursor, intermediate, or protected form thereof. Specific variations for each compound would be apparent to the skilled artisan and, in some instances, shown below.

### Synthesis of 2-(8-(2,4-difluorobenzyl)-3-mercaptoimidazo[1,2-a]pyrazin-6-yl)-5-fluoropyrimidin-4-ol (I-2)

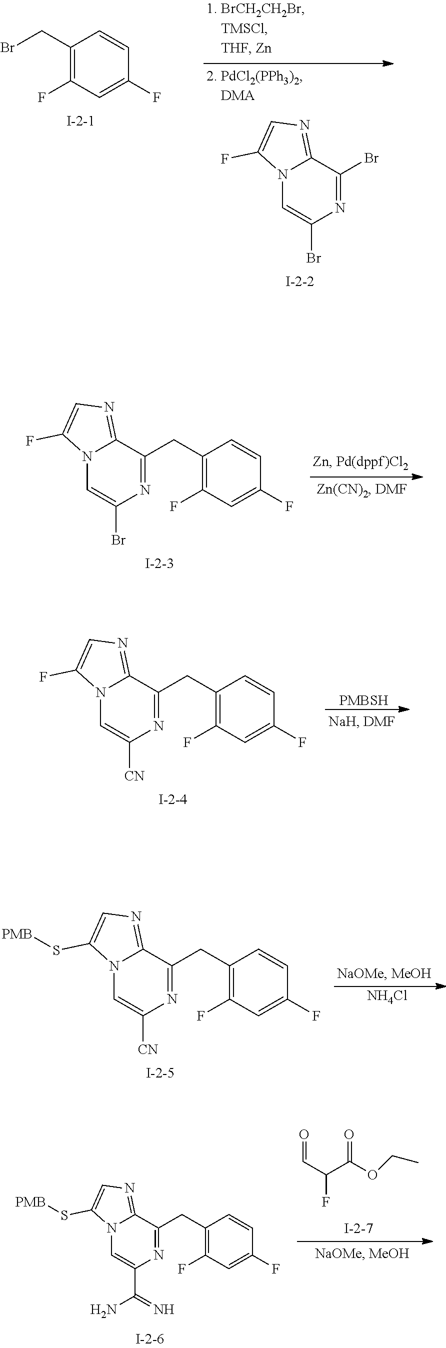

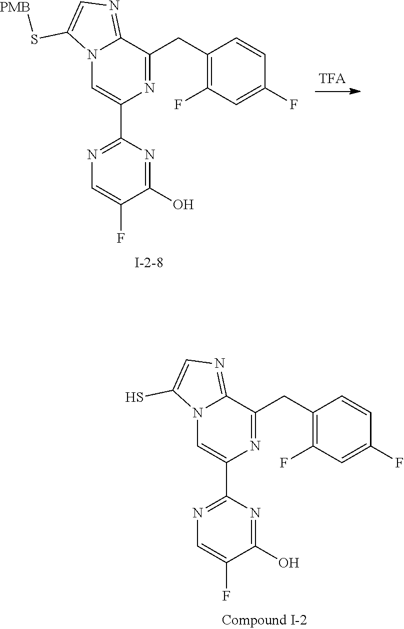

Compound I-2-1 can be transformed into the corresponding organozinc compound using zinc, dibromoethane, and TMSCL. The organozinc compound can undergo a Negishi coupling in the presence of compound I-2-2 to afford compound I-2-3. Compound I-2-3 can be further reacted in the presence of zinc, zinc cyanide, and (1,1′-bis(diphenylphosphino)ferrocene)palladium(II) dichloride to afford compound I-2-4. PMBSH can be added to compound I-2-4 in the presence of a strong base to afford compound I-2-5. The nitrile of compound I-2-5 can be reacted with ammonium chloride to afford compound I-2-6. The amidine of compound I-2-6 can be condensed with compound I-2-7 to afford cyclized compound I-2-8. Finally, the protected thiol of compound I-2-8 can be deprotected to afford Compound I-2.

### Synthesis of 6-(5-fluoro-4-hydroxypyrimidin-2-yl)-8-(2,4,5-trifluorobenzyl)imidazo[1,2-a]pyrazine-2-carbonitrile (I-3)

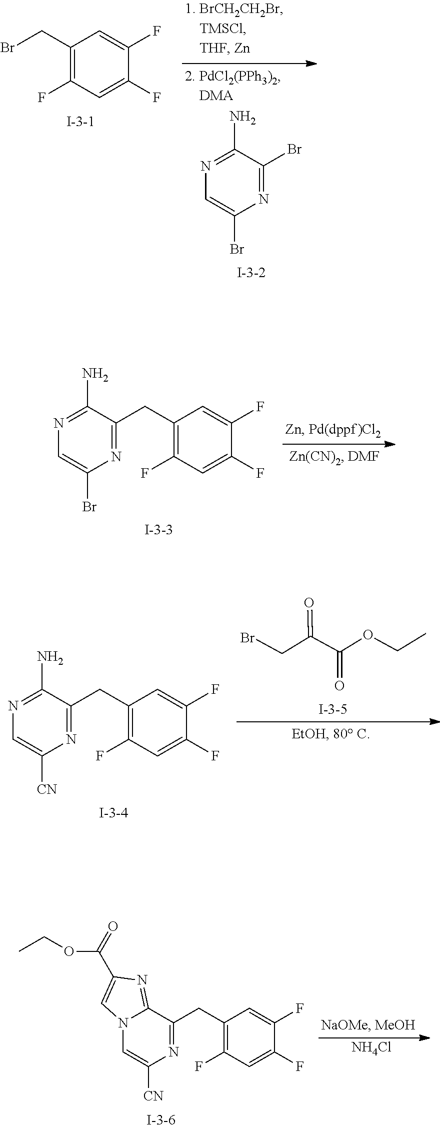

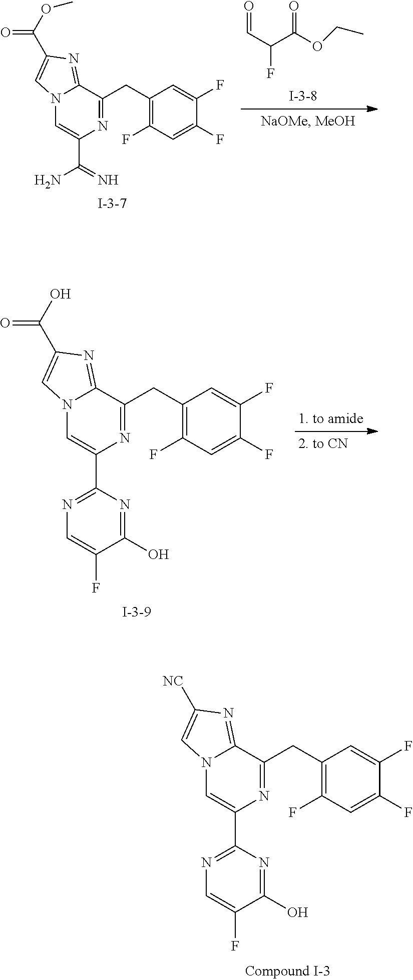

Compound I-3-1 was transformed into the corresponding organozinc compound using zinc, dibromoethane, and TMSCl. The organozinc compound underwent a Negishi coupling in the presence of compound I-3-2 to afford compound I-3-3. Compound I-3-3 was further reacted in the presence of zinc, zinc cyanide, and (1,1′-bis(diphenylphosphino)ferrocene)palladium(II) dichloride to afford compound I-3-4. Compound I-3-4 was condensed with compound compound I-3-5 in the presence of heat to afford cyclized compound compound I-3-6. The nitrile of compound I-3-6 was reacted with ammonium chloride to afford compound I-3-7. The amidine of compound I-3-7 was condensed with compound I-3-8 to afford the cyclised compound I-3-9. The carboxylic acid of compound I-3-9 was converted to the corresponding amide, which was then dehydrated to afford nitrile compound I-3.

### Synthesis of I-3-3

A solution of Zn (6.54 g, 100.031 mmol, 1.50 equiv) in THF (300 mL) was treated with 1,2-dibromoethane (0.63 g, 3.333 mmol, 0.05 equiv) for 10 min at 50° C. under a nitrogen atmosphere followed by the addition of TMSCl (0.43 mL, 3.364 mmol, 0.05 equiv), dropwise, at 50° C. 1-(bromomethyl)-2,4,5-trifluorobenzene (I-3-1, 15 g, 66.664 mmol, 1.00 equiv) was then added at room temperature. The resulting mixture was stirred for an additional 2 h at room temperature. To the above mixture were added 3,5-dibromopyrazin-2-amine (I-3-2, 13.49 g, 53.331 mmol, 0.8 equiv) and Pd(PPh₃)₂Cl₂ (0.94 g, 1.333 mmol, 0.02 equiv), at room temperature. The resulting mixture was stirred for an additional 2 h at 45° C. and the reaction was monitored by LCMS. The reaction was quenched with sat. NH₄Cl aq. (300 mL) at 0° C. and the resulting mixture was extracted with EtOAc (3×400 mL). The combined organic layers were washed with brine (2×200 mL) and dried over anhydrous Na₂SO₄. After filtration, the filtrate was concentrated under reduced pressure and the residue was purified by reversed-phase flash chromatography with the following conditions: column, C18 silica gel; mobile phase, MeCN in Water (0.1% FA), 0% to 100% gradient in 10 min; and detector UV 254 nm, to afford 5-bromo-3-[(2,4,5-trifluorophenyl)methyl]pyrazin-2-amine (I-3-3, 13.9 g, 65.5% yield) as a yellow solid. LC-MS: (ESI) m/z 318.00 [M+H].

### Synthesis of I-3-4

A solution of 5-bromo-3-[(2,4,5-trifluorophenyl)methyl]pyrazin-2-amine (I-3-3, 13.9 g, 43.697 mmol, 1 equiv), Zn(CN)₂ (5.13 g, 43.697 mmol, 1.00 equiv), Zn (0.57 g, 8.739 mmol, 0.20 equiv) and Pd(dppf)Cl₂ (0.80 g, 1.093 mmol, 0.03 equiv) in DMF (250 mL) was stirred for 2 h at 120° C. under a nitrogen atmosphere. The mixture was then allowed to cool down to room temperature. The reaction was quenched by the addition of Water (200 mL) at room temperature and the resulting mixture was extracted with EtOAc (3×200 mL). The combined organic layers were washed with water (2×100 mL) and dried over anhydrous Na₂SO₄. After filtration, the filtrate was concentrated under reduced pressure and the residue was purified by reverse-phase flash chromatography with the following conditions: column, C18 silica gel; mobile phase, MeCN in Water (0.1% FA), 0% to 100% gradient in 10 min; and detector UV 254 nm, to afford 5-amino-6-[(2,4,5-trifluorophenyl)methyl]pyrazine-2-carbonitrile (I-3-4, 10 g, 86.6% yield) as a brown solid. LC-MS: (ESI) m/z 262.9 [M−H].

### Synthesis of I-3-6

A solution of 5-amino-6-[(2,4,5-trifluorophenyl)methyl]pyrazine-2-carbonitrile (I-3-4, 10 g, 37.849 mmol, 1 equiv) and ethyl 3-bromo-2-oxopropanoate (I-3-5, 8.86 g, 45.419 mmol, 1.20 equiv) in EtOH (150 mL) was stirred overnight at 80° C. under a nitrogen atmosphere. The reaction was quenched by the addition of Water (100 mL) at room temperature. The resulting mixture was extracted with EtOAc (3×200 mL) and the combined organic layers were washed with brine (2×200 mL) and dried over anhydrous Na₂SO₄. After filtration, the filtrate was concentrated under reduced pressure and the residue was purified by reverse-phase flash chromatography with the following conditions: column, C18 silica gel; mobile phase, MeCN in Water (0.1% FA), 0% to 100% gradient in 8 min; and detector UV 254 nm, to afford ethyl 6-cyano-8-[(2,4,5-trifluorophenyl)methyl]imidazo[1,2-a]pyrazine-2-carboxylate (I-3-6, 5.7 g, 42% yield) as a yellow solid. LC-MS: (ESI) m/z 361.1[M+H].

### Synthesis of I-3-7

A solution of 2-[ethoxy(hydroxy)methyl]-8-[(2,4,5-trifluorophenyl)methyl]imidazo[1,2-a]pyrazine-6-carbonitrile (I-3-6, 5.7 g, 15.732 mmol, 1 equiv) and NaOMe (0.28 g, 1.573 mmol, 0.1 equiv, 30% wt in MeOH), in MeOH (120 mL), was stirred for 2 h at 45° C. under nitrogen atmosphere. To the above mixture was added NH₄Cl (1.68 g, 31.464 mmol, 2 equiv) at 45° C. and the resulting mixture was stirred for an additional 2 h at 45° C. The reaction was quenched with Water (50 mL) at room temperature. The aqueous layer was extracted with CH₂Cl₂/IPA at a ratio of 3/1 (3×150 mL). The combined organic extracts were then washed with brine (150 mL), dried over anhydrous Na₂SO₄, and concentrated under vacuum to yield methyl 6-carbamimidoyl-8-[(2,4,5-trifluorophenyl)methyl]imidazo[1,2-a]pyrazine-2-carboxylate (I-3-7, 5 g, crude) as a yellow solid. LC-MS: (ESI) m/z 364.1 [M+H].

### Synthesis of I-3-9

To a stirred solution of methyl 6-carbamimidoyl-8-[(2,4,5-trifluorophenyl)methyl]imidazo[1,2-a]pyrazine-2-carboxylate (I-3-7, 3.4 g, 9.359 mmol, 1 equiv) and ethyl 2-fluoro-3-oxopropanoate (I-3-8, 2.51 g, 18.718 mmol, 2.00 equiv) in MeOH (70 mL, 1728.918 mmol, 184.74 equiv) was added NaOMe (5.90 g, 32.757 mmol, 3.50 equiv, 30% wt in MeOH), dropwise at room temperature and under nitrogen atmosphere. The resulting mixture was stirred for an additional 4 h at 70° C. The mixture was then allowed to cool down to room temperature and it was acidified to pH 4 with 2 M HCl (10 mL). The resulting mixture was diluted with water (80 mL) and it was extracted with EtOAc (3×120 mL). The combined organic layers were washed with brine (2×120 mL) and dried over anhydrous Na₂SO₄. After filtration, the filtrate was concentrated under reduced pressure and the residue purified by reverse-phase flash chromatography with the following conditions: column, C18 silica gel; mobile phase, MeCN in Water (0.1% FA), 0% to 100% gradient in 10 min; and detector UV 254 nm, to afford 6-(5-fluoro-4-hydroxypyrimidin-2-yl)-8-[(2,4,5-trifluorophenyl)methyl]imidazo[1,2-a]pyrazine-2-carboxylic acid (I-3-9, 600 mg, 15.3% yield) as a yellow solid. LC-MS: MS (ESI) m/z 420.1 [M+H].

### Synthesis of I-3

To a stirred solution of 6-(5-fluoro-4-hydroxypyrimidin-2-yl)-8-[(2,4,5-trifluorophenyl)methyl]imidazo[1,2-a]pyrazine-2-carboxylic acid (I-3-9, 600 mg, 1.431 mmol, 1 equiv) and HATU (816 mg, 2.146 mmol, 1.50 equiv) in DMF (14 mL) were added TEA (434 mg, 4.289 mmol, 3.00 equiv) and NH₃ (g) (0.95 mL, 1.240 mmol, 4 equiv, 1.3 M in THF) dropwise at room temperature and under a nitrogen atmosphere. The resulting mixture was stirred for an additional 4 h at room temperature. The resulting mixture was then extracted with EtOAc (3×60 mL) and the combined organic layers were washed with brine (2×50 mL) and dried over anhydrous Na₂SO₄. After filtration, the filtrate was concentrated under reduced pressure and the residue was purified by reverse-phase flash chromatography with the following conditions: column, C18 silica gel; mobile phase, MeCN in Water (10 mmol/L NH₄HCO₃), 0% to 100% gradient in 10 min; and detector UV 254 nm, to afford 6-(5-fluoro-4-hydroxypyrimidin-2-yl)-8-[(2,4,5-trifluorophenyl)methyl]imidazo[1,2-a]pyrazine-2-carboxamide (amide intermediate, 200 mg, 33.5% yield) as a yellow solid. LC-MS: (ESI) m/z 419.15 [M+H].

A solution of the 6-(5-fluoro-4-hydroxypyrimidin-2-yl)-8-[(2,4,5-trifluorophenyl)methyl]imidazo[1,2-a]pyrazine-2-carboxamide produced as above (amide intermediate, 100 mg, 0.239 mmol, 1 equiv) in phosphorus oxychloride (2 mL) was stirred for 2 h at 90° C. under a nitrogen atmosphere. The resulting mixture was diluted with 1,4-dioxane (5 mL) and then concentrated under reduced pressure. To the above mixture were added NaOH (2.5 mL, 2 mmol) and dioxane (2 mL) at room temperature. The resulting mixture was stirred for an additional 2 h at room temperature. The resulting mixture was then extracted with EtOAc (3×20 mL) and the combined organic layers were washed with brine (2×20 mL) and dried over anhydrous Na₂SO₄. After filtration, the filtrate was concentrated under reduced pressure and the residue was purified by reverse-phase flash chromatography with the following conditions: column, C18 silica gel; mobile phase, MeCN in Water (0.1% FA), 0% to 100% gradient in 10 min; and detector UV 254 nm, to afford 6-(5-fluoro-4-hydroxypyrimidin-2-yl)-8-[(2,4,5-trifluorophenyl)methyl]imidazo[1,2-a]pyrazine-2-carbonitrile (1-3, 4.8 mg, 4% yield, purity 80.4%) as an off-white solid.

LC-MS: (ESI) m/z 410.10 [M+H].

¹H NMR: (400 MHz, Methanol-d₄) δ 9.39 (s, 1H), 8.78 (s, 1H), 8.09 (d, J=3.5 Hz, 1H), 7.50-7.41 (m, 1H), 7.18 (td, J=10.0, 6.7 Hz, 1H), 4.65 (s, 2H).

¹⁹F NMR: (400 MHz, Methanol-d₄) δ −119.26 (dd, J=15.3, 3.7 Hz), −137.90 (d, J=18.6 Hz), −145.38 (dd, J=21.2, 15.3 Hz), −153.03.

### Synthesis of 2-fluoro-4-((3-fluoro-6-(5-fluoro-4-hydroxypyrimidin-2-yl)imidazo[1,2-a]pyrazin-8-yl)methyl)-5-methylbenzonitrile (I-4) and 2-fluoro-4-((6-(5-fluoro-4-hydroxypyrimidin-2-yl)-3-methoxyimidazo[1,2-a]pyrazin-8-yl)methyl)-5-methylbenzonitrile (I-32)

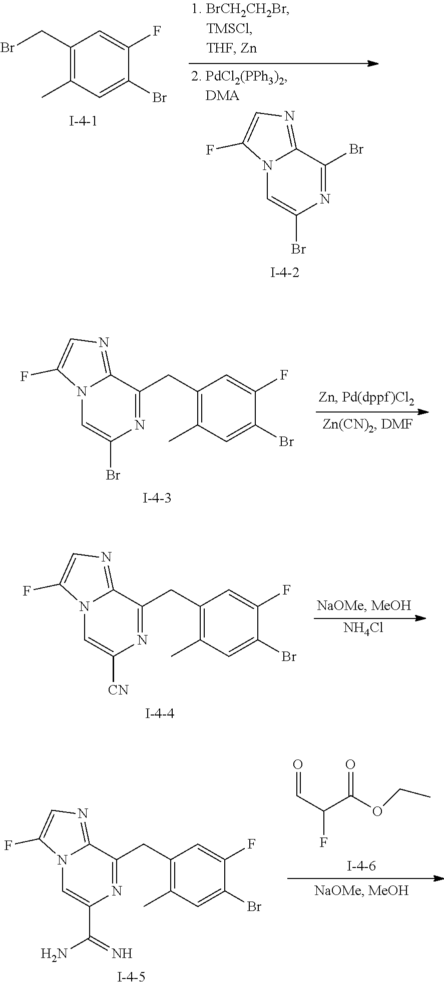

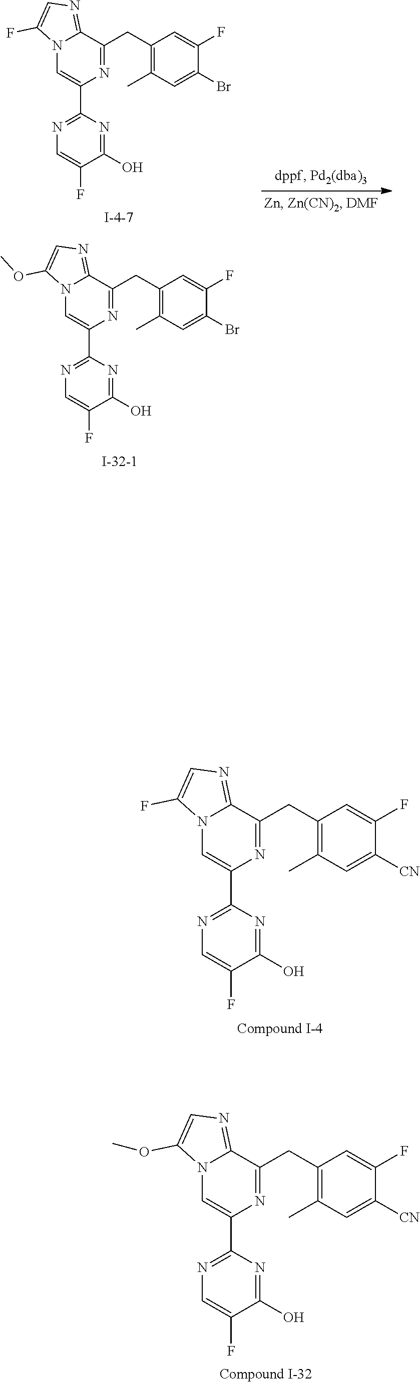 [SMILES: Cc1cc(Br)c(F)cc1Cc1nc(-c2ncc(F)c(O)n2)cn2c(F)cnc12 ; COc1cnc2c(Cc3cc(F)c(Br)cc3C)nc(-c3ncc(F)c(O)n3)cn12 ; Cc1cc(C#N)c(F)cc1Cc1nc(-c2ncc(F)c(O)n2)cn2c(F)cnc12 ; COc1cnc2c(Cc3cc(F)c(C#N)cc3C)nc(-c3ncc(F)c(O)n3)cn12]

Compound I-4-1 was transformed into the corresponding organozinc compound using zinc, dibromoethane, and TMSCl. The organozinc compound underwent a Negishi coupling in the presence of compound I-4-2 to afford compound I-4-3. Compound I-4-3 was further reacted in the presence of zinc, zinc cyanide, and (1,1′-bis(diphenylphosphino)ferrocene)palladium(II) dichloride to afford compound I-4-4. The nitrile of compound I-4-4 was reacted with ammonium chloride to afford amidine compound I-4-5. The amidine of compound I-4-5 was condensed with compound I-4-6 in NaOMe/MeOH to afford cyclized compound I-4-7 and compound I-32-1. Both cyclization products were taken forward. The aryl bromide of compound I-4-7 and compound I-32-1 were reacted in the presence of zinc, zinc cyanide, and Pd₂(dba)₃, and diphenylphosphinoferrocene to afford cyano compounds I-4 and I-32, respectivly.

### Synthesis of I-4-1

To a stirred solution of 4-bromo-5-fluoro-2-methylbenzoic acid (4.7 g, 20.169 mmol, 1 equiv) in THF (50 mL) was added BH₃-THF (30.25 mL, 30.254 mmol, 1.5 equiv, 1M in THF) dropwise at 0° C. The mixture was stirred for 3 h at 50° C. The reaction was quenched by the addition of Water (100 mL) at room temperature and the resulting mixture was extracted with EtOAc (3×100 mL). The combined organic layers were washed with brine (1×100 mL) and dried over anhydrous Na₂SO₄. After filtration, the filtrate was concentrated under reduced pressure and the residue was purified by silica gel column chromatography and eluted with petroleum ether/ethyl acetate (5:1) to afford (4-bromo-5-fluoro-2-methylphenyl)methanol (4.5 g, crude) as an off-white solid. ¹H NMR: (400 MHz, CDCl₃) δ 7.32 (d, J=6.8 Hz, 1H), 7.19 (d, J=9.4 Hz, 1H), 4.62 (s, 2H), 2.24 (s, 3H).

To a stirred solution of the (4-bromo-5-fluoro-2-methylphenyl)methanol produced above (1.5 g, 6.848 mmol, 1 equiv) in diethyl ether (20 mL) was added phosphorus tribromide (0.93 g, 3.424 mmol, 0.5 equiv) at 0° C. The mixture was stirred for 4 h at 25° C. The reaction was quenched by the addition of Water (50 mL) at room temperature and the resulting mixture was extracted with EtOAc (3×100 mL). The combined organic layers were washed with brine (1×200 mL) and dried over anhydrous Na₂SO₄. After filtration, the filtrate was concentrated under reduced pressure and the residue was purified by silica gel column chromatography, eluted with petroleum ether/ethyl acetate (5:1) to afford 1-bromo-4-(bromomethyl)-2-fluoro-5-methylbenzene (I-4-1, 2.1 g, crude) as an off-white solid. This was used directly in the next step without further purification.

### Synthesis of I-4-2

To a stirred solution of 6,8-dibromoimidazo[1,2-a]pyrazine (5 g, 18.056 mmol, 1 equiv) in acetonitrile (50 mL) was added Selectfluor® (9.59 g, 27.084 mmol, 1.5 equiv) at 25° C. The mixture was stirred for 10 h at 60° C. The reaction was quenched by the addition of Water (200 mL) at room temperature and the resulting mixture was extracted with EtOAc (3×100 mL). The combined organic layers were washed with brine (1×300 mL) and dried over anhydrous Na₂SO₄. After filtration, the filtrate was concentrated under reduced pressure and the residue was purified by silica gel column chromatography, eluted with petroleum ether/ethyl acetate (5:1) to afford 6,8-dibromo-3-fluoroimidazo[1,2-a]pyrazine (I-4-2, 1.5 g, 28% yield) as a light yellow solid. LC-MS: (ESI) m/z 295.9 [M+H]. ¹H NMR: (400 MHz, CDCl₃) δ 8.12 (s, 1H), 7.58 (d, J=6.8 Hz, 1H).

### Synthesis of I-4-3

To a stirred solution of Zn (221.7 mg, 3.390 mmol, 2 equiv) in anhydrous THF (5 mL) was added dibromoethane (31.8 mg, 0.170 mmol, 0.1 equiv) dropwise at 25° C. and under a nitrogen atmosphere. The mixture was stirred for 10 min at 50° C. under the nitrogen atmosphere. TMSCl (18.42 mg, 0.170 mmol, 0.1 equiv) was then added and the mixture was cooled to ambient temperature. Then 1-bromo-4-(bromomethyl)-2-fluoro-5-methylbenzene (I-4-1, 717.0 mg, 2.542 mmol, 1.5 equiv) was added dropwise at 0° C. still under the nitrogen atmosphere. The mixture was stirred for 2 h at 25° C. under the nitrogen atmosphere. 6,8-dibromo-3-fluoroimidazo[1,2-a]pyrazine (I-4-2, 500 mg, 1.695 mmol, 1 equiv) and Pd(PPh₃)₂Cl₂ (23.8 mg, 0.034 mmol, 0.02 equiv) were then added at 25° C. still under the nitrogen atmosphere. The mixture was stirred for 4 h at 50° C. under the nitrogen atmosphere. The reaction was quenched by the addition of Water (50 mL) at room temperature and the resulting mixture was extracted with EtOAc (3×100 mL). The combined organic layers were washed with brine (1×200 mL) and dried over anhydrous Na₂SO₄. After filtration, the filtrate was concentrated under reduced pressure and the residue was purified by silica gel column chromatography, eluted with petroleum ether/ethyl acetate (5:1) to afford 6-bromo-8-[(4-bromo-5-fluoro-2-methylphenyl)methyl]-3-fluoroimidazo[1,2-a]pyrazine (I-4-3, 600 mg, 85% yield) as a light yellow solid. LC-MS: (ESI) m/z 417.9 [M+H]. ¹H NMR: (400 MHz, CDCl₃) δ 7.98 (s, 1H), 7.40 (d, J=7.0 Hz, 1H), 7.33 (d, J=7.1 Hz, 1H), 7.14 (d, J=9.5 Hz, 1H), 4.44 (s, 2H), 2.45 (s, 3H).

### Synthesis of I-4-4

To a stirred solution of 6-bromo-8-[(4-bromo-5-fluoro-2-methylphenyl)methyl]-3-fluoroimidazo[1,2-a]pyrazine (I-4-3, 600 mg, 1.439 mmol, 1 equiv) in anhydrous DMF (5 mL) was added zinc cyanide (101.3 mg, 0.863 mmol, 0.6 equiv), Zn (18.8 mg, 0.288 mmol, 0.2 equiv) and Pd(dppf)Cl₂ (26.3 mg, 0.036 mmol, 0.025 equiv) at 25° C. under a nitrogen atmosphere. The mixture was stirred for 2 h at 120° C. under the nitrogen atmosphere. The reaction was then quenched by the addition of Water (100 mL) at room temperature and the resulting mixture was extracted with EtOAc (3×100 mL). The combined organic layers were washed with brine (1×200 mL) and dried over anhydrous Na₂SO₄. After filtration, the filtrate was concentrated under reduced pressure and the residue was purified by silica gel column chromatography, eluted with petroleum ether/ethyl acetate (5:1) to afford 8-[(4-bromo-5-fluoro-2-methylphenyl)methyl]-3-fluoroimidazo[1,2-a]pyrazine-6-carbonitrile (I-4-4, 365 mg, 70% yield) as an off-white solid. LC-MS: (ESI) m/z 363.0 [M+H]. ¹H NMR: (400 MHz, CDCl₃) δ 8.30 (s, 1H), 7.51 (d, J=7.1 Hz, 1H), 7.35 (d, J=7.0 Hz, 1H), 7.18 (d, J=9.4 Hz, 1H), 4.47 (s, 2H), 2.44 (s, 3H).

### Synthesis of I-4-5

To a stirred solution of 8-[(4-bromo-5-fluoro-2-methylphenyl)methyl]-3-fluoroimidazo[1,2-a]pyrazine-6-carbonitrile (I-4-4, 365 mg, 1.005 mmol, 1 equiv) in MeOH (5 mL) was added NaOMe (18.1 mg, 0.100 mmol, 0.1 equiv, 30% wt in MeOH) at 25° C. The mixture was stirred for 4 h at 70° C., at which point NH₄Cl (107.5 mg, 2.010 mmol, 2 equiv) was added and the mixture was stirred for an additional 4 h at 70° C. The reaction was quenched by the addition of Water (10 mL) at room temperature and the resulting mixture was extracted with DCM (3×100 mL). The combined organic layers were washed with brine (1×100 mL) and dried over anhydrous Na₂SO₄. After filtration, the filtrate was concentrated under reduced pressure to afford 8-[(4-bromo-5-fluoro-2-methylphenyl)methyl]-3-fluoroimidazo[1,2-a]pyrazine-6-carboximidamide (I-4-5, 392 mg, crude) as a brown solid. LC-MS: (ESI) m/z 380.0 [M+H].

### Synthesis of I-4-6

To a stirred solution of NaH (0.23 g, 9.425 mmol, 1 equiv) in Et₂O (50 mL) was added EtOH (0.05 mL, 0.943 mmol, 0.1 equiv) at 0° C. The mixture was stirred for 10 min at 0° C. Ethyl 2-fluoroacetate (1 g, 9.425 mmol, 1 equiv) and ethyl formate (0.70 g, 9.425 mmol, 1 equiv) were added at 0° C. The resulting mixture was stirred overnight at room temperature under nitrogen atmosphere and then concentrated under reduced pressure to afford ethyl 2-fluoro-3-oxo-2-sodiopropanoate (I-4-6, 1.5 g, crude) as a light yellow solid which was directly used in next step.

### Synthesis of I-4-7

To a stirred solution of 8-[(4-bromo-5-fluoro-2-methylphenyl)methyl]-3-fluoroimidazo[1,2-a]pyrazine-6-carboximidamide (I-4-5, 390 mg, 1.026 mmol, 1 equiv) in MeOH (5 mL) was added ethyl 2-fluoro-3-oxo-2-sodiopropanoate (I-4-6, 275.1 mg, 2.052 mmol, 2 equiv) and NaOMe (369.4 mg, 2.052 mmol, 2 equiv, 30% in MeOH) at 25° C. The mixture was stirred for 8 h at 70° C. The reaction was quenched by the addition of Water (50 mL) at room temperature and then acidified to pH 3-4 with conc HCl (10 mL). The resulting mixture was extracted with EtOAc (3×100 mL). The combined organic layers were washed with brine (1×200 mL) and dried over anhydrous Na₂SO₄. After filtration, the filtrate was concentrated under reduced pressure and the residue was purified by silica gel column chromatography, eluted with DCM/MeOH (30:1) to afford 2-{8-[(4-bromo-5-fluoro-2-methylphenyl)methyl]-3-fluoroimidazo[1,2-a]pyrazine-6-yl}-5-fluoropyrimidin-4-ol (I-4-7, 280 mg, 60.5% yield) as a brown solid.

LC-MS: (ESI) m/z 449.95 [M+H].

### Synthesis of I-4

To a stirred solution of 2-{8-[(4-bromo-5-fluoro-2-methylphenyl)methyl]-3-fluoroimidazo[1,2-a]pyrazin-6-yl}-5-fluoropyrimidin-4-ol (I-4-7, 265 mg, 0.589 mmol, 1 equiv) in anhydrous DMF (5 mL) was added Zn(CN)₂ (138.2 mg, 1.178 mmol, 2 equiv), Zn (7.7 mg, 0.118 mmol, 0.2 equiv) and Pd(dppf)Cl₂ (43.0 mg, 0.059 mmol, 0.1 equiv) at 25° C. under nitrogen atmosphere. The mixture was stirred for 2 h at 120° C. under the nitrogen atmosphere. The mixture was then allowed to cool down to room temperature. The reaction was quenched with Water (3 mL) at room temperature and the resulting mixture was purified by reverse-phase flash chromatography with the following conditions: column, C18 silica gel; mobile phase, MeCN in Water (10 mmol/L NH₄HCO₃), 5% to 80% gradient in 15 min; and detector, UV 254/220 nm, to afford 2-fluoro-4-{[3-fluoro-6-(5-fluoro-4-hydroxypyrimidin-2-yl)imidazo[1,2-a]pyrazin-8-yl]methyl}-5-methylbenzonitrile (I-4, 45.4 mg, 91% purity, 18% yield) as an off-white solid.

LC-MS: (ESI) m/z 397.05 [M+H]. ¹H NMR: (400 MHz, DMSO-d₆) δ 12.80 (s, 1H), 8.99 (s, 1H), 8.20 (d, J=3.6 Hz, 1H), 7.75 (d, J=6.9 Hz, 2H), 7.63 (d, J 10.6 Hz, 1H), 4.57 (s, 2H), 2.50 (s, 3H). ¹⁹F NMR: (376 MHz, DMSO-d₆) δ −113.32, −150.46, −152.09.

Compound I-32 can also be isolated according to the above scheme, as an impurity derived from I-32-1, which is formed in the step towards the synthesis of I-4-7 or, alternatively it can be made by a different process as described below.

### Synthesis of 2-fluoro-4-((6-(5-fluoro-4-hydroxypyrimidin-2-yl)-3-methoxyimidazo[1,2-a]pyrazin-8-yl)methyl)-5-methylbenzonitrile (I-32)

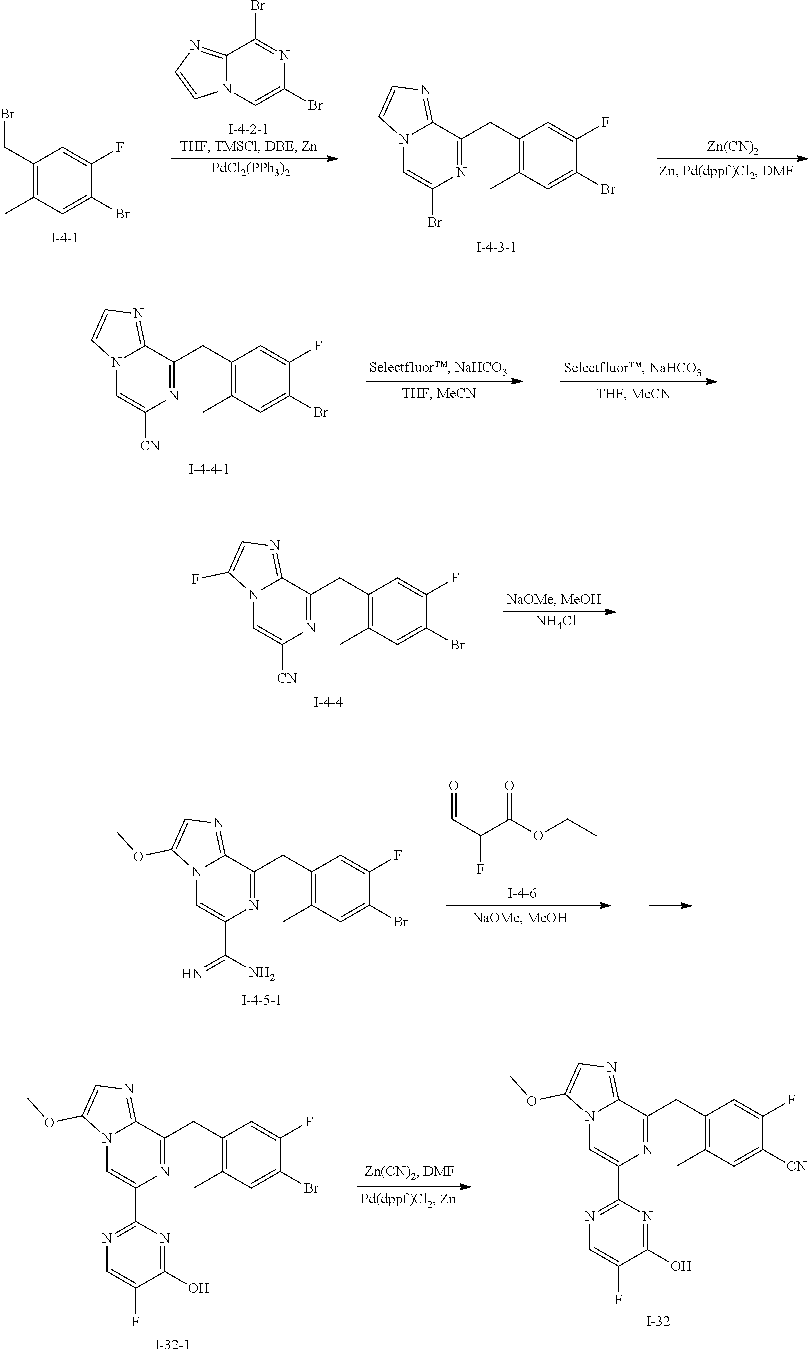

Compound I-32 can be made in the following way, as summarized in the above scheme. Compound I-4-1 was transformed into the corresponding organozinc compound using zinc, dibromoethane, and TMSCl. The organozinc compound underwent a Negishi coupling in the presence of compound I-4-2-1 to afford compound I-4-3-1. Compound I-4-3-1 was further reacted in the presence of zinc, zinc cyanide, and (1,1′-bis(diphenylphosphino)ferrocene)palladium(II) dichloride to afford compound I-4-4-1. Compound I-4-4-1 was fluorinated using Selectfluor™ to afford compound I-4-4. The nitrile of compound I-4-4 was reacted with ammonium chloride in the presence of sodium methoxide in methanol to afford amidine compound I-4-5-1. The amidine of compound I-4-5-1 was condensed with compound I-4-6 in NaOMe/MeOH to afford cyclized compound I-32-1. The aryl bromide of compound I-32-1 was reacted in the presence of zinc, zinc cyanide, and (1,1′-bis(diphenylphosphino)ferrocene)palladium(II) dichloride to afford cyano compound I-32.

### Synthesis of I-4-3-1

To a stirred solution of Zn (1.19 g, 18.128 mmol, 2 equiv) in anhydrous THF (40 mL) was added dibromoethane (DBE, 0.17 g, 0.906 mmol, 0.1 equiv) at 25° C. under nitrogen atmosphere. The mixture was stirred for 10 min at 50° C. under the nitrogen atmosphere. TMSCl (0.10 g, 0.906 mmol, 0.1 equiv) was added and the mixture was cooled to ambient temperature. Then, 1-bromo-4-(bromomethyl)-2-fluoro-5-methylbenzene (I-4-1, 3.83 g, 13.596 mmol, 1.5 equiv) was added dropwise at 0° C. under nitrogen atmosphere. The mixture was stirred for 2 h at 25° C. under the nitrogen atmosphere. 6,8-dibromoimidazo[1,2-a]pyrazine (I-4-2-1, 2.51 g, 9.064 mmol, 1 equiv) and Pd(PPh₃)₂Cl₂ (0.13 g, 0.181 mmol, 0.02 equiv) were then added at 25° C. under nitrogen atmosphere. The resulting mixture was stirred for 4 h at 50° C. under the nitrogen atmosphere. The reaction was then quenched by the addition of Water (100 mL) at room temperature and the resulting mixture was extracted with EtOAc (3×100 mL). The combined organic layers were washed with brine (1×200 mL) and dried over anhydrous Na₂SO₄. After filtration, the filtrate was concentrated under reduced pressure and the residue was purified by silica gel column chromatography, eluted with petroleum ether/ethyl acetate (5:1), to afford 6-bromo-8-[(4-bromo-5-fluoro-2-methylphenyl)methyl]imidazo [1,2-a]pyrazine (I-4-3-1, 3.46 g, 96% yield) as an off-white solid. LC-MS: (ESI) m/z 399.9 [M+H].

### Synthesis of I-4-4-1

To a stirred solution of 6-bromo-8-[(4-bromo-5-fluoro-2-methylphenyl)methyl]imidazo[1,2-a]pyrazine (I-4-3-1, 3.46 g, 8.670 mmol, 1 equiv) in anhydrous DMF (30 mL) were added zinc cyanide (0.51 g, 4.335 mmol, 0.5 equiv), Zn (0.11 g, 1.734 mmol, 0.2 equiv) and Pd(dppf)Cl₂ (0.13 g, 0.173 mmol, 0.02 equiv). The mixture was stirred for 2 h at 120° C. under a nitrogen atmosphere. The reaction was quenched by the addition of Water (200 mL) at room temperature and the resulting mixture was extracted with EtOAc (3×100 mL). The combined organic layers were washed with brine (1×200 mL) and dried over anhydrous Na₂SO₄. After filtration, the filtrate was concentrated under reduced pressure and the residue was purified by silica gel column chromatography, eluted with petroleum ether/ethyl acetate (5:1) to afford 8-[(4-bromo-5-fluoro-2-methylphenyl) methyl]imidazo[1,2-a]pyrazine-6-carbonitrile (I-4-4-1, 2.2 g, 73.5% yield) as a light yellow solid. LC-MS: (ESI) m/z 345.0 [M+H].

### Synthesis of I-4-4

To a stirred solution of 8-[(4-bromo-5-fluoro-2-methylphenyl)methyl]imidazo[1,2-a]pyrazine-6-carbonitrile (I-4-4-1, 2.2 g, 6.374 mmol, 1 equiv) in THF (10 mL) and MeCN (20 mL) were added Selectfluor® (4.52 g, 12.748 mmol, 2 equiv) and NaHCO₃ (1.34 g, 15.935 mmol, 2.5 equiv) at 25° C. under a nitrogen atmosphere. The mixture was stirred for 12 h at 80° C. under the nitrogen atmosphere. The reaction was quenched by the addition of Water (100 mL) at room temperature and the resulting mixture was extracted with EtOAc (3×100 mL). The combined organic layers were washed with brine (1×200 mL) and dried over anhydrous Na₂SO₄. After filtration, the filtrate was concentrated under reduced pressure and the residue was purified by silica gel column chromatography, eluted with petroleum ether/ethyl acetate (5:1) to afford 8-[(4-bromo-5-fluoro-2-methylphenyl)methyl]-3-fluoroimidazo[1,2-a]pyrazine-6-carbonitrile (I-4-4, 1.3 g, 56% yield) as an off-white solid. LC-MS: MS (ESI) m/z 363.0 [M+H].

### Synthesis of I-4-5-1

To a stirred solution of 8-[(4-bromo-5-fluoro-2-methylphenyl)methyl]-3-fluoroimidazo[1,2-a]pyrazine-6-carbonitrile (I-4-4, 980 mg, 2.698 mmol, 1 equiv) in MeOH (5 mL) was added NaOMe (48.5 mg, 0.270 mmol, 0.1 equiv, 30% wt in MeOH) at 25° C. The mixture was stirred for 3 h at 70° C. MeONa (437.3 mg, 8.094 mmol, 3 equiv) was then added and the mixture was stirred for an additional 4 h at 70° C. Then NH₄Cl (288.6 mg, 5.396 mmol, 2 equiv) was added and the mixture was stirred for another 5 h at 70° C. The reaction was quenched by the addition of sat. NaHCO₃ (aq.) (20 mL) at room temperature and the resulting mixture was extracted with IPA/DCM=1:3 (3×100 mL). The combined organic layers were washed with brine (1×200 mL) and dried over anhydrous Na₂SO₄. After filtration, the filtrate was concentrated under reduced pressure to afford 8-[(4-bromo-5-fluoro-2-methylphenyl)methyl]-3-methoxyimidazo[1,2-a]pyrazine-6-carboximidamide (I-4-5-1, 1.1 g, crude) as a brown solid. LC-MS (ESI) m/z 392.0 [M+H].

### Synthesis of I-32-1

To a stirred solution of 8-[(4-bromo-5-fluoro-2-methylphenyl)methyl]-3-methoxyimidazo[1,2-a]pyrazine-6-carboximidamide (I-4-5-1, 1.1 g, 2.804 mmol, 1 equiv) in MeOH (6 mL) were added ethyl 2-fluoro-3-oxopropanoate (I-4-6, 940.2 mg, 7.010 mmol, 2.5 equiv) and NaOMe (1.51 g, 8.412 mmol, 3 equiv, 30% wt in MeOH) at 25° C. The mixture was stirred for 8 h at 70° C. The reaction was then quenched by the addition of 1 M HCl (aq.) (10 mL) at room temperature and the resulting mixture was extracted with EtOAc (3×100 mL). The combined organic layers were washed with brine (lx 200 mL) and dried over anhydrous Na₂SO₄. After filtration, the filtrate was concentrated under reduced pressure and the residue was purified by silica gel column chromatography, eluted with DCM/MeOH (30:1) to afford 2-{8-[(4-bromo-5-fluoro-2-methylphenyl)methyl]-3-methoxyimidazo[1,2-a]pyrazin-6-yl}-5-fluoropyrimidin-4-ol (I-32-1, 402 mg, 31% yield) as an off-white solid. LC-MS (ESI) m/z 462.0 [M+H].

### Synthesis of I-32

To a stirred solution of 2-{8-[(4-bromo-5-fluoro-2-methylphenyl)methyl]-3-methoxyimidazo[1,2-a]pyrazin-6-yl}-5-fluoropyrimidin-4-ol (I-32-1, 200 mg, 0.433 mmol, 1 equiv) in anhydrous DMF (3 mL) were added Zn(CN)₂ (101.6 mg, 0.866 mmol, 2 equiv), Pd(dppf)Cl₂ (6.3 mg, 0.009 mmol, 0.02 equiv) and Zn (5.6 mg, 0.087 mmol, 0.2 equiv) at 25° C. under a nitrogen atmosphere. The mixture was stirred for 16 h at 120° C. under the nitrogen atmosphere. The mixture was then allowed to cool down to room temperature. The resulting mixture was purified by reversed-phase flash chromatography with the following conditions: column, C18 silica gel; mobile phase, MeCN in Water (0.1% TFA), 5% to 80% gradient in 10 min; and detector, UV 254/220 nm, to afford 2-fluoro-4-{[6-(5-fluoro-4-hydroxypyrimidin-2-yl)-3-methoxyimidazo [1,2-a]pyrazin-8-yl]methyl}-5-methylbenzonitrile (I-32, 40.1 mg, 22% yield) as an off-white solid. LC-MS (ESI) m/z 409.10 [M+H]. ¹H NMR: (400 MHz, DMSO-d₆) δ 8.68 (s, 1H), 8.08 (d, J=3.8 Hz, 1H), 7.58 (d, J=10.6 Hz, 1H), 7.46 (s, 1H), 4.52 (s, 2H), 4.12 (s, 3H), 2.50 (s, 3H). ¹⁹F NMR: (376 MHz, DMSO-d₆) δ −113.38, −153.49.

### Synthesis of 2-(8-(2,5-difluoro-4-methylbenzyl)-[1,2,4]triazolo[1,5-a]pyrazin-6-yl)-5-fluoro-6-(fluoromethyl)pyrimidin-4-ol (I-5) and 2-(8-(2,5-difluoro-4-methylbenzyl)-[1,2,4]triazolo[1,5-a]pyrazin-6-yl)-6-(ethoxymethyl)-5-fluoropyrimidin-4-ol (I-44)

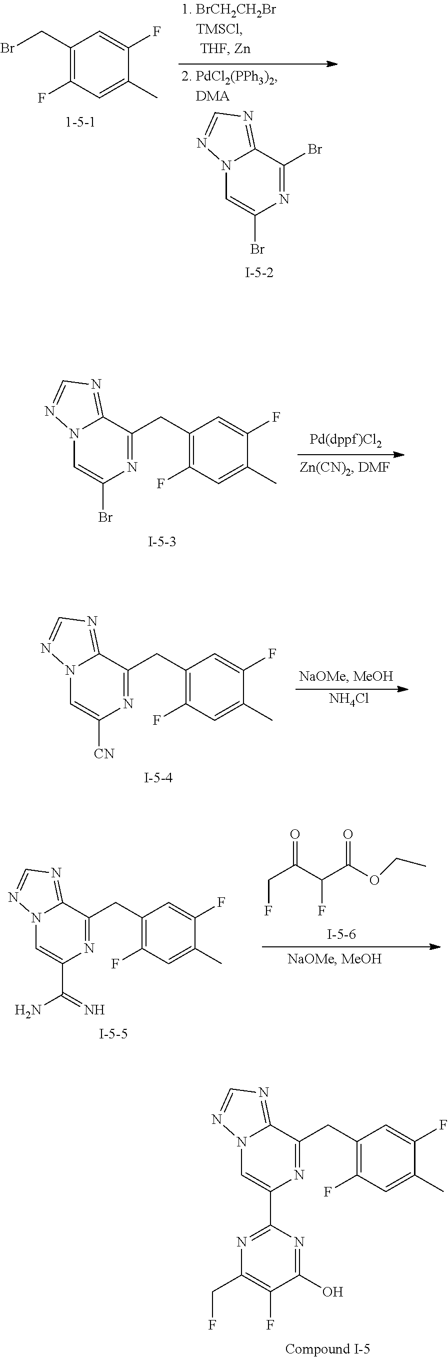

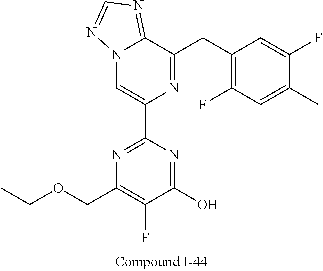 [SMILES: CCOCc1nc(-c2cn3ncnc3c(Cc3cc(F)c(C)cc3F)n2)nc(O)c1F]

Compound I-5-1 was transformed into the corresponding organozinc compound using zinc, dibromoethane, and TMSCl. The organozinc compound underwent a Negishi coupling in the presence of compound I-5-2 to afford compound I-5-3. Compound I-5-3 was further reacted in the presence of zinc, zinc cyanide, and (1,1′-bis(diphenylphosphino)ferrocene)palladium(II) dichloride to afford compound I-5-4. The nitrile of compound I-5-4 was reacted with ammonium chloride to afford amidine compound I-5-5. The amidine of compound I-5-5 was condensed with compound I-5-6 to afford cyclized compound I-5 and compound I-44.

### Synthesis of I-5-6

To a stirred solution of ethyl 2-fluoroacetate (500 mg, 4.713 mmol, 1 equiv) in Et₂O (10 mL) was added NaH (282.7 mg, 11.783 mmol, 2.5 equiv, 60% mineral oil) in portions at room temperature under a nitrogen atmosphere. The resulting mixture was stirred for 4 h at 40° C. under the nitrogen atmosphere. The mixture then was allowed to cool down to room temperature. The reaction was quenched by the addition of Water/Ice (40 mL) and concentrated H₂SO₄ (1 mL) at 0° C. The resulting mixture was extracted with Et₂O (3×20 mL) and the combined organic layers were washed with brine (2×10 mL) and dried over anhydrous Na₂SO₄. After filtration, the filtrate was concentrated under reduced pressure to afford crude product ethyl 2,4-difluoro-3-oxobutanoate (I-5-6, 600 mg, crude) as a light yellow oil.

### Synthesis of I-5-1

To a stirred solution of 2,5-difluoro-4-methylbenzoic acid (500 mg, 2.905 mmol, 1 equiv) in THF (2.5 mL) was added BH₃-THF (499.2 mg, 5.810 mmol, 2 equiv, 1M in THF) dropwise at 0° C. under nitrogen atmosphere. The resulting mixture was stirred for 2 h at 60° C. under a nitrogen atmosphere. The mixture was allowed to cool down to room temperature. The reaction was quenched by the addition of brine (10 mL) at room temperature. The resulting mixture was diluted with water (20 mL) and extracted with EtOAc (3×20 mL). The combined organic layers were dried over anhydrous Na₂SO₄. After filtration, the filtrate was concentrated under reduced pressure and the residue was purified by silica gel column chromatography, eluting with petroleum ether/ethyl acetate (5:1) to afford (2,5-difluoro-4-methylphenyl)methanol (380 mg, 83% yield) as an off-white solid. ¹H NMR: (400 MHz, DMSO-d₆) δ 7.20-7.07 (m, 2H), 5.32 (t, J=5.7 Hz, 1H), 4.49 (d, J=5.7 Hz, 2H), 2.21 (s, J=2.0 Hz, 3H).

To a stirred solution of (2,5-difluoro-4-methylphenyl)methanol produced as above (380 mg, 2.403 mmol, 1 equiv) in Et₂O (5 mL) was added phosphorus tribromide (260.2 mg, 0.961 mmol, 0.4 equiv) at 0° C. under a nitrogen atmosphere. The resulting mixture was stirred for 2 h at room temperature under a nitrogen atmosphere. The reaction was quenched by the addition of Water/Ice (20 mL) at room temperature. The organic layers were washed with sat. NaHCO₃ (1×10 mL) and dried over anhydrous Na₂SO₄. After filtration, the filtrate was concentrated under reduced pressure to afford 1-(bromomethyl)-2,5-difluoro-4-methylbenzene (I-5-1, 350 mg, crude) as a light yellow oil. ¹H NMR: (400 MHz, DMSO-d₆) δ 7.45-7.34 (m, 1H), 7.22 (dd, J=10.2, 6.3 Hz, 1H), 4.65 (s, J=1.1 Hz, 2H), 2.23 (s, J=2.1 Hz, 3H).

### Synthesis of I-5-3

To a stirred mixture of Zn (155.3 mg, 2.374 mmol, 1.5 equiv) in THF (5 mL) was added dibromoethane (14.9 mg, 0.079 mmol, 0.05 equiv) dropwise at 50° C. under a nitrogen atmosphere. The resulting mixture was stirred for 10 min at 50° C. under the nitrogen atmosphere. To the above mixture was then added TMSCl (8.6 mg, 0.079 mmol, 0.05 equiv) and the resulting mixture was stirred for an additional 10 min at room temperature. To this mixture was added 1-(bromomethyl)-2,5-difluoro-4-methylbenzene (I-5-1, 350 mg, 1.583 mmol, 1 equiv) at 0° C. The resulting mixture was then stirred for an additional 15 min at 0° C. and then 2 h at room temperature. To this mixture were then added 6,8-dibromo-[1,2,4]triazolo[1,5-a]pyrazine (1-5-2, 352.0 mg, 1.266 mmol, 0.8 equiv) and Pd(PPh₃)₂Cl₂ (33.3 mg, 0.047 mmol, 0.03 equiv) at room temperature. The final resulting mixture was stirred for an additional 1 h at 40° C., after which, it was filtered. The filter cake was washed with THF (1×4 mL), the filtrate was concentrated under reduced pressure and the residue was purified by reverse-phase flash chromatography with the following conditions: column, C18 silica gel; mobile phase, MeCN in Water (0.1% formic acid), 0% to 100% gradient in 10 min; and detector, UV 254 nm, to afford 6-bromo-8-[(2,5-difluoro-4-methylphenyl)methyl]-[1,2,4]triazolo[1,5-a]pyrazine (I-5-3, 240 mg, 45% yield) as a light yellow solid. LC-MS: (ESI) m/z 338.85 [M+H].

### Synthesis of I-5-4

To a stirred mixture of 6-bromo-8-[(2,5-difluoro-4-methylphenyl)methyl]-[1,2,4]triazolo[1,5-a]pyrazine (I-5-3, 240 mg, 0.708 mmol, 1 equiv) and Zn(CN)₂ (49.9 mg, 0.425 mmol, 0.6 equiv) in DMF (4 mL) were added Pd(dppf)Cl₂ (13.0 mg, 0.018 mmol, 0.025 equiv) and Zn (9.3 mg, 0.142 mmol, 0.2 equiv), at room temperature under a nitrogen atmosphere. The resulting mixture was stirred for 2 h at 120° C. under the nitrogen atmosphere. The mixture was then allowed to cool down to room temperature. The resulting mixture was filtered and the filtrate was purified by reverse-phase flash chromatography with the following conditions: column, C18 silica gel; mobile phase, MeCN in Water (0.1% formic acid), 0% to 100% gradient in 10 min; detector, UV 254 nm, to afford 8-[(2,5-difluoro-4-methylphenyl)methyl]-[1,2,4]triazolo[1,5-a]pyrazine-6-carbonitrile (136 mg, 67% yield) as a light yellow solid. LC-MS (ESI) m/z 286.10 [M+H].

### Synthesis of I-5-5

To a stirred solution of 8-[(2,5-difluoro-4-methylphenyl)methyl]-[1,2,4]triazolo[1,5-a]pyrazine-6-carbonitrile (I-5-4, 136 mg, 0.477 mmol, 1 equiv) in methanol (2 mL) was added NaOMe (2.6 mg, 0.048 mmol, 0.1 equiv, 30% wt in MeOH) at room temperature under a nitrogen atmosphere. The resulting mixture was stirred for 3 h at 40° C. under the nitrogen atmosphere. To the above mixture was then added NH₄Cl (51.0 mg, 0.954 mmol, 2 equiv) at room temperature and the resulting mixture was stirred overnight at 40° C. The mixture was allowed to cool down to room temperature. The reaction was quenched by the addition of sat. NaHCO₃ (aq.) (5 mL) at room temperature and the resulting mixture was diluted with water (20 mL) and extracted with CH₂Cl₂/IPA=3/1 (3×10 mL) with the combined organic layers were dried over anhydrous Na₂SO₄. After filtration, the filtrate was concentrated under reduced pressure to afford 8-[(2,5-difluoro-4-methylphenyl)methyl]-[1,2,4]triazolo[1,5-a]pyrazine-6-carboximidamide (I-5-5, 130 mg, crude) as a light yellow solid. LC-MS: (ESI) m/z 303.20 [M+H].

### Synthesis of Compounds I-5 and I-44

To a stirred mixture of 8-[(2,5-difluoro-4-methylphenyl)methyl]-[1,2,4]triazolo[1,5-a]pyrazine-6-carboximidamide (I-5-5, 130 mg, 0.430 mmol, 1 equiv) and ethyl 2,4-difluoro-3-oxobutanoate (I-5-6, 107.2 mg, 0.645 mmol, 1.5 equiv) in MeOH (2 mL) was added NaOMe (232.3 mg, 1.290 mmol, 3 equiv, 30% wt in MeOH) at room temperature under a nitrogen atmosphere. The resulting mixture was stirred for 1.5 h at 70° C. under the nitrogen atmosphere. The reaction was then quenched by the addition of Water (1 mL) at room temperature and the residue was purified by reverse-phase flash chromatography with the following conditions: column, C18 silica gel; mobile phase, MeCN in Water (0.1% formic acid), 0% to 100% gradient in 10 min; and detector, UV 254 nm, to afford 2-{8-[(2,5-difluoro-4-methylphenyl)methyl]-[1,2,4]triazolo[1,5-a]pyrazin-6-yl}-5-fluoro-6-(fluoromethyl)pyrimidin-4-ol (I-5, 44.0 mg, 25% yield) as an off-white solid and 2-{8-[(2,5-difluoro-4-methylphenyl)methyl]-[1,2,4]triazolo[1,5-a]pyrazin-6-yl}-6-(ethoxymethyl)-5-fluoropyrimidin-4-ol (I-44, 30.0 mg, 16% yield) as an off-white solid.

I-5=LC-MS: (ESI) m/z 405.15 [M+H].

¹H NMR: (400 MHz, DMSO-d₆) δ 9.55 (s, 1H), 8.82 (s, 1H), 7.34 (dd, J=9.9, 6.4 Hz, 1H), 7.16 (dd, J=9.9, 6.5 Hz, 1H), 5.44 (dd, J=46.9, 2.7 Hz, 2H), 4.57 (s, 2H), 2.18 (s, J=1.8 Hz, 3H).

¹⁹F NMR: (376 MHz, DMSO-d₆) δ −123.09 (d, J=18.1 Hz), −123.21 (d, J=18.3 Hz), −150.51, −220.47 (d, J=6.6 Hz).

I-44=LC-MS: (ESI) m/z 431.20 [M+H].

¹H NMR: (400 MHz, DMSO-d₆) δ 13.09 (s, 1H), 9.54 (s, 1H), 8.84 (s, 1H), 7.36 (dd, J=9.9, 6.4 Hz, 1H), 7.17 (dd, J=9.8, 6.6 Hz, 1H), 4.57 (s, 4H), 4.48 (d, J=3.0 Hz, 2H), 2.18 (s, J=1.9 Hz, 3H), 1.16 (t, J=7.0 Hz, 3H).

¹⁹F NMR: (376 MHz, DMSO-d₆) δ −123.10 (d, J=18.1 Hz), −123.21 (d, J=18.3 Hz), −151.04.

### Synthesis of 2-(8-(4-chloro-2,5-difluorobenzyl)-3-methylimidazo[1,2-a]pyrazin-6-yl)-5-fluoropyrimidine-4,6-diol (I-6) and 2,5-difluoro-4-((6-(5-fluoro-4,6-dihydroxypyrimidin-2-yl)-3-methylimidazo[1,2-a]pyrazin-8-yl)methyl)benzonitrile (I-42)

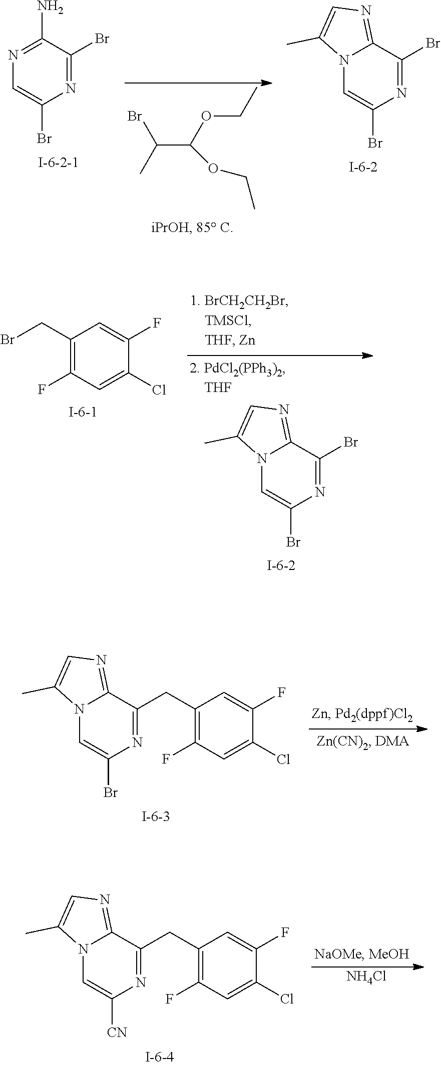

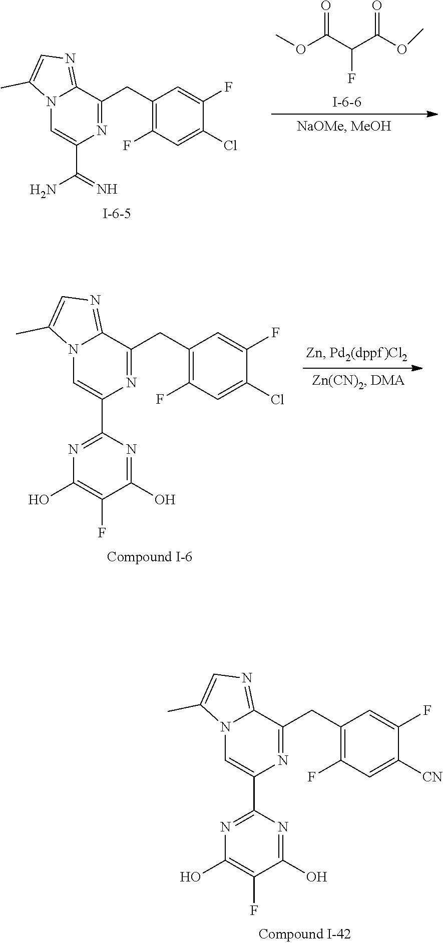

Compound I-6-1 can be transformed into the corresponding organozinc compound using zinc, dibromoethane, and TMSCl. Compound I-6-2 can be synthesized by cyclizing compound I-6-2-1 with 2-bromo-1,1-diethoxypropane. The organozinc compound can undergo a Negishi coupling in the presence of compound I-6-2 to afford compound I-6-3. Compound I-6-3 can be further reacted in the presence of zinc, zinc cyanide, and (1,1′-bis(diphenylphosphino)ferrocene)palladium(II) dichloride to afford compound I-6-4. The nitrile of compound I-6-4 can be reacted with ammonium chloride to afford amidine compound I-6-5. The amidine of compound I-6-5 can be condensed with compound I-6-6 to afford cyclized compound I-6. The aryl chloride of I-6 can be reacted in the presence of zinc, zinc cyanide, and Pd₂(dba)₃, and diphenylphosphinoferrocene to afford cyano Compound I-42.

### Synthesis of I-6-1

To a stirred solution of 4-chloro-2,5-difluorobenzoic acid (2 g, 10.387 mmol, 1.0 equiv) in anhydrous THF (30 mL) was added LiAlH₄ (867.2 mg, 22.851 mmol, 2.2 equiv), in portions, at 0° C. and the resulting mixture was stirred for 2 h at room temperature. Then the reaction was quenched by water (20 mL) and the aqueous layer was extracted with EtOAc (3×20 mL). The combined organic layers were washed with brine (20 mL), dried over anhydrous Na₂SO₄ and concentrated under vaccum. The residue was purified by silica gel column chromatography, eluted with petroleum ether/ethyl acetate (3:1) to afford (4-chloro-2,5-difluorophenyl)methanol (1.7 g, 92% yield) as a colorless oil. ¹H NMR (400 MHz, Chloroform-d) δ 7.34-7.22 (m, 1H), 7.12 (dd, J=9.0, 5.9 Hz, 1H), 4.71 (s, 2H).

To a stirred solution of (4-chloro-2,5-difluorophenyl)methanol produced as indicated above (1.7 g, 9.55 mmol, 1.0 equiv) in anhydrous Et₂O (25 mL) was added PBr₃ (1.28 g, 4.77 mmol, 0.5 equiv) at 0° C. The resulting mixture was stirred for 0.5 h at room temperature. Then the reaction was quenched by water (20 mL) and the aqueous layer was extracted with EtOAc (3×20 mL). The combined organic layers were washed with brine (20 mL), dried over anhydrous Na₂SO₄ and concentrated under vaccum to afford 1-(bromomethyl)-4-chloro-2,5-difluorobenzene (I-6-1, 2.28 g, crude) as a colorless oil. ¹H NMR (400 MHz, Chloroform-d) δ 7.23-7.13 (m, 2H), 4.43 (s, 2H).

### Synthesis of I-6-2

To a stirred solution of 3,5-dibromopyrazin-2-amine (I-6-2-1, 1 g, 3.954 mmol, 1 equiv) in anhydrous i-PrOH (15 mL) was added 2-bromo-1,1-diethoxypropane (1.25 g, 5.931 mmol, 1.5 equiv) at room temperature. The resulting mixture was stirred at 85° C. overnight. The mixture was allowed to cool down to room temperature and the precipitated solids were collected by filtration and washed with water (3×10 mL) to afford 6,8-dibromo-3-methylimidazo[1,2-a]pyrazine (1-6-2, 700 mg, crude) as a white solid. This crude was used in next step without further purification. LC-MS: MS (ESI) m/z 291.9 [M+H].

### Synthesis of I-6-3

To a stirred solution of Zn (211.2 mg, 3.23 mmol, 2.0 equiv) in anhydrous THF (4 mL) was added 1,2-dibromoethane (12.1 mg, 0.065 mmol, 0.04 equiv) at 50° C. The resulting mixture was stirred at 50° C. for a period of 10 min. To the above mixture was then added TMSCl (8.7 mg, 0.081 mmol, 0.05 equiv) dropwise at room temperature. The resulting mixture was stirred for an additional 10 min at room temperature and then allowed to cool down to 0° C. at which point 1-(bromomethyl)-4-chloro-2,5-difluorobenzene (I-6-1, 585.1 mg, 2.422 mmol, 1.5 equiv) was added and the resulting mixture was stirred at room temperature for 2 h. A solution of 6,8-dibromo-3-methylimidazo[1,2-a]pyrazine (1-6-2, 470 mg, 1.615 mmol, 1 equiv) and Pd(PPh₃)₂Cl₂ (45.4 mg, 0.065 mmol, 0.04 equiv) in THF (2 mL) was added to the above mixture and the resulting mixture was stirred for 2 h at 45° C. This mixture was allowed to cool down to room temperature and then diluted with water (5 mL). The aqueous layer was extracted with EtOAc (3×5 mL) and the combined organic layers were washed with brine (20 mL), dried over anhydrous Na₂SO₄ and concentrated under vacuum. The residue was purified by silica gel column chromatography, and eluted with petroleum ether/ethyl acetate (3:1) to afford 6-bromo-8-[(4-chloro-2,5-difluorophenyl)methyl]-3-methylimidazo[1,2-a]pyrazine (I-6-3, 380 mg, 63% yield) as a yellow solid. LC-MS (ESI) m/z 374.0 [M+H].

### Synthesis of I-6-4

To a stirred solution of 6-bromo-8-[(4-chloro-2,5-difluorophenyl)methyl]-3-methylimidazo[1,2-a]pyrazine (I-6-3, 370 mg, 0.993 mmol, 1.0 equiv) in anhydrous DMF (5 mL) were added Zn(CN)₂ (58.3 mg, 0.496 mmol, 0.5 equiv) and Pd(dppf) Cl₂ (20.1 mg, 0.025 mmol, 0.025 equiv) at room temperature. The resulting mixture was stirred for 2 h at 120° C. The mixture was then allowed to cool down to room temperature and quenched with Water (10 mL). The aqueous layer was extracted with EtOAc (3×10 mL) and the combined organic layers were washed with brine (20 mL), dried over anhydrous Na₂SO₄ and concentrated under vacuum. The residue was purified by silica gel column chromatography, eluted with petroleum ether/ethyl acetate (1:1) to afford 8-[(4-chloro-2,5-difluorophenyl)methyl]-3-methylimidazo[1,2-a]pyrazine-6-carbonitrile (I-6-4, 260 mg, 82% yield) as a white solid. LC-MS: (ESI) m/z 319.10 [M+H].

### Synthesis of I-6-5

To a stirred solution of 8-[(4-chloro-2,5-difluorophenyl)methyl]-3-methylimidazo[1,2-a]pyrazine-6-carbonitrile (I-6-4, 235 mg, 0.737 mmol, 1.0 equiv) in anhydrous methanol (5 mL) was added NaOMe (2.8 mg, 0.016 mmol, 0.10 equiv, 30% wt in MeOH) at room temperature and the resulting mixture was stirred for 2 h at 45° C. Then NH₄Cl (78.9 mg, 1.474 mmol, 2.0 equiv) was added and the mixture was stirred for another 2 h at 45° C. The reaction was quenched by the addition of Water (5 mL) at room temperature. The aqueous layer was extracted with EtOAc (3×10 mL) and the combined organic layers were washed with brine (20 mL), dried over anhydrous Na₂SO₄ and concentrated under vacuum. The combined organic layer was dried under vacuum to afford (8-[(4-chloro-2,5-difluorophenyl)methyl]-3-methylimidazo[1,2-a]pyrazine-6-carboximidamide (1-6-5, 240 mg, crude) as brown solid. LC-MS: (ESI) m/z 335.9 [M+H].

### Synthesis of Compound I-6

To a stirred solution of 8-[(4-chloro-2,5-difluorophenyl)methyl]-3-methylimidazo[1,2-a]pyrazine-6-carboximidamide (I-6-5, 230 mg, 0.685 mmol, 1 equiv) in anhydrous MeOH (5 mL) was added 1,3-dimethyl 2-fluoropropanedioate (1-6-6, 154.3 mg, 1.028 mmol, 1.5 equiv) and NaOMe (431.8 mg, 2.397 mmol, 3.5 equiv, 30% wt in MeOH) at room temperature and the resulting mixture was stirred for 2 h at 70° C. and then it was allowed to cool down to room temperature. The crude product was precipitated by the addition of 1 M HCl (5 mL) and purified by trituration with DMSO (5 mL) to afford 2-{8-[(4-chloro-2,5-difluorophenyl)methyl]-3-methylimidazo[1,2-a]pyrazin-6-yl}-5-fluoropyrimidine-4,6-diol (Compound I-6, 64.4 mg, 21% yield) as a white solid. LC-MS: (ESI) m/z 422.05 [M+H]. ¹H NMR: (400 MHz, DMSO-d₆) δ 12.25 (s, 2H), 8.90 (s, 1H), 7.72 (s, 1H), 7.72-7.68 (dd, J=9.0, 5.8 Hz, 1H), 7.62 (dd, J=9.1, 6.2 Hz, 1H), 4.54 (s, 2H), 2.56 (s, 3H). ¹⁹F NMR-PH-CYCN-CYC-006-0: (376 MHz, DMSO-d₆) δ −119.20 (d, J=15.9 Hz), −122.01 (d, J=15.8 Hz), −177.59.

Compound I-6 can be reacted in the presence of zinc, zinc cyanide, and Pd₂(dba)₃, and diphenylphosphinoferrocene to afford cyano Compound I-42.

### Synthesis of 2-(8-(2,5-difluoro-4-methylbenzyl)-3-methoxyimidazo[1,2-a]pyrazin-6-yl)-5-methylpyrimidin-4-ol (I-7) and Compound I-46

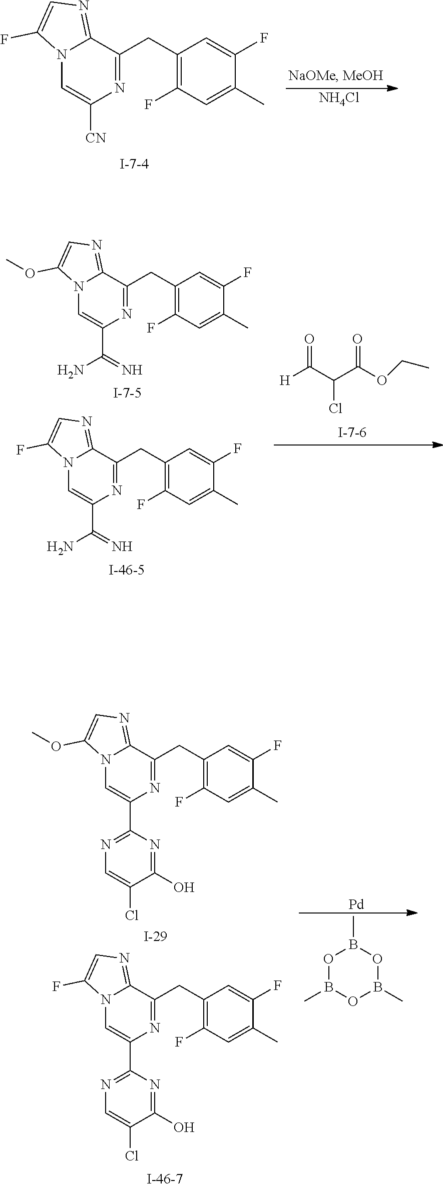

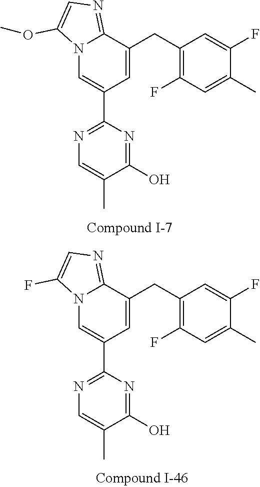 [SMILES: COc1cnc2c(Cc3cc(F)c(C)cc3F)cc(-c3ncc(C)c(O)n3)cn12 ; Cc1cc(F)c(Cc2cc(-c3ncc(C)c(O)n3)cn3c(F)cnc23)cc1F]

Compound I-7-4 was prepared via a method analogous to compound I-4-4. The nitrile of compound I-7-4 was reacted with ammonium chloride in NaOMe/MeOH to afford compound I-7-5 and compound I-46-5. The amidine of compounds I-7-5 and I-46-5 were condensed with compound I-7-6 to afford cyclized compounds I-29 and I-46-7. Compound I-29 and compound I-46-7 were reacted with 2,4,6-trimethyl-1,3,5,2,4,6-trioxatriborinane in the presence of palladium to afford compounds I-7 and I-46.

### Synthesis of Compound I-7

To a stirred mixture of 5-chloro-2-{8-[(2,5-difluoro-4-methylphenyl)methyl]-3-methoxyimidazo[1,2-a]pyrazin-6-yl}pyrimidin-4-ol (I-29, 200 mg, 0.479 mmol, 1 equiv) and trimethyl-1,3,5,2,4,6-trioxatriborinane (120.2 mg, 0.958 mmol, 2 equiv) in dioxane (4 mL) were added SPhos (19.7 mg, 0.048 mmol, 0.1 equiv), SPhos Palladacycle Gen.3 (37.35 mg, 0.048 mmol, 0.1 equiv) and K₂CO₃ (132.3 mg, 0.958 mmol, 2 equiv) at room temperature under a nitrogen atmosphere. The resulting mixture was stirred overnight at 90° C. under the nitrogen atmosphere and then allowed to cool down to room temperature. The resulting mixture was diluted with water (20 mL) and extracted with EtOAc (3×20 mL) and the combined organic layers were dried over anhydrous Na₂SO₄. After filtration, the filtrate was concentrated under reduced pressure and the residue was purified by reverse-phase flash chromatography with the following conditions: column, C18 silica gel; mobile phase, MeCN in Water (0.1% formic acid), 0% to 100% gradient in 10 min; and detector: UV 254 nm, to afford 2-{8-[(2,5-difluoro-4-methylphenyl)methyl]-3-methoxyimidazo[1,2-a]pyrazin-6-yl}-5-methylpyrimidin-4-ol (I-7, 17.4 mg, 9% yield) as an off-white solid. LC-MS (ESI): m/z 398.15 [M+H]. ¹H NMR: (400 MHz, DMSO-d₆) δ11.85 (s, 1H), 8.71 (s, 1H), 7.94 (s, 2H), 7.48 (s, 1H), 7.34 (s, 1H), 7.14 (t, J=8.4 Hz, 2H), 4.47 (s, 2H), 4.13 (s, 4H), 2.18 (s, 5H), 1.98 (s, 4H). ¹⁹FNMR: (376 MHz, DMSO-d₆) δ −123.12 (dd, J=26.2, 18.8 Hz), −129.25.

### Synthesis of 1-(8-(2,5-difluorobenzyl)-6-(5-fluoro-4-hydroxy-6-methylpyrimidin-2-yl)imidazo[1,2-a]pyrazin-3-yl)ethan-1-one (I-8) and 2-(8-(2,5-difluorobenzyl)-3-(1-hydroxyethyl)imidazo[1,2-a]pyrazin-6-yl)-5-fluoro-6-methylpyrimidin-4-ol (I-31)

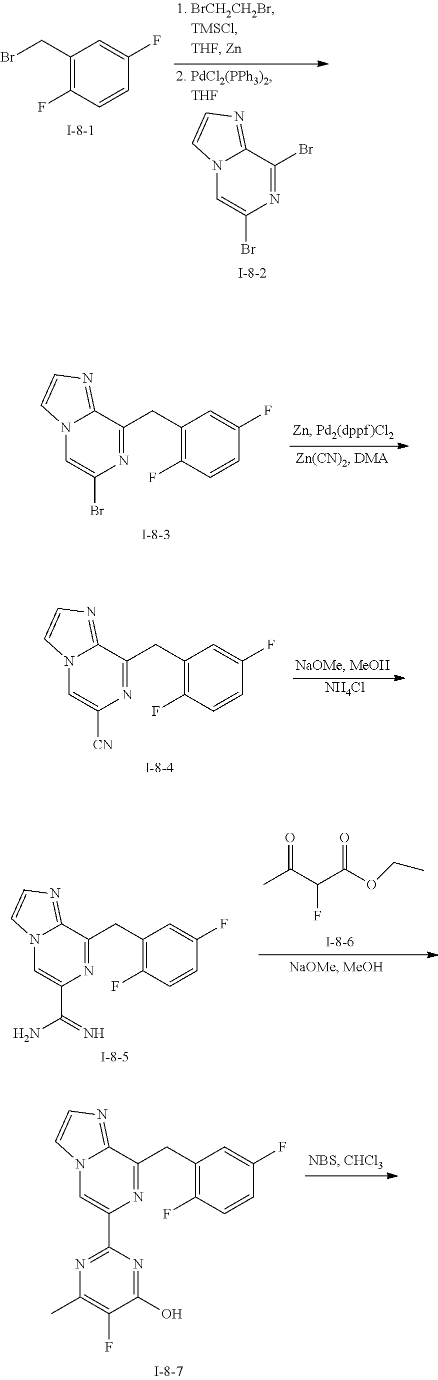

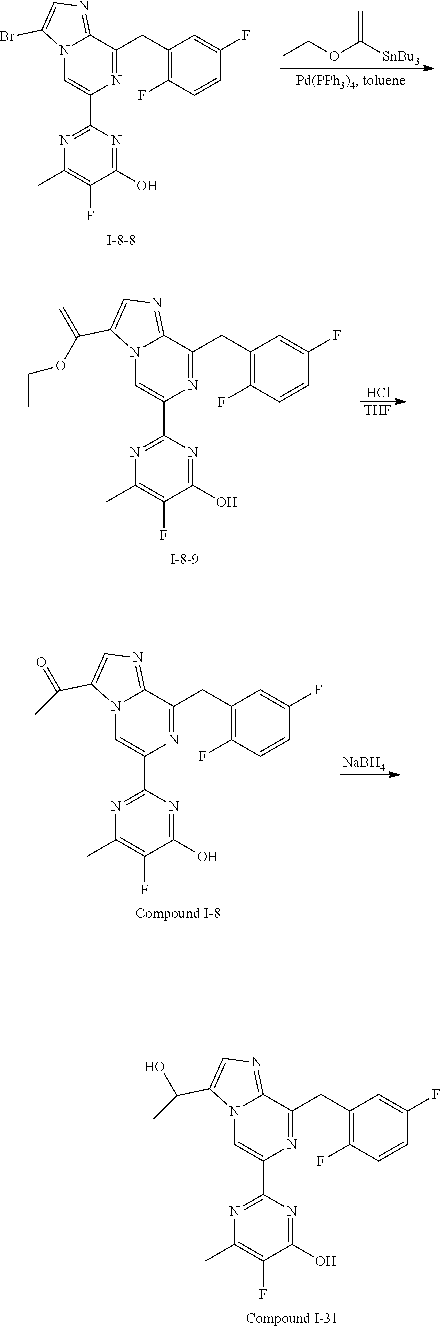

Compound I-8-1 was transformed into the corresponding organozinc compound using zinc, dibromoethane, and TMSCl. The organozinc compound underwent a Negishi coupling in the presence of compound I-8-2 to afford compound I-8-3. Compound I-8-3 was further reacted in the presence of zinc, zinc cyanide, and (1,1′-bis(diphenylphosphino)ferrocene)palladium(II) dichloride to afford compound I-8-4. The nitrile of compound I-8-4 was reacted with ammonium chloride to afford amidine compound I-8-5. The amidine of compound I-8-5 was condensed with compound I-8-6 to afford cyclized compound I-8-7. Compound I-8-7 was reacted with N-bromosuccinimide to afford the brominated product, compound I-8-8. Compound I-8-8 underwent a Stille reaction with stannous ethoxy vinyl to afford compound I-8-9. Compound I-8-9 was hydrolyzed into compound I-8 using hydrochloric acid, and compound I-8 was reduced to compound I-31 in the presense of sodium borohydride.

### Synthesis of I-8-3

To a stirred solution of Zn (0.95 g, 14.491 mmol, 1.5 equiv) in anhydrous THF (20 mL) was added dibromoethane (90.7 mg, 0.483 mmol, 0.05 equiv) at 25° C. under nitrogen atmosphere. The mixture was stirred for 10 min at 50° C. under the nitrogen atmosphere. TMSCl (52.4 mg, 0.483 mmol, 0.05 equiv) was added and the mixture was cooled to ambient temperature. Then, 2-(bromomethyl)-1,4-difluorobenzene (I-8-1, 2 g, 9.661 mmol, 1 equiv) was added dropwise at 0° C. under the nitrogen atmosphere. The mixture was stirred for 2 h at 25° C. under the nitrogen atmosphere. 6,8-dibromoimidazo[1,2-a]pyrazine (1-8-2, 1.87 g, 6.763 mmol, 0.7 equiv) and Pd(PPh₃)₂Cl₂ (135.6 mg, 0.193 mmol, 0.02 equiv) were then added at 25° C. under the nitrogen atmosphere. The mixture was stirred for 2 h at 50° C. under nitrogen atmosphere. The reaction was then quenched by the addition of Water (50 mL) at room temperature and the resulting mixture was extracted with EtOAc (3×100 mL). The combined organic layers were washed with brine (1×200 mL) and dried over anhydrous Na₂SO₄. After filtration, the filtrate was concentrated under reduced pressure and the residue was purified by silica gel column chromatography, eluted with petroleum ether/ethyl acetate (5:1) to afford 6-bromo-8-[(2,5-difluorophenyl)methyl]imidazo[1,2-a]pyrazine (I-8-3, 1.82 g, 58% yield) as an off-white solid. LC-MS: (ESI) m/z 323.9 [M+H]. ¹H NMR: (400 MHz, CDCl₃) δ 8.18 (s, 1H), 7.79 (d, J=0.9 Hz, 1H), 7.67 (d, J=0.9 Hz, 1H), 7.10-7.04 (m, 1H), 7.00 (h, J=8.9, 4.5 Hz, 1H), 6.92-6.81 (m, 1H), 4.58 (s, 2H).

### Synthesis of I-8-4

To a stirred solution of 6-bromo-8-[(2,5-difluorophenyl)methyl]imidazo[1,2-a]pyrazine (I-8-3, 2 g, 6.170 mmol, 1 equiv) in anhydrous DMF (40 mL) were added Zn (80.6 mg, 1.234 mmol, 0.2 equiv), Zn(CN)₂ (434.7 mg, 3.702 mmol, 0.6 equiv) and Pd(dppf)Cl₂ (112.8 mg, 0.154 mmol, 0.025 equiv) at 25° C. under nitrogen atmosphere and the resulting mixture was stirred for 2 h at 120° C. under the nitrogen atmosphere. The reaction was quenched by the addition of Water (200 mL) at room temperature and the resulting mixture was extracted with EtOAc (3×100 mL). The combined organic layers were washed with brine (1×200 mL) and dried over anhydrous Na₂SO₄. After filtration, the filtrate was concentrated under reduced pressure and the residue was purified by silica gel column chromatography, eluted with petroleum ether/ethyl acetate (3:1) to afford 8-[(2,5-difluorophenyl)methyl]imidazo[1,2-a]pyrazine-6-carbonitrile (I-8-4, 1.72 g, crude) as a light yellow solid. This crude was used in the next step without additional purification. LC-MS: (ESI) m/z 271.0 [M+H].

### Synthesis of I-8-5

To a stirred solution of 8-[(2,5-difluorophenyl)methyl]imidazo[1,2-a]pyrazine-6-carbonitrile (I-8-4, 1.7 g, 6.291 mmol, 1 equiv) in MeOH (20 mL) was added NaOMe (0.11 g, 0.629 mmol, 0.1 equiv, 30% wt in MeOH) at 25° C. The mixture was stirred for 4 h at 50° C. and NH₄Cl (0.67 g, 12.582 mmol, 2 equiv) was then added. The resulting mixture was stirred for 4 h at 50° C. The reaction was then quenched by the addition of sat. NaHCO₃ (aq.) (100 mL) at room temperature and the resulting mixture was extracted with IPA/DCM=1:3 (3×100 mL). The combined organic layers were washed with brine (1×100 mL) and dried over anhydrous Na₂SO₄. After filtration, the filtrate was concentrated under reduced pressure to afford 8-[(2,5-difluorophenyl)methyl]imidazo[1,2-a]pyrazine-6-carboximidamide (1-8-5, 1.34 g, 74% yield) as a brown oil. This was used in the next step without further purification. LC-MS: (ESI) m/z 288.1 [M+H].

### Synthesis of I-8-7

To a stirred solution of 8-[(2,5-difluorophenyl)methyl]imidazo[1,2-a]pyrazine-6-carboximidamide (I-8-5, 1.3 g, 4.525 mmol, 1 equiv) in MeOH (20 mL) were added ethyl 2-fluoro-3-oxobutanoate (I-8-6, 1.01 g, 6.788 mmol, 1.5 equiv) and NaOMe (0.18 g, 0.996 mmol, 0.22 equiv, 30% wt in MeOH) at 25° C. The mixture was stirred for 8 h at 70° C. The reaction was quenched by the addition of Water (50 mL) at room temperature and then the mixture acidified to pH 3-4 with 1 M HCl (10 mL). The resulting mixture was extracted with EtOAc (3×100 mL) and the combined organic layers were washed with brine (1×200 mL) and dried over anhydrous Na₂SO₄. After filtration, the filtrate was concentrated under reduced pressure and the residue purified by reverse-phase flash chromatography with the following conditions: column, C18 silica gel; mobile phase, MeCN in Water (0.1% NH₃·H₂O), 10% to 80% gradient in 10 min; and detector, UV 254/220 nm, to afford 2-{8-[(2,5-difluorophenyl)methyl]imidazo[1,2-a]pyrazine-6-yl}-5-fluoro-6-methylpyrimidin-4-ol (1-8-7, 720 mg, 42% yield) as a light yellow solid. LC-MS: (ESI) m/z 372.1 [M+H].

### Synthesis of I-8-8

To a stirred solution of 2-{8-[(2,5-difluorophenyl)methyl]imidazo[1,2-a]pyrazin-6-yl}-5-fluoro-6-methylpyrimidin-4-ol (1-8-7, 600 mg, 1.616 mmol, 1 equiv) in CHCl₃ (10 mL) was added NBS (316.3 mg, 1.778 mmol, 1.1 equiv) at 25° C. The mixture was stirred for 3 h at 25° C. The reaction was quenched by the addition of Water (50 mL) at room temperature and the resulting mixture was extracted with EtOAc (3×100 mL). The combined organic layers were washed with brine (1×200 mL) and dried over anhydrous Na₂SO₄. After filtration, the filtrate was concentrated under reduced pressure to afford 2-{3-bromo-8-[(2,5-difluorophenyl)methyl]imidazo[1,2-a]pyrazin-6-yl}-5-fluoro-6-methylpyrimidin-4-ol (I-8-8, 756 mg, crude) as a yellow solid. LC-MS: (ESI) m/z 450.0 [M+H].

### Synthesis of I-8-9

To a stirred solution of 2-{3-bromo-8-[(2,5-difluorophenyl)methyl]imidazo[1,2-a]pyrazin-6-yl}-5-fluoro-6-methylpyrimidin-4-ol (1-8-8, 700 mg, 1.555 mmol, 1 equiv) in toluene (10 mL) were added tributyl(1-ethoxyethenyl)stannane (673.8 mg, 1.866 mmol, 1.2 equiv) and Pd(PPh₃)₄ (179.6 mg, 0.155 mmol, 0.1 equiv) at 25° C. under a nitrogen atmosphere. The mixture was stirred for 12 h at 80° C. under a nitrogen atmosphere. The reaction was quenched by the addition of Water (50 mL) at room temperature and the resulting mixture was extracted with EtOAc (3×100 mL). The combined organic layers were washed with brine (1×200 mL) and dried over anhydrous Na₂SO₄. After filtration, the filtrate was concentrated under reduced pressure to afford 2-{8-[(2,5-difluorophenyl)methyl]-3-(1-ethoxyethenyl)imidazo[1,2-a]pyrazin-6-yl}-5-fluoro-6-methylpyrimidin-4-ol (I-8-9, 600 mg, 87% yield) as a brown solid.

LC-MS: (ESI) m/z 442.1 [M+H].

### Synthesis of Compound I-8

To a stirred solution of 2-{8-[(2,5-difluorophenyl)methyl]-3-(1-ethoxyethenyl)imidazo[1,2-a]pyrazin-6-yl}-5-fluoro-6-methylpyrimidin-4-ol (I-8-9, 600 mg, 1.359 mmol, 1 equiv) in tetrahydrofuran (10 mL) was added 2M HCl (10 mL) at 25° C. under a nitrogen atmosphere. The mixture was stirred for 4 h at 25° C. The reaction was quenched by the addition of Water (50 mL) at room temperature. The resulting mixture was extracted with DCM (3×100 mL) and the combined organic layers were washed with brine (1×50 mL) and dried over anhydrous Na₂SO₄. After filtration, the filtrate was concentrated under reduced pressure. The residue was purified by reverse-phase flash chromatography with the following conditions: column, C18 silica gel; mobile phase, MeCN in Water (0.1% NH₃·H₂O), 10% to 80% gradient in 20 min; and detector, UV 254/220 nm, to afford 1-{8-[(2,5-difluorophenyl) methyl]-6-(5-fluoro-4-hydroxy-6-methylpyrimidin-2-yl)imidazo[1,2-a]pyrazin-3-yl}ethanone (I-8, 50.8 mg, 18% yield) as an off-white solid. LC-MS: (ESI) m/z 414.15 [M+H]. v¹H NMR: (400 MHz, CDCl₃) δ 10.75 (s, 1H), 10.33 (s, 1H), 8.47 (s, 1H), 7.15-7.01 (m, J=13.6, 9.0, 5.0 Hz, 2H), 7.01-6.84 (m, 1H), 4.70 (s, 2H), 2.71 (s, 3H), 2.42 (d, J=3.7 Hz, 3H).

¹⁹F NMR: (376 MHz, CDCl₃) δ −118.49 (d, J=17.6 Hz), −122.25 (d, J=17.5 Hz), −149.61.

### Synthesis of I-31

To a stirred solution of 1-{8-[(2,5-difluorophenyl)methyl]-6-(5-fluoro-4-hydroxy-6-methylpyrimidin-2-yl) imidazo[1,2-a]pyrazin-3-yl}ethanone (I-8, 55 mg, 0.133 mmol, 1 equiv) in THF (1 mL) and MeOH (0.2 mL) was added NaBH₄ (15.1 mg, 0.399 mmol, 3 equiv) at 0° C. The mixture was stirred for 2 h at 25° C. The reaction was quenched by the addition of Water (2 mL) at room temperature. The resulting mixture was purified by reverse-phase flash chromatography with the following conditions: column, C18 silica gel; mobile phase, ACN in Water (0.1% TFA), 10% to 80% gradient in 10 min; and detector, UV 254/220 nm, to afford 2-{8-[(2,5-difluorophenyl)methyl]-3-(1-hydroxyethyl) imidazo[1,2-a]pyrazin-6-yl}-5-fluoro-6-methylpyrimidin-4-ol (I-31, 35.1 mg, 63% yield) as a white solid. LC-MS: (ESI) m/z 416.20 [M+H]. ¹H NMR: (400 MHz, CDCl₃) δ 10.78 (s, 1H), 9.29 (s, 1H), 7.78 (s, 1H), 7.06 (td, J=8.9, 4.9 Hz, 2H), 6.98-6.88 (m, 1H), 5.36 (q, J=6.6 Hz, 1H), 4.62 (s, 2H), 2.39 (d, J=3.7 Hz, 3H), 1.86 (d, J=6.6 Hz, 3H). ¹⁹F NMR: (376 MHz, CDCl³) δ −118.77 (d, J=17.9 Hz), −122.41 (d, J=17.9 Hz), −150.57.

### Synthesis of 1-(4-((3-chloro-6-(5-fluoro-4-hydroxypyrimidin-2-yl)imidazo[1,2-a]pyrazin-8-yl)methyl)-3-fluorophenyl)ethan-1-one (I-9) and 2-(3-chloro-8-(2-fluoro-4-(1-hydroxyethyl)benzyl)imidazo[1,2-a]pyrazin-6-yl)-5-fluoropyrimidin-4-ol (I-33)

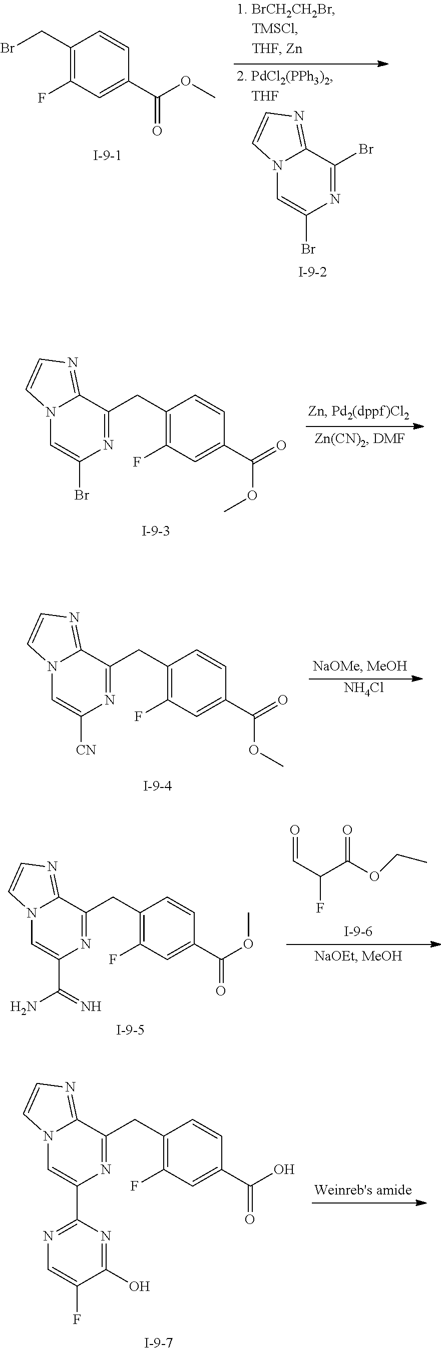

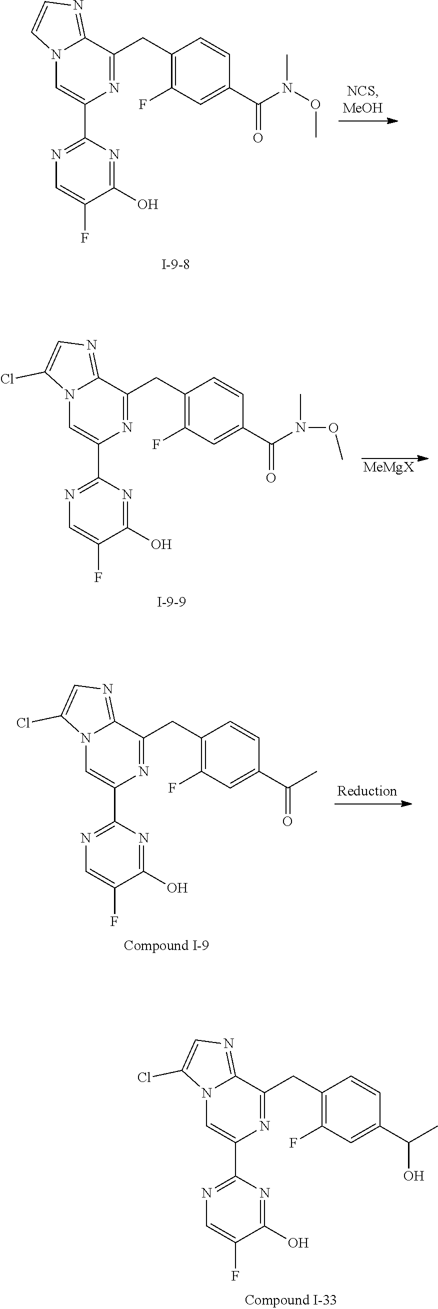

Compound I-9-1 was transformed into the corresponding organozinc compound using zinc, dibromoethane, and TMSCl. The organozinc compound underwent a Negishi coupling in the presence of compound I-9-2 to afford compound I-9-3. Compound I-9-3 was further reacted in the presence of zinc, zinc cyanide, and (1,1′-bis(diphenylphosphino)ferrocene)palladium(II) dichloride to afford compound I-9-4. The nitrile of compound I-9-4 was reacted with ammonium chloride to afford compound I-9-5. The amidine of compound I-9-5 was condensed with compound I-9-6 to afford cyclized compound I-9-7. Compound I-9-7, in the presense of Weinreb's amide, was converted to intermediate I-9-8. Compound I-9-8 was chlorinated using N-chlorosuccinimide to afford compound I-9-9. The amide moiety of compound I-9-9 was reacted with a methyl Grignard reagent to afford compound I-9. The resulting ketone of compound I-9 was reduced to the secondary alcohol to afford compound I-33.

### Synthesis of I-9-3

To a stirred solution of Zn (1.59 g, 24.285 mmol, 1.5 equiv) in THF (25 mL) was added 1,2-dibromoethane (0.15 g, 0.810 mmol, 0.05 equiv) dropwise at room temperature under a nitrogen atmosphere. The resulting mixture was stirred for 10 min at 50° C. under the nitrogen atmosphere. To the above mixture was then added TMSCl (0.09 g, 0.810 mmol, 0.05 equiv) dropwise. The mixture was allowed to cool down to 0° C. To this above mixture was then added methyl 4-(bromomethyl)-3-fluorobenzoate (I-9-1, 4 g, 16.190 mmol, 1 equiv) dropwise at 0° C. and the resulting mixture was stirred for 10 min at 0° C. under a nitrogen atmosphere and then stirred for an additional 2 h at room temperature, still under a nitrogen atmosphere. To the above mixture were added 6,8-dibromoimidazo[1,2-a]pyrazine (I-9-2, 3.14 g, 11.333 mmol, 0.7 equiv) and Pd(PPh₃)₂Cl₂ (0.23 g, 0.324 mmol, 0.02 equiv) at room temperature and the resulting mixture was stirred for an additional 1 h at 40° C. The mixture was allowed to cool down to room temperature. The reaction was quenched with sat. NH₄Cl (aq.) (30 mL) at room temperature and the resulting mixture was extracted with EtOAc (3×50 mL). The combined organic layers were washed with brine (2×50 mL) and dried over anhydrous Na₂SO₄. After filtration, the filtrate was concentrated under reduced pressure and the residue was purified by reverse-phase flash chromatography with the following conditions: column, C18 silica gel; mobile phase, MeCN in Water (0.1% formic acid), 0% to 100% gradient in 10 min; and detector, UV 254 nm, to afford methyl 4-({6-bromoimidazo[1,2-a]pyrazin-8-yl}methyl)-3-fluorobenzoate (1-9-3, 2.18 g, 37% yield) as a yellow solid. LC-MS (ESI) m/z 364.00 [M+H].

### Synthesis of I-9-4

To a stirred solution of methyl 4-({6-bromoimidazo[1,2-a]pyrazin-8-yl}methyl)-3-fluorobenzoate (I-9-3, 2 g, 5.492 mmol, 1 equiv), Zn(CN)₂ (0.39 g, 3.295 mmol, 0.6 equiv) and Zn (0.07 g, 1.098 mmol, 0.2 equiv) in DMF (40 mL) was added Pd(dppf)Cl₂ (0.10 g, 0.137 mmol, 0.025 equiv) at room temperature under a nitrogen atmosphere. The resulting mixture was stirred for 2 h at 120° C. under the nitrogen atmosphere. The reaction was quenched with Water at room temperature and the resulting mixture was extracted with EtOAc (3×50 mL). The combined organic layers were washed with water (3×100 mL) and brine (3×100 mL) and dried over anhydrous Na₂SO₄. After filtration, the filtrate was concentrated under reduced pressure and the residue was purified by silica gel column chromatography, eluted with petroleum ether/ethyl acetate (2:1) to afford methyl 4-({6-cyanoimidazo[1,2-a]pyrazin-8-yl}methyl)-3-fluorobenzoate (I-9-4, 1.26 g, 74% yield) as an off-white solid. LC-MS: (ESI) m/z 311.09 [M+H].

### Synthesis of I-9-5

To a stirred solution of methyl 4-({6-cyanoimidazo[1,2-a]pyrazin-8-yl}methyl)-3-fluorobenzoate (I-9-4, 1.09 g, 3.513 mmol, 1 equiv) in MeOH (22 mL) was added NaOMe (0.06 g, 0.351 mmol, 0.1 equiv, 30% wt in MeOH) dropwise at room temperature under a nitrogen atmosphere. The resulting mixture was stirred for 3 h at 40° C. under the nitrogen atmosphere. To the above mixture was added NH₄Cl (0.38 g, 7.026 mmol, 2 equiv) at 40° C. and the resulting mixture was stirred overnight at 40° C. The reaction was quenched with sat. NaHCO₃ (aq.) (20 mL) at room temperature. The resulting mixture was extracted with DCM:IPA (3:1) (3×40 mL) and the combined organic layers were dried over anhydrous Na₂SO₄. After filtration, the filtrate was concentrated under reduced pressure to afford methyl 4-({6-carbamimidoylimidazo[1,2-a]pyrazin-8-yl}methyl)-3-fluorobenzoate (I-9-5, 822 mg, 71.5%) as a yellow solid. LC-MS: (ESI) m/z 328.11 [M+H].

### Synthesis of 1-9-7

To a stirred solution of methyl 4-({6-carbamimidoylimidazo[1,2-a]pyrazin-8-yl}methyl)-3-fluorobenzoate (I-9-5, 372 mg, 1.137 mmol, 1 equiv) and ethyl 2-fluoro-3-oxopropanoate (I-9-6, 457.2 mg, 3.411 mmol, 3 equiv) in MeOH (7 mL) was added NaOMe (716.3 mg, 3.979 mmol, 3.5 equiv, 30% wt in MeOH) dropwise at room temperature under a nitrogen atmosphere. The resulting mixture was stirred for 2 h at 80° C. under the nitrogen atmosphere. The mixture was then acidified to pH 4 with 1M HCl (aq.) (2 mL) and the resulting mixture was extracted with EtOAc (3×30 mL). The combined organic layers were washed with brine (1×60 mL) and dried over anhydrous Na₂SO₄. After filtration, the filtrate was concentrated under reduced pressure and the residue was purified by reverse-phase flash chromatography with the following conditions: column, C18 silica gel; mobile phase, MeCN in Water (0.1% formic acid), 0% to 100% gradient in 10 min; and detector, UV 254 nm, to afford 3-fluoro-4-{[6-(5-fluoro-4-hydroxypyrimidin-2-yl)imidazo[1,2-a]pyrazin-8-yl]methyl}benzoic acid (I-9-7, 120 mg, 27.5% yield) as a light yellow solid. LC-MS: (ESI) m/z 384.08 [M+H].

### Synthesis of 1-9-8

To a stirred solution of 3-fluoro-4-{[6-(5-fluoro-4-hydroxypyrimidin-2-yl)imidazo[1,2-a]pyrazin-8-yl]methyl}benzoic acid (I-9-7, 100 mg, 0.261 mmol, 1 equiv), HATU (119.1 mg, 0.313 mmol, 1.2 equiv) and DIEA (101.2 mg, 0.783 mmol, 3 equiv) in DMF (1 mL) was added Weinreb's amine (19.1 mg, 0.313 mmol, 1.2 equiv) at room temperature under a nitrogen atmosphere. The resulting mixture was stirred for 1 h at room temperature under the nitrogen atmosphere. The resulting mixture was purified by reverse-phase flash chromatography with the following conditions: column, C18 silica gel; mobile phase, MeCN in Water (0.1% formic acid), 0% to 100% gradient in 10 min; and detector, UV 254 nm to afford 3-fluoro-4-{[6-(5-fluoro-4-hydroxypyrimidin-2-yl)imidazo[1,2-a]pyrazin-8-yl]methyl}-N-methoxy-N-methylbenzamide (1-9-8, 33 mg, 30% yield) as a yellow solid. LC-MS: (ESI) m/z 427.13 [M+H].

### Synthesis of I-9-9

To a stirred solution of 3-fluoro-4-{[6-(5-fluoro-4-hydroxypyrimidin-2-yl)imidazo[1,2-a]pyrazin-8-yl]methyl}-N-methoxy-N-methylbenzamide (I-9-8, 171 mg, 0.401 mmol, 1 equiv) in DMF (3 mL) was added NCS (32.1 mg, 0.241 mmol, 0.6 equiv) at room temperature under a nitrogen atmosphere. The resulting mixture was stirred overnight at room temperature under the nitrogen atmosphere. The resulting mixture was purified by reverse-phase flash chromatography with the following conditions: column, C18 silica gel; mobile phase, MeCN in Water (0.1% formic acid), 0% to 100% gradient in 10 min; and detector, UV 254 nm, to afford 4-{[3-chloro-6-(5-fluoro-4-hydroxypyrimidin-2-yl)imidazo[1,2-a]pyrazin-8-yl]methyl}-3-fluoro-N-methoxy-N-methylbenzamide (I-9-9, 94 mg, 51% yield) as an off-white solid. LC-MS: (ESI) m/z 461.09 [M+H].

### Synthesis of I-9

To a stirred solution of bromo(methyl)magnesium (0.25 mL, 0.760 mmol, 5 equiv, 2M in THF) was added a solution of 4-{[3-chloro-6-(5-fluoro-4-hydroxypyrimidin-2-yl)imidazo[1,2-a]pyrazin-8-yl]methyl}-3-fluoro-N-methoxy-N-methylbenzamide (I-9-9, 70 mg, 0.152 mmol, 1 equiv) in THF (2 mL), dropwise, at 0° C. and under a nitrogen atmosphere. The resulting mixture was stirred overnight at room temperature under the nitrogen atmosphere. The reaction was then quenched by the addition of sat. NH₄Cl (aq.) (5 mL) at room temperature and the resulting mixture was extracted with EtOAc (3×10 mL). The combined organic layers were washed with brine (1×20 mL) and dried over anhydrous Na₂SO₄. After filtration, the filtrate was concentrated under reduced pressure and the residue was purified by reverse-phase flash chromatography with the following conditions: column, C18 silica gel; mobile phase, MeCN in Water (0.1% formic acid), 0% to 100% gradient in 10 min; and detector, UV 254 nm, to afford 1-(4-{[3-chloro-6-(5-fluoro-4-hydroxypyrimidin-2-yl)imidazo[1,2-a]pyrazin-8-yl]methyl}-3-fluorophenyl)ethanone (I-9, 20 mg, 32% yield) as a yellow solid. The reaction was repeated 3 more times, and a total of 53 mg of product was obtained. LC-MS: (ESI) m/z 416.05 [M+H]. ¹H NMR: (400 MHz, DMSO-d₆) δ 12.85 (s, 1H), 8.95 (s, 1H), 8.21 (s, 1H), 8.06 (s, 1H), 7.77-7.67 (m, 2H), 7.66-7.60 (m, 1H), 4.67 (s, 2H), 2.56 (s, 3H). ¹⁹F NMR: (376 MHz, DMSO-d₆) δ −115.84, −151.89.

### Synthesis of 2-(8-(4-ethyl-2,5-difluorobenzyl)-[1,2,4]triazolo[1,5-a]pyrazin-6-yl)-5-fluoro-6-methoxypyrimidin-4-ol (I-10) and 2-(8-(4-ethyl-2,5-difluorobenzyl)-[1,2,4]triazolo[1,5-a]pyrazin-6-yl)-5-fluoro-6-(methylthio)pyrimidin-4-ol (I-34)

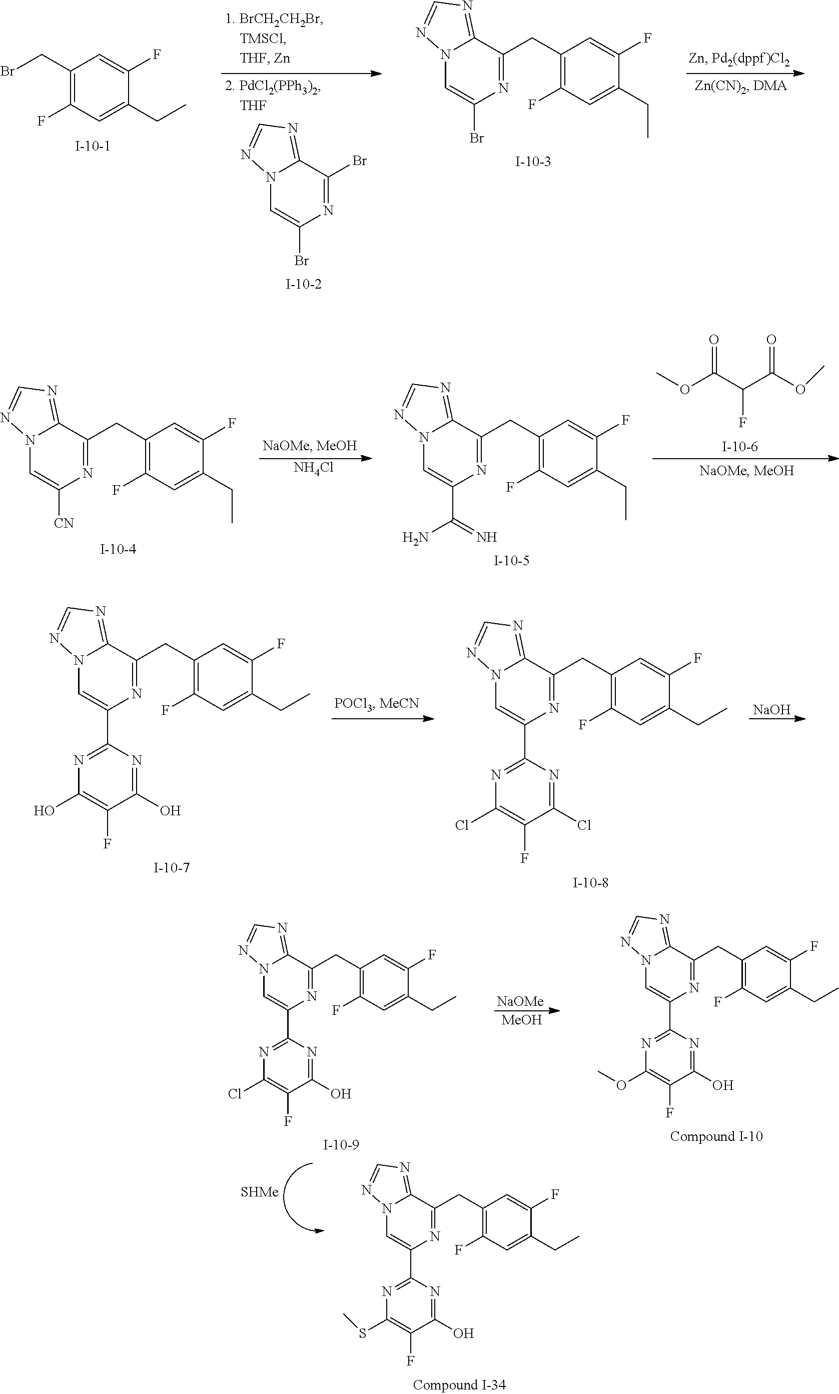

Compound I-10-1 can be transformed into the corresponding organozinc compound using zinc, dibromoethane, and TMSCL. The organozinc compound can undergo a Negishi coupling in the presence of compound I-10-2 to afford compound I-10-3. Compound I-10-3 can be further reacted in the presence of zinc, zinc cyanide, and (1,1′-bis(diphenylphosphino)ferrocene)palladium(II) dichloride to afford compound I-10-4. The nitrile of compound I-10-4 can be reacted with ammonium chloride to afford amidine compound I-10-5. The amidine of compound I-10-5 can be condensed with compound I-10-6 to afford cyclized compound I-10-7. The aryl alcohols of compound I-10-7, in the presence of POCl₃, can be converted to aryl chlorides to afford compound I-10-8. Compound I-10-8 can be partially hydroxylated in the presence of sodium hydroxide to afford compound I-10-9. The remaining aryl chloride of compound I-10-9 can be either be converted to compound I-10 using sodium methoxide or compound I-34 using SHMe.

### Synthesis of 2-(8-(2,5-difluoro-4-(methylthio)benzyl)-3-(hydroxymethyl)imidazo[1,2-a]pyrazin-6-yl)-5-fluoropyrimidin-4-ol (I-39), 2-(8-(2,5-difluoro-4-(methylthio)benzyl)-3-(fluoromethyl)imidazo[1,2-a]pyrazin-6-yl)-5-fluoropyrimidin-4-ol (I-11), 2-(8-(4,5-difluoro-2-(methylthio)benzyl)-3-(fluoromethyl)imidazo[1,2-a]pyrazin-6-yl)-5-fluoropyrimidin-4-ol (I-40), and 2-(8-(4,5-difluoro-2-(methylthio)benzyl)-3-(hydroxymethyl)imidazo[1,2-a]pyrazin-6-yl)-5-fluoropyrimidin-4-ol (I-41)

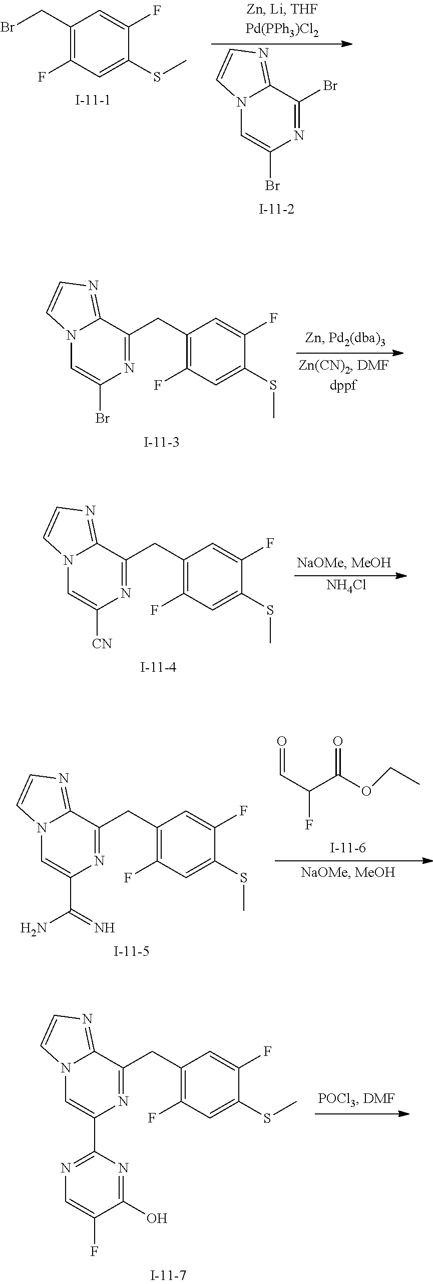

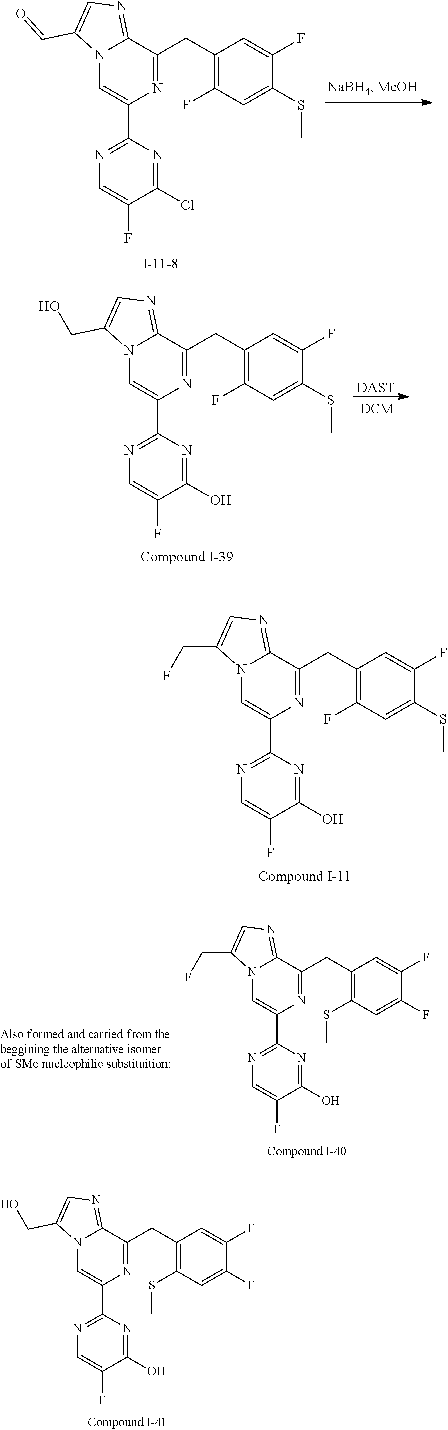

Compound I-11-1 can be transformed into the corresponding organozinc compound using zinc, dibromoethane, and TMSCl. The organozinc compound can undergo a Negishi coupling with compound I-11-2 to afford compound I-11-3. Compound I-11-3 can be further reacted in the presence of zinc, zinc cyanide, and tris(dibenzylideneacetone)dipalladium(0) to afford compound I-11-4. The nitrile of compound I-11-4 can be reacted with ammonium chloride to afford amidine compound I-11-5. The amidine of compound I-11-5 can be condensed with compound I-11-6 to afford cyclized compound I-11-7. Compound I-11-7, in the presence of POCl₃, can be converted to aryl aldehyde to afford compound I-11-8. Compound I-11-8 can be reduced with sodium borohydride to afford compound I-39. The primary alcohol of compound I-39 can be halogenated with DAST to afford compound I-11. Compounds I-40 and I-41 can be made in an analogous manner.

### Synthesis of 6-bromo-2-(8-(2,5-difluoro-4-methylbenzyl)-3-(methylthio)imidazo[1,2-a]pyrazin-6-yl)-5-methylpyrimidin-4-ol (I-12) and 2-(8-(2,5-difluoro-4-methylbenzyl)-3-(methylthio)imidazo[1,2-a]pyrazin-6-yl)-5-methylpyrimidine-4,6-diol (I-38)

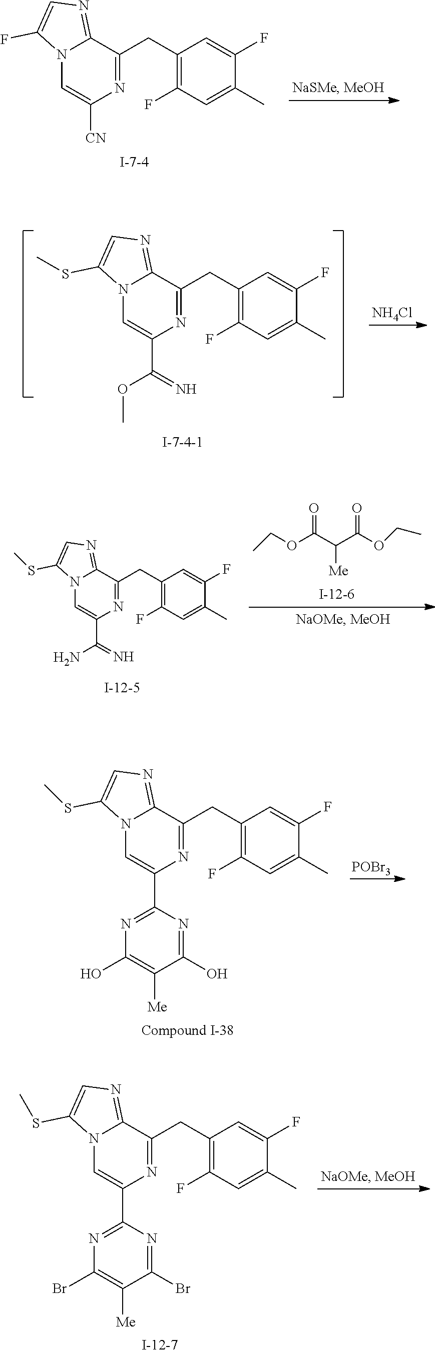

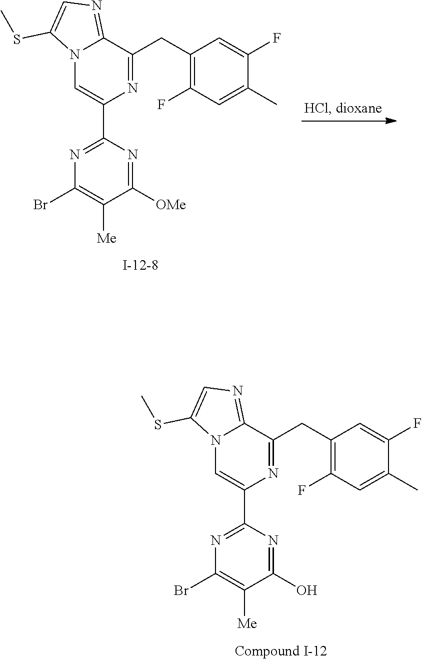

The nitrile of compound I-7-4 was reacted with NaSMe to produce advanced intermediate I-7-4-1, which was reacted in situ with ammonium chloride to afford amidine compound I-12-5. The amidine of compound I-12-5 was condensed with compound I-12-6 to afford cyclized compound I-38. The aryl alchols on the pyrimidine of I-38 were converted to bromine using POBr₃ to afford dibromo compound I-12-7. A single aryl bromide on the pyrimidine ring of compound I-12-7 was converted to —OMe in the presense of sodium methoxide to afford compound I-12-8. Compound I-12-8 was dealkylated to afford hydroxy compound I-12.

### Synthesis of I-12-5

To a stirred solution of 8-[(2,5-difluoro-4-methylphenyl)methyl]-3-fluoroimidazo[1,2-a]pyrazine-6-carbonitrile (I-7-4, 400 mg, 1.324 mmol, 1.0 equiv) in anhydrous MeOH (6 mL) was added sodium methanethiolate (185.3 mg, 2.648 mmol, 2.0 equiv) at room temperature, and the resulting mixture was stirred for 2 h at 45° C. NH₄Cl (141.6 mg, 2.648 mmol, 2.0 equiv) was added to the reaction and the mixture was stirred for 2 h at 45° C. The reaction was then quenched with Water (20 mL) at room temperature and the aqueous layer was extracted with CH₂Cl₂ (3×20 mL). The combined organic layers were washed with brine (20 mL), dried over anhydrous Na₂SO₄ and concentrated under vaccum to afford (8-[(2,5-difluoro-4-methylphenyl)methyl]-3-(methylsulfanyl)imidazo[1,2-a]pyrazine-6-carboximidamide (I-12-5, 360 mg, crude) as a brown solid. This was used in the next step without additional purification. LC-MS: (ESI) m/z 348.05 [M+H].

### Synthesis of I-38

To a stirred solution of 8-[(2,5-difluoro-4-methylphenyl)methyl]-3-(methylsulfanyl)imidazo[1,2-a]pyrazine-6-carboximidamide (I-12-5, 360 mg, 1.036 mmol, 1.0 equiv) in anhydrous MeOH (6 mL) was added 1,3-diethyl 2-methylpropanedioate (I-12-6, 270.8 mg, 1.556 mmol, 1.5 equiv) and NaOMe (653.1 mg, 3.628 mmol, 3.5 equiv, 30% wt in MeOH) at room temperature and the resulting mixture stirred overnight at 70° C. The mixture was allowed to cool down to room temperature and the crude product was precipitated by the addition of 1 M HCl (10 mL) to afford 2-{8-[(2,5-difluoro-4-methylphenyl)methyl]-3-(methylsulfanyl)imidazo[1,2-a]pyrazin-6-yl}-5-methylpyrimidine-4,6-diol (I-38, 400 mg, crude) as a white solid. LC-MS: (ESI) m/z 430.05 [M+H].

### Synthesis of I-12-7

To a stirred solution of 2-{8-[(2,5-difluoro-4-methylphenyl)methyl]-3-(methylsulfanyl)imidazo[1,2-a]pyrazin-6-yl}-5-methylpyrimidine-4,6-diol (I-38, 400 mg, 0.94 mmol, 1.0 equiv) in anhydrous acetonitrile (10 mL) was added POBr₃ (1.08 g, 3.76 mmol, 4.0 equiv) at room temperature and the resulting mixture was refluxed for 6 h. The mixture was allowed to cool down to room temperature and quenched with Water (20 mL). The aqueous layer was extracted with EtOAc (3×20 mL). The combined organic layers were washed with brine (20 mL), dried over anhydrous Na₂SO₄ and concentrated under vaccum. The residue was purified by silica gel column chromatography, eluted with petroleum ether/ethyl acetate (1:1) to afford 6-(4,6-dibromo-5-methylpyrimidin-2-yl)-8-(2,5-difluoro-4-methylbenzyl)-3-(methylthio)imidazo[1,2-a]pyrazine (I-12-7, 440 mg, 85 yield %) as a yellow solid. LC-MS: (ESI) m/z 556.00 [M+H].

### Synthesis of I-12 directly from I-12-7

To a stirred solution of 4,6-dibromo-2-{8-[(2,5-difluoro-4-methylphenyl)methyl]-3-(methylsulfanyl)imidazo[1,2-a]pyrazin-6-yl}-5-methylpyrimidine (I-12-7, 280 mg, 0.504 mmol, 1.0 equiv) in dioxane (5 mL) and THF (5 mL) was added 2 M NaOH aq. (5.60 mL, 11.194 mmol, 22.21 equiv) at room temperature, and the resulting mixture was stirred for 48 h at room temperature. The mixture was acidified with 1 M HCl (10 mL) and the aqueous layer extracted with CH₂Cl₂ (3×10 mL). The combined organic layers were washed with brine (20 mL), dried over anhydrous Na₂SO₄ and concentrated under vaccum. The residue was purified by silica gel column chromatography, eluted with CH₂Cl₂/ethyl acetate (10:1) to afford 6-bromo-2-{8-[(2,5-difluoro-4-methylphenyl)methyl]-3-(methylsulfanyl)imidazo[1,2-a]pyrazin-6-yl}-5-methylpyrimidin-4-ol (I-12, 100 mg, 40% yield) as a white solid. LC-MS: (ESI) m/z 493.95 [M+H]. ¹H NMR: (300 MHz, Chloroform-d) δ 10.67 (s, 1H), 9.26 (s, 1H), 7.99 (s, 1H), 7.01 (dd, J=9.1, 6.5 Hz, 1H), 6.96-6.86 (m, 1H), 4.60 (s, 2H), 2.43 (s, 3H), 2.26 (s, 3H), 2.24 (s, 3H). ¹⁹F NMR: (282 MHz, Chloroform-d) 6-123.03 (d, J=16.8 Hz), −123.09 (d, J=17.8 Hz).

### Synthesis of 2-(8-(2,5-difluoro-4-methylbenzyl)-3-(methoxymethyl)imidazo[1,2-a]pyrazin-6-yl)-5-fluoropyrimidin-4-ol (I-13) and 2-(8-(2,5-difluoro-4-methylbenzyl)-3-(hydroxymethyl)imidazo[1,2-a]pyrazin-6-yl)-5-fluoropyrimidin-4-ol (I-35)

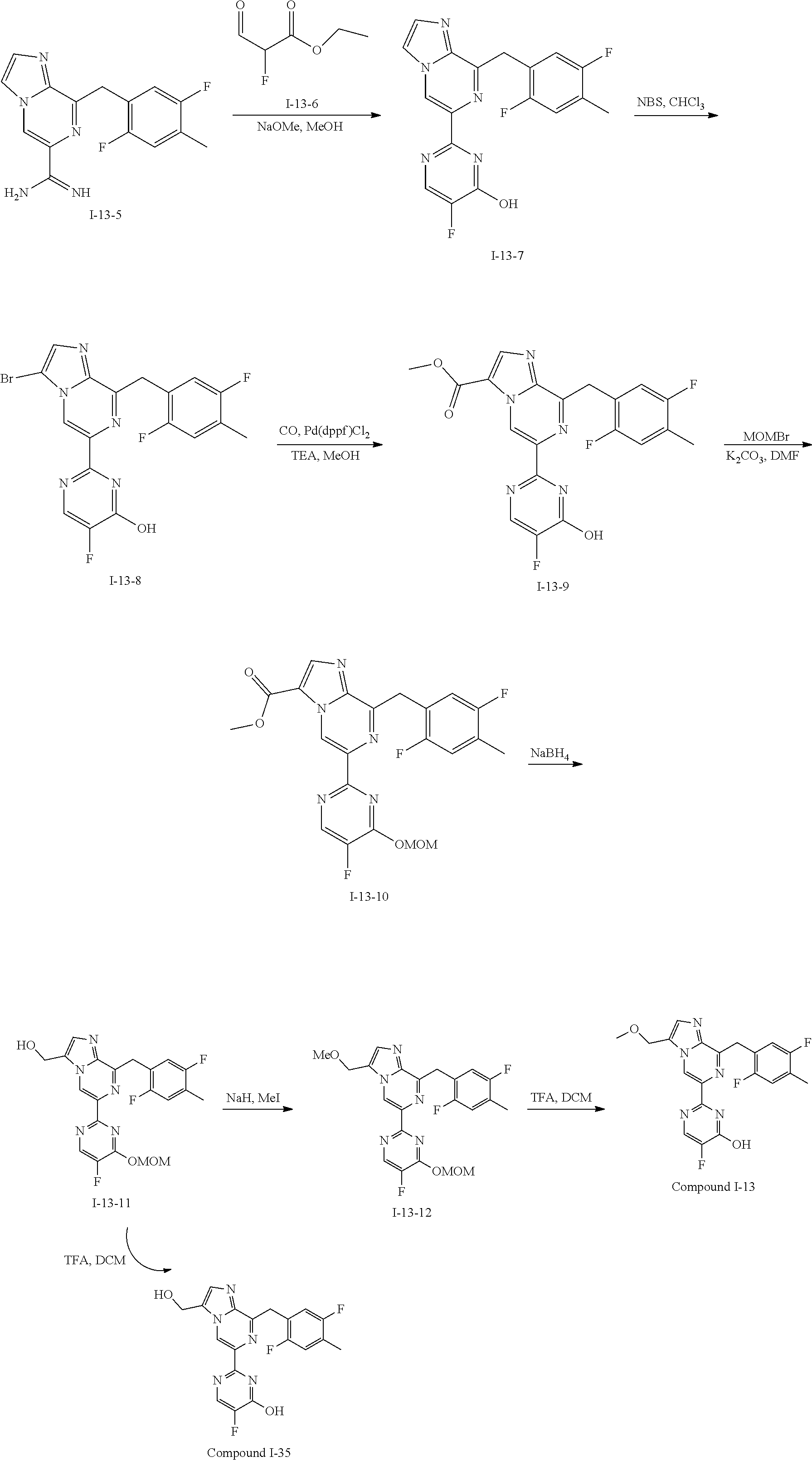

Compound I-13-5 can be synthesized in a manner analogous to compound I-8-5. The amidine of compound I-13-5 can be condensed with compound I-12-6 to afford cyclized compound I-13-7, which can be then converted to the bromide using N-bromosuccinimide. Bromide compound I-13-8 can be esterified using carbon monoxide and methanol in the presence of a palladium catalyst to afford compound I-13-9. The aryl alcohol of the pyrimidine of I-13-9 can be protected using MOMBr to afford compound I-13-10. The ester of compound I-13-10 can be reduced with sodium borohydride to afford alcohol compound I-13-11, which can be then alkylated on the oxygen to afford compound I-13-12. Deprotection of the MOM group of compound I-13-12 can afford compound I-13. Alternatively, compound I-13-11 could be deprotected to afford compound I-35.

### Synthesis of 2-(8-(2,5-difluoro-4-methylbenzyl)-3-(methoxymethyl)imidazo[1,2-a]pyrazin-6-yl)-5-fluoropyrimidin-4-ol (I-13)

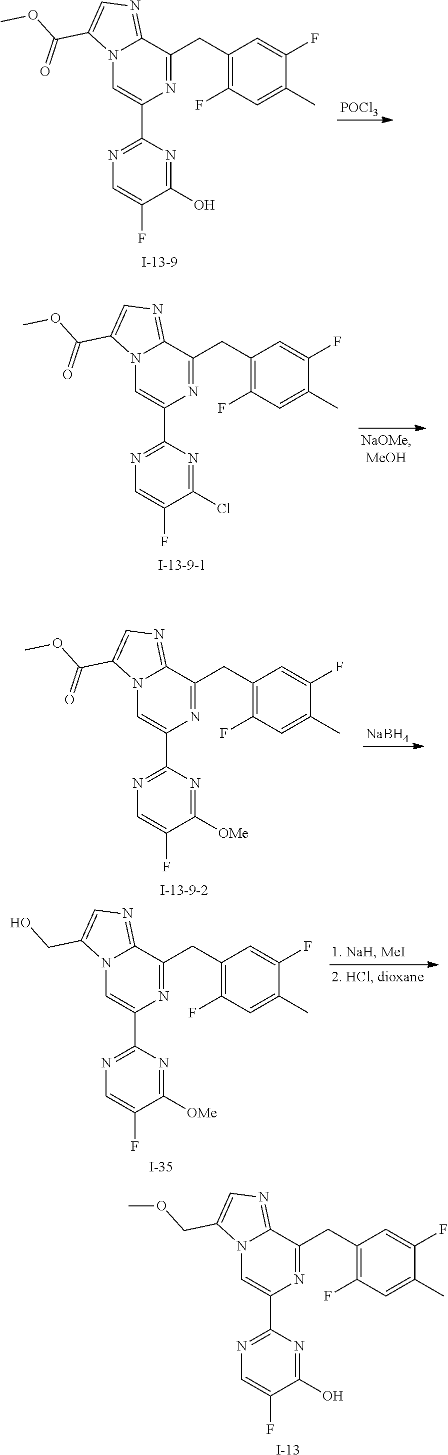

Alternatively, the compound I-13 can be made in the following way. The aryl alchol on the pyrimidine of I-13-9 was converted to chlorine using POCl₃ to afford chloro compound I-13-9-1. The aryl chloride of compound I-13-9-1 was converted to the corresponding methoxide using sodium methoxide in methanol to afford compound I-13-9-2. The ester of compound I-13-9-2 was reduced with sodium borohydride to afford compound I-35. Finally, the alcohol of compound I-13-9-3 was methylated with Mel and NaH, followed by dealkylateion of the aryl methoxy on the pyrimidine with HCl to afford compound I-13.

### Synthesis of Compound I-47 from Compound I-35

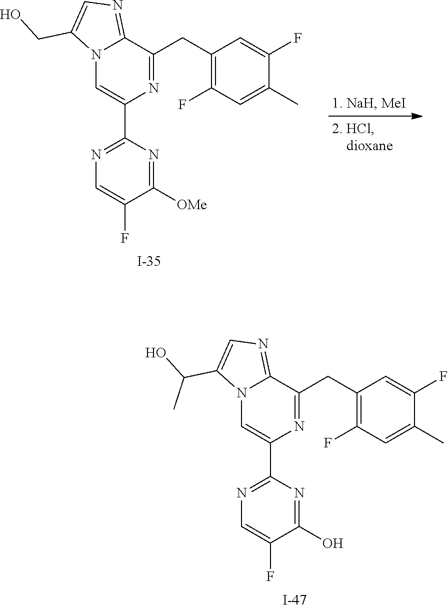

Compound I-35 was prepared as indicated below in the next protocol. This compound could be converted into I-13 by the last step of the above protocol. Alternatively, it was converted to I-47 by using the method described below.

To a stirred solution of 2-{8-[(2,5-difluoro-4-methylphenyl)methyl]-3-(hydroxymethyl)imidazo[1,2-a]pyrazin-6-yl}-5-fluoropyrimidin-4-ol (I-35, 52 mg, 0.130 mmol, 1 equiv) in DMF (2 mL) were added NaH (4.6 mg, 0.195 mmol, 1.5 equiv, 60% in mineral oil) and Mel (36.7 mg, 0.260 mmol, 2 equiv) at 0° C. The mixture was stirred for 4 h at 25° C. The reaction was quenched by the addition of Water (2 mL) at room temperature. The resulting mixture was purified by reverse-phase flash chromatography with the following conditions: column, C18 silica gel; mobile phase, MeCN in Water (0.1% TFA), 5% to 80% gradient in 10 min; and detector, UV 254/220 nm, to afford 2-{8-[(2,5-difluoro-4-methylphenyl)methyl]-3-(methoxymethyl)imidazo[1,2-a]pyrazin-6-yl}-5-fluoropyrimidin-4-ol (1-47, 2 mg, 4% yield) as an off-white solid. LC-MS: (ESI) m/z 416.05 [M+H].

¹H NMR: (400 MHz, CDCl₃) δ 10.97 (s, 1H), 9.19 (s, 1H), 7.90 (d, J=2.6 Hz, 1H), 7.74 (s, 1H), 6.92 (dd, J=9.8, 6.1 Hz, 1H), 6.79 (dd, J=10.1, 6.3 Hz, 1H), 5.31 (q, J=7.1 Hz, 1H), 5.02 (s, 2H), 2.14 (d, J=1.8 Hz, 3H), 1.74 (d, J=7.1 Hz, 3H). ¹⁹F NMR: (376 MHz, CDCl₃) δ −122.51 (d, J=17.4 Hz), −123.36 (d, J=17.6 Hz), −149.44.

### Synthesis of 2-(8-(2,5-difluoro-4-methylbenzyl)-3-(hydroxymethyl)imidazo[1,2-a]pyrazin-6-yl)-5-fluoropyrimidin-4-ol (I-35)

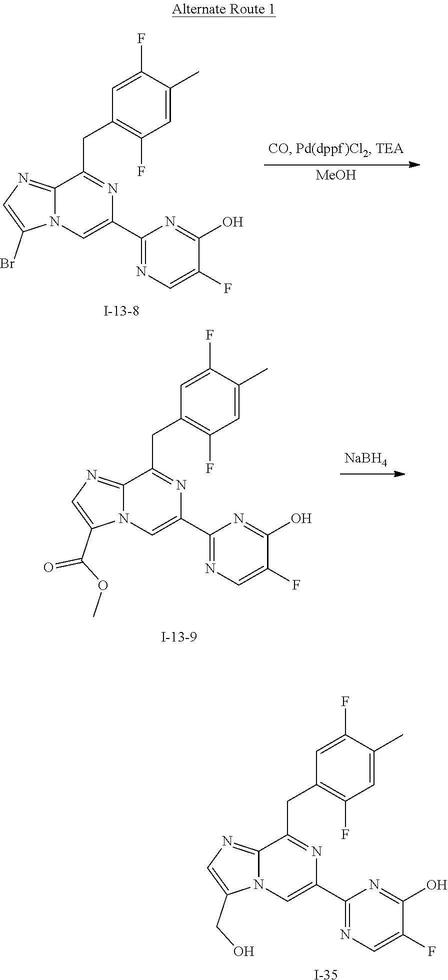

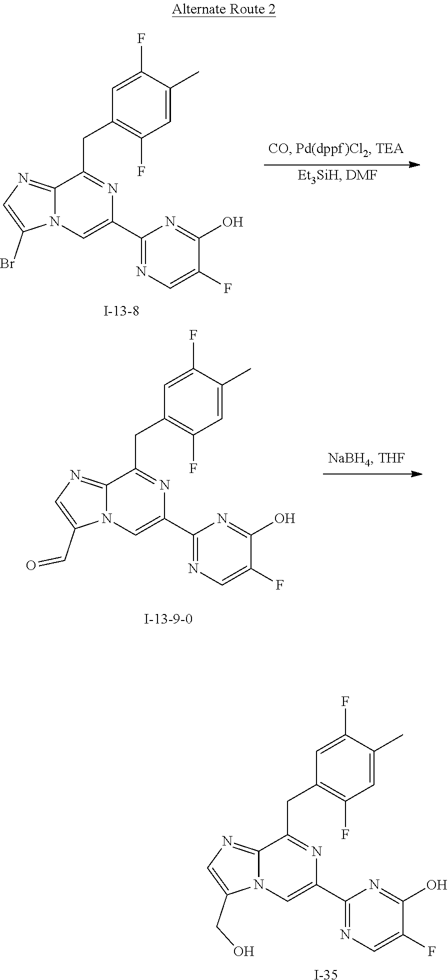

Alternatively, compound I-35 was made by either using alternate route 1 or alternate route 2 from compound I-13-8.

Alternate Route 1: I-13-8 was esterified using carbon monoxide and methanol in the presense of palladium catalyst (1,1′-bis(diphenylphosphino)ferrocene)palladium(II) dichloride to afford compound I-13-9. The ester of compound I-13-9 was reduced with sodium borohydride to afford alcohol compound I-35 directly.

Alternate route 2: I-13-8 was carbonylated using carbon monoxide and triethylsilane in the presense of palladium catalyst (1,1′-bis(diphenylphosphino)ferrocene)palladium(II) dichloride to afford compound I-13-9-0. The aldehyde of compound I-13-9-0 was reduced with sodium borohydride to afford alcohol compound I-35 directly.

### Synthesis of 1-13-7

To a stirred solution of 8-[(2,5-difluoro-4-methylphenyl)methyl]imidazo[1,2-a]pyrazine-6-carboximidamide (I-13-5, 2 g, 6.638 mmol, 1 equiv) in MeOH (20 mL) were added ethyl 2-fluoro-3-oxo-2-sodiopropanoate (I-13-6, 2.23 g, 16.595 mmol, 2.5 equiv) and NaOMe (3.59 g, 19.914 mmol, 3 equiv, 30% wt in MeOH) at 25° C. The mixture was stirred for 8 h at 70° C. The reaction was quenched by the addition of 1 M HCl (aq.) (50 mL) at room temperature and the resulting mixture was extracted with IPA/DCM=1:3 (3×100 mL). The combined organic layers were washed with brine (1×200 mL) and dried over anhydrous Na₂SO₄. After filtration, the filtrate was concentrated under reduced pressure and the residue was purified by silica gel column chromatography, eluted with DCM/MeOH (30:1) to afford 2-{8-[(2,5-difluoro-4-methylphenyl) methyl]imidazo[1,2-a]pyrazin-6-yl}-5-fluoropyrimidin-4-ol (1-13-7, 2.4 g, 97.5% yield) as a yellow solid. LC-MS: (ESI) m/z 372.1 [M+H].

### Synthesis of 1-13-8

To a stirred solution of 2-{8-[(2,5-difluoro-4-methylphenyl)methyl]imidazo[1,2-a]pyrazin-6-yl}-5-fluoropyrimidin-4-ol (1-13-7, 2.4 g, 6.463 mmol, 1 equiv) in CHCl₃ (50 mL) was added NBS (1.27 g, 7.109 mmol, 1.1 equiv) at 25° C. The mixture was stirred for 8 h at 25° C. The reaction was then quenched by the addition of Water (100 mL) at room temperature and the resulting mixture was extracted with DCM (3×100 mL). The combined organic layers were washed with brine (1×200 mL) and dried over anhydrous Na₂SO₄. After filtration, the filtrate was concentrated under reduced pressure and the residue was purified by trituration with ACN (10 mL), the precipitated solids were collected by filtration and washed with ACN (3 mL) and the resulting solid was dried under vacuum to afford 2-{3-bromo-8-[(2,5-difluoro-4-methylphenyl)methyl]imidazo[1,2-a]pyrazin-6-yl}-5-fluoropyrimidin-4-ol (I-13-8, 1.4 g, 48% yield) as a light yellow solid. LC-MS: (ESI) m/z 450.0 [M+H].

### Synthesis of I-13-9-0 (Alternative Route 2)

To a solution of 2-{3-bromo-8-[(2,5-difluoro-4-methylphenyl)methyl]imidazo[1,2-a]pyrazin-6-yl}-5-fluoropyrimidin-4-ol (I-13-8, 1.1 g, 2.443 mmol, 1 equiv) in anhydrous DMF (10 mL) were added Et₃SiH (0.85 g, 7.329 mmol, 3 equiv), Pd(dppf)Cl₂ (0.18 g, 0.244 mmol, 0.1 equiv) and TEA (0.74 g, 7.329 mmol, 3 equiv) in a pressure tank. The mixture was purged with nitrogen for 1 min and then was pressurized to 10 atm with carbon monoxide at 80° C. for 12 h. The reaction mixture was cooled to room temperature and filtered to remove insoluble solids and the resulting mixture was concentrated under reduced pressure. The residue was purified by silica gel column chromatography, eluted with DCM/MeOH (30:1) to afford 8-[(2,5-difluoro-4-methylphenyl)methyl]-6-(5-fluoro-4-hydroxypyrimidin-2-yl)imidazo[1,2-a]pyrazine-3-carbaldehyde (I-13-9-0, 700 mg, 25% yield, 35% purity) as a light yellow solid. LC-MS: (ESI) m/z 400.05 [M+H].

### Synthesis of Compound I-35

To a stirred solution of 8-[(2,5-difluoro-4-methylphenyl)methyl]-6-(5-fluoro-4-hydroxypyrimidin-2-yl)imidazo[1,2-a]pyrazine-3-carbaldehyde crude obtained above (I-13-9-0, 700 mg, 1.753 mmol, 1 equiv) in THF (20 mL) was added NaBH₄ (198.9 mg, 5.259 mmol, 3 equiv) at 0° C. The mixture was stirred for 2 h at 25° C. The reaction was quenched by the addition of Water (10 mL) at room temperature and the resulting mixture was concentrated under reduced pressure. The residue was purified by reverse-phase flash chromatography with the following conditions: column, C18 silica gel; mobile phase, MeCN in Water (0.1% formic acid), 5% to 80% gradient in 10 min; and detector, UV 254/220 nm, to afford 2-{8-[(2,5-difluoro-4-methylphenyl) methyl]-3-(hydroxymethyl)imidazo[1,2-a]pyrazin-6-yl}-5-fluoropyrimidin-4-ol (I-35, 47.2 mg, 6.4% yield) as an off-white solid. LC-MS: (ESI) m/z 402.10 [M+H]. ¹H NMR: (400 MHz, DMSO-d₆) δ 12.69 (s, 1H), 9.19 (s, 1H), 8.21 (s, 1H), 7.83 (s, 1H), 7.35 (s, 1H), 7.14 (dd, J=9.9, 6.6 Hz, 1H), 5.55 (s, 1H), 4.91 (d, J=5.2 Hz, 2H), 4.53 (s, 2H), 2.18 (s, 3H). ¹⁹F NMR: (376 MHz, DMSO-d₆) δ −123.08 (d, J 17.7 Hz), −123.35, −152.58.

### Synthesis of 2-(8-(2,5-difluoro-4-methylbenzyl)-3-(hydroxymethyl)imidazo[1,2-a]pyrazin-6-yl)-5-fluoro-6-(methylthio)pyrimidin-4-ol (I-15)

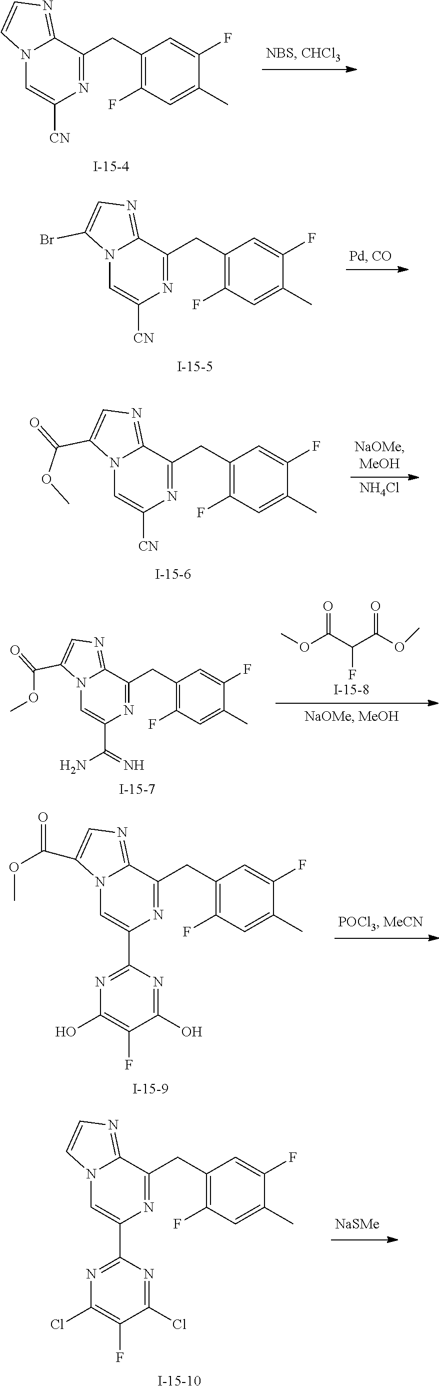

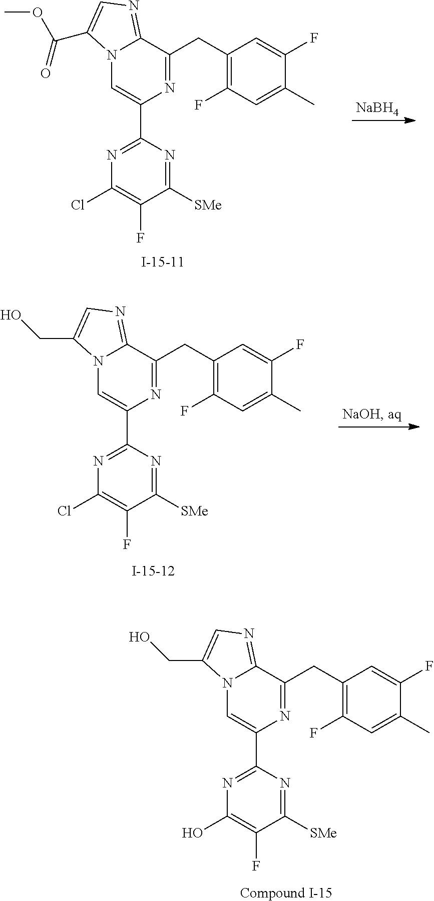

Compound I-15-4 can be synthesized analogously to compound I-8-4. Compound I-15-4 can be brominated in the presence of N-bromosuccinimide to afford compound I-15-5. Compound I-15-5 can be esterified in the presence of carbon monoxide, palladium, and methanol to afford compound I-15-6. The nitrile of compound I-15-6 can be reacted with ammonium chloride to afford amidine compound I-15-7. The amidine of compound I-15-7 can be condensed with compound I-15-8 to afford cyclized compound I-15-9. The aryl alcohols of compound I-15-9, in the presence of POCl₃, can be converted to aryl chlroides to afford compound I-15-10. One of the pyrimidine chlorines of compound I-15-10 can be converted into methyl thioether in the presense of NaSMe to afford compound I-15-11, which can be reduced in the presense of sodium borohydride to afford compound I-15-12. Compound I-15-12 can be hydroxylated in the presence of sodium hydroxide to afford compound I-15.

### Synthesis of 2-(8-(2,5-difluoro-4-hydroxybenzyl)-3-fluoroimidazo[1,2-a]pyrazin-6-yl)-5-fluoro-6-hydroxypyrimidine-4-carbonitrile (I-16)

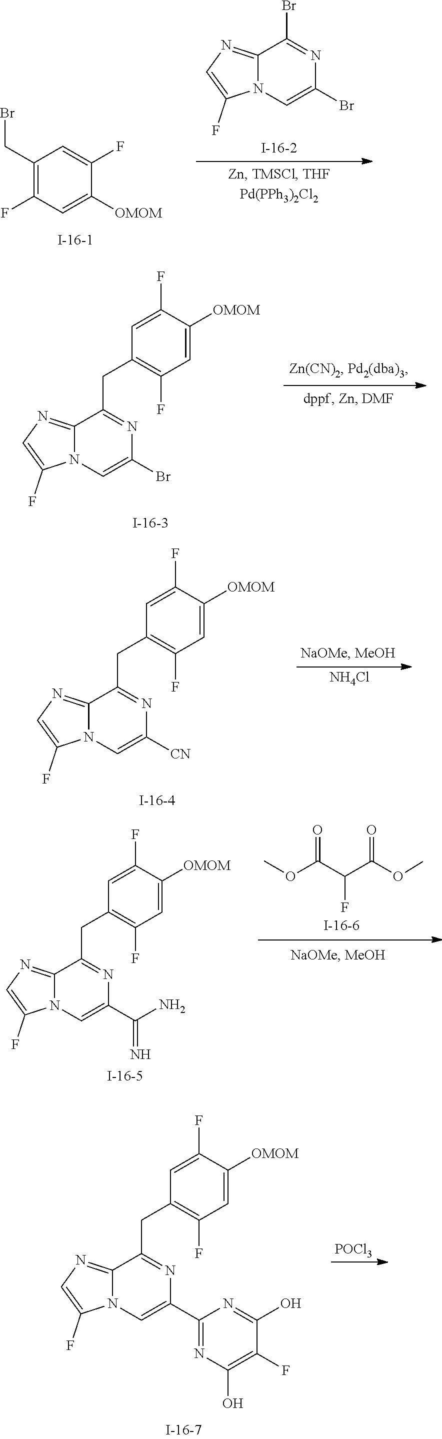

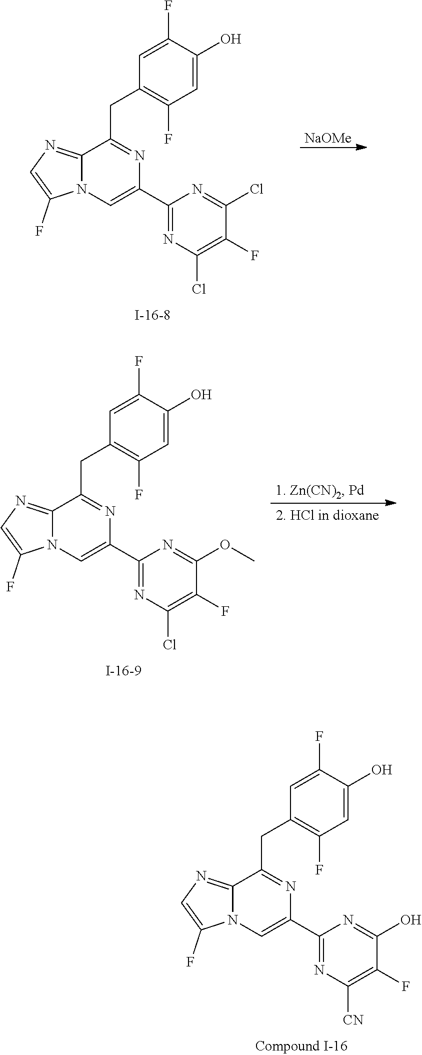 [SMILES: Oc1cc(F)c(Cc2nc(-c3nc(Cl)c(F)c(Cl)n3)cn3c(F)cnc23)cc1F ; COc1nc(-c2cn3c(F)cnc3c(Cc3cc(F)c(O)cc3F)n2)nc(Cl)c1F ; "1. Zn(CN)2, Pd" ; "2. HCl in dioxane" ; "NaOMe" ; N#Cc1nc(-c2cn3c(F)cnc3c(Cc3cc(F)c(O)cc3F)n2)nc(O)c1F]

Compound I-16-1 can be transformed into the corresponding organozinc compound using zinc, dibromoethane, and TMSCl. The organozinc compound can undergo a Negishi coupling in the presence of compound I-16-2 to afford compound I-16-3. Compound I-16-3 can be further reacted in the presence of zinc, zinc cyanide, and tris(dibenzylideneacetone)dipalladium(0) to afford compound I-16-4. The nitrile of compound I-16-4 can be reacted with ammonium chloride to afford amidine compound I-16-5. The amidine of compound I-16-5 can be condensed with compound I-16-6 to afford cyclized compound I-16-7. The aryl alcohols of compound I-16-7, in the presence of POCl3, can be converted to aryl chlorides to afford compound I-16-8. A single aryl chloride on the pyrimidine ring of compound I-16-8 can be converted to —OMe in the presence of sodium methoxide to afford compound I-16-9. The remaining aryl chloride of compound I-16-9 can be converted to compound I-16 using zinc cyanide and hydrochloric acid.

### Synthesis of 2-(8-(2,5-difluoro-4-methylbenzyl)-3-fluoroimidazo[1,2-a]pyrazin-6-yl)-4-hydroxypyrimidine-5-carbonitrile (I-17)

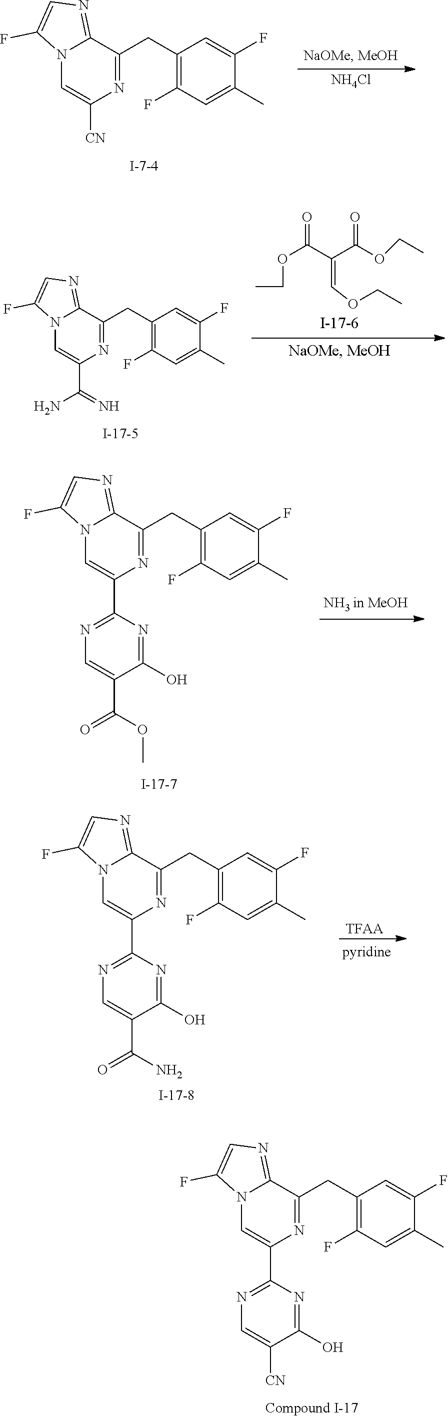

The nitrile of compound I-7-4 was reacted with ammonium chloride to afford amidine compound I-17-5. The amidine of compound I-17-5 was reacted with compound I-17-6 to afford cyclized compound I-17-7. The ester of compound I-17-7 was converted to the amide in the presense of ammonia to afford compound I-17-8, and subsequently converted to the cyano in the presense of TFAA to afford compound I-17.

### Synthesis of I-17-7

To a stirred solution of 8-[(2,5-difluoro-4-methylphenyl)methyl]-3-fluoroimidazo[1,2-a]pyrazine-6-carboximidamide, obtained as indicated elsewhere in this disclosure (I-17-5, 370 mg, 1.159 mmol, 1 equiv) in MeOH (5 mL) were added 1,3-diethyl 2-(ethoxymethylidene)propanedioate (I-17-6, 375.8 mg, 1.739 mmol, 1.5 equiv) and NaOMe (417.3 mg, 2.318 mmol, 2 equiv, 30% in MeOH) at 25° C. The mixture was stirred for 8 h at 70° C. The mixture was allowed to cool down to room temperature and then it was acidified to pH 3 with 1M HCl (10 mL). The resulting mixture was extracted with EtOAc (3×50 mL) and the combined organic layers were washed with brine (1×100 mL) and dried over anhydrous Na₂SO₄. After filtration, the filtrate was concentrated under reduced pressure and the residue was purified by silica gel column chromatography and eluted with DCM/MeOH (30:1) to afford methyl 2-{8-[(2,5-difluoro-4-methylphenyl)methyl]-3-fluoroimidazo[1,2-a]pyrazin-6-yl}-4-hydroxypyrimidine-5-carboxylate (I-17-7, 422 mg, 85% yield) as a yellow solid. LC-MS: (ESI) m/z 430.1 [M+H].

### Synthesis of I-17-8

To a stirred solution of methyl 2-{8-[(2,5-difluoro-4-methylphenyl)methyl]-3-fluoroimidazo[1,2-a]pyrazin-6-yl}-4-hydroxypyrimidine-5-carboxylate (I-17-7, 365 mg, 0.850 mmol, 1 equiv) in MeOH (10 mL) was added NH₃ (g) (60 mL, 420.000 mmol, 494.06 equiv, 7 M in MeOH) at 25° C. The mixture was stirred for 24 h at 90° C. The mixture was then allowed to cool down to room temperature and the resulting mixture was concentrated under reduced pressure to afford 2-{8-[(2,5-difluoro-4-methylphenyl)methyl]-3-fluoroimidazo[1,2-a]pyrazin-6-yl}-4-hydroxypyrimidine-5-carboxamide (1-17-8, 420 mg, crude) as a yellow solid which was directly used in next step. LC-MS: (ESI) m/z 415.1 [M+H].

### Synthesis of Compound I-17

POCl₃ (5 mL) was added to 2-{8-[(2,5-difluoro-4-methylphenyl)methyl]-3-fluoroimidazo[1,2-a]pyrazin-6-yl}-4-hydroxypyrimidine-5-carboxamide (I-17-8, 420 mg, 1.014 mmol, 1 equiv). The mixture was stirred for 2 h at 90° C. and the resulting mixture was concentrated under reduced pressure. The residue was dissolved in dioxane (5 mL) and H₂O (2 mL). Then 2 M NaOH (1.1 mL, 2.028 mmol, 2 equiv) was added and the mixture was stirred for 4 h at 25° C. The resulting mixture was then diluted with water (10 mL) and acidified to pH 3 with 1 M HCl (10 mL). The resulting mixture was extracted with EtOAc (3×100 mL). The combined organic layers were washed with brine (1×100 mL) and dried over anhydrous Na₂SO₄. After filtration, the filtrate was concentrated under reduced pressure and the residue was purified by silica gel column chromatography, eluted with petroleum ether/ethyl acetate (3:1) to afford 2-{8-[(2,5-difluoro-4-methylphenyl)methyl]-3-fluoroimidazo[1,2-a]pyrazin-6-yl}-4-hydroxypyrimidine-5-carbonitrile (I-17, 55.1 mg, 97% purity, 13% yield) as a light yellow solid. LC-MS: (ESI) m/z 397.15 [M+H].

¹H NMR: (400 MHz, DMSO-d₆) δ 13.17 (s, 1H), 9.60 (s, 1H), 8.75 (s, 1H), 8.14 (d, J=7.8 Hz, 1H), 7.34 (t, J=8.1 Hz, 1H), 7.16 (t, J=8.2 Hz, 1H), 4.45 (s, 2H), 2.18 (s, 3H).

¹⁹F NMR: (376 MHz, DMSO-d₆) δ −122.44, −123.15.

### Synthesis of 2-(8-(2,5-difluoro-4-(methoxymethyl)benzyl)imidazo[1,2-a]pyrazin-6-yl)-5-(methylthio)pyrimidin-4-ol (I-18) and 2-(8-(4-bromo-2,5-difluorobenzyl)imidazo[1,2-a]pyrazin-6-yl)-5-(methylthio)pyrimidin-4-ol (I-36)

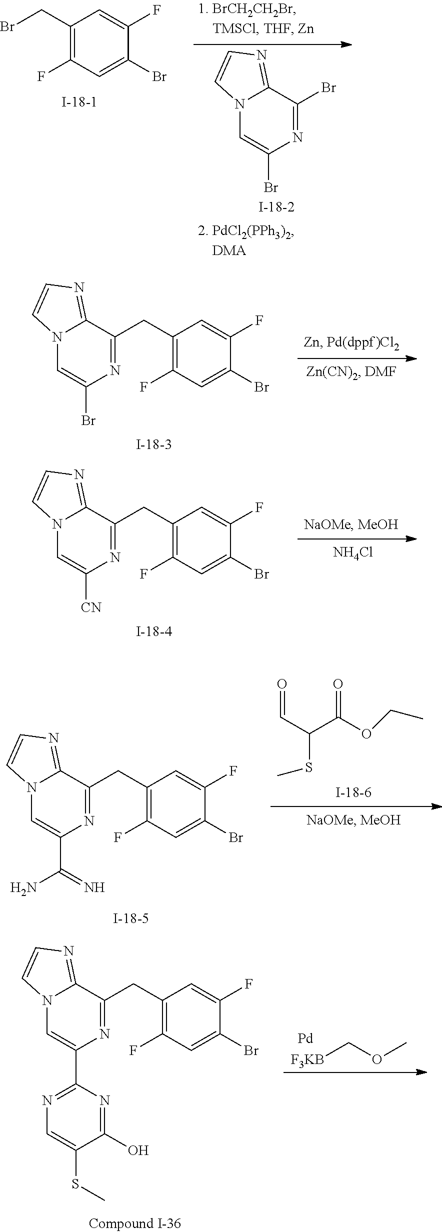

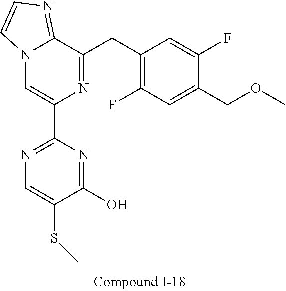 [SMILES: COCc1cc(F)c(Cc2nc(-c3ncc(SC)c(O)n3)cn3ccnc23)cc1F]

Compound I-18-1 can be transformed into the corresponding organozinc compound using zinc, dibromoethane, and TMSCl. The organozinc compound can undergo a Negishi coupling in the presence of compound I-18-2 to afford compound I-18-3. Compound I-18-3 can be further reacted in the presence of zinc, zinc cyanide, and (1,1′-bis(diphenylphosphino)ferrocene)palladium(II) dichloride to afford compound I-18-4. The nitrile of compound I-18-4 can be reacted with ammonium chloride to afford amidine compound I-18-5. The amidine of compound I-18-5 can be condensed with compound I-18-6 to afford cyclized compound I-36. Compound I-36 can be alkylated in a cross-coupling reaction in the presence of palladium to afford compound I-18.

### Synthesis of 6-chloro-5-(difluoromethyl)-2-(3-fluoro-8-(3-fluoro-4-(hydroxymethyl)benzyl)imidazo[1,2-a]pyrazin-6-yl)pyrimidin-4-ol (I-20)

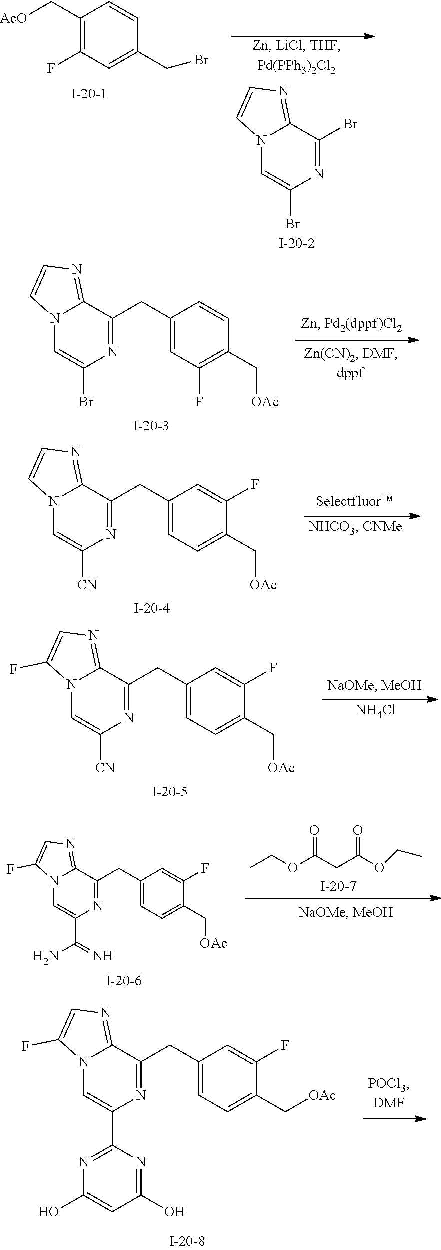

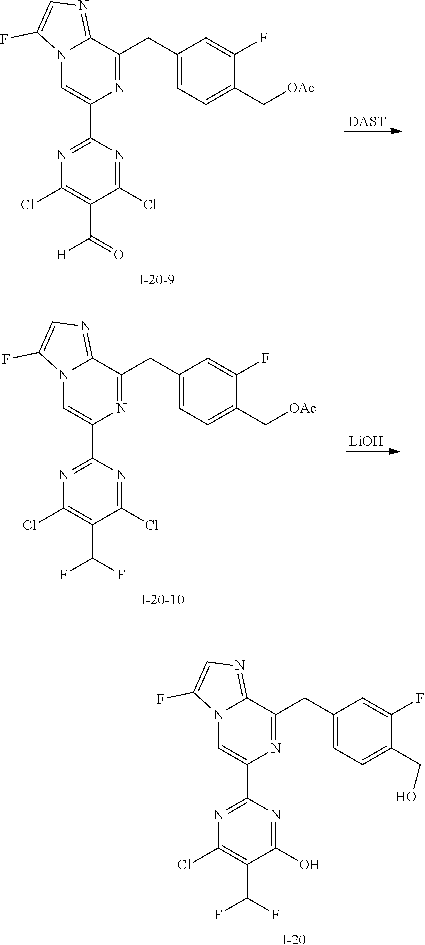

Compound I-20-1 can be transformed into the corresponding organozinc compound using zinc, dibromoethane, and TMSCl. The organozinc compound can undergo a Negishi coupling in the presence of compound I-20-2 to afford compound I-20-3. Compound I-20-3 can be further reacted in the presence of zinc, zinc cyanide, and (1,1′-bis(diphenylphosphino)ferrocene)palladium(II) dichloride to afford compound I-20-4. Compound I-20-4 can be fluorinated in the presence of Selctfluor™ to afford compound I-20-5. The nitrile of compound I-20-5 can be reacted with ammonium chloride to afford amidine compound I-20-6. The amidine of compound I-20-6 can be condensed with compound I-20-7 to afford cyclized compound I-20-8. The aryl hydroxyl groups of I-20-8 can be coverted to chlorine in the presence of POCl₃ to afford compound I-20-9. The acyl substitution of compound I-20-9 can be fluorinated in the presence of DAST to afford compound I-20-10, which was then deproteced in the presence of strong base to afford compound I-20.

### Synthesis of 2-(8-(2,5-difluoro-4-methylbenzyl)imidazo[1,2-a]pyrazin-6-yl)-6-(fluoromethyl)-5-methoxypyrimidin-4-ol (I-21) and 2-(8-(2,5-difluoro-4-methylbenzyl)imidazo[1,2-a]pyrazin-6-yl)-5-methoxy-6-(methoxymethyl)pyrimidin-4-ol (I-27)

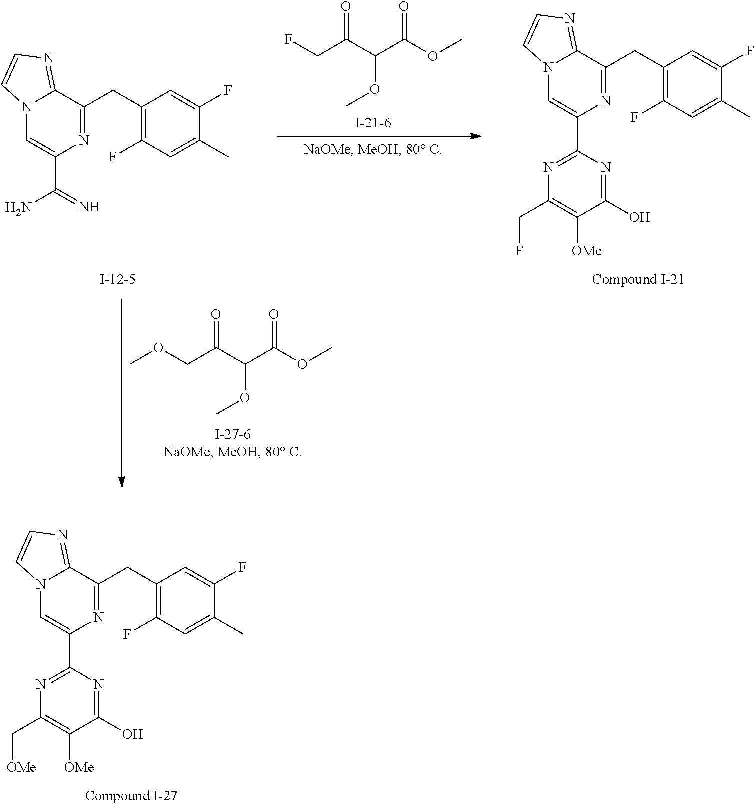

Compound I-12-5 was synthesized in a manner analogous to compound I-8-5. The amidine of compound I-12-5 was condensed with compounds I-21-6 or I-27-6 to afford compounds I-21 or I-27, respectively.

### Synthesis of I-21-6

To a stirred solution of methyl methoxyacetate (2 g, 19.211 mmol, 1 equiv) in THF (40 mL) were added LDA (19.2 mL, 38.422 mmol, 2 equiv, 2 M in THF) and ethyl 2-fluoroacetate (1.85 g, 17.482 mmol, 0.91 equiv) dropwise at −78° C. under a nitrogen atmosphere. The resulting mixture was stirred for 4 h at room temperature under a the nitrogen atmosphere. The reaction was quenched with 1M HCl (aq.) (10 mL) at room temperature and the resulting mixture was extracted with Et₂O (3×40 mL). The combined organic layers were washed with brine (2×100 mL) and dried over anhydrous Na₂SO₄. After filtration, the filtrate was concentrated under reduced pressure to afford methyl 4-fluoro-2-methoxy-3-oxobutanoate (I-21-6, 1.17 g, 37% yield) as a yellow oil. The crude product was used in the next step directly without further purification.

### Synthesis of Compound I-21

To a stirred solution of 8-[(2,5-difluoro-4-methylphenyl)methyl]imidazo[1,2-a]pyrazine-6-carboximidamide, prepared as indicated elsewhere in this disclosure (I-12-5, 20 mg, 0.066 mmol, 1.00 equiv) and methyl 4-fluoro-2-methoxy-3-oxobutanoate (I-21-6, 43.6 mg, 0.264 mmol, 4 equiv) in MeOH (1 mL) was added NaOMe (41.8 mg, 0.231 mmol, 3.5 equiv, 30% wt in MeOH) dropwise at room temperature under a nitrogen atmosphere. The resulting mixture was stirred for 2 h at 80° C. under the nitrogen atmosphere. The resulting mixture was then concentrated under vacuum and the residue was purified by reverse-phase flash chromatography with the following conditions: column, C18 silica gel; mobile phase, MeCN in Water (0.1% formic acid), 0% to 100% gradient in 10 min; and detector, UV 254 nm, to afford 2-{8-[(2,5-difluoro-4-methylphenyl)methyl]imidazo[1,2-a]pyrazin-6-yl}-6-(fluoromethyl)-5-methoxypyrimidin-4-ol (Compound I-21, 15 mg, 54% yield) as an off-white solid. The reaction was repeated 2 times, and a total of 27.5 mg of product was obtained. LC-MS: (ESI) m/z 416.20 [M+H].

¹H NMR: (300 MHz, DMSO-d₆) δ 12.28 (s, 1H), 9.46 (s, 1H), 8.35 (d, J=1.1 Hz, 1H), 7.88 (d, J=1.1 Hz, 1H), 7.38 (dd, J=10.0, 6.3 Hz, 1H), 7.16 (dd, J=9.9, 6.5 Hz, 1H), 5.44 (s, 1H), 5.28 (s, 1H), 4.54 (s, 2H), 3.92 (s, 3H), 2.19 (d, J=1.9 Hz, 3H).

¹⁹F NMR: (376 MHz, DMSO-d₆) δ −73.40, −123.13 (d, J=18.1 Hz), −123.39 (d, J=18.1 Hz).

### Synthesis of I-27-6

To a stirred solution of methyl methoxyacetate (500 mg, 4.803 mmol, 1 equiv) in THF (10 mL) was added LDA (4.8 mL, 9.606 mmol, 2 equiv, 2 M in THF) dropwise at −78° C. under a nitrogen atmosphere. The resulting mixture was stirred for 4 h at room temperature under the nitrogen atmosphere. The reaction was quenched with 1M HCl (aq.) (10 mL) at room temperature and the resulting mixture was extracted with Et₂O (3×15 mL). The combined organic layers were washed with brine (2×40 mL) and dried over anhydrous Na₂SO₄. After filtration, the filtrate was concentrated under reduced pressure to afford methyl 2,4-dimethoxy-3-oxobutanoate (I-27-6, 190 mg, 22% yield, crude) as a yellow oil. The crude product was used in the next step directly without further purification.

### Synthesis of Compound I-27

To a stirred solution of 8-[(2,5-difluoro-4-methylphenyl)methyl]imidazo[1,2-a]pyrazine-6-carboximidamide (I-12-5, 56 mg, 0.186 mmol, 1 equiv) and methyl 2,4-dimethoxy-3-oxobutanoate (1-27-6, 131 mg, 0.744 mmol, 4 equiv) in MeOH (1 mL) was added NaOMe (117.1 mg, 0.651 mmol, 3.5 equiv, 30% wt in MeOH) dropwise at room temperature under a nitrogen atmosphere. The resulting mixture was stirred for 2 h at 80° C. under the nitrogen atmosphere. The mixture was then acidified to pH 4 with 1M HCl (aq.) (1 mL) and it was extracted with EtOAc (3×10 mL) and dried over anhydrous Na₂SO₄. After filtration, the filtrate was concentrated under reduced pressure and the residue was purified by trituration with MeOH (2 mL) to afford 2-{8-[(2,5-difluoro-4-methylphenyl)methyl]imidazo[1,2-a]pyrazin-6-yl}-5-methoxy-6-(methoxymethyl)pyrimidin-4-ol (1-27, 53.5 mg, 75.5% yield) as an off-white solid. LC-MS: (ESI) m/z 428.05 [M+H]. ¹H NMR: (400 MHz, DMSO-d₆) δ 12.08 (s, 1H), 9.43 (s, 1H), 8.35 (d, J=1.2 Hz, 1H), 7.88 (d, J=1.2 Hz, 1H), 7.36 (dd, J=10.1, 6.3 Hz, 1H), 7.16 (dd, J=9.9, 6.4 Hz, 1H), 4.53 (s, 2H), 4.37 (s, 2H), 3.86 (s, 3H), 3.35 (s, 3H), 2.19 (d, J=2.0 Hz, 3H).

¹⁹F NMR: (377 MHz, DMSO-d₆) δ −74.24, −123.27 (dd, J=98.5, 18.1 Hz).

### Synthesis of 2-(8-(2,5-difluoro-4-methylbenzyl)imidazo[1,2-a]pyrazin-6-yl)-5-fluoro-6-(methoxymethyl)pyrimidin-4-ol (I-22)

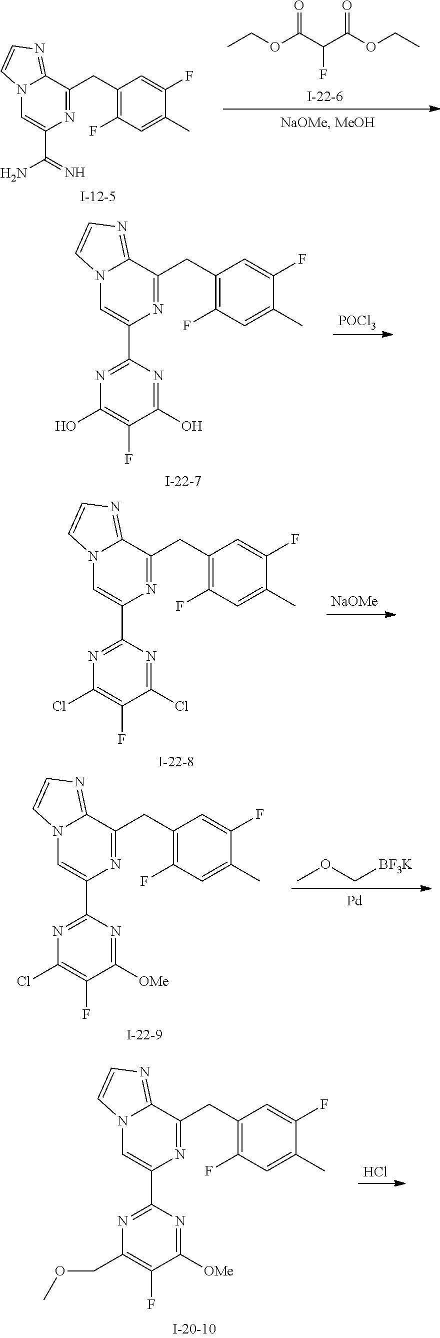

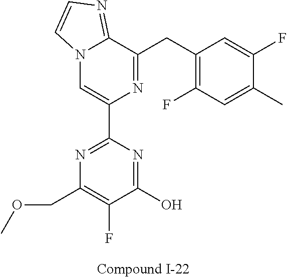

Compound I-12-5 was synthesized in a manner analogous to compound I-8-5. The amidine of compound I-12-5 was condensed with compound I-22-6 to afford cyclized compound I-22-7. The aryl hydroxyl groups of compound I-22-7 were converted to chlorine in the presence of POCl₃ to afford compound I-22-8. A single aryl chlorine of I-22-8 was converted to the methoxy ether in the presence of sodium methoxide to afford compound I-22-9. Compound I-22-9 was alkylated in a cross-coupling reaction in the presence of palladium to afford compound I-22-10, which was delkylated to afford compound I-22.

### Synthesis of I-22-7

To a stirred mixture of 8-[(2,5-difluoro-4-methylphenyl)methyl]imidazo[1,2-a]pyrazine-6-carboximidamide prepared as described elsewhere in this disclosure (I-12-5, 2 g, 6.638 mmol, 1 equiv) and 1,3-dimethyl 2-fluoropropanedioate (1-22-6, 1.49 g, 9.957 mmol, 1.5 equiv) in MeOH (20 mL) was added NaOMe (3.59 g, 19.914 mmol, 3 equiv, 30% in MeOH) at room temperature under a nitrogen atmosphere. The resulting mixture was stirred for 1.5 h at 70° C. under the nitrogen atmosphere. The mixture was then allowed to cool down to room temperature and diluted with water (50 mL). The residue was acidified to pH 5 with HCl (aq.) and the precipitated solids were collected by filtration and washed with water (2×50 mL) to afford 2-{8-[(2,5-difluoro-4-methylphenyl)methyl]imidazo[1,2-a]pyrazin-6-yl}-5-fluoropyrimidine-4,6-diol (I-22-7, 1.6 g, 62%) as a light yellow solid. LC-MS: (ESI) m/z 388.10 [M+H].

### Synthesis of I-22-8

To a stirred solution of 2-{8-[(2,5-difluoro-4-methylphenyl)methyl]imidazo[1,2-a]pyrazin-6-yl}-5-fluoropyrimidine-4,6-diol (I-22-7, 500 mg, 1.291 mmol, 1 equiv) in ACN (10.00 mL) was added phosphorus oxychloride (989.6 mg, 6.455 mmol, 5 equiv), in portions, at room temperature under a nitrogen atmosphere. The resulting mixture was stirred for 2 h at 90° C. under the nitrogen atmosphere. The mixture was allowed to cool down to room temperature. The reaction was then quenched by the addition of Water (20 mL) at 0° C. and the resulting mixture was extracted with EtOAc (3×20 mL). The combined organic layers were washed with brine (2×20 mL) and dried over anhydrous Na₂SO₄. After filtration, the filtrate was concentrated under reduced pressure to afford 4,6-dichloro-2-{8-[(2,5-difluoro-4-methylphenyl)methyl]imidazo[1,2-a]pyrazin-6-yl}-5-fluoropyrimidine (I-22-8, 500 mg, crude) as a light yellow solid. LC-MS: (ESI) m/z 424.0 [M+H].

### Synthesis of I-22-9

To a stirred solution of 4,6-dichloro-2-{8-[(2,5-difluoro-4-methylphenyl)methyl]imidazo[1,2-a]pyrazin-6-yl}-5-fluoropyrimidine (I-22-8, 600 mg, 1.414 mmol, 1 equiv) in MeOH (12 mL) was added sodium methoxide (382.0 mg, 2.121 mmol, 1.5 equiv, 30% wt in MeOH), in portions, at room temperature and under a nitrogen atmosphere. The resulting mixture was stirred for 2 h at room temperature and under the nitrogen atmosphere. The resulting mixture was concentrated under reduced pressure and then it was diluted with water (20 mL). The resulting mixture was extracted with EtOAc (3×20 mL). The combined organic layers were washed with brine (2×10 mL) and dried over anhydrous Na₂SO₄. After filtration, the filtrate was concentrated under reduced pressure to afford 4-chloro-2-{8-[(2,5-difluoro-4-methylphenyl)methyl]imidazo[1,2-a]pyrazin-6-yl}-5-fluoro-6-methoxypyrimidine (I-22-9, 460 mg, crude) as a light yellow solid. LC-MS: (ESI) m/z 420.10 [M+H].

### Synthesis of 1-22-10

To a stirred mixture of 4-chloro-2-{8-[(2,5-difluoro-4-methylphenyl)methyl]imidazo[1,2-a]pyrazin-6-yl}-5-fluoro-6-methoxypyrimidine (I-22-9, 200 mg, 0.476 mmol, 1 equiv) and trifluoro(methoxymethyl)-lambda4-borane potassium (144.8 mg, 0.952 mmol, 2 equiv) in dioxane (5 mL) and H₂O (1 mL) were added K₃PO₄ (202.2 mg, 0.952 mmol, 2 equiv) and Pd(Amphos)₂Cl₂ (67.4 mg, 0.095 mmol, 0.2 equiv) at room temperature and under a nitrogen atmosphere. The resulting mixture was stirred overnight at 90° C. under the nitrogen atmosphere. The mixture was then allowed to cool down to room temperature and it was filtered. The filter cake was washed with THF (3×5 mL) and the filtrate was concentrated under reduced pressure and purified by reverse-phase flash chromatography with the following conditions: column, C18 silica gel; mobile phase, MeCN in Water (0.1% formic acid), 0% to 100% gradient in 10 min; and detector, UV 254 nm, to afford 2-{8-[(2,5-difluoro-4-methylphenyl)methyl]imidazo[1,2-a]pyrazin-6-yl}-5-fluoro-4-methoxy-6-(methoxymethyl)pyrimidine (I-22-10, 80 mg, 39%) as a light yellow solid. LC-MS: (ESI) m/z 430.20 [M+H].

### Synthesis of Compound I-22

A solution of 2-{8-[(2,5-difluoro-4-methylphenyl)methyl]imidazo[1,2-a]pyrazin-6-yl}-5-fluoro-4-methoxy-6-(methoxymethyl)pyrimidine (1-22-10, 80 mg, 0.186 mmol, 1 equiv) in HCl(gas) in 1,4-dioxane (2 mL) was stirred overnight at 60° C. under a nitrogen atmosphere. The mixture was allowed to cool down to room temperature and concentrated under reduced pressure. The residue was then purified by reverse-phase flash chromatography with the following conditions: column, C18 silica gel; mobile phase, MeCN in Water (0.1% formic acid), 0% to 100% gradient in 10 min; and detector, UV 254 nm, to afford 2-{8-[(2,5-difluoro-4-methylphenyl)methyl]imidazo[1,2-a]pyrazin-6-yl}-5-fluoro-6-(methoxymethyl)pyrimidin-4-ol (1-22, 42.1 mg, 54.5% yield) as an off-white solid. LC-MS: (ESI) m/z 416.15 [M+H].

¹H NMR: (400 MHz, DMSO-d₆) δ 12.62 (s, 1H), 9.45 (s, 1H), 8.34 (d, J=1.2 Hz, 1H), 7.87 (d, J=1.1 Hz, 1H), 7.36 (dd, J=10.1, 6.2 Hz, 1H), 7.15 (dd, J=10.0, 6.4 Hz, 1H), 4.52 (s, 2H), 4.42 (d, J=3.0 Hz, 2H), 3.37 (s, 3H), 2.18 (d, J=2.0 Hz, 3H).

### Synthesis of 5-chloro-2-(8-(2,5-difluorobenzyl)-3-methoxyimidazo[1,2-a]pyrazin-6-yl)pyrimidin-4-ol (I-29)

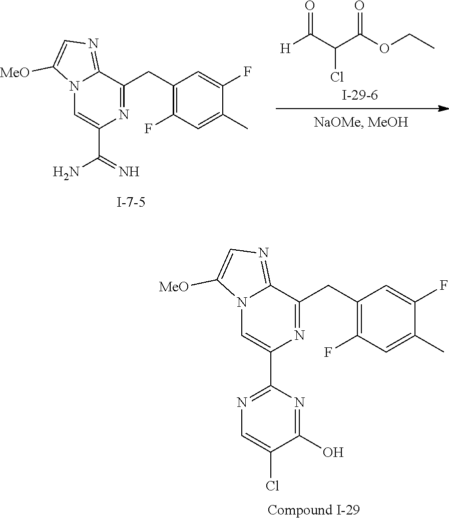

The amidine of compound I-7-5 was condensed with compound I-29-6 to afford cyclized compound I-29.

### Synthesis of I-7-5

To a stirred solution of 8-[(2,5-difluoro-4-methylphenyl)methyl]-3-fluoroimidazo[1,2-a]pyrazine-6-carbonitrile (I-7-4, 560 mg, 1.853 mmol, 1 equiv, prepared as described above) in MeOH (10 mL) was added NaOMe (1.0 g, 5.559 mmol, 3 equiv, 30% in MeOH), at room temperature under a nitrogen atmosphere. The resulting mixture was stirred overnight at 50° C. under the nitrogen atmosphere. To the above mixture was added NH₄Cl (198.2 mg, 3.706 mmol, 2 equiv) at 50° C. The resulting mixture was stirred for an additional 2 h at 70° C. The mixture was then allowed to cool down to room temperature. The reaction was quenched by the addition of sat. NaHCO₃ (aq.) (40 mL) at room temperature and the resulting mixture was extracted with CH₂Cl₂/IPA (3/1) (3×20 mL). The combined organic layers were dried over anhydrous Na₂SO₄. After filtration, the filtrate was concentrated under reduced pressure to afford crude product 8-[(2,5-difluoro-4-methylphenyl)methyl]-3-methoxyimidazo[1,2-a]pyrazine-6-carboximidamide (I-7-5, 500 mg, crude) as a brown solid. This was used directly in the next step without additional purification. LC-MS: (ESI) m/z 332.05 [M+H].

### Synthesis of Compound I-29

To a stirred solution of 8-[(2,5-difluoro-4-methylphenyl)methyl]-3-methoxyimidazo[1,2-a]pyrazine-6-carboximidamide (1-7-5, 160 mg, 0.483 mmol, 1 equiv) and ethyl 2-chloro-3-oxopropanoate (I-29-6, 109.06 mg, 0.724 mmol, 1.5 equiv) in MeOH (3 mL) was added NaOMe (173.9 mg, 0.966 mmol, 2 equiv, 30% wt in MeOH) at room temperature under a nitrogen atmosphere. The resulting mixture was stirred for 3 h at 70° C. under the nitrogen atmosphere. The mixture was allowed to cool down to room temperature and the reaction was quenched by the addition of 1M HCl (5 mL) at room temperature. The precipitated solids were collected by filtration and washed with water (3×5 mL) to afford 5-chloro-2-{8-[(2,5-difluoro-4-methylphenyl)methyl]-3-methoxyimidazo[1,2-a]pyrazin-6-yl}pyrimidin-4-ol (I-29, 51.0 mg, 25%) as a light yellow solid. LC-MS: (ESI) m/z 418.15 [M+H]. ¹H NMR: (400 MHz, Chloroform-d) δ 10.88 (s, 1H), 8.92 (s, 1H), 8.13 (s, 1H), 7.28 (s, 1H), 7.04-6.97 (m, 1H), 6.92 (t, J=8.0 Hz, 1H), 4.55 (s, 2H), 4.17 (s, 3H), 2.23 (s, 3H). ¹⁹FNMR: (376 MHz, Chloroform-d) 6-123.12 (d, J=2.8 Hz), −150.71.

### Synthesis of 1-(2-(8-(2,5-difluoro-4-methylbenzyl)imidazo[1,2-a]pyrazin-6-yl)-4-hydroxypyrimidin-5-yl)ethan-1-one (I-24) and 2-(8-(2,5-difluoro-4-methylbenzyl)imidazo[1,2-a]pyrazin-6-yl)-5-(1-hydroxyethyl)pyrimidin-4-ol (I-28)

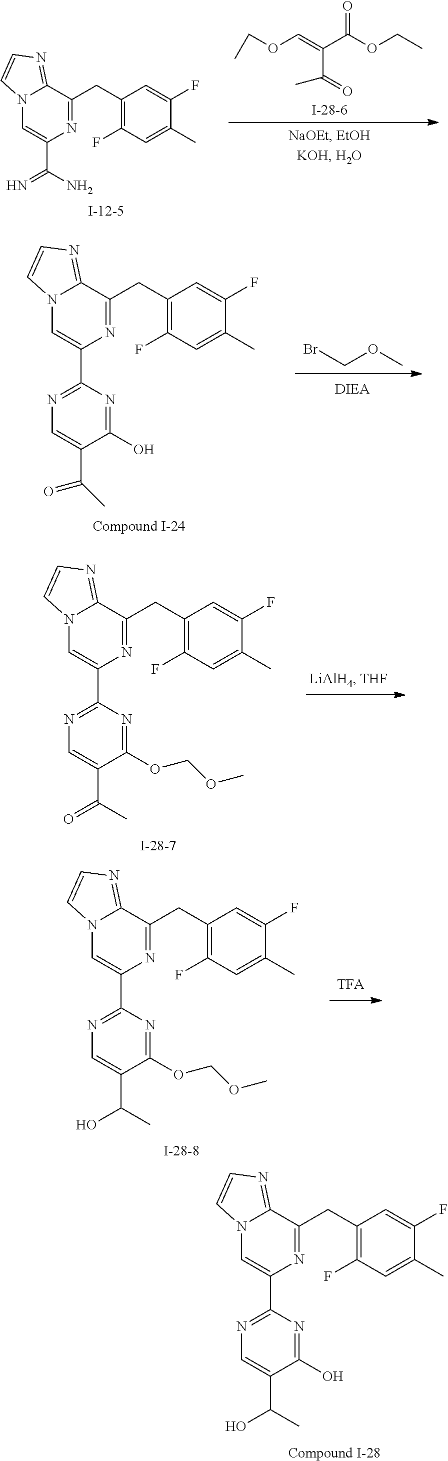

Compound I-12-5 was synthesized in a manner analogous to compound I-8-5. The amidine of compound I-12-5 was cyclized with compound I-28-6 to afford compound I-24. The aryl hydroxyl of compound I-24 was alkylated in the presence of bromo dimethyl ether to afford compound I-28-7. The aryl ketone of compound I-28-7 was reduced in the presence of lithium aluminium hydride to afford compound I-28-8, which was then deprotected in the presence of TFA to afford compound I-28.

### Synthesis of Compound I-24

A solution of 8-[(2,5-difluoro-4-methylphenyl)methyl]imidazo[1,2-a]pyrazine-6-carboximidamide, synthesized as described elsewhere in this disclosure (I-12-5, 200 mg, 0.664 mmol, 1 equiv) in MeOH (4 mL) was treated with ethyl (2E)-2-(ethoxymethylidene)-3-oxobutanoate (I-28-6, 124 mg, 0.666 mmol, 1.00 equiv) for 5 min at room temperature under a nitrogen atmosphere followed by the addition of NaOMe (418 mg, 2.321 mmol, 3.50 equiv, 30% wt in MeOH) dropwise at 70° C. The resulting mixture was stirred for an additional 4 h at 70° C. and it was then concentrated under reduced pressure. The residue was acidified to pH 4 with 1M HCl aq. (3 mL) and then purified by trituration with THF (50 mL) to afford 1-(2-{8-[(2,5-difluoro-4-methylphenyl)methyl]imidazo[1,2-a]pyrazin-6-yl}-4-hydroxypyrimidin-5-yl)ethanone (I-24, 85.6 mg, 30% yield) as a yellow solid. LC-MS: (ESI) m/z 396.15 [M+H]. ¹H NMR: (400 MHz, DMSO-d₆) δ 13.70 (s, 1H), 9.64 (s, 1H), 9.19 (s, 1H), 8.34 (d, J=1.2 Hz, 1H), 7.86 (d, J=1.2 Hz, 1H), 7.17 (ddd, J=20.9, 10.1, 6.4 Hz, 2H), 4.55 (s, 2H), 2.83 (s, 3H), 2.18 (d, J=2.0 Hz, 3H). ¹⁹F NMR: (377 MHz, DMSO-d₆) δ −123.03 (d, J=18.1 Hz), −123.46 (d, J=18.0 Hz).

### Synthesis of Compound I-28-7

To a stirred solution of 1-(2-{8-[(2,5-difluoro-4-methylphenyl)methyl]imidazo[1,2-a]pyrazin-6-yl}-4-hydroxypyrimidin-5-yl)ethanone (I-24, 1.4 g, 3.541 mmol, 1 equiv) and bromomethoxy-methane (0.88 g, 7.082 mmol, 2 equiv) in DCM (25 mL) was added DIEA (1.14 g, 8.852 mmol, 2.5 equiv) dropwise at 0° C. under a nitrogen atmosphere. The resulting mixture was stirred for an additional 4 h at room temperature. The reaction was then quenched by the addition of Water (50 mL) at room temperature. The resulting mixture was extracted with DCM (3×50 mL) and the combined organic layers were washed with brine (2×50 mL) and dried over anhydrous Na₂SO₄. After filtration, the filtrate was concentrated under reduced pressure and the residue was purified by reverse-phase flash chromatography with the following conditions: column, C18 silica gel; mobile phase, MeCN in Water (0.1% FA), 0% to 100% gradient in 10 min; and detector, UV 254 nm, to afford 1-(2-{8-[(2,5-difluoro-4-methylphenyl)methyl]imidazo[1,2-a]pyrazin-6-yl}-4-(methoxymethoxy)pyrimidin-5-yl)ethanone (I-28-7, 680 mg, 44% yield) as a white solid. LC-MS: (ESI) m/z 440.2 [M+H].

### Synthesis of Compound I-28-8

To a stirred solution of 1-(2-{8-[(2,5-difluoro-4-methylphenyl)methyl]imidazo[1,2-a]pyrazin-6-yl}-4-(methoxymethoxy)pyrimidin-5-yl)ethanone (1-28-7, 680 mg, 1.547 mmol, 1 equiv) in THF (14 mL) was added NaBH₄ (176 mg, 4.652 mmol, 3.01 equiv) in portions at 0° C. under nitrogen atmosphere. The resulting mixture was stirred for an additional 2 h at room temperature. The reaction was quenched by the addition of Water (40 mL) at 0° C. and the resulting mixture was extracted with EtOAc (3×40 mL). The combined organic layers were washed with brine (2×40 mL) and dried over anhydrous Na₂SO₄. After filtration, the filtrate was concentrated under reduced pressure and the residue was purified by reverse-phase flash chromatography with the following conditions: column, C18 silica gel, mobile phase, MeCN in Water (10 mmol/L NH₄HCO₃), 0% to 100% gradient in 10 min, detector, UV 254 nm to afford 1-(2-{8-[(2,5-difluoro-4-methylphenyl)methyl]imidazo[1,2-a]pyrazin-6-yl}-4-(methoxymethoxy)pyrimidin-5-yl)ethanol (I-28-8, 110 mg, 16%) as a yellow solid.

LC-MS: (ESI) m/z 442.0 [M+H].

### Synthesis of Compound I-28

Into a 50 mL round-bottom flask were added 1-(2-{8-[(2,5-difluoro-4-methylphenyl)methyl]imidazo[1,2-a]pyrazin-6-yl}-4-(methoxymethoxy)pyrimidin-5-yl)ethanol (I-28-8, 110 mg, 0.249 mmol, 1 equiv), TFA (1 mL) and DCM (3 mL) at room temperature. The resulting mixture was stirred for 1 h at room temperature. The resulting mixture was then diluted with DCM (2×10 mL) and concentrated under reduced pressure. The residue was purified by reverse-phase flash chromatography with the following conditions: column, C18 silica gel; mobile phase, MeCN in Water (10 mmol/L NH₄HCO₃), 0% to 100% gradient in 10 min; and detector, UV 254 nm, to afford 2-{8-[(2,5-difluoro-4-methylphenyl)methyl]imidazo[1,2-a]pyrazin-6-yl}-5-(1-hydroxyethyl)pyrimidin-4-ol (I-28, 50 mg, 47.5%) as a yellow solid. LC-MS (ESI) m/z 398.05 [M+H]. ¹H NMR: (400 MHz, DMSO-d₆) δ 11.86 (s, 1H), 9.24 (s, 1H), 8.27 (s, 1H), 7.86 (d, J=1.1 Hz, 1H), 7.23 (dd, J=9.9, 6.3 Hz, 1H), 7.17 (dd, J=9.8, 6.5 Hz, 1H), 4.52 (s, 2H), 4.24 (s, 2H), 2.24 (s, 3H), 2.19 (d, J=1.9 Hz, 3H). ¹⁹F NMR: (376 MHz, DMSO-d₆) δ −73.40, −123.12 (d, J=17.7 Hz).

### Synthesis of 2-(8-(2,5-difluoro-4-methylbenzyl)imidazo[1,2-a]pyrazin-6-yl)-5-(hydroxymethyl)pyrimidin-4-ol (I-25)

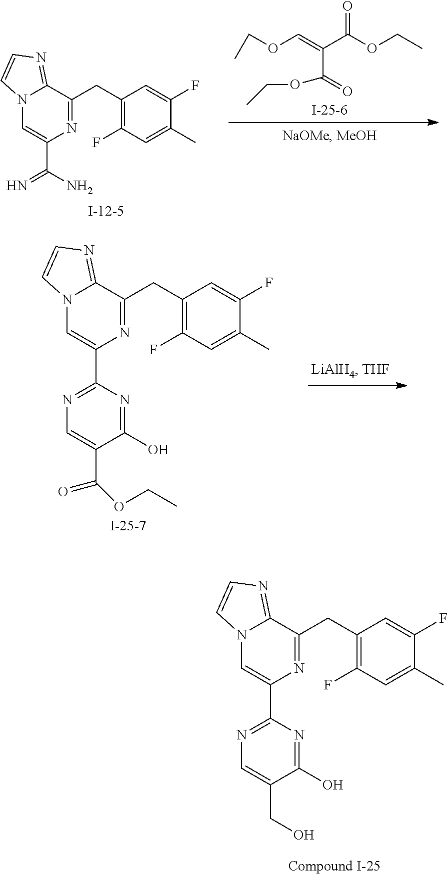

Compound I-12-5 was synthesized in a manner analogous to compound I-8-5. The amidine of compound I-12-5 was condensed with compound I-25-6 to afford cyclized compound I-25-7. The aryl ester of compound I-25-7 was reduced in the presense of lithium aluminium hydride to afford compound I-25.

### Synthesis of I-25-7

A solution of 8-[(2,5-difluoro-4-methylphenyl)methyl]imidazo[1,2-a]pyrazine-6-carboximidamide (2 g, 6.638 mmol, 1 equiv) in MeOH (40 mL) was treated with 1,3-diethyl 2-(ethoxymethylidene)propanedioate (I-25-6, 1.44 g, 6.638 mmol, 1 equiv) for 5 min at room temperature under a nitrogen atmosphere followed by the addition of NaOMe (4.18 g, 23.233 mmol, 3.5 equiv, 30% wt in MeOH) dropwise at 70° C. The resulting mixture was stirred for an additional 4 h at 70° C. The mixture was allowed to cool down to room temperature. The resulting mixture was concentrated under reduced pressure. The residue was acidified to pH 4 with 2M HCl aq. (6 mL) and then the mixture was filtered and the filter cake was washed with water (3×50 mL). The filtrate was concentrated under reduced pressure to afford methyl 2-{8-[(2,5-difluoro-4-methylphenyl)methyl]imidazo[1,2-a]pyrazin-6-yl}-4-hydroxypyrimidine-5-carboxylate (I-25-7, 2.09 g, 76.5% yield) as a yellow solid. LC-MS: (ESI) m/z 412.2 [M+H].

### Synthesis of I-25

To a stirred solution of methyl 2-{8-[(2,5-difluoro-4-methylphenyl)methyl]imidazo[1,2-a]pyrazin-6-yl}-4-hydroxypyrimidine-5-carboxylate (I-25-7, 500 mg, 1.215 mmol, 1 equiv) in THF (10 mL) was added LiAlH₄ (184.5 mg, 4.860 mmol, 4 equiv) in portions at 0° C., under a nitrogen atmosphere. The resulting mixture was stirred for an additional 1 h at room temperature. The reaction was then quenched with Na₂SO₄·10H₂O (50 mg) at room temperature and the resulting mixture was filtered. The filter cake was washed with THF (3×10 mL), the filtrate was concentrated under reduced pressure and the residue was purified by reverse-phase flash chromatography with the following conditions: column, C18 silica gel; mobile phase, MeCN in Water (0.1% formic acid), 0% to 100% gradient in 10 min; and detector, UV 254 nm, to afford 2-{8-[(2,5-difluoro-4-methylphenyl)methyl]imidazo[1,2-a]pyrazin-6-yl}-5-(hydroxymethyl)pyrimidin-4-ol (I-25, 80.6 mg, 17%) as a white solid. LC-MS: (ESI) m/z 384.15 [M+H]. ¹H NMR: (400 MHz, DMSO-d₆) δ 11.90 (s, 1H), 9.52 (s, 1H), 8.31 (d, J=1.2 Hz, 1H), 7.98 (s, 1H), 7.89 (d, J=1.2 Hz, 1H), 7.35 (s, 1H), 7.16 (dd, J=9.9, 6.5 Hz, 1H), 5.18 (s, 1H), 4.54 (s, 2H), 4.37 (s, 2H), 2.18 (d, J=1.9 Hz, 3H). ¹⁹F NMR-PH-CYCN-CYC-025-0: (376 MHz, DMSO-d₆) δ −123.12 (d, J=18.2 Hz), −123.22-−123.54 (m).

### Synthesis of 2-(8-(2,5-difluoro-4-methylbenzyl)imidazo[1,2-a]pyrazin-6-yl)-6-(2-hydroxyethyl)pyrimidin-4-ol (I-26)

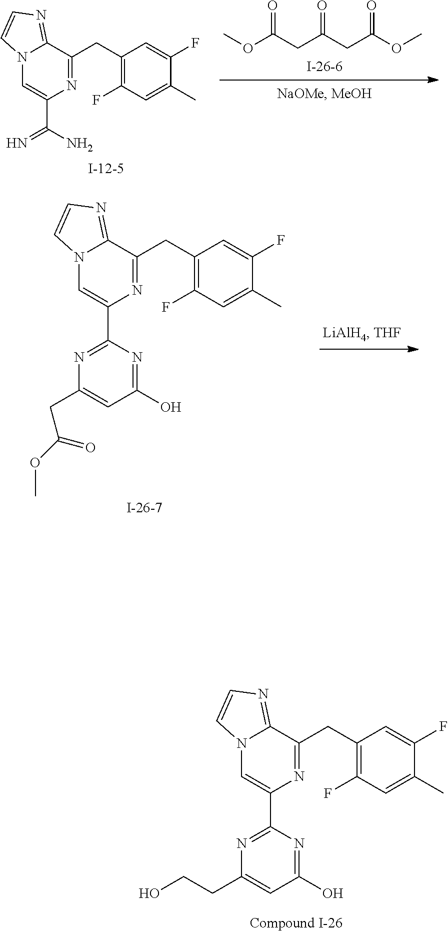

Compound I-12-5 was synthesized in a manner analogous to compound I-8-5. The amidine of compound I-12-5 was condensed with compound I-26-6 to afford cyclized compound I-26-7. The aryl ester of compound I-26-7 was reduced in the presense of lithium aluminium hydride to afford compound I-26.

### Synthesis of I-26-7

To a stirred solution of 8-[(2,5-difluoro-4-methylphenyl)methyl]imidazo[1,2-a]pyrazine-6-carboximidamide (I-12-5, 500 mg, 1.66 mmol, 1.0 equiv) in anhydrous MeOH (10 mL) were added 1,5-dimethyl 3-oxopentanedioate (I-26-6, 578.0 mg, 3.32 mmol, 2.0 equiv) and NaOMe (224.1 mg, 4.15 mmol, 2.5 equiv) at room temperature and the resulting mixture was stirred overnight at 70° C. The mixture was allowed to cool down to room temperature and the crude product was precipitated by the addition of 1 M HCl (10 mL) to afford methyl 2-(2-{8-[(2,5-difluoro-4-methylphenyl)methyl]imidazo[1,2-a]pyrazin-6-yl}-6-hydroxypyrimidin-4-yl)acetate (I-26-7, 470 mg, crude) as a white solid. MS (ESI) m/z 426.15 [M+H].

### Synthesis of I-26

To a solution of methyl 2-(2-{8-[(2,5-difluoro-4-methylphenyl)methyl]imidazo[1,2-a]pyrazin-6-yl}-6-hydroxypyrimidin-4-yl)acetate (I-26-7, 470 mg, 1.105 mmol, 1 equiv) in THF (10 mL) was added LiAlH₄ (98.1 mg, 2.60 mmol, 2.2 equiv) in portions at 0° C. The mixture was stirred for 2 h at room temperature. The reaction was then quenched by the addition of Water (10 mL) at 0° C. The aqueous layer was extracted with CH₂Cl₂ (3×10 mL) and the combined organic layer was washed with brine (20 mL), dried over anhydrous Na₂SO₄ and concentrated under vaccum. The residue was purified by silica gel column chromatography, eluted with petroleum ether/ethyl acetate (1:2) to afford 2-{8-[(2,5-difluoro-4-methylphenyl)methyl]imidazo[1,2-a]pyrazin-6-yl}-6-(2-hydroxyethyl)pyrimidin-4-ol (I-26, 130 mg, 29.5%) as a white solid. LC-MS-PH-CYCN-CYC-026-0: MS (ESI) m/z 398.14 [M+H]. ¹H NMR-PH-CYCN-CYC-026-0: (400 MHz, Chloroform-d) δ=9.20 (s, 1H), 7.92 (s, 1H), 7.88 (s, 1H), 7.01 (dd, J=9.3, 6.3 Hz, 1H), 6.93 (dd, J=9.4, 6.5 Hz, 1H), 6.31 (s, 1H), 4.62 (s, 2H), 4.03 (t, J=5.7 Hz, 2H), 2.86 (t, J=5.6 Hz, 2H), 2.24 (d, J=1.8 Hz, 3H). ¹⁹F NMR-PH-CYCN-CYC-026-0: (376 MHz, Chloroform-d) δ −123.11, −123.14.

### Synthesis of Compounds 2-(8-(2,5-difluoro-4-methylbenzyl)imidazo[1,2-a]pyrazin-6-yl)-6-(ethoxymethyl)-5-fluoropyrimidin-4-ol (I-43) and 2-(8-(2,5-difluoro-4-methylbenzyl)imidazo[1,2-a]pyrazin-6-yl)-5-fluoro-6-(fluoromethyl)pyrimidin-4-ol (I-45)

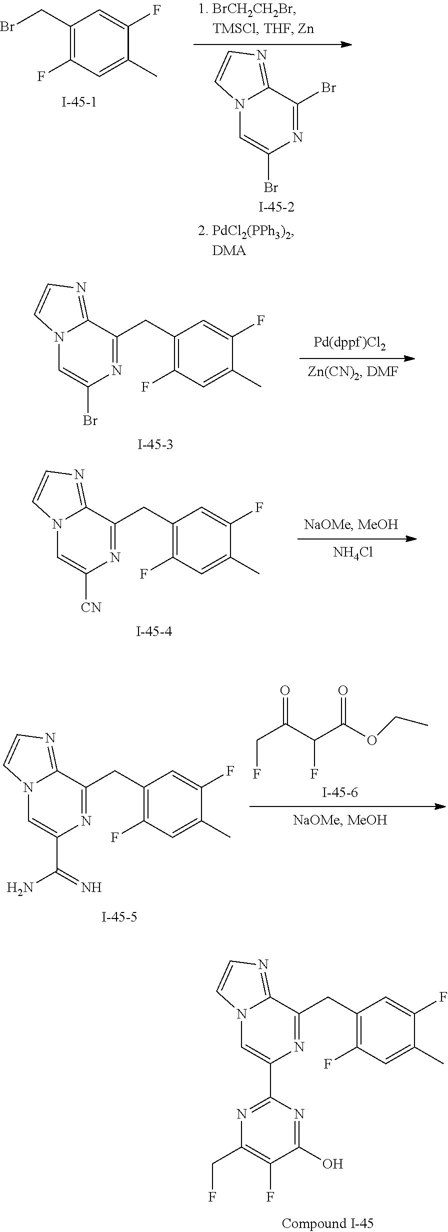

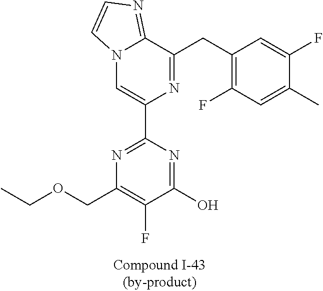 [SMILES: CCOCc1nc(-c2cn3ccnc3c(Cc3cc(F)c(C)cc3F)n2)nc(O)c1F]

Compound I-45-1 was transformed into the corresponding organozinc compound using zinc, dibromoethane, and TMSCl. The organozinc compound underwent a Negishi coupling in the presence of compound I-45-2 to afford compound I-45-3. Compound I-45-3 was further reacted in the presence of zinc, zinc cyanide, and (1,1′-bis(diphenylphosphino)ferrocene)palladium(II) dichloride to afford compound I-45-4. The nitrile of compound I-45-4 was reacted with ammonium chloride to afford amidine compound I-45-5. The amidine of compound I-45-5 was condensed with compound I-45-6 to afford cyclized compound I-45 and by-product compound I-43.

These compounds were made by procedures analogous to those utilized for the syntheses of Compounds I-5 and I-44, but starting from I-4-2-1 instead of the aza version of the core I-5-2. These procedures are also analogous to those used to make compounds I-21 and I-27, in each case using different condensation reagents.

### Compound I-30

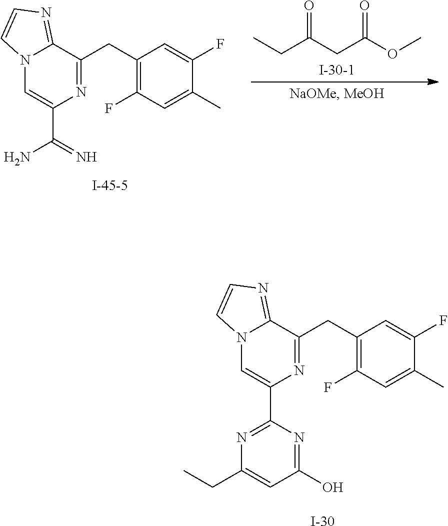

To a stirred solution of 8-[(2,5-difluoro-4-methylphenyl)methyl]imidazo[1,2-a]pyrazine-6-carboximidamide (I-45-5, 500 mg, 1.659 mmol, 1.0 equiv) in anhydrous MeOH (5 mL) was added NaOMe (1045.9 mg, 5.806 mmol, 3.5 equiv, 30% wt in MeOH). To the above mixture was added methyl 3-oxopentanoate (I-30-1, 324.0 mg, 2.489 mmol, 1.5 equiv) at room temperature. The resulting mixture was stirred for 2 h at 70° C. The reaction was quenched by the addition of 1 M HCl (5 mL) at room temperature and the precipitated solids were collected by filtration and washed with water (3×5 mL). The solid was purified by trituration with acetonitrile (10 mL) to afford 2-{8-[(2,5-difluoro-4-methylphenyl)methyl]imidazo[1,2-a]pyrazin-6-yl}-6-ethylpyrimidin-4-ol (I-30, 100 mg, 23% yield) as a light yellow solid. The reaction was repeated 3 more times and a total of 381.5 mg of product were obtained. LC-MS: (ESI) m/z 382.15 [M+H]. ¹H NMR: (400 MHz, DMSO-d₆) δ 11.65 (s, 1H), 9.50 (s, 1H), 8.35 (d, J=1.1 Hz, 1H), 7.89 (d, J=1.1 Hz, 1H), 7.36 (dd, J=10.1, 6.3 Hz, 1H), 7.16 (dd, J=10.0, 6.4 Hz, 1H), 6.21 (s, 1H), 4.54 (s, 2H), 2.57 (q, J=7.5 Hz, 2H), 2.19 (d, J=1.9 Hz, 3H), 1.23 (t, J=7.5 Hz, 3H). ¹⁹F NMR: (377 MHz, DMSO-d₆) δ −123.13 (d, J=18.1 Hz), −123.40 (d, J=18.1 Hz).

### Example 2: cGMP Measurement in Rat Primary Neurons

Rat primary neurons were isolated from timed-pregnant female Sprague-Dawley (SD) rats, washed once with HBSS with calcium and magnesium and incubated with a solution containing 0.5 mM 3-isobutyl-1-methylxanthine (IBMX) in HBSS (80 μL/well) at 37° C. for 15 min. Next, a 5× stock (20 l) of test compound with a fixed concentration of DETA (Diethylenetriamine NONOate) was added to the wells to make x nM concentration for test compound solutions and a 30 M concentration for DETA solution, where x was 1 of the following final concentrations: 0.029, 0.114, 0.460, 1.83, 7.32, 29.29, 117.2, 468.8, 1875, 7500 and 30,000 nM. Then, the cells were incubated for 20 min at 37° C. At the end of incubation, 100 μL lysis buffer (Molecular Devices) was added to cells. CatchPoint™ Cyclic-GMP Fluorescent Assay Kit was used with Molecular Devices' ID3 multi-mode plate reading. Sample cGMP concentrations were extrapolated from standard curve using Softmax Pro 7 Software. A well-characterized, potent sGC stimulator was included in all assay runs and used as a positive control.

Test compound (“agonist”) concentrations vs extrapolated cGMP concentrations (“response”) were plotted and analyzed using GraphPad Prism version 9.4.1. Curve fit was used for each dataset; non-linear log(agonist) vs. response-variable slope (four parameters). The EC₅₀ is interpolated from the curve fit and is defined as the concentration at which the compound elicits 50% of its maximal response. When the experiment is carried out more than two times, the geometric mean is reported, otherwise the arithmetic mean is shown. To increase the accuracy of the EC₅₀ calculations, top and bottom parameters were constrained when consistent with the observed data in a given assay run. For instance, in assay runs where the cGMP response values in the absence of test article were consistent across plates, an average across all of these wells was used as a lower constraint in the calculations. In assay runs where the maximal cGMP responses were consistent with that of the positive control, a top constraint equal to the maximal cGMP response of the positive control was applied. If there was significant variability in cGMP response in no compound wells or variation in maximal responses across plates, no constraints were imposed. For compounds that did not achieve a response >50% of the positive control (if a top constraint was applied) or did not appear to plateau in response at higher concentrations (in the absence of a top constraint), the test article EC₅₀ was defined as >30 M (the highest concentration tested). EC₅₀ values for compounds that did not show a dose response were characterized as ND (not determined).

TABLE A

| | sGC Neuron |
|---|---|
| Compound No. | EC₅₀ (nM) |
| I-3 | A |
| I-4 | D |
| I-5 | C |
| I-6 | D |
| I-7 | D |
| I-8 | D |
| I-9 | D |
| I-12 | D |
| I-17 | A |
| I-21 | B |
| I-22 | B |
| I-24 | B |
| I-25 | B |
| I-26 | D |
| I-27 | D |
| I-28 | B |
| I-29 | B |
| I-30 | C |
| I-31 | D |
| I-32 | D |
| I-35 | C |
| I-44 | D |
| I-45 | B |
| I-47 | B |

Neuronal-based cell assay.
EC₅₀ ≤ 100 nM = A;
100 nM < EC₅₀ ≤ 1000 nM = B;
1000 nM < EC₅₀ = C ≤ 5000 nM;
EC₅₀ > 5000 nM = D

1. A compound represented by Formula I: 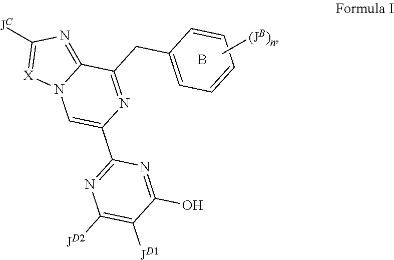
or a pharmaceutically acceptable salt thereof, wherein:
X is N or C(J^{C1});
J^{C} is selected from the group consisting of hydrogen, halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted with 1 to 3 substituents independently selected from the group consisting of halogen, —OH, —OR, —SR and —C(O)R;
J^{C1} is selected from the group consisting of hydrogen, halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted by 1 to 3 substituents independently selected from the group consisting of halogen, —OH, —OR, —SR and —C(O)R;
n is an integer selected from 0, 1, 2 or 3;
each J^{B} is independently selected from the group consisting of halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted by 1 to 3 substituents independently selected from the group consisting of halogen, —OH, —OR, —SR and —C(O)R;
J^{D1} is selected from the group consisting of hydrogen, halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted by 1 to 3 substituents independently selected from the group consisting of halogen, —OH, —OR, —SR and —C(O)R;
J^{D2} is selected from the group consisting of hydrogen, halogen, C₁₋₆ alkyl, —OH, —OR, —SH, —SR, —CN, —C(O)R, and C₃₋₅ cycloalkyl, wherein the C₃₋₅ cycloalkyl is optionally and independently substituted with 1 to 3 halogen atoms and the C₁₋₆ alkyl is optionally substituted by 1 to 3 substituents independently selected from the group consisting of halogen, —OH, —OR, —SR and —C(O)R;
R, for each occurrence, is independently C₁₋₄ alkyl optionally substituted with 1 to 3 independently selected halogen atoms;
wherein, when J^{D2} is hydrogen and n is 1, 2 or 3, then at least one of J^{C}, J^{C1}, J^{D1}, and J^{B} is —OH, —OR, —SH, —SR, —CN, —C(O)R, a C₃₋₅ cycloalkyl optionally and independently substituted with 1 to 3 halogen atoms, or a C₁₋₆ alkyl substituted with 1 to 3 substituents independently selected from the group consisting of —OH, —OR, —SR and —C(O)R;
when J^{D2} is hydrogen and n is 0, then at least one of J^{C}, J^{C1} and J^{D1} is —OH, —OR, —SH, —SR, —CN, —C(O)R, a C₃₋₅ cycloalkyl optionally and independently substituted with 1 to 3 halogen atoms, or a C₁₋₆ alkyl substituted with 1 to 3 substituents independently selected from the group consisting of —OH, —OR, —SR and —C(O)R; and
provided that the compound is not one of the following:

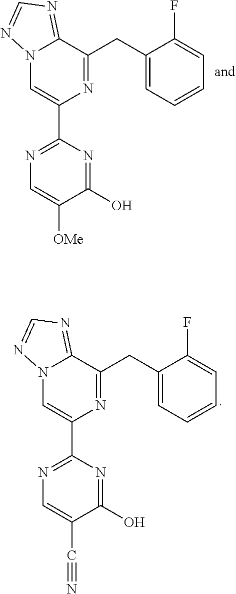 [SMILES: COc1cnc(-c2cn3ncnc3c(Cc3ccccc3F)n2)nc1O ; N#Cc1cnc(-c2cn3ncnc3c(Cc3ccccc3F)n2)nc1O]

2. The compound of claim 1, wherein the compound is represented by Formula IA: 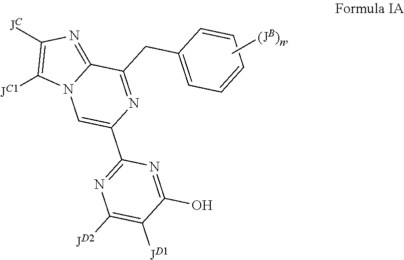
or a pharmaceutically acceptable salt thereof.

3. The compound of claim 1 or 2, or a pharmaceutically acceptable salt thereof, wherein J^{C1} is selected from the group consisting of hydrogen, halogen, C₁₋₃ alkyl, —CN, —SH, —SR, —OR, and —C(O)R, wherein the C₁₋₃ alkyl is optionally substituted by 1 to 3 substituents independently selected from the group consisting of halogen, —OH and —OR.

4. The compound of claim 3, or a pharmaceutically acceptable salt thereof, wherein J^{C1} is selected from the group consisting of hydrogen, —F, —Cl, —CN, —CH₃, —CH₂F, —SH, —CH₂OH, —CH₂OCH₃, —SCH₃, —CH(OH)CH₃, —C(O)CH₃, and —OCH₃.

5. The compound of claim 3, or a pharmaceutically acceptable salt thereof, wherein J^{C1} is selected from the group consisting of hydrogen, halogen, and C₁₋₃ alkyl, wherein the C₁₋₃ alkyl is optionally substituted by 1 to 3 independently selected halogen substituents.

6. The compound of claim 3, or a pharmaceutically acceptable salt thereof, wherein J^{C1} is selected from the group consisting of hydrogen, —F, —Cl, —CH₃, and —CH₂F.

7. The compound of claim 3, or a pharmaceutically acceptable salt thereof, wherein J^{C1} is hydrogen or —F.

8. The compound of claim 1, wherein the compound is represented by Formula IB: 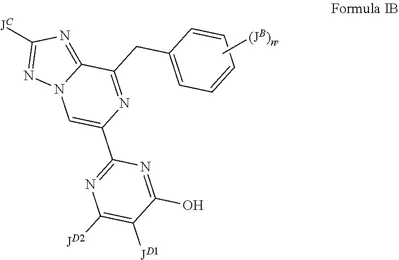
or a pharmaceutically acceptable salt thereof.

9. The compound of any one of claims 1-8, or a pharmaceutically acceptable salt thereof, wherein n is 2 or 3.

10. The compound of any one of claims 1-8, or a pharmaceutically acceptable salt thereof, wherein n is 2.

11. The compound of any one of claims 1-8, or a pharmaceutically acceptable salt thereof, wherein n is 3.

12. The compound of any one of claims 1-8, or a pharmaceutically acceptable salt thereof, wherein n is 0 or 1.

13. The compound of any one of claims 1-8, or a pharmaceutically acceptable salt thereof, wherein n is 1.

14. The compound of any one of claims 1-8, or a pharmaceutically acceptable salt thereof, wherein n is 0.

15. The compound of any one of claims 1-14, or a pharmaceutically acceptable salt thereof, wherein each J^{B} is independently selected from a group consisting of halogen, —CN, —OH, —OR, —SR, —C(O)R and C₁₋₃ alkyl, wherein the C₁₋₃ alkyl is optionally substituted by 1 to 3 substituents independently selected form halogen, —OH and —OR.

16. The compound of claim 15, or a pharmaceutically acceptable salt thereof, wherein each J^{B} is independently selected from a group consisting of —F, —Cl, —Br, —CN, —CH₃, —CF₃, —CH₂F, —CHFCH₃, —CH₂CH₂F, —C(O)CH₃, —CH(OH)CH₃, —CH₂CH₃, —SCH₃, —OCH₃, —OH, —CH₂OCH₃, and —CH₂OH.

17. The compound of claim 15, or a pharmaceutically acceptable salt thereof, wherein n is 2 or 3, and each J^{B} is independently selected from the group consisting of halogen, —OR, —CN, —OH, and C₁₋₃ alkyl, wherein the C₁₋₃ alkyl is optionally substituted by 1 to 3 independently selected halogen substituents.

18. The compound of claim 15 or a pharmaceutically acceptable salt thereof, wherein n is 2 or 3, and each J^{B} is independently selected from the group consisting of —F, —Cl, —CH₃, —CF₃, —CN, —OH and —OCH₃.

19. The compound of claim 15, or a pharmaceutically acceptable salt thereof, wherein n is 2; and each J^{B} is independently selected from the group consisting of —F, —CH₃ and —CF₃.

20. The compound of claim 15, or a pharmaceutically acceptable salt thereof, wherein n is 2; and one of J^{B} is —CH₃ or —CF₃ and the other J^{B} is —F.

21. The compound of claim 15, or a pharmaceutically acceptable salt thereof, wherein n is 2; and both J^{B} are —F.

22. The compound of claim 15, or a pharmaceutically acceptable salt thereof, wherein n is 3; and each J^{B} is independently selected from the group consisting of —F, —OH, —CH₃, —CH₂F and —CF₃.

23. The compound of claim 15, or a pharmaceutically acceptable salt thereof, wherein n is 3; and one of J^{B} is —F, a second J^{B} is —CH₃ or —CH₂F, and the other is selected from the group consisting of —F, —OH, —CH₃ and —CH₂F.

24. The compound of claim 15, or a pharmaceutically acceptable salt thereof, wherein n is 1; and J^{B} is selected from the group consisting of halogen, —OR, and C₁₋₃ alkyl optionally substituted with 1 to 3 independently selected halogen substituents.

25. The compound of claim 15, or a pharmaceutically acceptable salt thereof, wherein n is 1; and J^{B} is selected from the group consisting of —F, —OR, and C₁₋₃ alkyl optionally substituted with 1 to 3 independently selected halogen substituents.

26. The compound of claim 15, or a pharmaceutically acceptable salt thereof, wherein n is 1; and J^{B} is selected from the group consisting of —F, —CH₃, —CF₃ and —OCH₃.

27. The compound of any one of claims 1-26, or a pharmaceutically acceptable salt thereof, wherein J^{D2} is selected from the group consisting of hydrogen, halogen, —OH, —OR, —SR, —C(O)R, and C₁₋₃ alkyl optionally substituted by 1 to 3 substituents independently selected from the group consisting of halogen, —OH, —OR and —C(O)R.

28. The compound of claim 27, or a pharmaceutically acceptable salt thereof, wherein J^{D2} is selected from the group consisting of hydrogen, —F, —Cl, —Br, —CH₃, —SCH₃, —OH, —CH₂F, —CH₂OCH₂CH₃, —OCH₃, —CH₂OCH₃, and —CH₂CH₂OH.

29. The compound of claim 27, or a pharmaceutically acceptable salt thereof, wherein J^{D2} is selected from the group consisting of hydrogen, halogen, —OR and C₁₋₃ alkyl optionally substituted with 1 to 3 substituents independently selected from halogen and —C(O)R.

30. The compound of claim 27, or a pharmaceutically acceptable salt thereof, wherein J^{D2} is selected from the group consisting of hydrogen, —F and —Cl.

31. The compound of claim 27, or a pharmaceutically acceptable salt thereof, wherein J^{D2} is hydrogen or —F.

32. The compound of claim 27, or a pharmaceutically acceptable salt thereof, wherein J^{D2} is hydrogen.

33. The compound of any one of claims 1-32, or a pharmaceutically acceptable salt thereof, wherein J^{C} is selected from the group consisting of hydrogen, —F, —Cl, —CH₃, —CH₂F, —OCH₃, —SCH₃, —C(O)CH₃, —CH₂OCH₃, —CH₂OH and —CN.

34. The compound of claim 33, or a pharmaceutically acceptable salt thereof, wherein J^{C} selected from the group consisting of is hydrogen, —F, —Cl, —CH₃ and —CH₂F.

35. The compound of any one of claims 1-7, 9-26, 33 and 34, wherein the compound is represented by Formula IIA: 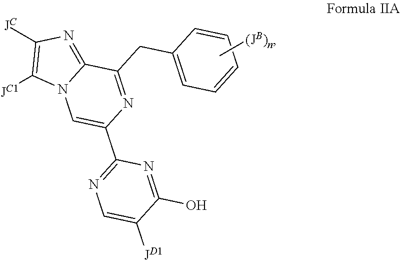
or a pharmaceutically acceptable salt thereof.

36. The compound of any one of claims 1 and 8-26, 33 and 34, wherein the compound is represented by Formula IIB: 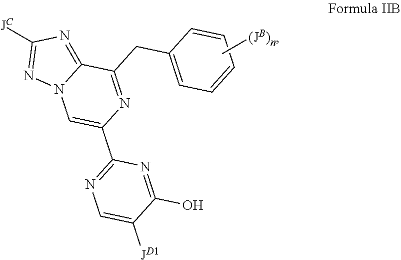
or a pharmaceutically acceptable salt thereof.

37. The compound of any one of claims 1-36, or a pharmaceutically acceptable salt thereof, wherein J^{D1} is selected from the group consisting of hydrogen, halogen, —CN, —OH, —OR, —SR, —C(O)R, and C₁₋₃ alkyl optionally substituted by 1 to 3 substituents independently selected from the group consisting of halogen, —OH and —OR.

38. The compound of claim 37, or a pharmaceutically acceptable salt thereof, wherein J^{D1} is selected from the group consisting of hydrogen, —F, —CH₃, —CN, —SCH₃, —CHF₂, —OCH₃, —C(O)CH₃, —CH(OH)CH₃, or —CH₂CH₂OH.

39. The compound of claim 37, or a pharmaceutically acceptable salt thereof, wherein J^{D1} is selected from the group consisting of hydrogen, halogen, —OR and C₁₋₃ alkyl optionally substituted with 1 to 3 substituents independently selected from halogen and —C(O)R.

40. The compound of claim 37, or a pharmaceutically acceptable salt thereof, wherein J^{D1} is selected from the group consisting of hydrogen, —F and —Cl.

41. The compound of claim 37, or a pharmaceutically acceptable salt thereof, wherein J^{D1} is halogen.

42. The compound of claim 37, or a pharmaceutically acceptable salt thereof, wherein J^{D1} is —F.

43. The compound of any one of claims 1-42, or a pharmaceutically acceptable salt thereof, wherein R, for each occurrence, is independently C₁₋₄ alkyl.

44. The compound of claim 1, wherein the compound is selected from: 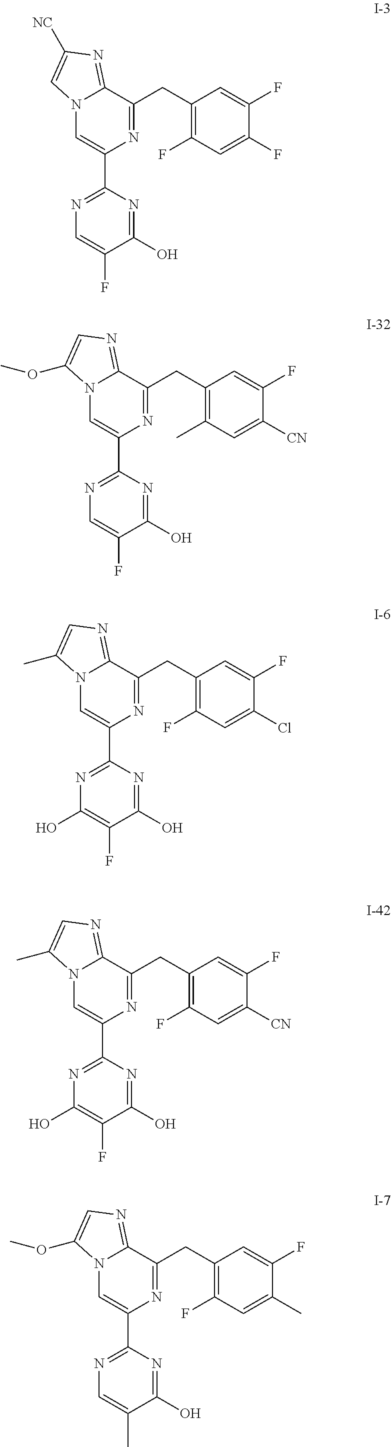 [SMILES: N#Cc1cn2cc(-c3ncc(F)c(O)n3)nc(Cc3cc(F)c(F)cc3F)c2n1 ; COc1cnc2c(Cc3cc(F)c(C#N)cc3C)nc(-c3ncc(F)c(O)n3)cn12 ; Cc1cnc2c(Cc3cc(F)c(Cl)cc3F)nc(-c3nc(O)c(F)c(O)n3)cn12 ; Cc1cnc2c(Cc3cc(F)c(C#N)cc3F)nc(-c3nc(O)c(F)c(O)n3)cn12 ; COc1cnc2c(Cc3cc(F)c(C)cc3F)nc(-c3ncc(C)c(O)n3)cn12] 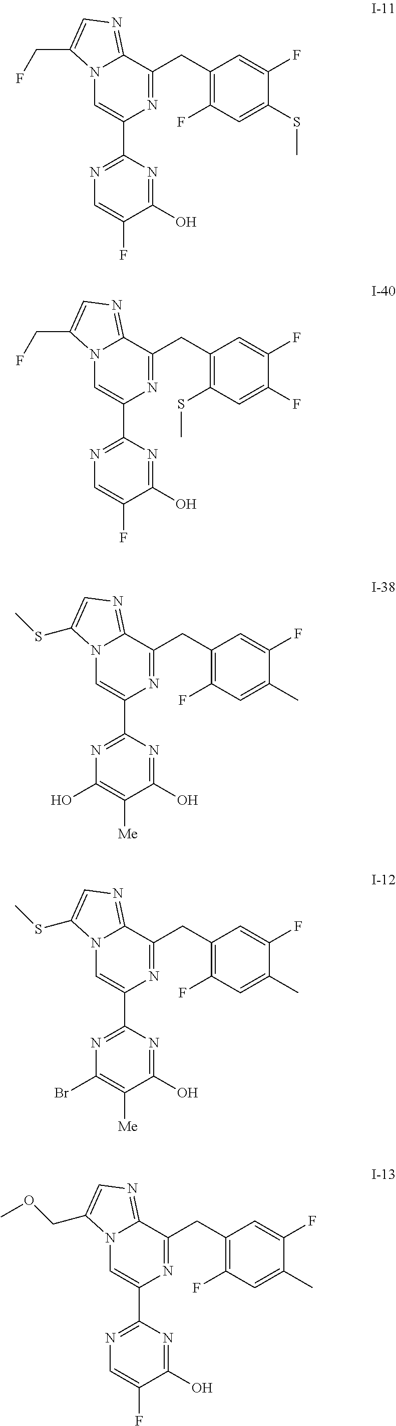 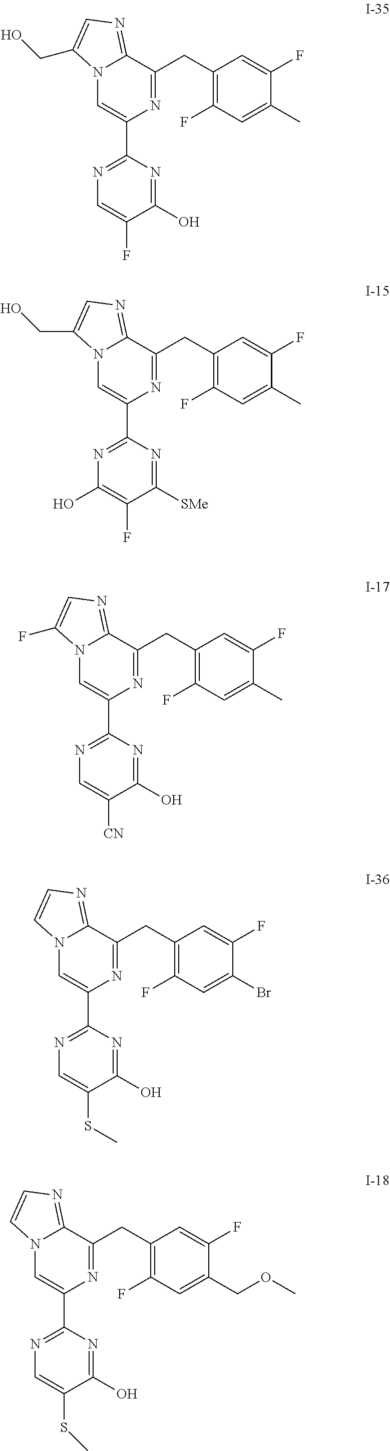 [SMILES: Cc1cc(F)c(Cc2nc(-c3ncc(F)c(O)n3)cn3c(CO)cnc23)cc1F ; CSc1nc(-c2cn3c(CO)cnc3c(Cc3cc(F)c(C)cc3F)n2)nc(O)c1F ; Cc1cc(F)c(Cc2nc(-c3ncc(C#N)c(O)n3)cn3c(F)cnc23)cc1F ; CSc1cnc(-c2cn3ccnc3c(Cc3cc(F)c(Br)cc3F)n2)nc1O ; COCc1cc(F)c(Cc2nc(-c3ncc(SC)c(O)n3)cn3ccnc23)cc1F] 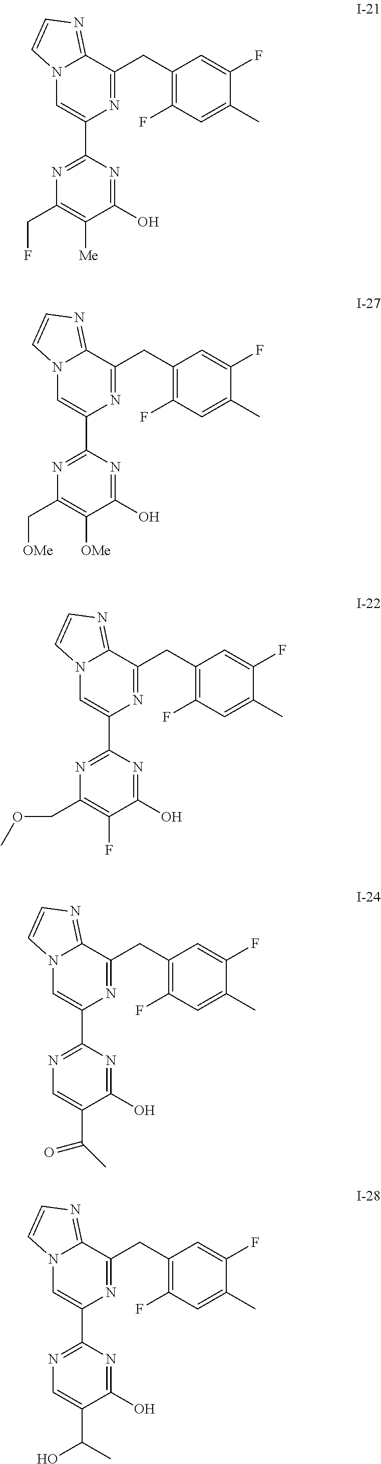 [SMILES: Cc1cc(F)c(Cc2nc(-c3nc(O)c(C)c(CF)n3)cn3ccnc23)cc1F ; COCc1nc(-c2cn3ccnc3c(Cc3cc(F)c(C)cc3F)n2)nc(O)c1OC ; COCc1nc(-c2cn3ccnc3c(Cc3cc(F)c(C)cc3F)n2)nc(O)c1F ; CC(=O)c1cnc(-c2cn3ccnc3c(Cc3cc(F)c(C)cc3F)n2)nc1O ; Cc1cc(F)c(Cc2nc(-c3ncc(C(C)O)c(O)n3)cn3ccnc23)cc1F] 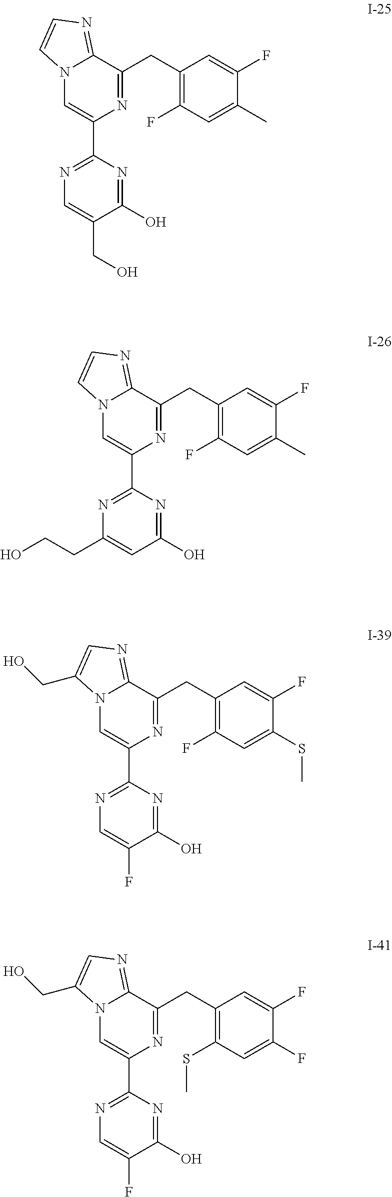 [SMILES: Cc1cc(F)c(Cc2nc(-c3ncc(CO)c(O)n3)cn3ccnc23)cc1F ; Cc1cc(F)c(Cc2nc(-c3nc(O)cc(CCO)n3)cn3ccnc23)cc1F ; CSc1cc(F)c(Cc2nc(-c3ncc(F)c(O)n3)cn3c(CO)cnc23)cc1F ; CSc1cc(F)c(F)cc1Cc1nc(-c2ncc(F)c(O)n2)cn2c(CO)cnc12] 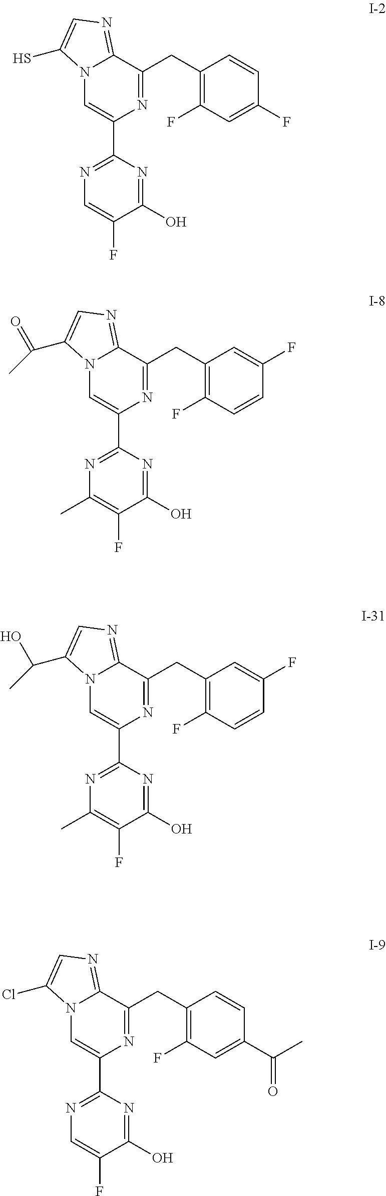 [SMILES: Oc1nc(-c2cn3c(S)cnc3c(Cc3ccc(F)cc3F)n2)ncc1F ; CC(=O)c1cnc2c(Cc3cc(F)ccc3F)nc(-c3nc(C)c(F)c(O)n3)cn12 ; Cc1nc(-c2cn3c(C(C)O)cnc3c(Cc3cc(F)ccc3F)n2)nc(O)c1F ; CC(=O)c1ccc(Cc2nc(-c3ncc(F)c(O)n3)cn3c(Cl)cnc23)c(F)c1] 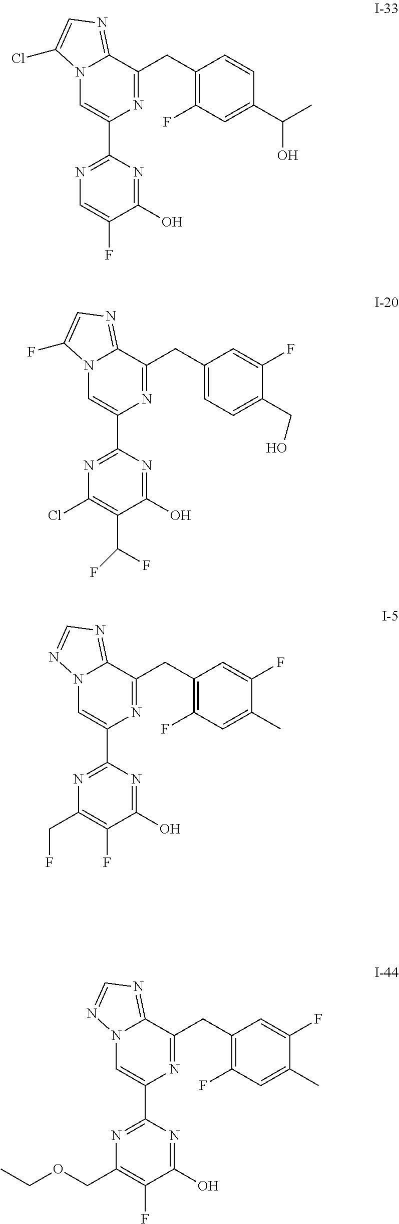 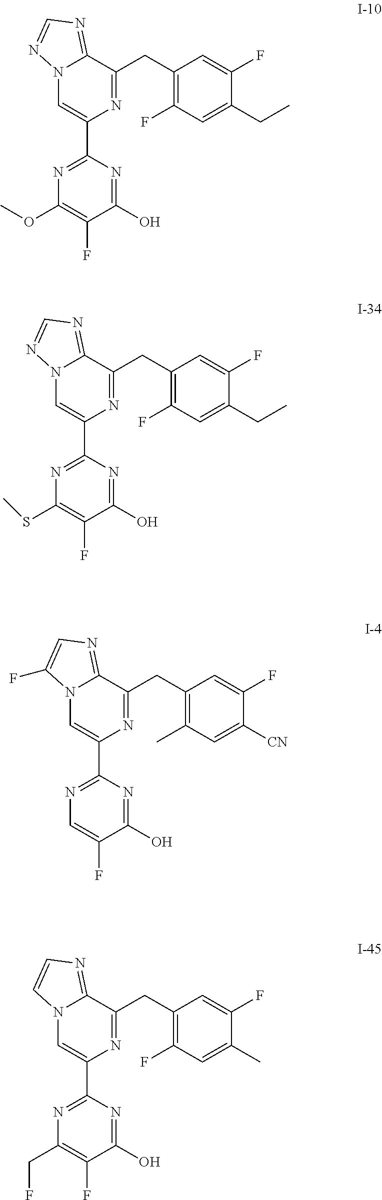 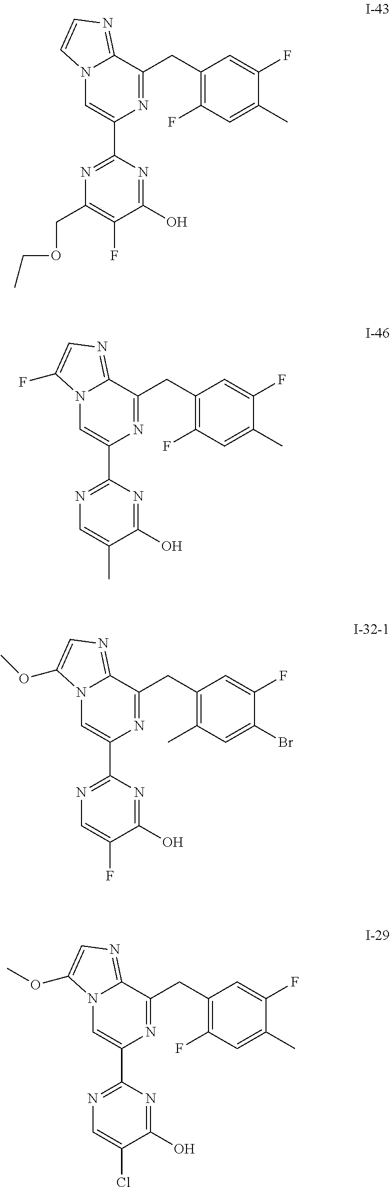 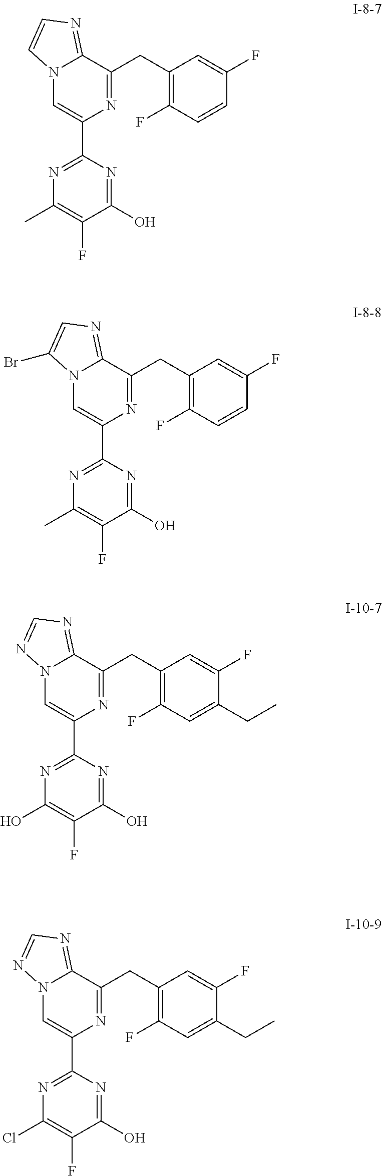 [SMILES: Cc1nc(-c2cn3ccnc3c(Cc3cc(F)ccc3F)n2)nc(O)c1F ; Cc1nc(-c2cn3c(Br)cnc3c(Cc3cc(F)ccc3F)n2)nc(O)c1F ; CCc1cc(F)c(Cc2nc(-c3nc(O)c(F)c(O)n3)cn3ncnc23)cc1F ; CCc1cc(F)c(Cc2nc(-c3nc(O)c(F)c(Cl)n3)cn3ncnc23)cc1F] 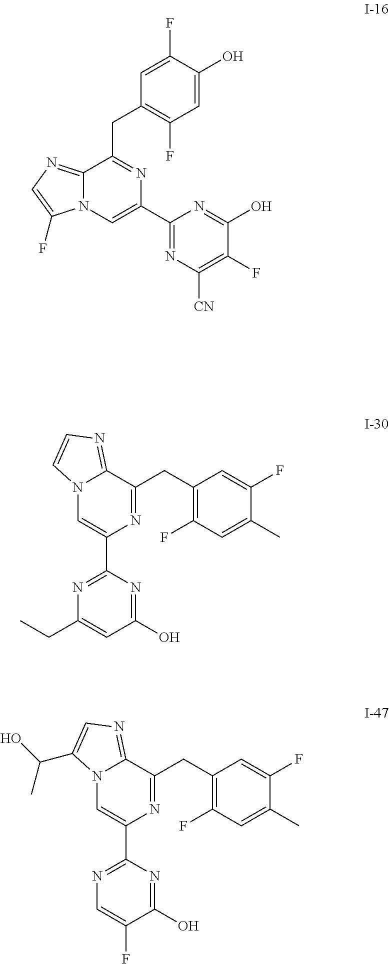 [SMILES: N#Cc1nc(-c2cn3c(F)cnc3c(Cc3cc(F)c(O)cc3F)n2)nc(O)c1F ; CCc1cc(O)nc(-c2cn3ccnc3c(Cc3cc(F)c(C)cc3F)n2)n1 ; Cc1cc(F)c(Cc2nc(-c3ncc(F)c(O)n3)cn3c(C(C)O)cnc23)cc1F]
or a pharmaceutically acceptable salt thereof.

45. The compound of claim 44, wherein the compound is selected from: 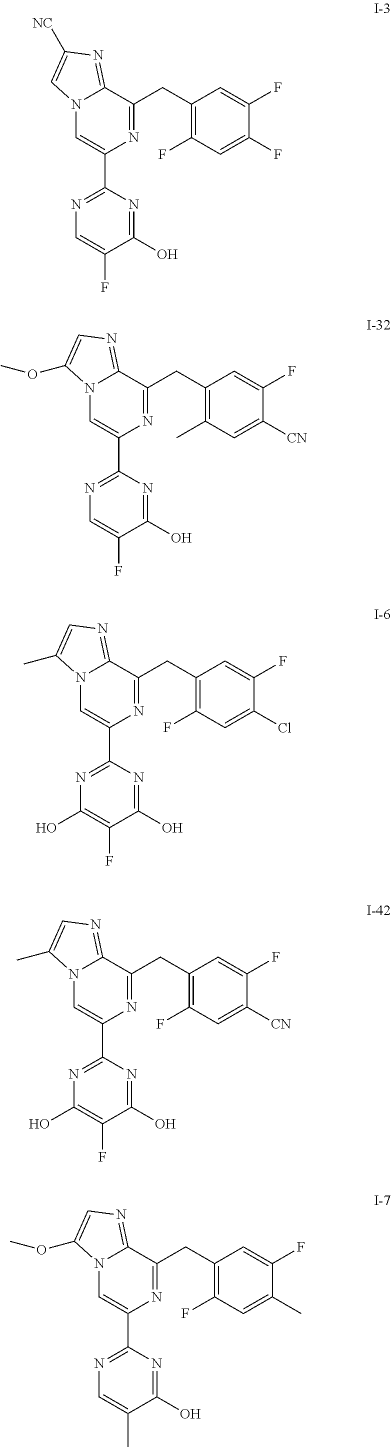 [SMILES: N#Cc1cn2cc(-c3ncc(F)c(O)n3)nc(Cc3cc(F)c(F)cc3F)c2n1 ; COc1cnc2c(Cc3cc(F)c(C#N)cc3C)nc(-c3ncc(F)c(O)n3)cn12 ; Cc1cnc2c(Cc3cc(F)c(Cl)cc3F)nc(-c3nc(O)c(F)c(O)n3)cn12 ; Cc1cnc2c(Cc3cc(F)c(C#N)cc3F)nc(-c3nc(O)c(F)c(O)n3)cn12 ; COc1cnc2c(Cc3cc(F)c(C)cc3F)nc(-c3ncc(C)c(O)n3)cn12] 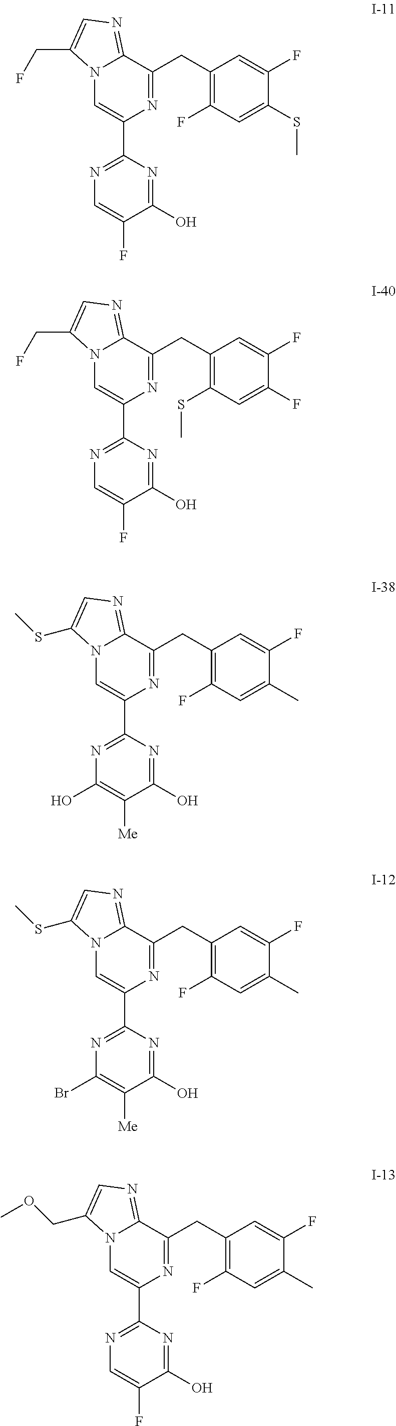 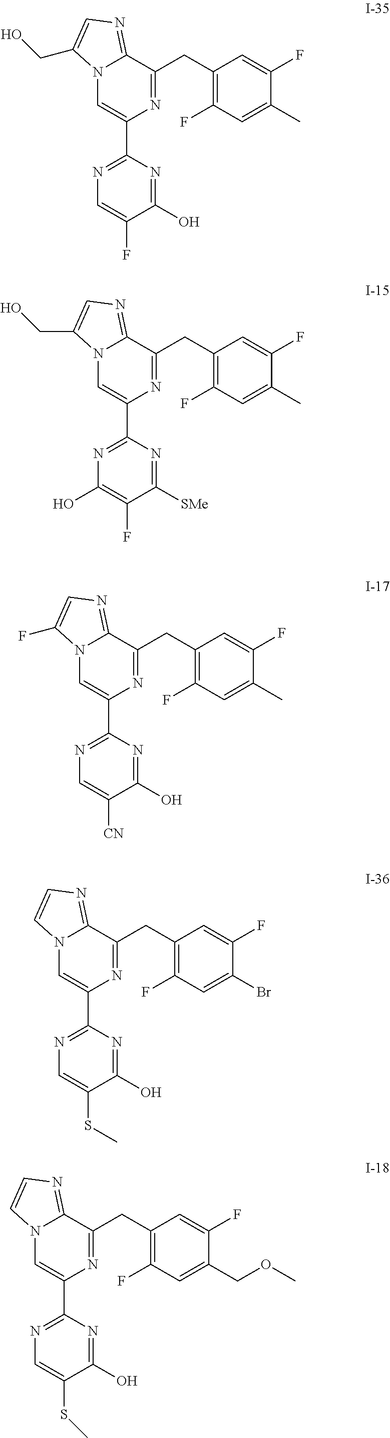 [SMILES: Cc1cc(F)c(Cc2nc(-c3ncc(F)c(O)n3)cn3c(CO)cnc23)cc1F ; CSc1nc(-c2cn3c(CO)cnc3c(Cc3cc(F)c(C)cc3F)n2)nc(O)c1F ; Cc1cc(F)c(Cc2nc(-c3ncc(C#N)c(O)n3)cn3c(F)cnc23)cc1F ; CSc1cnc(-c2cn3ccnc3c(Cc3cc(F)c(Br)cc3F)n2)nc1O ; COCc1cc(F)c(Cc2nc(-c3ncc(SC)c(O)n3)cn3ccnc23)cc1F] 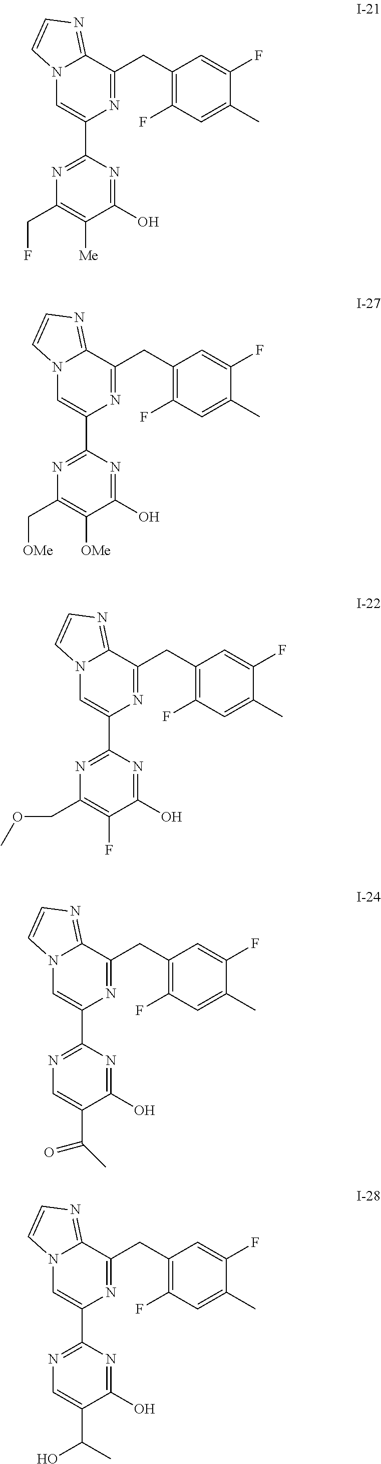 [SMILES: Cc1cc(F)c(Cc2nc(-c3nc(O)c(C)c(CF)n3)cn3ccnc23)cc1F ; COCc1nc(-c2cn3ccnc3c(Cc3cc(F)c(C)cc3F)n2)nc(O)c1OC ; COCc1nc(-c2cn3ccnc3c(Cc3cc(F)c(C)cc3F)n2)nc(O)c1F ; CC(=O)c1cnc(-c2cn3ccnc3c(Cc3cc(F)c(C)cc3F)n2)nc1O ; Cc1cc(F)c(Cc2nc(-c3ncc(C(C)O)c(O)n3)cn3ccnc23)cc1F] 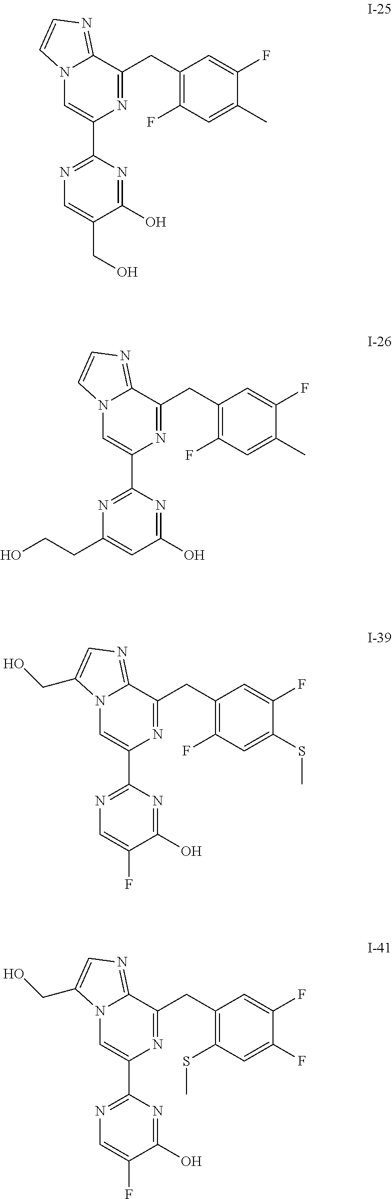 [SMILES: Cc1cc(F)c(Cc2nc(-c3ncc(CO)c(O)n3)cn3ccnc23)cc1F ; Cc1cc(F)c(Cc2nc(-c3nc(O)cc(CCO)n3)cn3ccnc23)cc1F ; CSc1cc(F)c(Cc2nc(-c3ncc(F)c(O)n3)cn3c(CO)cnc23)cc1F ; CSc1cc(F)c(F)cc1Cc1nc(-c2ncc(F)c(O)n2)cn2c(CO)cnc12] 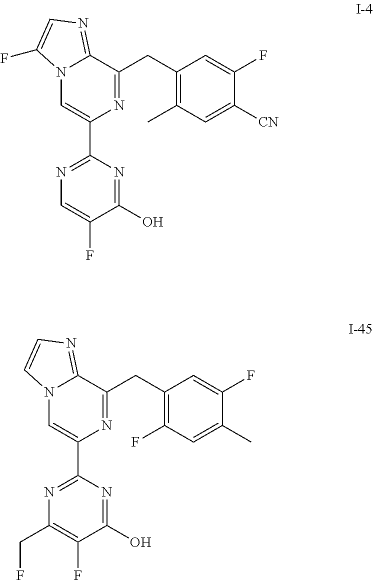 [SMILES: Cc1cc(C#N)c(F)cc1Cc1nc(-c2ncc(F)c(O)n2)cn2c(F)cnc12 ; Cc1cc(F)c(Cc2nc(-c3nc(O)c(F)c(CF)n3)cn3ccnc23)cc1F] 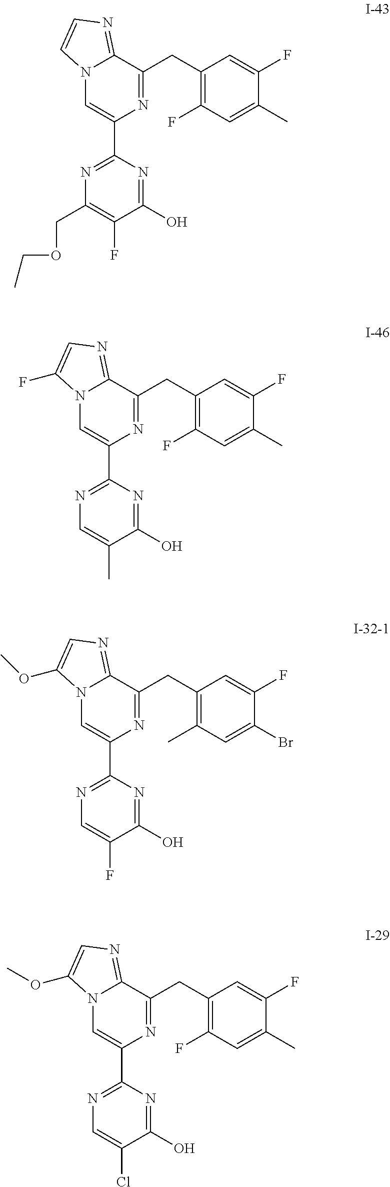 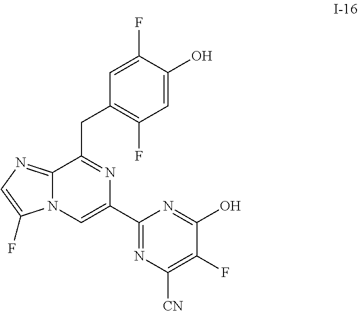 [SMILES: N#Cc1nc(-c2cn3c(F)cnc3c(Cc3cc(F)c(O)cc3F)n2)nc(O)c1F]
or a pharmaceutically acceptable salt thereof.

46. The compound of claim 44, wherein the compound is selected from: 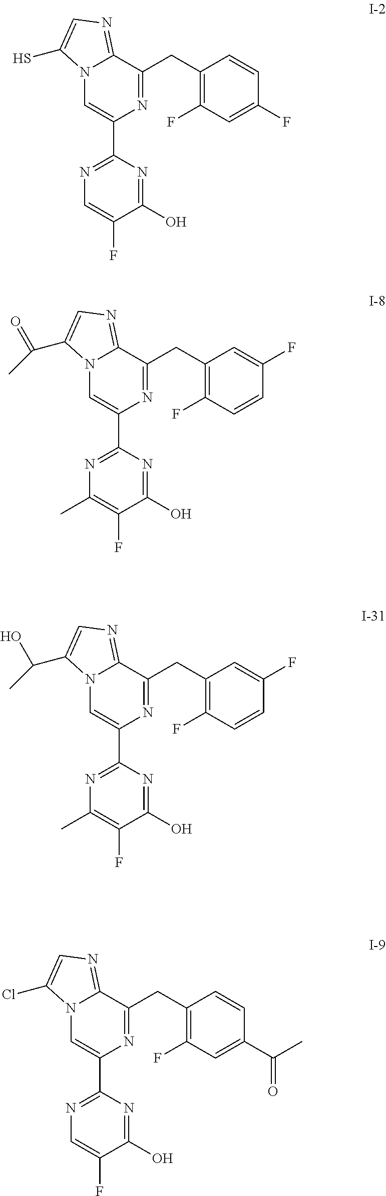 [SMILES: Oc1nc(-c2cn3c(S)cnc3c(Cc3ccc(F)cc3F)n2)ncc1F ; CC(=O)c1cnc2c(Cc3cc(F)ccc3F)nc(-c3nc(C)c(F)c(O)n3)cn12 ; Cc1nc(-c2cn3c(C(C)O)cnc3c(Cc3cc(F)ccc3F)n2)nc(O)c1F ; CC(=O)c1ccc(Cc2nc(-c3ncc(F)c(O)n3)cn3c(Cl)cnc23)c(F)c1] 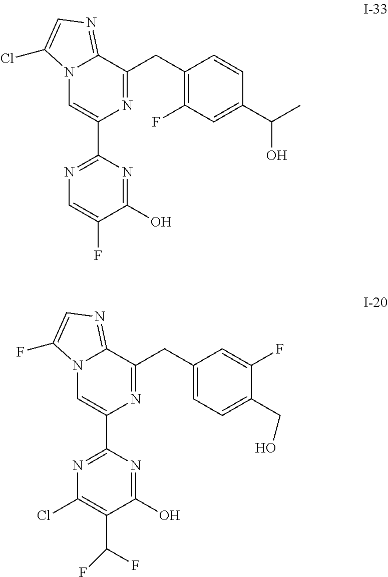 [SMILES: CC(O)c1ccc(Cc2nc(-c3ncc(F)c(O)n3)cn3c(Cl)cnc23)c(F)c1 ; OCc1ccc(Cc2nc(-c3nc(O)c(C(F)F)c(Cl)n3)cn3c(F)cnc23)cc1F] 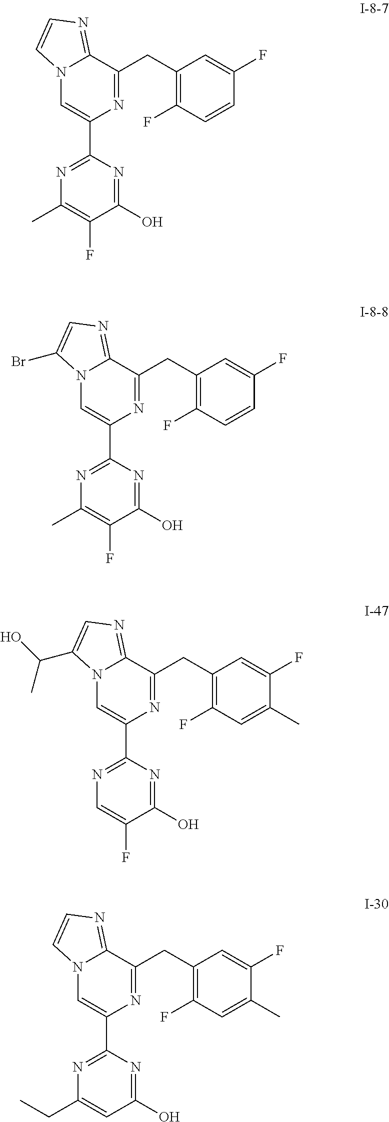
or a pharmaceutically acceptable salt thereof.

47. The compound of claim 44, wherein the compound is selected from: 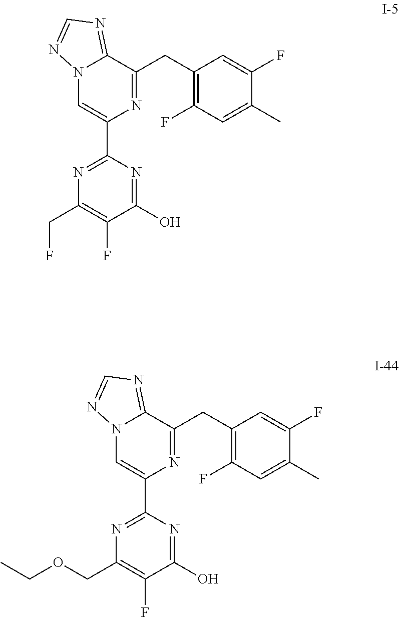 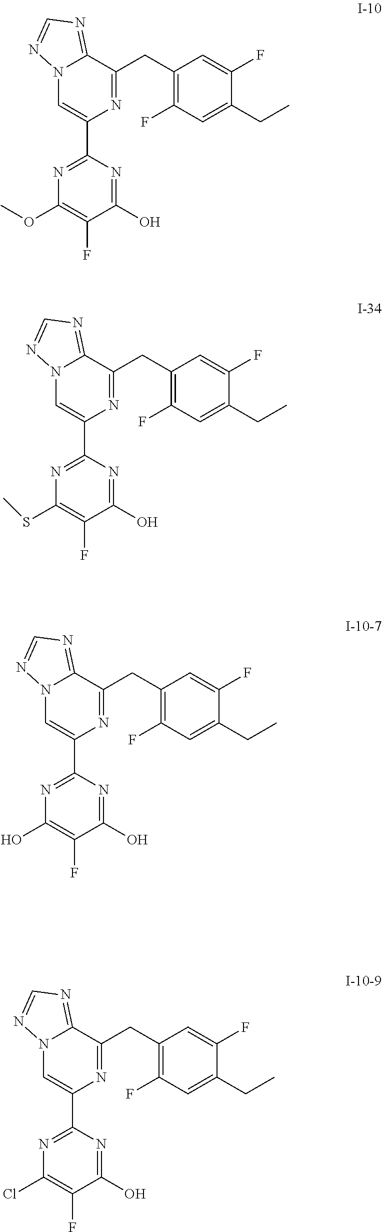
or a pharmaceutically acceptable salt thereof.

48. A pharmaceutical composition comprising a compound of any one of claims 1-47, or a pharmaceutically acceptable salt thereof, and a pharmaceutically acceptable excipient or carrier.

49. A method of treating a disease in a subject in need thereof, comprising administering to the subject, alone or in combination therapy, a therapeutically effective amount of a compound of any one of claims 1-47, or a pharmaceutically acceptable salt thereof, or a pharmaceutical composition of claim 48; wherein the disease or disorder is one that would benefit from sGC stimulation or from an increase in the concentration of NO and/or cGMP.

50. The method of claim 49, wherein the method is for treating a CNS disease, a CNS health condition or a CNS disorder.

51. The method of claim 50, wherein the CNS disease is Alzheimer's disease.

52. The method of claim 51, wherein the Alzheimer's disease is mild to moderate Alzheimer's disease or moderate to severe Alzheimer's disease.

53. The method of claim 50, wherein the CNS disease is cognitive impairment.

54. The method of claim 50, wherein the CNS disease is dementia.

55. The method of claim 50, wherein the CNS disease is subjective cognitive impairment (SCI).

56. The method of claim 50, wherein the CNS disease is cognitive ageing.

57. The method of claim 50, wherein the CNS disease is vascular dementia.

58. The method of claim 50, wherein the CNS disease is mixed dementia.

59. The method of claim 50, wherein the CNS disease is Parkinson's disease.

60. The method of claim 50, wherein the CNS disease is mild cognitive impairment.

61. The method of claim 50, wherein the CNS disease is traumatic (closed or open) penetrating head injuries, traumatic brain injury (TBI), nontraumatic stroke, aneurism, hypoxia, or other injuries to the brain.

62. The method of claim 50, wherein the CNS disease is stroke.

63. The method of claim 62, wherein the CNS disease is ischemic stroke.

64. The method of claim 50, wherein the CNS disease is cognitive impairment associated with schizophrenia (CIAS).

65. The method of claim 49, wherein the method is for treating a mitochondrial disease.

66. The method of claim 65, wherein the mitochondrial disease is a mitochondrial disease of genetic origin.

67. The method of claim 66, wherein the mitochondrial disease of genetic disorder is selected from the group comprising of Alpers, Carnitine-acyl-carnitine deficiency, Carnitine deficiency, Complex I, II, III, IV deficiency, CPEO, CPT II deficiency, Creatine deficiency syndrome, KSS, LCHAD, Leigh syndrome, Leukodystrophy, LHON, MELAS, MEPAN, MERRF, MIRAS, Mitochondrial DNA depletion, MNGIE, NARP, Pearson syndrome, and POLG mutations.

68. The method of any one of claims 49-67, wherein the method further comprising administering to the subject an additional therapeutic agent.
